(19) United States
(12) Patent Application Publication
(10) Pub. No.: US 2013/0252944 A1
(43) Pub. Date: Sep. 26, 2013

(54) NOVEL FUSED PYRAZOLE DERIVATIVES AND THEIR USE AS ALLOSTERIC MODULATORS OF METABOTROPIC GLUTAMATE RECEPTORS

(71) Applicants: Christelle Bolea, Geneva (CH); Sylvain Celanire, Geneva (CH); Lam Tang, Geneva (CH); Philip Jones, Houston, TX (US); Nigel J. Liverton, Harleysville, PA (US); Richard Soll, Shanghai (CN); Jingchao Dong, Shanghai (CN); Zhiyu Lei, Shanghai (CN); Fuqing Liu, Shanghai (CN); Yanling Kong, Shanghai (CN); Yi Ju, Shanghai (CN); Haoyong Zhang, Shanghai (CN); Jieping Wei, Shanghai (CN); Qiaoxin Wei, Shanghai (CN); Minxiang Zhang, Shanghai (CN); Yunfu Luo, Shanghai (CN)

(72) Inventors: Christelle Bolea, Geneva (CH); Sylvain Celanire, Geneva (CH); Lam Tang, Geneva (CH); Philip Jones, Houston, TX (US); Nigel J. Liverton, Harleysville, PA (US); Richard Soll, Shanghai (CN); Jingchao Dong, Shanghai (CN); Zhiyu Lei, Shanghai (CN); Fuqing Liu, Shanghai (CN); Yanling Kong, Shanghai (CN); Yi Ju, Shanghai (CN); Haoyong Zhang, Shanghai (CN); Jieping Wei, Shanghai (CN); Qiaoxin Wei, Shanghai (CN); Minxiang Zhang, Shanghai (CN); Yunfu Luo, Shanghai (CN)

(21) Appl. No.: 13/809,583

(22) PCT Filed: Jul. 11, 2011
(86) PCT No.: PCT/US11/01206

§ 371 Date: Jun. 12, 2013

Related U.S. Application Data
(60) Provisional application No. 61/571,071, filed on Jun. 20, 2011.

(30) Foreign Application Priority Data
Jul. 14, 2010 (GB) 1011831.3

(51) Int. Cl. C07D 513/04; A61K 31/433; A61K 31/4439; A61K 31/506; C07D 513/14; A61K 45/06

(52) U.S. Cl. CPC C07D 513/04; C07D 513/14; A61K 45/06; A61K 31/4439; A61K 31/506; A61K 31/433
USPC 514/217; 546/270.1; 544/331; 548/128; 514/338; 514/275; 514/361; 514/317; 514/224.8

(57) ABSTRACT

The present invention relates to novel compounds of Formula (I), wherein M, A and Y are defined as in Formula (I); invention compounds are modulators of metabotropic glutamate receptors—subtype 4 (“mGluR₄”) which are useful for the treatment or prevention of central nervous system disorders as well as other disorders modulated by mGluR₄ receptors. The invention is also directed to pharmaceutical compositions and the use of such compounds in the manufacture of medicaments, as well as to the use of such compounds for the prevention and treatment of such diseases in which mGluR₄ is involved.

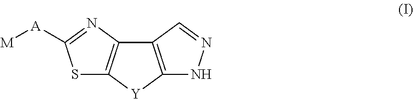

### SUMMARY OF THE INVENTION

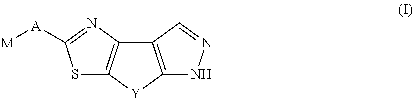

The present invention relates to novel compounds of Formula (I), wherein M, A and Y are defined as in Formula (I); invention compounds are modulators of metabotropic glutamate receptors—subtype 4 (“mGluR₄”) which are useful for the treatment or prevention of central nervous system disorders as well as other disorders modulated by mGluR₄ receptors. The invention is also directed to pharmaceutical compositions and the use of such compounds in the manufacture of medicaments, as well as to the use of such compounds for the prevention and treatment of such diseases in which mGluR₄ is involved.

### BACKGROUND OF THE INVENTION

Glutamate is the major amino-acid transmitter in the mammalian central nervous system (CNS). Glutamate plays a major role in numerous physiological functions, such as learning and memory but also sensory perception, development of synaptic plasticity, motor control, respiration and regulation of cardiovascular function. Furthermore, glutamate is at the center of several different neurological and psychiatric diseases, where there is an imbalance in glutamatergic neurotransmission.

Glutamate mediates synaptic neurotransmission through the activation of ionotropic glutamate receptor channels (iGluRs), namely the NMDA, AMPA and kainate receptors which are responsible for fast excitatory transmission (Nakanishi et al., (1998) Brain Res. Rev., 26:230-235).

In addition, glutamate activates metabotropic glutamate receptors (mGluRs) which have a more modulatory role that contributes to the fine-tuning of synaptic efficacy.

The mGluRs are G protein-coupled receptors (GPCRs) with seven-transmembrane spanning domains and belong to GPCR family 3 along with the calcium-sensing, GABAb and pheromone receptors.

The mGluR family is composed of eight members. They are classified into three groups (group I comprising mGluR₁ and mGluR₅; group II comprising mGluR₂ and mGluR₃; group III comprising mGluR₄, mGluR₆, mGluR₇ and mGluR₈) according to sequence homology, pharmacological profile and nature of intracellular signalling cascades activated (Schoepp et al., (1999) Neuropharmacology, 38:1431-1476).

Glutamate activates the mGluRs through binding to the large extracellular amino-terminal domain of the receptor, herein called the orthosteric binding site. This activation induces a conformational change of the receptor which results in the activation of the G-protein and intracellular signalling pathways.

In the central nervous system, mGluR₄ receptors are expressed most intensely in the cerebellar cortex, basal ganglia, sensory relay nuclei of the thalamus and hippocampus (Bradley et al., (1999) Journal of Comparative Neurology, 407:33-46; Corti et al., (2002) Neuroscience, 110:403-420). The mGluR₄ subtype is negatively coupled to adenylate cyclase via activation of the Gαi/o protein, is expressed primarily on presynaptic terminals, functioning as an autoreceptor or heteroceptor and activation of mGluR₄ leads to decreases in transmitter release from presynaptic terminals (Corti et al., (2002) Neuroscience, 110:403-420; Millan et al., (2002) Journal of Biological Chemistry, 277:47796-47803; Valenti et al., (2003) Journal of Neuroscience, 23:7218-7226).

Orthosteric agonists of mGluR₄ are not selective and activate the other Group III mGluRs (Schoepp et al., (1999) Neuropharmacology, 38:1431-1476). The Group III orthosteric agonist L-AP4 (L-2-amino-4-phosphonobutyrate) was able to reduce motor deficits in animal models of Parkinson's disease (Valenti et al., (2003) J. Neurosci., 23:7218-7226) and decrease excitotoxicity (Bruno et al., (2000) J. Neurosci., 20; 6413-6420) and these effects appear to be mediated through mGluR₄ (Marino et al., (2005) Curr. Topics Med. Chem., 5:885-895). In addition to L-AP4, ACPT-1, another selective group III mGluR agonist has been shown to caused a dose and structure-dependent decrease in haloperidol-induced catalepsy and attenuated haloperidol-increased Proenkephalin mRNA expression in the striatum (Konieczny et al., (2007) Neuroscience, 145:611-620). Furthemore, Lopez et al. (2007, J. Neuroscience, 27:6701-6711) have shown that bilateral infusions of ACPT-I or L-AP4 into the globus pallidus fully reversed the severe akinetic deficits produced by 6-hydroxydopamine lesions of nigrostriatal dopamine neurons in a reaction-time task without affecting the performance of controls. In addition, the reversal of haloperidol-induced catalepsy by intrapallidal ACPT-1 was prevented by concomitant administration of a selective group III receptor antagonist (RS)-alpha-cyclopropyl-4-phosphonophenylglycine. The opposite effects produced by group III mGluR activation in the SNr strongly suggest a role of mGluR₄ rather than others mGluR receptor sub-types in normalizing basal ganglia activity (Lopez et al. 2007).

These results suggest that, among mGluR subtypes, mGluR₄ is believed to be the most interesting novel drug target for the treatment of Parkinson's disease (for a review see Conn et al., (2005) Nature Review Neuroscience, 6:787-798).

Symptoms of Parkinson's disease appear to be due to an imbalance in the direct and indirect output pathways of the basal ganglia, and reduction of transmission at the inhibitory GABAergic striato-pallidal synapse in the indirect pathway may result in alleviation of these symptoms (Marino et al., (2002) Amino Acids, 23:185-191).

mGluR₄ is more abundant in striato-pallidal synapses than in striato-nigral synapses, and its localization suggests function as a presynaptic heteroreceptor on GABAergic neurons (Bradley et al., (1999) Journal of Comparative Neurology, 407:33-46) suggesting that selective activation or positive modulation of mGluR₄ would decrease GABA release in this synapse thereby decreasing output of the indirect pathway and reducing or eliminating the Parkinson's disease symptoms. Classical treatment of Parkinsonism typically involves the use of levodopa combined with carbidopa (SINEMET™) or benserazide (MADOPAR™). Dopamine agonists such as bromocriptine (PARLODEL™), lisuride and pergolide (CELANCE™) act directly on dopamine receptors and are also used for the treatment of Parkinsonism. These molecules have the same side-effect profile as levodopa.

A new avenue for developing selective compounds acting at mGluRs is to identify molecules that act through allosteric mechanisms, modulating the receptor by binding to a site different from the highly conserved orthosteric binding site.

Positive allosteric modulators of mGluRs have emerged recently as novel pharmacological entities offering this attractive alternative. This type of molecule has been discovered for mGluR₁, mGluR₂, mGluR₄, mGluR₅, mGluR₇ and mGluR₈ (Knoflach F. et al. (2001) Proc. Natl. Acad. Sci. USA, 98:13402-13407; Johnson M. P. et al., (2002) Neuropharmacology, 43:799-808; O'Brien J. A. et al., (2003) Mol. Pharmacol., 64:731-740; Johnson M. P. et al., (2003) J. Med. Chem., 46:3189-3192; Marino M. J. et al., (2003) Proc. Natl. Acad. Sci. USA, 100:13668-13673; Mitsukawa K. et al., (2005) Proc. Natl. Acad. Sci. USA, 102(51):18712-18717; Wilson J. et al., (2005) Neuropharmacology, 49:278; for a review see Mutel V., (2002) Expert Opin. Ther. Patents, 12:1-8; Kew J. N., (2004) Pharmacol. Ther., 104(3):233-244; Johnson M. P. et al., (2004) Biochem. Soc. Trans., 32:881-887; recently Ritzen A., Mathiesen, J. M. and Thomsen C., (2005) Basic Clin. Pharmacol. Toxicol., 97:202-213).

In particular molecules have been described as mGluR₄ positive allosteric modulators (Maj et al., (2003) Neuropharmacology, 45:895-906; Mathiesen et al., (2003) British Journal of Pharmacology, 138:1026-1030). It has been demonstrated that such molecules have been characterized in in vitro systems as well as in rat brain slices where they potentiated the effect of L-AP4 in inhibiting transmission at the striatopallidal synapse. These compounds do not activate the receptor by themselves (Marino et al., (2003) Proc. Nat. Acad. Sci. USA, 100:13668-13673). Rather, they enable the receptor to produce a maximal response to a concentration of glutamate or the Group III orthosteric agonist L-AP4 which by itself induces a minimal response.

PHCCC (N-phenyl-7-(hydroxyimino)cyclopropa[b]chromen-1a-carboxamide), a positive allosteric modulator of mGluR₄ not active on other mGluRs (Maj et al., (2003) Neuropharmacology, 45:895-906), has been shown to be efficacious in animal models of Parkinson's disease thus representing a potential novel therapeutic approach for Parkinson's disease as well as for other motor disorders and disturbances (Marino et al., (2003) Proc. Nat. Acad. Sci. USA, 100:13668-13673), neurodegeneration in Parkinson's disease (Marino et al., (2005) Curr. Topics Med. Chem., 5:885-895; Valenti et al., (2005) J. Pharmacol. Exp. Ther., 313:1296-1304; Vernon et al., (2005) Eur. J. Neurosci., 22:1799-1806, Battaglia et al., (2006) J. Neurosci., 26:7222-7229), and neurodegeneration in Alzheimer's disease or due to ischemic or traumatic insult (Maj et al., (2003) Neuropharmacology, 45:895-906).

PHCCC also has been shown to be active in an animal model of anxiety (Stachowicz et al., (2004) Eur. J. Pharmacol., 498:153-156). Previously, ACPT-1 has been shown to produce a dose-dependent anti-conflict effect after intrahippocampal administration and anti-depressant-like effects in rats after intracerebroventricular administration (Tatarczynska et al., (2002) Pol. J. Pharmacol., 54(6):707-710). More recently, ACPT-1 has also been shown to have anxiolytic-like effects in the stress-induced hyperthermia, in the elevated-plus maze in mice and in the Vogel conflict test in rats when injected intraperitoneally (Stachowicz et al., (2009) Neuropharmacology, 57(3): 227-234).

Activation of mGluR₄ receptors which are expressed in α- and F-cells in the islets of Langerhans inhibits glucagon secretion. Molecules which activate or potentiate the agonist activity of these receptors may be an effective treatment for hyperglycemia, one of the symptoms of type 2 diabetes (Uehara et al., (2004) Diabetes, 53:998-1006).

The β-chemokine RANTES is importantly involved in neuronal inflammation and has been implicated in the pathophysiology of multiple sclerosis. Activation of Group III mGluRs with L-AP4 reduced the synthesis and release of RANTES in wild-type cultured astrocytes, whereas the ability of L-AP4 to inhibit RANTES was greatly decreased in astrocyte cultures from mGluR₄ knockout mice (Belong et al., (2002) Journal of Neuroscience, 22:5403-5411). These data suggest that positive allosteric modulators of mGluR₄ may be an effective treatment for neuroinflammatory disorders of the central nervous system, including multiple sclerosis and related disorders.

Two different variants of the mGluR₄ receptor are expressed in taste tissues and may function as receptors for the umami taste sensation (Monastyrskaia et al., (1999) Br. J. Pharmacol., 128:1027-1034; Toyono et al., (2002) Arch. Histol. Cytol., 65:91-96). Thus positive allosteric modulators of mGluR₄ may be useful as taste agents, flavour agents, flavour enhancing agents or food additives.

There is anatomical evidence that the majority of vagal afferents innervating gastric muscle express group III mGluRs (mGluR₄, mGluR₆, mGluR₇ and mGluR₈) and actively transport receptors to their peripheral endings (Page et al., (2005) Gastroenterology, 128:402-10). Recently, it was shown that the activation of peripheral group III mGluRs inhibited vagal afferents mechanosensitivity in vitro which translates into reduced triggering of transient lower esophageal sphincter relaxations and gastroesophageal reflux in vivo (Young et al., (2008) Neuropharmacol, 54:965-975). Labelling for mGluR₄ and mGluR₈ was abundant in gastric vagal afferents in the nodose ganglion, at their termination sites in the nucleus tractus solitarius and in gastric vagal motoneurons. These data suggest that positive allosteric modulators of mGluR₄ may be an effective treatment for gastroesophageal reflux disease (GERD) and lower esophageal disorders and gastro-intestinal disorders.

International patent publication WO2005/007096 has described mGluR₄ receptor positive allosteric modulator useful, alone or in combination with a neuroleptic agent, for treating or preventing movement disorders. However, none of the specifically disclosed compounds are structurally related to the compounds of the invention.

Recently, new mGluR₄ receptor positive allosteric modulators have been described: pyrazolo[3,4-d]pyrimidine derivatives (Niswender et al., (2008) Bioorganic & Medicinal Chemistry Letters, 18(20):5626-5630), functionalized benzylidene hydrazinyl-3-methylquinazoline and bis-2,3-dihydroquinazolin-4(1H)-one (Williams et al., (2009) Bioorganic & Medicinal Chemistry Letters, 19:962-966) and heterobiarylamides (Engers et al, (2009) Journal of Medicinal Chemistry, 52 (14), 4115-4118). Niswender et al., described (±)-cis-2-(3,5-dichlorophenylcarbamoyl)cyclohexane carboxylic acid (2008) Molecular Pharmacology, 74(5):1345-1358), as a positive allosteric modulator of mGluR₄ also having agonist activity. This moderately active molecule has demonstrated evidence of efficacy following icy injection in rat models of Parkinson's disease. International patent publications WO2009/010454 and WO2009/010455 have mentioned amido derivatives and novel heteroaromatic derivatives, respectively, as positive allosteric modulators of metabotropic glutamate receptors. The subject of the latter case has been examined in the following article East Stephen P. et al., (2010) Expert Opin. Ther. Patents, 20 (3) 441-445. Finally, Williams R. et al., described in (2010) ACS Chemical Neuroscience, 1(6): 411-419, the “Re-exploration of the PHCCC scaffold”.

International patent publication WO2010/079238 has described novel tricyclic heteroaromatic derivatives and their use as positive allosteric modulators of mGluRs. More recently, a review on recent progress on the identification of metabotropic glutamate 4 receptor ligands and their potential utility as CNS therapeutics (Robichaud A. et al., (14 Jun. 2011) ACS Chemical Neuroscience, DOI: 10.1021/cn200043e, http://pubs.acs.org) has cited some of the examples described in the WO2010/079238 patent application; Hong S.-P et al, (20 Jun. 2011) J. Med. Chem., DOI: 10.1021/jm200290z, http://pubs.acs.org) have described tricyclic thiazolopyrazole derivatives as metabotropic glutamate receptor 4 positive allosteric modulators.

The present inventors have discovered novel thiazole compounds of general Formula (I) which, surprisingly, show potent activity and selectivity on the mGluR₄ receptor. The compounds of the invention demonstrate advantageous properties over compounds of the prior art. Improvements have been observed in one or more of the following characteristics of the compounds of the invention: the potency on the target, the selectivity for the target, the bioavailability, the brain penetration, and the activity in behavioural models.

Such aminothiazole derivatives are useful for treating or preventing a condition in a mammal, including a human, the treatment or prevention of which is affected or facilitated by the neuromodulatory effect of mGluR₄ modulators. In the case of the treatment of movement disorders such as Parkinson's disease, the compounds of the invention can be used alone or in combination with an agent selected from the group consisting of: levodopa, levodopa with a selective extracerebral decarboxylase inhibitor, carbidopa, entacapone, a COMT inhibitor, a dopamine agonist, an anticholinergic, a cholinergic agonist, a butyrophenone neuroleptic agent, a diphenylbutylpiperidine neuroleptic agent, a heterocyclic dibenzazepine neuroleptic agent, an indolone neuroleptic agent, a phenothiazine neuroleptic agent, a thioxanthene neuroleptic agent, an NMDA receptor antagonist, an MAO-B inhibitor, an mGluR₅ antagonist or an A_{2A} antagonist.

### DETAILED DESCRIPTION OF THE INVENTION

The invention relates to compounds having metabotropic glutamate receptor 4 modulator activity. In its most general compound aspect, the present invention provides a compound according to Formula (I),

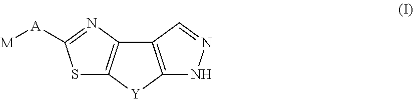

a pharmaceutically acceptable acid or base addition salt thereof, a stereochemically isomeric form thereof and an N-oxide form thereof, wherein:
M is an optionally substituted heteroaryl;

### A is NH or O;

Y is selected from the group of —CR¹R²—, —CR¹R²—CR³R⁴—, —CR¹R²—CR³R⁴—CR⁵R⁶—, —CR¹R²—CR³R⁴—CR⁵R⁶—CR⁷R⁸—, —CR¹═CR²—, —CO—, —CO—CR¹R²—CR³R⁴—, —CR¹R²—CR³R⁴—CO—, —CO—CR¹R²—O—, —O—CR¹R²—CO—, —NR⁹—CR¹R²—CO—, —CO—NR⁹—, —NR⁹—CO—, —O—CR¹R²—, —CR¹R²—O—, —O—CR¹R²—CR³R⁴—, —CR¹R²—O—CR³R⁴—, —CR¹R²—CR³R⁴—O—, —NR⁹—CR¹R²—, —CR¹R²—NR⁹—, —NR⁹—CR¹R²—CR³R⁴—, —CR¹R²—NR⁹—CR³R⁴—, —S—CR¹R²—, —CR¹R²—S—, —S—CR¹R²—CR³R⁴—, —CR¹R²—S—CR³R⁴—, —CR¹R²—CR³R⁴—S—, —SO—CR¹R²—, —CR¹R²—SO—, —SO—CR¹R²—CR³R⁴—, —CR¹R²—SO—CR³R⁴—, —CR¹R²—CR³R⁴—SO—, —SO₂—CR¹R²—, —CR¹R²—SO₂—, —SO₂—CR¹R²—CR³R⁴—, —CR¹R²—SO₂—CR³R⁴— and —CR¹R²—CR³R⁴—SO₂—;
R¹, R², R³, R⁴, R⁵, R⁶, R⁷ and R⁸ are each independently selected from the group of hydrogen, halogen, —CN, —CF₃ or an optionally substituted radical selected from the group of —(C₁-C₆)alkyl, —(C₁-C₆)haloalkyl, —(C₃-C₇)cycloalkyl, aryl, heteroaryl, heterocycle, —(C₁-C₆)allylene-aryl, —(C₁-C₆)alkylene-heteroaryl, —(C₁-C₆)alkylene-heterocycle, —O—(C₀-C₆)alkyl, —N—((C₀-C₆)alkyl)₂, —(C₁-C₆)alkyl-O—(C₀-C₆)alkyl, and —(C₁-C₆)alkyl-N—((C₀-C₆)alkyl)₂;
Any two radicals of R (R¹, R², R³, R⁴, R⁵, R⁶, R⁷ or R⁸) may be taken together to form an optionally substituted 3 to 10 membered carbocyclic or heterocyclic ring; and
R⁹ is selected from the group of hydrogen or an optionally substituted radical selected from the group of —(C₁-C₆)alkyl, —(C₁-C₆)haloalkyl, —(C₃-C₇)cycloalkyl, —(C₁-C₆)alkylene-(C₁-C₆)haloalkyl, —(C₁-C₆)alkylene-(C₃-C₇)halocycloalkyl, aryl, heteroaryl, heterocycle, —(C₁-C₆)alkylene-aryl, —(C₁-C₆)alkylene-heteroaryl, —(C₁-C₆)alkylene-heterocycle, —(C₂-C₆)alkyl-O—(C₀-C₆)alkyl, and —(C₂-C₆)alkyl-N—((C₀-C₆)alkyl)₂.

In a more preferred aspect of Formula (I), the invention provides a compound according to Formula (II):

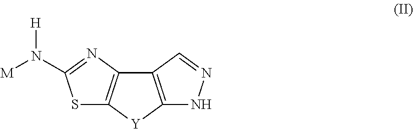

a pharmaceutically acceptable acid or base addition salt thereof, a stereochemically isomeric form thereof and an N-oxide form thereof;
provided that according to proviso (i) the compound is not:
N-(Pyridin-2-yl)-5,6-dihydro-4H-thiazolo[4,5-e]indazol-2-amine
N-(6-Methylpyridin-2-yl)-5,6-dihydro-4H-thiazolo[4,5-d]indazol-2-amine
N-(6-Chloropyridin-2-yl)-5,6-dihydro-4H-thiazolo[4,5-e]indazol-2-amine
N-(6-Fluoropyridin-2-yl)-5,6-dihydro-4H-thiazolo[4,5-e]indazol-2-amine
4-Methyl-N-(pyridin-2-yl)-5,6-dihydro-4H-thiazolo[4,5-e]indazol-2-amine
Pyridin-2-yl-(4,5,6,7-tetrahydro-3-thia-1,7,8-triaza-cyclopenta[e]azulen-2-yl)-amine
4-Ethyl-N-(pyridin-2-yl)-5,6-dihydro-4H-thiazolo[4,5-e]indazol-2-amine
N-(3-Fluoropyridin-2-yl)-5,6-dihydro-4H-thiazolo[4,5-e]indazol-2-amine
(6-Methyl-pyridin-2-yl)-(4,5,6,7-tetrahydro-3-thia-1,7,8-triaza-cyclopenta[e]azulen-2-yl)-amine
(5-Fluoro-pyridin-2-yl)-(4,5,6,7-tetrahydro-3-thia-1,7,8-triaza-cyclopenta[e]azulen-2-yl)-amine
(3-Fluoro-pyridin-2-yl)-(4,5,6,7-tetrahydro-3-thia-1,7,8-triaza-cyclopenta[e]azulen-2-yl)-amine
(6-Methoxy-pyridin-2-yl)-(4,5,6,7-tetrahydro-3-thia-1,7,8-triaza-cyclopenta[e]azulen-2-yl)-amine
N-(5-Fluoropyridin-2-yl)-5,6-dihydro-4H-thiazolo[4,5-e]indazol-2-amine
(6-Chloro-pyridin-2-yl)-(4,5,6,7-tetrahydro-3-thia-1,7,8-triaza-cyclopenta[e]azulen-2-yl)-amine
N-(Pyrazin-2-yl)-5,6-dihydro-4H-thiazolo[4,5-e]indazol-2-amine
(6-Fluoro-pyridin-2-yl)-(4,5,6,7-tetrahydro-3-thia-1,7,8-triaza-cyclopenta[e]azulen-2-yl)-amine
Pyrimidin-2-yl-(4,5,6,7-tetrahydro-3-thia-1,7,8-triaza-cyclopenta[e]azulen-2-yl)-amine
N-(Pyrimidin-2-yl)-5,6-dihydro-4H-thiazolo[4,5-e]indazol-2-amine
(1-Methyl-1H-pyrazol-3-yl)-(4,5,6,7-tetrahydro-3-thia-1,7,8-triaza-cyclopenta[e]azulen-2-yl)-amine
N-(1-Methyl-1H-pyrazol-3-yl)-5,6-dihydro-4H-thiazolo[4,5-e]indazol-2-amine
N-(4-Methylpyrimidin-2-yl)-5,6-dihydro-4H-thiazolo[4,5-e]indazol-2-amine
(4-Methyl-pyrimidin-2-yl)-(4,5,6,7-tetrahydro-3-thia-1,7,8-triaza-cyclopenta[e]azulen-2-yl)-amine
4,4-Dimethyl-N-(pyridin-2-yl)-5,6-dihydro-4H-thiazolo[4,5-e]indazol-2-amine
4,4-Dimethyl-N-(pyrimidin-2-yl)-5,6-dihydro-4H-thiazolo[4,5-e]indazol-2-amine
4-Methyl-N-(pyrimidin-2-yl)-5,6-dihydro-4H-thiazolo[4,5-e]indazol-2-amine and
5,5-Dimethyl-N-(pyrimidin-2-yl)-5,6-dihydro-4H-thiazolo[4,5-e]indazol-2-amine;
   provided that according to proviso (ii) the compound is not:
4,4-Dimethyl-N-(4-methylpyrimidin-2-yl)-5,6-dihydro-4H-[1,3]thiazolo[4,5-e]indazol-2-amine
N-(4-Methylpyrimidin-2-yl)-6H-[1,3]thiazolo[4,5-e]indazol-2-amine
N-(Pyrimidin-2-yl)-6H-[1,3]thiazolo[4,5-e]indazol-2-amine
N-(5-Fluoropyrimidin-2-yl)-4,4-dimethyl-5,6-dihydro-4H-[1,3]thiazolo[4,5-e]indazol-2-amine
N-(5-Fluoro-4-methylpyrimidin-2-yl)-4,4-dimethyl-5,6-dihydro-4H-[1,3]thiazolo[4,5-e]indazol-2-amine
5-Bromo-N-(pyrimidin-2-yl)-6H-[1,3]thiazolo[4,5-e]indazol-2-amine
5-Chloro-N-(pyrimidin-2-yl)-6H-[1,3]thiazolo[4,5-e]indazol-2-amine
5-Methyl-N-(4-methylpyrimidin-2-yl)-6H-[1,3]thiazolo[4,5-e]indazol-2-amine
5-Methyl-N-(pyrimidin-2-yl)-5,6-dihydro-4H-[1,3]thiazolo[4,5-e]indazol-2-amine
5-Methyl-N-(4-methylpyrimidin-2-yl)-5,6-dihydro-4H-[1,3]thiazolo[4,5-e]indazol-2-amine
N-(4-Methylpyrimidin-2-yl)-5′,6′-dihydrospiro[cyclopropane-1,4′-[1,3]thiazolo[4,5-e]indazol]-2′-amine
N-(Pyrimidin-2-yl)-5′,6′-dihydrospiro[cyclopropane-1,4′-[1,3]thiazolo[4,5-d]indazol]-2′-amine
N-(5-Fluoropyrimidin-2-yl)-5′,6′-dihydrospiro[cyclopropane-1,4′-[1,3]thiazolo[4,5-e]indazol]-2′-amine
N-(Pyrimidin-2-yl)-1,7-dihydropyrazolo[3′,4′:4,5]cyclopenta[1,2-d][1,3]thiazol-5-amine
N-(Pyrimidin-2-yl)-6,7-dihydro-4H-pyrazolo[4′,3′:5,6]oxepino[4,3-d][1,3]thiazol-2-amine
N-(5-Fluoropyrimidin-2-yl)-6,7-dihydro-4H-pyrazolo[4′,3′:5,6]oxepino[4,3-d][1,3]thiazol-2-amine
N-(4-Methylpyrimidin-2-yl)-6,7-dihydro-4H-pyrazolo[4′,3′:5,6]oxepino[4,3-d][1,3]thiazol-2-amine
(6-Methyl-6,7-dihydro-4H-5-oxa-3-thia-1,7,8-triaza-cyclopenta[e]azulen-2-yl)-(4-methyl-pyrimidin-2-yl)-amine
((R)-6-Methyl-6,7-dihydro-4H-5-oxa-3-thia-1,7,8-triaza-cyclopentarelazulen-2-yl)-(4-methyl-pyrimidin-2-yl)-amine
((S)-6-Methyl-6,7-dihydro-4H-5-oxa-3-thia-1,7,8-triaza-cyclopenta[e]azulen-2-yl)-(4-methyl-pyrimidin-2-yl)-amine
(6,7-Dihydro-4H-5-oxa-3-thia-1,7,8-triaza-cyclopenta[e]azulen-2-yl)-(4-methoxy-pyrimidin-2-yl)-amine
(4-Isopropyl-pyrimidin-2-yl)-(6-methyl-6,7-dihydro-4H-5-oxa-3-thia-1,7,8-triaza-cyclopenta[e]azulen-2-yl)-amine
(4-Ethyl-pyrimidin-2-yl)-(6-methyl-6,7-dihydro-4H-5-oxa-3-thia-1,7,8-triaza-cyclopenta[e]azulen-2-yl)-amine
(4-Cyclopropyl-pyrimidin-2-yl)-(6-methyl-6,7-dihydro-4H-5-oxa-3-thia-1,7,8-triaza-cyclopenta[e]azulen-2-yl)-amine
(N-(5-Fluoro-4-methylpyrimidin-2-yl)-6-methyl-6,7-dihydro-4H-pyrazolo[4′,3′:5,6]oxepino[4,3-d][1,3]thiazol-2-amine)
(N-(5-Fluoropyrimidin-2-yl)-6-methyl-6,7-dihydro-4H-pyrazolo[4′,3′:5,6]oxepino[4,3-d][1,3]thiazol-2-amine)
4-Methyl-N-(4-methylpyrimidin-2-yl)-6,7-dihydro-4H-pyrazolo[4′,3′:5,6]oxepino[4,3-d][1,3]thiazol-2-amine
4,6-Dimethyl-N-(4-methylpyrimidin-2-yl)-6,7-dihydro-4H-pyrazolo[4′,3′:5,6]oxepino-[4,3-d][1,3]thiazol-2-amine
N-(Pyrimidin-2-yl)-4,5,6,7-tetrahydropyrazolo[3,4-c][1,3]thiazolo[4,5-e]azepin-2-amine
5-Methyl-N-(4-methylpyrimidin-2-yl)-4,5,6,7-tetrahydropyrazolo[3,4-c][1,3]thiazolo[4,5-e]azepin-2-amine
N-(5-Fluoropyrimidin-2-yl)-4,5,6,7-tetrahydropyrazolo[3′,4′:6,7]cyclohepta[1,2-d][1,3]thiazol-2-amine
(5,5-Dimethyl-4,5,6,7-tetrahydro-3-thia-1,7,8-triaza-cyclopenta[e]azulen-2-yl)-(5-fluoro-pyrimidin-2-yl)-amine
6,6-Difluoro-N-(4-methylpyrimidin-2-yl)-4,5,6,7-tetrahydropyrazolo[3′,4′:6,7]cyclohepta[1,2-d][1,3]thiazol-2-amine
2-[(4-Methylpyrimidin-2-yl)amino]-4,7-dihydropyrazolo[3′,4′:6,7]cyclohepta[1,2-d][1,3]thiazol-6(5H)-one
5,5-Dimethyl-2-[(4-methylpyrimidin-2-yl)amino]-4,7-dihydropyrazolo[3′,4′:6,7]cyclohepta[1,2-d][1,3]thiazol-6(5H)-one
(6-Methyl-6,7-dihydro-4H-5-oxa-3-thia-1,7,8-triaza-cyclopenta[e]azulen-2-yl)-(5-methyl-[1,2,4]thiadiazol-3-yl)-amine and
(5-Fluoro-4,5,6,7-tetrahydro-3-thia-1,7,8-triaza-cyclopenta[e]azulen-2-yl)-(5-methyl-pyrimidin-2-yl)-amine;
   provided that according to proviso (iii) the compound is not:
5,6-Dihydro-4H-3-thia-1,6,7-triaza-as-indacen-2-yl)-pyridin-4-yl-amine
(5,6-Dihydro-4H-3-thia-1,6,7-triaza-as-indacen-2-yl)-pyridin-3-yl-amine
(5,6-Dihydro-4H-3-thia-1,6,7-triaza-as-indacen-2-yl)-(5-methyl-pyridin-2-yl)-amine
(5,6-Dihydro-4H-3-thia-1,6,7-triaza-as-indacen-2-yl)-(4-methyl-pyridin-2-yl)-amine
(5,6-Dihydro-4H-3-thia-1,6,7-triaza-as-indacen-2-yl)-(4,6-dimethyl-pyridin-2-yl)-amine
Pyridin-3-yl-(4,5,6,7-tetrahydro-3-thia-1,7,8-triaza-cyclopenta[e]azulen-2-yl)-amine
Pyrimidin-4-yl-(4,5,6,7-tetrahydro-3-thia-1,7,8-triaza-cyclopenta[e]azulen-2-yl)-amine
Pyrazin-2-yl-(4,5,6,7-tetrahydro-3-thia-1,7,8-triaza-cyclopenta[e]azulen-2-yl)-amine
(5-Methyl-1H-pyrazol-3-yl)-(4,5,6,7-tetrahydro-3-thia-1,7,8-triaza-cyclopenta[e]azulen-2-yl)-amine
(1H-Pyrazol-3-yl)-(4,5,6,7-tetrahydro-3-thia-1,7,8-triaza-cyclopenta[e]azulen-2-yl)-amine
(4,5,6,7-Tetrahydro-3-thia-1,7,8-triaza-cyclopenta[e]azulen-2-yl)-thiazol-2-yl-amine
(6-Methyl-pyridazin-3-yl)-(4,5,6,7-tetrahydro-3-thia-1,7,8-triaza-cyclopenta[e]azulen-2-yl)-amine
(3-Methyl-pyridin-2-yl)-(4,5,6,7-tetrahydro-3-thia-1,7,8-triaza-cyclopenta[e]azulen-2-yl)-amine
6-(4,5,6,7-Tetrahydro-3-thia-1,7,8-triaza-cyclopenta[e]azulen-2-ylamino)-pyridine-2-carbonitrile
(5-Methyl-pyridin-2-yl)-(4,5,6,7-tetrahydro-3-thia-1,7,8-triaza-cyclopenta[e]azulen-2-yl)-amine
[6-(4,5,6,7-Tetrahydro-3-thia-1,7,8-triaza-cyclopenta[e]azulen-2-ylamino)-pyridin-2-yl]-methanol
6-(4,5,6,7-Tetrahydro-3-thia-1,7,8-triaza-cyclopenta[e]azulen-2-ylamino)-pyridine-2-carboxylic acid methyl ester
(4-Methoxy-pyrimidin-2-yl)-(4,5,6,7-tetrahydro-3-thia-1,7,8-triaza-cyclopenta[e]azulen-2-yl)-amine
(6-Pyrrolidin-1-yl-pyridin-2-yl)-(4,5,6,7-tetrahydro-3-thia-1,7,8-triaza-cyclopenta[e]azulen-2-yl)-amine
[4-(2-Methoxy-ethoxy)-pyrimidin-2-yl]-(4,5,6,7-tetrahydro-3-thia-1,7,8-triaza-cyclopenta[e]azulen-2-yl)-amine
N,N-Dimethyl-N′-(4,5,6,7-tetrahydro-3-thia-1,7,8-triaza-cyclopenta[e]azulen-2-yl)-pyridine-2,6-diamine and
N-Ethyl-N′-(4,5,6,7-tetrahydro-3-thia-1,7,8-triaza-cyclopenta[e]azulen-2-yl)-pyridine-2,6-diamine.

In a more preferred aspect of Formula (II), the invention provides a compound wherein:

Y is selected from the group of —CR¹R²—, —CR¹R²—CR³R⁴—, —CR¹R²—CR³R⁴—CR⁵R⁶— and —CR¹R²—CR³R⁴—CR⁵R⁶—CR⁷R⁸;
R¹, R², R³, R⁴, R⁵, R⁶, R⁷ and R⁸ are each independently selected from the group of hydrogen, halogen, —CN, —CF₃ or an optionally substituted radical selected from the group of —(C₁-C₆)alkyl, —(C₁-C₆)haloalkyl, —(C₃-C₇)cycloalkyl, aryl, heteroaryl, heterocycle, —(C₁-C₆)allylene-aryl, —(C₁-C₆)alkylene-heteroaryl, —(C₁-C₆)alkylene-heterocycle, —O—(C₀-C₆)alkyl, —N—((C₀-C₆)alkyl)₂, —(C₁-C₆)alkyl-O—(C₀-C₆)alkyl, and —(C₁-C₆)alkyl-N—((C₀-C₆)alkyl)₂;
Any two radicals of R (R¹, R², R³, R⁴, R⁵, R⁶, R⁷ or R⁸) may be taken together to form an optionally substituted 3 to 10 membered carbocyclic or heterocyclic ring;
provided that according to proviso (i) the compound is not:
N-(Pyridin-2-yl)-5,6-dihydro-4H-thiazolo[4,5-e]indazol-2-amine
N-(6-Methylpyridin-2-yl)-5,6-dihydro-4H-thiazolo[4,5-e]indazol-2-amine
N-(6-Chloropyridin-2-yl)-5,6-dihydro-4H-thiazolo[4,5-e]indazol-2-amine
N-(6-Fluoropyridin-2-yl)-5,6-dihydro-4H-thiazolo[4,5-e]indazol-2-amine
4-Methyl-N-(pyridin-2-yl)-5,6-dihydro-4H-thiazolo[4,5-e]indazol-2-amine
Pyridin-2-yl-(4,5,6,7-tetrahydro-3-thia-1,7,8-triaza-cyclopenta[e]azulen-2-yl)-amine
4-Ethyl-N-(pyridin-2-yl)-5,6-dihydro-4H-thiazolo[4,5-e]indazol-2-amine
N-(3-Fluoropyridin-2-yl)-5,6-dihydro-4H-thiazolo[4,5-e]indazol-2-amine
(6-Methyl-pyridin-2-yl)-(4,5,6,7-tetrahydro-3-thia-1,7,8-triaza-cyclopenta[e]azulen-2-yl)-amine
(5-Fluoro-pyridin-2-yl)-(4,5,6,7-tetrahydro-3-thia-1,7,8-triaza-cyclopenta[e]azulen-2-yl)-amine
(3-Fluoro-pyridin-2-yl)-(4,5,6,7-tetrahydro-3-thia-1,7,8-triaza-cyclopenta[e]azulen-2-yl)-amine
(6-Methoxy-pyridin-2-yl)-(4,5,6,7-tetrahydro-3-thia-1,7,8-triaza-cyclopenta[e]azulen-2-yl)-amine
N-(5-Fluoropyridin-2-yl)-5,6-dihydro-4H-thiazolo[4,5-e]indazol-2-amine
(6-Chloro-pyridin-2-yl)-(4,5,6,7-tetrahydro-3-thia-1,7,8-triaza-cyclopenta[e]azulen-2-yl)-amine
N-(Pyrazin-2-yl)-5,6-dihydro-4H-thiazolo[4,5-e]indazol-2-amine
(6-Fluoro-pyridin-2-yl)-(4,5,6,7-tetrahydro-3-thia-1,7,8-triaza-cyclopenta[e]azulen-2-yl)-amine
Pyrimidin-2-yl-(4,5,6,7-tetrahydro-3-thia-1,7,8-triaza-cyclopenta[e]azulen-2-yl)-amine
N-(Pyrimidin-2-yl)-5,6-dihydro-4H-thiazolo[4,5-e]indazol-2-amine
(1-Methyl-1H-pyrazol-3-yl)-(4,5,6,7-tetrahydro-3-thia-1,7,8-triaza-cyclopenta[e]azulen-2-yl)-amine
N-(1-Methyl-1H-pyrazol-3-yl)-5,6-dihydro-4H-thiazolo[4,5-e]indazol-2-amine
N-(4-Methylpyrimidin-2-yl)-5,6-dihydro-4H-thiazolo[4,5-e]indazol-2-amine
(4-Methyl-pyrimidin-2-yl)-(4,5,6,7-tetrahydro-3-thia-1,7,8-triaza-cyclopenta[e]azulen-2-yl)-amine
4,4-Dimethyl-N-(pyridin-2-yl)-5,6-dihydro-4H-thiazolo[4,5-e]indazol-2-amine
4,4-Dimethyl-N-(pyrimidin-2-yl)-5,6-dihydro-4H-thiazolo[4,5-e]indazol-2-amine
4-Methyl-N-(pyrimidin-2-yl)-5,6-dihydro-4H-thiazolo[4,5-e]indazol-2-amine and
5,5-Dimethyl-N-(pyrimidin-2-yl)-5,6-dihydro-4H-thiazolo[4,5-e]indazol-2-amine;
   provided that according to proviso (iv) the compound is not:
4,4-Dimethyl-N-(4-methylpyrimidin-2-yl)-5,6-dihydro-4H-[1,3]thiazolo[4,5-e]indazol-2-amine
N-(5-Fluoropyrimidin-2-yl)-4,4-dimethyl-5,6-dihydro-4H-[1,3]thiazolo[4,5-e]indazol-2-amine
N-(5-Fluoro-4-methylpyrimidin-2-yl)-4,4-dimethyl-5,6-dihydro-4H-[1,3]thiazolo[4,5-e]indazol-2-amine
5-Methyl-N-(pyrimidin-2-yl)-5,6-dihydro-4H-[1,3]thiazolo[4,5-e]indazol-2-amine
5-Methyl-N-(4-methylpyrimidin-2-yl)-5,6-dihydro-4H-[1,3]thiazolo[4,5-e]indazol-2-amine
N-(4-Methylpyrimidin-2-yl)-5′,6′-dihydrospiro[cyclopropane-1,4′-[1,3]thiazolo[4,5-e]indazol]-2′-amine
N-(Pyrimidin-2-yl)-5′,6′-dihydrospiro[cyclopropane-1,4′-[1,3]thiazolo[4,5-e]indazol]-2′-amine
N-(5-Fluoropyrimidin-2-yl)-5′,6′-dihydrospiro[cyclopropane-1,4′-[1,3]thiazolo[4,5-e]indazol]-2′-amine
N-(Pyrimidin-2-yl)-1,7-dihydropyrazolo[3′,4′:4,5]cyclopenta[1,2-d][1,3]thiazol-5-amine
N-(5-Fluoropyrimidin-2-yl)-4,5,6,7-tetrahydropyrazolo[3′,4′:6,7]cyclohepta[1,2-d][1,3]thiazol-2-amine
(5,5-Dimethyl-4,5,6,7-tetrahydro-3-thia-1,7,8-triaza-cyclopenta[e]azulen-2-yl)-(5-fluoro-pyrimidin-2-yl)-amine
6,6-Difluoro-N-(4-methylpyrimidin-2-yl)-4,5,6,7-tetrahydropyrazolo[3′,4′:6,7]cyclohepta[1,2-d][1,3]thiazol-2-amine and
(5-Fluoro-4,5,6,7-tetrahydro-3-thia-1,7,8-triaza-cyclopenta[e]azulen-2-yl)-(5-methyl-pyrimidin-2-yl)-amine;
   provided that according to proviso (iii) the compound is not:
5,6-Dihydro-4H-3-thia-1,6,7-triaza-as-indacen-2-yl)-pyridin-4-yl-amine
(5,6-Dihydro-4H-3-thia-1,6,7-triaza-as-indacen-2-yl)-pyridin-3-yl-amine
(5,6-Dihydro-4H-3-thia-1,6,7-triaza-as-indacen-2-yl)-(5-methyl-pyridin-2-yl)-amine
(5,6-Dihydro-4H-3-thia-1,6,7-triaza-as-indacen-2-yl)-(4-methyl-pyridin-2-yl)-amine
(5,6-Dihydro-4H-3-thia-1,6,7-triaza-as-indacen-2-yl)-(4,6-dimethyl-pyridin-2-yl)-amine
Pyridin-3-yl-(4,5,6,7-tetrahydro-3-thia-1,7,8-triaza-cyclopenta[e]azulen-2-yl)-amine
Pyrimidin-4-yl-(4,5,6,7-tetrahydro-3-thia-1,7,8-triaza-cyclopenta[e]azulen-2-yl)-amine
Pyrazin-2-yl-(4,5,6,7-tetrahydro-3-thia-1,7,8-triaza-cyclopenta[e]azulen-2-yl)-amine
(5-Methyl-1H-pyrazol-3-yl)-(4,5,6,7-tetrahydro-3-thia-1,7,8-triaza-cyclopenta[e]azulen-2-yl)-amine
(1H-Pyrazol-3-yl)-(4,5,6,7-tetrahydro-3-thia-1,7,8-triaza-cyclopenta[e]azulen-2-yl)-amine
(4,5,6,7-Tetrahydro-3-thia-1,7,8-triaza-cyclopenta[e]azulen-2-yl)-thiazol-2-yl-amine
(6-Methyl-pyridazin-3-yl)-(4,5,6,7-tetrahydro-3-thia-1,7,8-triaza-cyclopenta[e]azulen-2-yl)-amine
(3-Methyl-pyridin-2-yl)-(4,5,6,7-tetrahydro-3-thia-1,7,8-triaza-cyclopenta[e]azulen-2-yl)-amine
6-(4,5,6,7-Tetrahydro-3-thia-1,7,8-triaza-cyclopenta[e]azulen-2-ylamino)-pyridine-2-carbonitrile
(5-Methyl-pyridin-2-yl)-(4,5,6,7-tetrahydro-3-thia-1,7,8-triaza-cyclopenta[e]azulen-2-yl)-amine
[6-(4,5,6,7-Tetrahydro-3-thia-1,7,8-triaza-cyclopenta[e]azulen-2-ylamino)-pyridin-2-yl]-methanol
6-(4,5,6,7-Tetrahydro-3-thia-1,7,8-triaza-cyclopenta[e]azulen-2-ylamino)-pyridine-2-carboxylic acid methyl ester
(4-Methoxy-pyrimidin-2-yl)-(4,5,6,7-tetrahydro-3-thia-1,7,8-triaza-cyclopenta[e]azulen-2-yl)-amine
(6-Pyrrolidin-1-yl-pyridin-2-yl)-(4,5,6,7-tetrahydro-3-thia-1,7,8-triaza-cyclopenta[e]azulen-2-yl)-amine
[4-(2-Methoxy-ethoxy)-pyrimidin-2-yl]-(4,5,6,7-tetrahydro-3-thia-1,7,8-triaza-cyclopenta[e]azulen-2-yl)-amine
N,N-Dimethyl-N′-(4,5,6,7-tetrahydro-3-thia-1,7,8-triaza-cyclopenta[e]azulen-2-yl)-pyridine-2,6-diamine and
N-Ethyl-N′-(4,5,6,7-tetrahydro-3-thia-1,7,8-triaza-cyclopenta[e]azulen-2-yl)-pyridine-2,6-diamine.
In a more preferred aspect of Formula (II), the invention provides a compound wherein:
   Y is selected from the group of —CR¹R²—, —CR¹R²—CR³R⁴—, —CR¹R²—CR³R⁴—CR⁵R⁶— and —CR¹R²—CR³R⁴—CR⁵R⁶—CR⁷R⁸;
   M is an optionnally substituted pyrimidinyl;
   R¹, R², R³ or R⁴ are each independently selected from the group of hydrogen, fluoro, CF₃, OMe, methyl and propyl;
   provided that according to proviso (v) the compound is not:
Pyrimidin-2-yl-(4,5,6,7-tetrahydro-3-thia-1,7,8-triaza-cyclopenta[e]azulen-2-yl)-amine
N-(Pyrimidin-2-yl)-5,6-dihydro-4H-thiazolo[4,5-e]indazol-2-amine
N-(4-Methylpyrimidin-2-yl)-5,6-dihydro-4H-thiazolo[4,5-e]indazol-2-amine
(4-Methyl-pyrimidin-2-yl)-(4,5,6,7-tetrahydro-3-thia-1,7,8-triaza-cyclopenta[e]azulen-2-yl)-amine
4,4-Dimethyl-N-(pyrimidin-2-yl)-5,6-dihydro-4H-thiazolo[4,5-e]indazol-2-amine
4-Methyl-N-(pyrimidin-2-yl)-5,6-dihydro-4H-thiazolo[4,5-e]indazol-2-amine and
5,5-Dimethyl-N-(pyrimidin-2-yl)-5,6-dihydro-4H-thiazolo[4,5-e]indazol-2-amine;
   provided that according to proviso (vi) the compound is not:
4,4-Dimethyl-N-(4-methylpyrimidin-2-yl)-5,6-dihydro-4H-[1,3]thiazolo[4,5-e]indazol-2-amine
N-(5-Fluoropyrimidin-2-yl)-4,4-dimethyl-5,6-dihydro-4H-[1,3]thiazolo[4,5-e]indazol-2-amine
N-(5-Fluoro-4-methylpyrimidin-2-yl)-4,4-dimethyl-5,6-dihydro-4H-[1,3]thiazolo[4,5-e]indazol-2-amine
5-Methyl-N-(pyrimidin-2-yl)-5,6-dihydro-4H-[1,3]thiazolo[4,5-e]indazol-2-amine
5-Methyl-N-(4-methylpyrimidin-2-yl)-5,6-dihydro-4H-[1,3]thiazolo[4,5-e]indazol-2-amine
N-(Pyrimidin-2-yl)-1,7-dihydropyrazolo[3′,4′:4,5]cyclopenta[1,2-d][1,3]thiazol-5-amine
N-(5-Fluoropyrimidin-2-yl)-4,5,6,7-tetrahydropyrazolo[3′,4′:6,7]cyclohepta[1,2-d][1,3]thiazol-2-amine
(5,5-Dimethyl-4,5,6,7-tetrahydro-3-thia-1,7,8-triaza-cyclopenta[e]azulen-2-yl)-(5-fluoro-pyrimidin-2-yl)-amine
6,6-Difluoro-N-(4-methylpyrimidin-2-yl)-4,5,6,7-tetrahydropyrazolo[3′,4′:6,7]cyclohepta[1,2-d][1,3]thiazol-2-amine and
(5-Fluoro-4,5,6,7-tetrahydro-3-thia-1,7,8-triaza-cyclopenta[e]azulen-2-yl)-(5-methyl-pyrimidin-2-yl)-amine;
   provided that according to proviso (vii) the compound is not:
Pyrimidin-4-yl-(4,5,6,7-tetrahydro-3-thia-1,7,8-triaza-cyclopenta[e]azulen-2-yl)-amine
(4-Methoxy-pyrimidin-2-yl)-(4,5,6,7-tetrahydro-3-thia-1,7,8-triaza-cyclopenta[e]azulen-2-yl)-amine and
[4-(2-Methoxy-ethoxy)-pyrimidin-2-yl]-(4,5,6,7-tetrahydro-3-thia-1,7,8-triaza-cyclopenta[e]azulen-2-yl)-amine.

In a more preferred aspect of Formula (II), the invention provides a compound wherein:

Y is selected from the group of —O—CR¹R²—, —O—CR¹R²—CR³R⁴—, —NR⁹—CR¹R²— and NR⁹—CR¹R²—CR³R⁴—;
R¹, R², R³ or R⁴ are each independently selected from the group of hydrogen, halogen, —CN, —CF₃ or an optionally substituted radical selected from the group of —(C₁-C₆)alkyl, —(C₁-C₆)haloalkyl, —(C₃-C₇)cycloalkyl, aryl, heteroaryl, heterocycle, —(C₁-C₆)alkylene-aryl, —(C₁-C₆)alkylene-heteroaryl, —(C₁-C₆)alkylene-heterocycle, —O—(C₀-C₆)alkyl, —N—((C₀-C₆)alkyl)₂, —(C₁-C₆)alkyl-O—(C₀-C₆)alkyl, and —(C₁-C₆)alkyl-N—((C₀-C₆)alkyl)₂;
Any two radicals of R (R¹, R², R³ or R⁴) may be taken together to form an optionally substituted 3 to 10 membered carbocyclic or heterocyclic ring; and
R⁹ is selected from the group of hydrogen or an optionally substituted radical selected from the group of —(C₁-C₆)alkyl, —(C₁-C₆)haloalkyl, —(C₃-C₇)cycloalkyl, —(C₁-C₆)alkylene-(C₁-C₆)haloalkyl, —(C₁-C₆)alkylene-(C₃-C₇)halocycloalkyl, aryl, heteroaryl, heterocycle, —(C₁-C₆)alkylene-aryl, —(C₁-C₆)alkylene-heteroaryl, —(C₁-C₆)alkylene-heterocycle, —(C₁-C₆)alkylene-(C₃-C₇)cycloalkyl, —(C₂-C₆)alkyl-O—(C₀-C₆)alkyl, and —(C₂-C₆)alkyl-N—((C₀-C₆)alkyl)₂.

In a more preferred aspect of Formula (II), the invention provides a compound wherein:

Y is selected from the group of —O—CR¹R²—, —O—CR¹R²—CR³R⁴—, —NR⁹—CR¹R²— and —NR⁹—CR¹R²—CR³R⁴—;
R¹, R², R³ or R⁴ are each independently selected from the group of hydrogen, halogen, —CN, —CF₃ or an optionally substituted radical selected from the group of —(C₁-C₆)alkyl, —(C₁-C₆)haloalkyl, —(C₃-C₇)cycloalkyl, aryl, heteroaryl, heterocycle, —(C₁-C₆)alkylene-aryl, —(C₁-C₆)alkylene-heteroaryl, —(C₁-C₆)alkylene-heterocycle, —O—(C₀-C₆)alkyl, —N—((C₀-C₆)alkyl)₂, —(C₁-C₆)alkyl-O—(C₀-C₆)alkyl, and —(C₁-C₆)alkyl-N—((C₀-C₆)alkyl)₂;
Any two radicals of R (R¹, R², R³ or R⁴) may be taken together to form an optionally substituted 3 to 10 membered carbocyclic or heterocyclic ring; and
R⁹ is selected from the group of hydrogen or an optionally substituted radical selected from the group of —(C₁-C₆)alkyl, —(C₁-C₆)haloalkyl, —(C₃-C₇)cycloalkyl, —(C₁-C₆)alkylene-(C₁-C₆)haloalkyl, —(C₁-C₆)alkylene-(C₃-C₇)halocycloalkyl, aryl, heteroaryl, heterocycle, —(C₁-C₆)alkylene-aryl, —(C₁-C₆)alkylene-heteroaryl, —(C₁-C₆)alkylene-heterocycle, —(C₂-C₆)alkyl-O—(C₀-C₆)alkyl, and —(C₂-C₆)alkyl-N—((C₀-C₆)alkyl)₂.

In a more preferred aspect of Formula (II), the invention provides a compound wherein:

Y is selected from the group of —CR¹R²—O—CR³R⁴— and —CR¹R²—NR⁹—CR³R⁴—;
R¹, R², R³ or R⁴ are each independently selected from the group of hydrogen, —CN, —CF₃ or an optionally substituted radical selected from the group of —(C₁-C₆)alkyl, —(C₁-C₆)haloalkyl, —(C₃-C₇)cycloalkyl, aryl, heteroaryl, heterocycle, —(C₁-C₆)alkylene-aryl, —(C₁-C₆)alkylene-heteroaryl, —(C₁-C₆)alkylene-heterocycle, —(C₁-C₆)alkyl-O—(C₀-C₆)allyl, and —(C₁-C₆)alkyl-N—((C₀-C₆)alkyl)₂;
Any two radicals of R (R¹, R², R³ or R⁴) may be taken together to form an optionally substituted 3 to 10 membered carbocyclic or heterocyclic ring; and
R⁹ is selected from the group of hydrogen or an optionally substituted radical selected from the group of —(C₁-C₆)alkyl, —(C₁-C₆)haloalkyl, —(C₃-C₇)cycloalkyl, —(C₁-C₆)alkylene-(C₁-C₆)haloalkyl, —(C₁-C₆)alkylene-(C₃-C₇)halocycloalkyl, aryl, heteroaryl, heterocycle, —(C₁-C₆)allylene-aryl, —(C₁-C₆)alkylene-heteroaryl, —(C₁-C₆)alkylene-heterocycle, —(C₁-C₆)alkylene-(C₃-C₇)cycloalkyl, —(C₂-C₆)alkyl-O—(C₀-C₆)alkyl, and —(C₂-C₆)alkyl-N—((C₀-C₆)alkyl)₂;
provided that according to proviso (viii) the compound is not:
N-(Pyrimidin-2-yl)-6,7-dihydro-4H-pyrazolo[4′,3′:5,6]oxepino[4,3-d][1,3]thiazol-2-amine
N-(5-Fluoropyrimidin-2-yl)-6,7-dihydro-4H-pyrazolo[4′,3′:5,6]oxepino[4,3-d][1,3]thiazol-2-amine
N-(4-Methylpyrimidin-2-yl)-6,7-dihydro-4H-pyrazolo[4′,3′:5,6]oxepino[4,3-d][1,3]thiazol-2-amine
(6-Methyl-6,7-dihydro-4H-5-oxa-3-thia-1,7,8-triaza-cyclopenta[e]azulen-2-yl)-(4-methyl-pyrimidin-2-yl)-amine
((R)-6-Methyl-6,7-dihydro-4H-5-oxa-3-thia-1,7,8-triaza-cyclopenta[e]azulen-2-yl)-(4-methyl-pyrimidin-2-yl)-amine
((S)-6-Methyl-6,7-dihydro-4H-5-oxa-3-thia-1,7,8-triaza-cyclopenta[e]azulen-2-yl)-(4-methyl-pyrimidin-2-yl)-amine
(6,7-Dihydro-4H-5-oxa-3-thia-1,7,8-triaza-cyclopenta[e]azulen-2-yl)-(4-methoxy-pyrimidin-2-yl)-amine
(4-Isopropyl-pyrimidin-2-yl)-(6-methyl-6,7-dihydro-4H-5-oxa-3-thia-1,7,8-triaza-cyclopenta[e]azulen-2-yl)-amine
(4-Ethyl-pyrimidin-2-yl)-(6-methyl-6,7-dihydro-4H-5-oxa-3-thia-1,7,8-triaza-cyclopenta[e]azulen-2-yl)-amine
(4-Cyclopropyl-pyrimidin-2-yl)-(6-methyl-6,7-dihydro-4H-5-oxa-3-thia-1,7,8-triaza-cyclopenta[e]azulen-2-yl)-amine
(N-(5-Fluoro-4-methylpyrimidin-2-yl)-6-methyl-6,7-dihydro-4H-pyrazolo[4′,3′:5,6]oxepino[4,3-d][1,3]thiazol-2-amine)
(N-(5-Fluoropyrimidin-2-yl)-6-methyl-6,7-dihydro-4H-pyrazolo[4′,3′:5,6]oxepino[4,3-d][1,3]thiazol-2-amine)
4-Methyl-N-(4-methylpyrimidin-2-yl)-6,7-dihydro-4H-pyrazolo[4′,3′:5,6]oxepino[4,3-d][1,3]thiazol-2-amine
4,6-Dimethyl-N-(4-methylpyrimidin-2-yl)-6,7-dihydro-4H-pyrazolo[4′,3′:5,6]oxepino-[4,3-d][1,3]thiazol-2-amine
N-(Pyrimidin-2-yl)-4,5,6,7-tetrahydropyrazolo[3,4-c][1,3]thiazolo[4,5-e]azepin-2-amine
5-Methyl-N-(4-methylpyrimidin-2-yl)-4,5,6,7-tetrahydropyrazolo[3,4-c][1,3]thiazolo[4,5-e]azepin-2-amine and
(6-Methyl-6,7-dihydro-4H-5-oxa-3-thia-1,7,8-triaza-cyclopenta[e]azulen-2-yl)-(5-methyl-[1,2,4]thiadiazol-3-yl)-amine.

In a more preferred aspect of Formula (II), the invention provides a compound wherein:

Y is selected from the group of —CR¹R²—O—CR³R⁴— and —CR¹R²—NR⁹—CR³R⁴—;
R¹, R², R³ or R⁴ are each independently selected from the group of hydrogen, —CN, —CF₃ or an optionally substituted radical selected from the group of —(C₁-C₆)allyl, —(C₁-C₆)haloalkyl, —(C₃-C₇)cycloalkyl, aryl, heteroaryl, heterocycle, —(C₁-C₆)alkylene-aryl, —(C₁-C₆)alkylene-heteroaryl, —(C₁-C₆)alkylene-heterocycle, —(C₁-C₆)alkyl-O—(C₀-C₆)alkyl, and —(C₁-C₆)alkyl-N—((C₀-C₆)alkyl)₂;
Any two radicals of R (R¹, R², R³ or R⁴) may be taken together to form an optionally substituted 3 to 10 membered carbocyclic or heterocyclic ring; and
R⁹ is selected from the group of hydrogen or an optionally substituted radical selected from the group of —(C₁-C₆)alkyl, —(C₁-C₆)haloalkyl, —(C₃-C₇)cycloalkyl, —(C₁-C₆)alkylene-(C₁-C₆)haloalkyl, —(C₁-C₆)alkylene-(C₃-C₇)halocycloalkyl, aryl, heteroaryl, heterocycle, —(C₁-C₆)alkylene-aryl, —(C₁-C₆)alkylene-heteroaryl, —(C₁-C₆)alkylene-heterocycle, —(C₂-C₆)alkyl-O—(C₀-C₆)alkyl, and —(C₂-C₆)alkyl-N—((C₀-C₆)alkyl)₂;
provided that according to proviso (viii) the compound is not:
N-(Pyrimidin-2-yl)-6,7-dihydro-4H-pyrazolo[4′,3′:5,6]oxepino[4,3-d][1,3]thiazol-2-amine
N-(5-Fluoropyrimidin-2-yl)-6,7-dihydro-4H-pyrazolo[4′,3′:5,6]oxepino[4,3-d][1,3]thiazol-2-amine
N-(4-Methylpyrimidin-2-yl)-6,7-dihydro-4H-pyrazolo[4′,3′:5,6]oxepino[4,3-d][1,3]thiazol-2-amine
(6-Methyl-6,7-dihydro-4H-5-oxa-3-thia-1,7,8-triaza-cyclopenta[e]azulen-2-yl)-(4-methyl-pyrimidin-2-yl)-amine
((R)-6-Methyl-6,7-dihydro-4H-5-oxa-3-thia-1,7,8-triaza-cyclopenta[e]azulen-2-yl)-(4-methyl-pyrimidin-2-yl)-amine
((S)-6-Methyl-6,7-dihydro-4H-5-oxa-3-thia-1,7,8-triaza-cyclopenta[e]azulen-2-yl)-(4-methyl-pyrimidin-2-yl)-amine
(6,7-Dihydro-4H-5-oxa-3-thia-1,7,8-triaza-cyclopenta[e]azulen-2-yl)-(4-methoxy-pyrimidin-2-yl)-amine
(4-Isopropyl-pyrimidin-2-yl)-(6-methyl-6,7-dihydro-4H-5-oxa-3-thia-1,7,8-triaza-cyclopenta[e]azulen-2-yl)-amine
(4-Ethyl-pyrimidin-2-yl)-(6-methyl-6,7-dihydro-4H-5-oxa-3-thia-1,7,8-triaza-cyclopenta[e]azulen-2-yl)-amine
(4-Cyclopropyl-pyrimidin-2-yl)-(6-methyl-6,7-dihydro-4H-5-oxa-3-thia-1,7,8-triaza-cyclopenta[e]azulen-2-yl)-amine
(N-(5-Fluoro-4-methylpyrimidin-2-yl)-6-methyl-6,7-dihydro-4H-pyrazolo[4′,3′:5,6]oxepino[4,3-d][1,3]thiazol-2-amine)
(N-(5-Fluoropyrimidin-2-yl)-6-methyl-6,7-dihydro-4H-pyrazolo[4′,3′:5,6]oxepino[4,3-d][1,3]thiazol-2-amine)
4-Methyl-N-(4-methylpyrimidin-2-yl)-6,7-dihydro-4H-pyrazolo[4′,3′:5,6]oxepino[4,3-d][1,3]thiazol-2-amine
4,6-Dimethyl-N-(4-methylpyrimidin-2-yl)-6,7-dihydro-4H-pyrazolo[4′,3′:5,6]oxepino-[4,3-d][1,3]thiazol-2-amine
N-(Pyrimidin-2-yl)-4,5,6,7-tetrahydropyrazolo[3,4-c][1,3]thiazolo[4,5-e]azepin-2-amine
5-Methyl-N-(4-methylpyrimidin-2-yl)-4,5,6,7-tetrahydropyrazolo[3,4-c][1,3]thiazolo[4,5-e]azepin-2-amine and
(6-Methyl-6,7-dihydro-4H-5-oxa-3-thia-1,7,8-triaza-cyclopenta[e]azulen-2-yl)-(5-methyl-[1,2,4]thiadiazol-3-yl)-amine.

In a more preferred aspect of Formula (II), the invention provides a compound according to Formula (III):

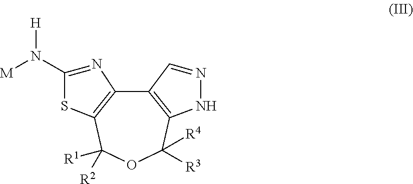

M is an optionnally substituted pyridinyl, pyrimidinyl, thiadiazolyl, triazinyl, thiazolyl and oxadiazolyl;
R¹, R², R³ or R⁴ are each independently selected from the group of hydrogen or an optionally substituted radical selected from the group of —(C₁-C₆)alkyl, —(C₁-C₆)haloalkyl, —(C₃-C₇)cycloalkyl, aryl, heteroaryl, heterocycle, —(C₁-C₆)alkylene-aryl, —(C₁-C₆)alkylene-heteroaryl, —(C₁-C₆)alkylene-heterocycle, —(C₁-C₆)allyl-O—(C₀-C₆)alkyl, and —(C₁-C₆)alkyl-N—((C₀-C₆)alkyl)₂; and
Any two radicals of R (R¹, R², R³ or R⁴) may be taken together to form an optionally substituted 3 to 10 membered carbocyclic or heterocyclic ring;
provided that according to proviso (ix) the compound is not:
N-(Pyrimidin-2-yl)-6,7-dihydro-4H-pyrazolo[4′,3′:5,6]oxepino[4,3-d][1,3]thiazol-2-amine
N-(5-Fluoropyrimidin-2-yl)-6,7-dihydro-4H-pyrazolo[4′,3′:5,6]oxepino[4,3-d][1,3]thiazol-2-amine
N-(4-Methylpyrimidin-2-yl)-6,7-dihydro-4H-pyrazolo[4′,3′:5,6]oxepino[4,3-d][1,3]thiazol-2-amine
(6-Methyl-6,7-dihydro-4H-5-oxa-3-thia-1,7,8-triaza-cyclopenta[e]azulen-2-yl)-(4-methyl-pyrimidin-2-yl)-amine
((R)-6-Methyl-6,7-dihydro-4H-5-oxa-3-thia-1,7,8-triaza-cyclopenta[e]azulen-2-yl)-(4-methyl-pyrimidin-2-yl)-amine
((S)-6-Methyl-6,7-dihydro-4H-5-oxa-3-thia-1,7,8-triaza-cyclopenta[e]azulen-2-yl)-(4-methyl-pyrimidin-2-yl)-amine
(6,7-Dihydro-4H-5-oxa-3-thia-1,7,8-triaza-cyclopenta[e]azulen-2-yl)-(4-methoxy-pyrimidin-2-yl)-amine
(4-Isopropyl-pyrimidin-2-yl)-(6-methyl-6,7-dihydro-4H-5-oxa-3-thia-1,7,8-triaza-cyclopenta[e]azulen-2-yl)-amine
(4-Ethyl-pyrimidin-2-yl)-(6-methyl-6,7-dihydro-4H-5-oxa-3-thia-1,7,8-triaza-cyclopenta[e]azulen-2-yl)-amine
(4-Cyclopropyl-pyrimidin-2-yl)-(6-methyl-6,7-dihydro-4H-5-oxa-3-thia-1,7,8-triaza-cyclopenta[e]azulen-2-yl)-amine
(N-(5-Fluoro-4-methylpyrimidin-2-yl)-6-methyl-6,7-dihydro-4H-pyrazolo[4′,3′:5,6]oxepino[4,3-d][1,3]thiazol-2-amine)
(N-(5-Fluoropyrimidin-2-yl)-6-methyl-6,7-dihydro-4H-pyrazolo[4′,3′:5,6]oxepino[4,3-d][1,3]thiazol-2-amine)
4-Methyl-N-(4-methylpyrimidin-2-yl)-6,7-dihydro-4H-pyrazolo[4′,3′:5,6]oxepino[4,3-d][1,3]thiazol-2-amine
4,6-Dimethyl-N-(4-methylpyrimidin-2-yl)-6,7-dihydro-4H-pyrazolo[4′,3′:5,6]oxepino-[4,3-d][1,3]thiazol-2-amine and
(6-Methyl-6,7-dihydro-4H-5-oxa-3-thia-1,7,8-triaza-cyclopenta[e]azulen-2-yl)-(5-methyl-[1,2,4]thiadiazol-3-yl)-amine.

In a more preferred aspect of Formula (III), the invention provides a compound wherein:

M is an optionally substituted pyridinyl, pyrimidinyl and thiadiazolyl;
R¹, R², R³ or R⁴ are each independently selected from the group of hydrogen, CF₃, methyl, ethyl, isopropyl, and an optionally substituted pyridinyl;
provided that according to proviso (ix) the compound is not:
N-(Pyrimidin-2-yl)-6,7-dihydro-4H-pyrazolo[4′,3′:5,6]oxepino[4,3-d][1,3]thiazol-2-amine
N-(5-Fluoropyrimidin-2-yl)-6,7-dihydro-4H-pyrazolo[4′,3′:5,6]oxepino[4,3-d][1,3]thiazol-2-amine
N-(4-Methylpyrimidin-2-yl)-6,7-dihydro-4H-pyrazolo[4′,3′:5,6]oxepino[4,3-d][1,3]thiazol-2-amine
(6-Methyl-6,7-dihydro-4H-5-oxa-3-thia-1,7,8-triaza-cyclopenta[e]azulen-2-yl)-(4-methyl-pyrimidin-2-yl)-amine
((R)-6-Methyl-6,7-dihydro-4H-5-oxa-3-thia-1,7,8-triaza-cyclopenta[e]azulen-2-yl)-(4-methyl-pyrimidin-2-yl)-amine
((S)-6-Methyl-6,7-dihydro-4H-5-oxa-3-thia-1,7,8-triaza-cyclopenta[e]azulen-2-yl)-(4-methyl-pyrimidin-2-yl)-amine
(6,7-Dihydro-4H-5-oxa-3-thia-1,7,8-triaza-cyclopenta[e]azulen-2-yl)-(4-methoxy-pyrimidin-2-yl)-amine
(4-Isopropyl-pyrimidin-2-yl)-(6-methyl-6,7-dihydro-4H-5-oxa-3-thia-1,7,8-triaza-cyclopenta[e]azulen-2-yl)-amine
(4-Ethyl-pyrimidin-2-yl)-(6-methyl-6,7-dihydro-4H-5-oxa-3-thia-1,7,8-triaza-cyclopenta[e]azulen-2-yl)-amine
(4-Cyclopropyl-pyrimidin-2-yl)-(6-methyl-6,7-dihydro-4H-5-oxa-3-thia-1,7,8-triaza-cyclopenta[e]azulen-2-yl)-amine
(N-(5-Fluoro-4-methylpyrimidin-2-yl)-6-methyl-6,7-dihydro-4H-pyrazolo[4′,3′:5,6]oxepino[4,3-d][1,3]thiazol-2-amine)
(N-(5-Fluoropyrimidin-2-yl)-6-methyl-6,7-dihydro-4H-pyrazolo[4′,3′:5,6]oxepino[4,3-d][1,3]thiazol-2-amine)
4-Methyl-N-(4-methylpyrimidin-2-yl)-6,7-dihydro-4H-pyrazolo[4′,3′:5,6]oxepino[4,3-d][1,3]thiazol-2-amine
4,6-Dimethyl-N-(4-methylpyrimidin-2-yl)-6,7-dihydro-4H-pyrazolo[4′,3′:5,6]oxepino-[4,3-d][1,3]thiazol-2-amine and
(6-Methyl-6,7-dihydro-4H-5-oxa-3-thia-1,7,8-triaza-cyclopenta[e]azulen-2-yl)-(5-methyl-[1,2,4]thiadiazol-3-yl)-amine.

In a more preferred aspect of Formula (II), the invention provides a compound wherein:

Y is selected from the group of —CR¹R²—O—, —CR¹R²—CR³R⁴—O— and —CR¹R²—NR⁹—;
R¹, R², R³ or R⁴ are each independently selected from the group of hydrogen, halogen, —CN, —CF₃ or an optionally substituted radical selected from the group of —(C₁-C₆)allyl, —(C₁-C₆)haloalkyl, —(C₃-C₇)cycloalkyl, aryl, heteroaryl, heterocycle, —(C₁-C₆)alkylene-aryl, —(C₁-C₆)alkylene-heteroaryl, —(C₁-C₆)alkylene-heterocycle, —O—(C₀-C₆)alkyl, —N—((C₀-C₆)alkyl)₂, —(C₁-C₆)alkyl-O—(C₀-C₆)alkyl, and —(C₁-C₆)alkyl-N—((C₀-C₆)alkyl)₂;
Any two radicals of R (R¹, R², R³ or R⁴) may be taken together to form an optionally substituted 3 to 10 membered carbocyclic or heterocyclic ring; and
R⁹ is selected from the group of hydrogen or an optionally substituted radical selected from the group of —(C₁-C₆)alkyl, —(C₁-C₆)haloalkyl, —(C₃-C₇)cycloalkyl, —(C₁-C₆)alkylene-(C₁-C₆)haloalkyl, —(C₁-C₆)alkylene-(C₃-C₇)halocycloalkyl, aryl, heteroaryl, heterocycle, —(C₁-C₆)alkylene-aryl, —(C₁-C₆)alkylene-heteroaryl, —(C₁-C₆)alkylene-heterocycle, —(C₁-C₆)allylene-(C₃-C₇)cycloalkyl, —(C₂-C₆)alkyl-O—(C₀-C₆)alkyl, and —(C₂-C₆)alkyl-N—((C₀-C₆)alkyl)₂.

In a more preferred aspect of Formula (II), the invention provides a compound wherein:

Y is selected from the group of —CR¹R²—O—, —CR¹R²—CR³R⁴—O— and —CR¹R²—NR⁹—;
R¹, R², R³ or R⁴ are each independently selected from the group of hydrogen, halogen, —CN, —CF₃ or an optionally substituted radical selected from the group of —(C₁-C₆)alkyl, —(C₁-C₆)haloalkyl, —(C₃-C₇)cycloalkyl, aryl, heteroaryl, heterocycle, —(C₁-C₆)alkylene-aryl, —(C₁-C₆)alkylene-heteroaryl, —(C₁-C₆)alkylene-heterocycle, —O—(C₀-C₆)alkyl, —N—((C₀-C₆)alkyl)₂, —(C₁-C₆)alkyl-O—(C₀-C₆)alkyl, and —(C₁-C₆)alkyl-N—((C₀-C₆)alkyl)₂;
Any two radicals of R (R¹, R², R³ or R⁴) may be taken together to form an optionally substituted 3 to 10 membered carbocyclic or heterocyclic ring; and
R⁹ is selected from the group of hydrogen or an optionally substituted radical selected from the group of —(C₁-C₆)alkyl, —(C₁-C₆)haloalkyl, —(C₃-C₇)cycloalkyl, —(C₁-C₆)alkylene-(C₁-C₆)haloalkyl, —(C₁-C₆)alkylene-(C₃-C₇)halocycloalkyl, aryl, heteroaryl, heterocycle, —(C₁-C₆)alkylene-aryl, —(C₁-C₆)alkylene-heteroaryl, —(C₁-C₆)alkylene-heterocycle, —(C₂-C₆)alkyl-O—(C₀-C₆)alkyl, and —(C₂-C₆)alkyl-N—((C₀-C₆)alkyl)₂.

Provisos (i) and (v) are based on the disclosures of claims 6 and 7 respectively in WO2010/079238.

Provisos (ii), (iv), (vi), (viii) and (ix) are based on compounds described in co-pending unpublished U.S. Provisional application No. 61/364,195 filed 14 Jul. 2010.

Provisos (iii) and (vii) are based on compounds described in Hong S.-P et al, (20 Jun. 2011) J. Med. Chem., DOI: 10.1021/jm200290z, http://pubs.acs.org.

Particular preferred compounds of the invention are compounds as mentioned in the following list (List of Particular Preferred Compounds), as well as a pharmaceutically acceptable acid or base addition salt thereof, a stereochemically isomeric form thereof and an N-oxide form thereof:
(4-Methyl-6H-3-thia-1,6,7-triaza-as-indacen-2-yl)-pyridin-2-yl-amine
(4-Methyl-pyrimidin-2-yl)-(4,4,6-trimethyl-6,7-dihydro-4H-5-oxa-3-thia-1,7,8-triaza-cyclopenta[e]azulen-2-yl)-amine
2-(4-Methyl-pyrimidin-2-ylamino)-4,5,6,7-tetrahydro-3-thia-1,7,8-triaza-cyclopenta[e]azulen-6-ol
(4,4-Dimethyl-6,7-dihydro-4H-5-oxa-3-thia-1,7,8-triaza-cyclopenta[e]azulen-2-yl)-(4-methyl-pyrimidin-2-yl)-amine
(4-Methyl-pyrimidin-2-yl)-(5-propyl-5,6-dihydro-4H-3-thia-1,6,7-triaza-as-indacen-2-yl)-amine
Dimethyl-carbamic acid 2-(4-methyl-pyrimidin-2-ylamino)-4,5,6,7-tetrahydro-3-thia-1,7,8-triaza-cyclopenta[e]azulen-5-yl ester
(4-Methyl-pyrimidin-2-yl)-(5,6,7,8-tetrahydro-4H-3-thia-1,8,9-triaza-dicyclopenta[a,c]cycloocten-2-yl)-amine
(6-Ethyl-6,7-dihydro-4H-5-oxa-3-thia-1,7,8-triaza-cyclopenta[e]azulen-2-yl)-(4-methyl-pyrimidin-2-yl)-amine
(6,6-Difluoro-5,5-dimethyl-4,5,6,7-tetrahydro-3-thia-1,7,8-triaza-cyclopenta[e]azulen-2-yl)-(4-methyl-pyrimidin-2-yl)-amine
(6,6-Dimethyl-6,7-dihydro-4H-5-oxa-3-thia-1,7,8-triaza-cyclopenta[e]azulen-2-yl)-(4-methyl-pyrimidin-2-yl)-amine
(4-Methyl-pyrimidin-2-yl)-(5-methyl-4,5,6,7-tetrahydro-3-thia-1,7,8-triaza-cyclopenta[e]azulen-2-yl)-amine
(4-Methyl-pyrimidin-2-yl)-(6-methyl-4,5,6,7-tetrahydro-3-thia-1,7,8-triaza-cyclopenta[e]azulen-2-yl)-amine
(4-Methyl-pyrimidin-2-yl)-(6-trifluoromethyl-6,7-dihydro-4H-5-oxa-3-thia-1,7,8-triaza-cyclopenta[e]azulen-2-yl)-amine
(4-Methoxy-pyrimidin-2-yl)-(6-methyl-6,7-dihydro-4H-5-oxa-3-thia-1,7,8-triaza-cyclopenta[e]azulen-2-yl)-amine
(5-Methoxy-4,5,6,7-tetrahydro-3-thia-1,7,8-triaza-cyclopenta[e]azulen-2-yl)-(4-methyl-pyrimidin-2-yl)-amine
(6-Isopropyl-6,7-dihydro-4H-5-oxa-3-thia-1,7,8-triaza-cyclopenta[e]azulen-2-yl)-(4-methyl-pyrimidin-2-yl)-amine
(4,7-Dihydro-5H-6-oxa-3-thia-1,7,8-triaza-cyclop enta[e]azulen-2-yl)-(4-methyl-pyrimidin-2-yl)-amine
(5,5-Difluoro-4,5,6,7-tetrahydro-3-thia-1,7,8-triaza-cyclopenta[e]azulen-2-yl)-(4-methyl-pyrimidin-2-yl)-amine
(6-Ethyl-6,7-dihydro-4H-5-oxa-3-thia-1,7,8-triaza-cyclopenta[e]azulen-2-yl)-(5-fluoro-pyrimidin-2-yl)-amine
(6-Ethyl-6,7-dihydro-4H-5-oxa-3-thia-1,7,8-triaza-cyclopenta[e]azulen-2-yl)-(5-methyl-[1,2,4]thiadiazol-3-yl)-amine
(6-Ethyl-6,7-dihydro-4H-5-oxa-3-thia-1,7,8-triaza-cyclopenta[e]azulen-2-yl)-(5-fluoro-4-methyl-pyrimidin-2-yl)-amine
(6-Ethyl-6,7-dihydro-4H-5-oxa-3-thia-1,7,8-triaza-cyclopenta[e]azulen-2-yl)-(4-methoxy-pyrimidin-2-yl)-amine
(6-Fluoro-pyridin-2-yl)-(6-methyl-6,7-dihydro-4H-5-oxa-3-thia-1,7,8-triaza-cyclopenta[e]azulen-2-yl)-amine
(6-Methoxy-4,5,6,8-tetrahydro-3-thia-1,7,8-triaza-cyclopenta[e]azulen-2-yl)-(4-methyl-pyrimidin-2-yl)-amine
(6-Isopropyl-6,7-dihydro-4H-5-oxa-3-thia-1,7,8-triaza-cyclopenta[e]azulen-2-yl)-(5-methyl-[1,2,4]thiadiazol-3-yl)-amine
(6-Fluoro-pyridin-2-yl)-(6-isopropyl-6,7-dihydro-4H-5-oxa-3-thia-1,7,8-triaza-cyclopenta[e]azulen-2-yl)-amine
(4-Methyl-pyrimidin-2-yl)-(6-trifluoromethyl-4,5,6,7-tetrahydro-3-thia-1,7,8-triaza-cyclopenta[e]azulen-2-yl)-amine
(5-Fluoro-pyrimidin-2-yl)-(6-isopropyl-6,7-dihydro-4H-5-oxa-3-thia-1,7,8-triaza-cyclopenta[e]azulen-2-yl)-amine
(5-Fluoro-4-methyl-pyrimidin-2-yl)-(6-isopropyl-6,7-dihydro-4H-5-oxa-3-thia-1,7,8-triaza-cyclopenta[e]azulen-2-yl)-amine
2-(5-Fluoro-4-methyl-pyrimidin-2-ylamino)-6,7-dihydro-5H-3-thia-1,7,8-triaza-cyclopenta[e]azulen-4-one and
(4-Methyl-pyrimidin-2-yl)-(6-pyridin-2-yl-6,7-dihydro-4H-5-oxa-3-thia-1,7,8-triaza-cyclopenta[e]azulen-2-yl)-amine.

The following Reference Compounds are described in co-pending unpublished U.S. Provisional application No. 61/364,195 filed 14 Jul. 2010. The Reference compounds do not form part of the present invention as such: they are described here to demonstrate support for the claimed invention which provides a class of compounds which are modulators of metabotropic glutamate receptors—subtype 4 (“mGluR₄”) which are useful for the treatment or prevention of central nervous system disorders as well as other disorders modulated by mGluR₄ receptors:
4,4-Dimethyl-N-(4-methylpyrimidin-2-yl)-5,6-dihydro-4H-[1,3]thiazolo[4,5-e]indazol-2-amine
N-(4-Methylpyrimidin-2-yl)-6H-[1,3]thiazolo[4,5-e]indazol-2-amine
N-(Pyrimidin-2-yl)-6H-[1,3]thiazolo[4,5-e]indazol-2-amine
N-(5-Fluoropyrimidin-2-yl)-4,4-dimethyl-5,6-dihydro-4H-[1,3]thiazolo[4,5-e]indazol-2-amine
N-(5-Fluoro-4-methylpyrimidin-2-yl)-4,4-dimethyl-5,6-dihydro-4H-[1,3]thiazolo[4,5-e]indazol-2-amine
5-Bromo-N-(pyrimidin-2-yl)-6H-[1,3]thiazolo[4,5-e]indazol-2-amine
5-Chloro-N-(pyrimidin-2-yl)-6H-[1,3]thiazolo[4,5-e]indazol-2-amine
5-Methyl-N-(4-methylpyrimidin-2-yl)-6H-[1,3]thiazolo[4,5-e]indazol-2-amine
5-Methyl-N-(pyrimidin-2-yl)-5,6-dihydro-4H-[1,3]thiazolo[4,5-e]indazol-2-amine
5-Methyl-N-(4-methylpyrimidin-2-yl)-5,6-dihydro-4H-[1,3]thiazolo[4,5-e]indazol-2-amine
N-(4-Methylpyrimidin-2-yl)-5′,6′-dihydrospiro[cyclopropane-1,4′-[1,3]thiazolo[4,5-e]indazol]-2′-amine
N-(Pyrimidin-2-yl)-5′,6′-dihydrospiro[cyclopropane-1,4′-[1,3]thiazolo[4,5-e]indazol]-2′-amine
N-(5-Fluoropyrimidin-2-yl)-5′,6′-dihydrospiro[cyclopropane-1,4′-[1,3]thiazolo[4,5-e]indazol]-2′-amine
N-(Pyrimidin-2-yl)-1,7-dihydropyrazolo[3′,4′:4,5]cyclopenta[1,2-d][1,3]thiazol-5-amine
N-(Pyrimidin-2-yl)-6,7-dihydro-4H-pyrazolo[4′,3′:5,6]oxepino[4,3-d][1,3]thiazol-2-amine
N-(5-Fluoropyrimidin-2-yl)-6,7-dihydro-4H-pyrazolo[4′,3′:5,6]oxepino[4,3-d][1,3]thiazol-2-amine
N-(4-Methylpyrimidin-2-yl)-6,7-dihydro-4H-pyrazolo[4′,3′:5,6]oxepino[4,3-d][1,3]thiazol-2-amine
(6-Methyl-6,7-dihydro-4H-5-oxa-3-thia-1,7,8-triaza-cyclopenta[e]azulen-2-yl)-(4-methyl-pyrimidin-2-yl)-amine
((R)-6-Methyl-6,7-dihydro-4H-5-oxa-3-thia-1,7,8-triaza-cyclopenta[e]azulen-2-yl)-(4-methyl-pyrimidin-2-yl)-amine
((S)-6-Methyl-6,7-dihydro-4H-5-oxa-3-thia-1,7,8-triaza-cyclopenta[e]azulen-2-yl)-(4-methyl-pyrimidin-2-yl)-amine
(6,7-Dihydro-4H-5-oxa-3-thia-1,7,8-triaza-cyclopenta[e]azulen-2-yl)-(4-methoxy-pyrimidin-2-yl)-amine
(4-Isopropyl-pyrimidin-2-yl)-(6-methyl-6,7-dihydro-4H-5-oxa-3-thia-1,7,8-triaza-cyclopenta[e]azulen-2-yl)-amine
(4-Ethyl-pyrimidin-2-yl)-(6-methyl-6,7-dihydro-4H-5-oxa-3-thia-1,7,8-triaza-cyclopenta[e]azulen-2-yl)-amine
(4-Cyclopropyl-pyrimidin-2-yl)-(6-methyl-6,7-dihydro-4H-5-oxa-3-thia-1,7,8-triaza-cyclopenta[e]azulen-2-yl)-amine
(N-(5-Fluoro-4-methylpyrimidin-2-yl)-6-methyl-6,7-dihydro-4H-pyrazolo[4′,3′:5,6]oxepino[4,3-d][1,3]thiazol-2-amine)
(N-(5-Fluoropyrimidin-2-yl)-6-methyl-6,7-dihydro-4H-pyrazolo[4′,3′:5,6]oxepino[4,3-d][1,3]thiazol-2-amine)
4-Methyl-N-(4-methylpyrimidin-2-yl)-6,7-dihydro-4H-pyrazolo[4′,3′:5,6]oxepino[4,3-d][1,3]thiazol-2-amine
4,6-Dimethyl-N-(4-methylpyrimidin-2-yl)-6,7-dihydro-4H-pyrazolo[4′,3′:5,6]oxepino-[4,3-d][1,3]thiazol-2-amine
N-(Pyrimidin-2-yl)-4,5,6,7-tetrahydropyrazolo[3,4-c][1,3]thiazolo[4,5-e]azepin-2-amine
5-Methyl-N-(4-methylpyrimidin-2-yl)-4,5,6,7-tetrahydropyrazolo[3,4-c][1,3]thiazolo[4,5-e]azepin-2-amine
N-(5-Fluoropyrimidin-2-yl)-4,5,6,7-tetrahydropyrazolo[3′,4′:6,7]cyclohepta[1,2-d][1,3]thiazol-2-amine
(5,5-Dimethyl-4,5,6,7-tetrahydro-3-thia-1,7,8-triaza-cyclopenta[e]azulen-2-yl)-(5-fluoro-pyrimidin-2-yl)-amine
6,6-Difluoro-N-(4-methylpyrimidin-2-yl)-4,5,6,7-tetrahydropyrazolo[3′,4′:6,7]cyclohepta[1,2-d][1,3]thiazol-2-amine
2-[(4-Methylpyrimidin-2-yl)amino]-4,7-dihydropyrazolo[3′,4′:6,7]cyclohepta[1,2-d][1,3]thiazol-6(5H)-one
5,5-Dimethyl-2-[(4-methylpyrimidin-2-yl)amino]-4,7-dihydropyrazolo[3′,4′:6,7]cyclohepta[1,2-d][1,3]thiazol-6(5H)-one
(6-Methyl-6,7-dihydro-4H-5-oxa-3-thia-1,7,8-triaza-cyclopenta[e]azulen-2-yl)-(5-methyl-[1,2,4]thiadiazol-3-yl)-amine and
(5-Fluoro-4,5,6,7-tetrahydro-3-thia-1,7,8-triaza-cyclopenta[e]azulen-2-yl)-(5-methyl-pyrimidin-2-yl)-amine.

Particularly relevant to the present invention is the tautomeric pair that exists for the pyrazole ring, illustrated below:

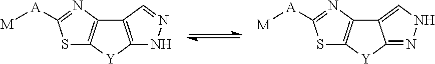

In this specification, reference to a generic formula or a compound as such indicating one tautomer is to be understood to refer to the tautomeric pair and the other tautomer thereof.

The disclosed compounds also include all pharmaceutically acceptable isotopic variations, in which at least one atom is replaced by an atom having the same atomic number, but an atomic mass different from the atomic mass usually found in nature. Examples of isotopes suitable for inclusion in the disclosed compounds include, without limitation, isotopes of hydrogen, such as ²H and ³H; isotopes of carbon, such as ¹³C and ¹⁴C; isotopes of nitrogen, such as ¹⁵N; isotopes of oxygen, such as ¹⁷O and ¹⁸O; isotopes of phosphorus, such as ³²P and ³³P; isotopes of sulfur, such as ³⁵S; isotopes of fluorine, such as ¹⁸F; and isotopes of chlorine, such as ³⁶Cl. Use of isotopic variations (e.g., deuterium, ²H) may afford certain therapeutic advantages resulting from greater metabolic stability, for example, increased in vivo half-life or reduced dosage requirements. Additionally, certain isotopic variations of the disclosed compounds may incorporate a radioactive isotope (e.g., tritium, ³H, or ¹⁴C), which may be useful in drug and/or substrate tissue distribution studies. Substitution with positron emitting isotopes, such as ¹¹C, ¹⁸F, ¹⁵O and ¹³N, can be useful in Positron Emission Topography (PET) studies for examining substrate receptor occupancy. Isotopically-labelled compounds of Formula (I) to (III) can generally be prepared by conventional techniques known to those skilled in the art or by processes analogous to those described in the accompanying Examples using appropriate isotopically-labeled reagents in place of the non-labeled reagent previously employed.

### DEFINITION OF TERMS

Listed below are definitions of various terms used in the specification and claims to describe the present invention.

For the avoidance of doubt it is to be understood that in this specification “(C₁-C₆)” means a carbon radical having 1, 2, 3, 4, 5 or 6 carbon atoms. “(C₀-C₆)” means a carbon radical having 0, 1, 2, 3, 4, 5 or 6 carbon atoms. In this specification “C” means a carbon atom, “N” means a nitrogen atom, “O” means an oxygen atom and “S” means a sulphur atom.

In the case where a subscript is the integer 0 (zero) the radical to which the subscript refers, indicates that the radical is absent, i.e. there is a direct bond between the radicals.

In the case where a subscript is the integer 0 (zero) and the radical to which the subscript refers is alkyl, this indicates the radical is a hydrogen atom.

In this specification, unless stated otherwise, the term “bond” refers to a saturated covalent bond. When two or more bonds are adjacent to one another, they are assumed to be equal to one bond. For example, a radical -A-B-, wherein both A and B may be a bond, the radical is depicting a single bond.

In this specification, unless stated otherwise, the term “alkyl” includes both straight and branched chain alkyl radicals and may be methyl, ethyl, n-propyl, i-propyl, n-butyl, i-butyl, s-butyl, t-butyl, n-pentyl, i-pentyl, t-pentyl, neo-pentyl, n-hexyl, i-hexyl or t-hexyl. The term “(C₀-C₃)alkyl” refers to an alkyl radical having 0, 1, 2 or 3 carbon atoms and may be methyl, ethyl, n-propyl and i-propyl.

In this specification, unless stated otherwise, the term “alkylene” includes both straight and branched difunctional saturated hydrocarbon radicals and may be methylene, ethylene, n-propylene, i-propylene, n-butylene, i-butylene, s-butylene, t-butylene, n-pentylene, i-pentylene, t-pentylene, neo-pentylene, n-hexylene, i-hexylene or t-hexylene.

In this specification, unless stated otherwise, the term “cycloalkyl” refers to an optionally substituted carbocycle containing no heteroatoms, including mono-, bi-, and tricyclic saturated carbocycles, as well as fused ring systems. Such fused ring systems can include one ring that is partially or fully unsaturated such as a benzene ring to form fused ring systems such as benzo-fused carbocycles. Cycloalkyl includes such fused ring systems as spirofused ring systems. Examples of cycloalkyl include cyclopropyl, cyclobutyl, cyclopentyl, cyclohexyl, decahydronaphthalene, adamantane, indanyl, fluorenyl and 1,2,3,4-tetrahydronaphthalene and the like. The term “(C₃-C₇)cycloalkyl” may be cyclopropyl, cyclobutyl, cyclopentyl, cyclohexyl, cycloheptyl and the like.

The term “aryl” refers to an optionally substituted monocyclic or bicyclic hydrocarbon ring system containing at least one unsaturated aromatic ring. Examples and suitable values of the term “aryl” are phenyl, naphthyl, 1,2,3,4-tetrahydronaphthyl, indyl, indenyl and the like.

In this specification, unless stated otherwise, the term “heteroaryl” refers to an optionally substituted monocyclic or bicyclic unsaturated, aromatic ring system containing at least one heteroatom selected independently from N, O or S. Examples of “heteroaryl” may be, but are not limited to thienyl, pyridinyl, thiazolyl, isothiazolyl, furyl, pyrrolyl, triazolyl, imidazolyl, triazinyl, oxadiazolyl, oxazolyl, isoxazolyl, pyrazolyl, imidazolonyl, oxazolonyl, thiazolonyl, tetrazolyl, thiadiazolyl, benzoimidazolyl, benzooxazolyl, benzothiazolyl, tetrahydrotriazolopyridinyl, tetrahydrotriazolopyrimidinyl, benzofuryl, benzothiophenyl, thionaphthyl, indolyl, isoindolyl, pyridonyl, pyridazinyl, pyrazinyl, pyrimidinyl, quinolyl, phtalazinyl, naphthyridinyl, quinoxalinyl, quinazolyl, imidazopyridinyl, oxazolopyridinyl, thiazolopyridinyl, imidazopyridazinyl, oxazolopyridazinyl, thiazolopyridazinyl, cynnolyl, pteridinyl, furazanyl, benzotriazolyl, pyrazolopyridinyl and purinyl.

In this specification, unless stated otherwise, the term “alkylene-aryl”, “alkylene-heteroaryl” and “alkylene-cycloalkyl” refers respectively to a substituent that is attached via the alkyl radical to an aryl, heteroaryl or cycloalkyl radical, respectively. The term “(C₁-C₆)alkylene-aryl” includes aryl-C₁-C₆-alkyl radicals such as benzyl, 1-phenylethyl, 2-phenylethyl, 1-phenylpropyl, 2-phenylpropyl, 3-phenylpropyl, 1-naphthylmethyl and 2-naphthylmethyl. The term “(C₁-C₆)alkylene-heteroaryl” includes heteroaryl-C₁-C₆-alkyl radicals, wherein examples of heteroaryl are the same as those illustrated in the above definition, such as 2-furylmethyl, 3-furylmethyl, 2-thienylmethyl, 3-thienylmethyl, 1-imidazolylmethyl, 2-imidazolylmethyl, 3-imidazolylmethyl, 2-oxazolylmethyl, 3-oxazolylmethyl, 2-thiazolylmethyl, 3-thiazolylmethyl, 2-pyridinylmethyl, 3-pyridinylmethyl, 4-pyridinylmethyl, 1-quinolylmethyl or the like.

In this specification, unless stated otherwise, the term “heterocycle” refers to an optionally substituted, monocyclic or bicyclic saturated, partially saturated or unsaturated ring system containing at least one heteroatom selected independently from N, O and S.

In this specification, unless stated otherwise, a 5- or 6-membered ring containing one or more atoms independently selected from C, N, O and S, includes aromatic and heteroaromatic rings as well as carbocyclic and heterocyclic rings which may be saturated or unsaturated. Examples of such rings may be, but are not limited to, furyl, isoxazolyl, isothiazolyl, oxazolyl, pyrazinyl, pyrazolyl, pyridazinyl, pyridinyl, pyrimidinyl, pyrrolyl, thiazolyl, thienyl, imidazolyl, imidazolidinyl, imidazolinyl, triazolyl, morpholinyl, piperazinyl, piperidyl, piperidonyl, pyrazolidinyl, pyrazolinyl, pyrrolidinyl, pyrrolinyl, tetrahydropyranyl, tetrahydrothiopyranyl, oxazolidinonyl, thiomorpholinyl, oxadiazolyl, thiadiazolyl, tetrazolyl, phenyl, cyclohexyl, cyclopentyl, cyclohexenyl and cyclopentenyl.

In this specification, unless stated otherwise, a 3- to 10-membered ring containing one or more atoms independently selected from C, N, O and S, includes aromatic and heteroaromatic rings as well as carbocyclic and heterocyclic rings which may be saturated or unsaturated. Examples of such rings may be, but are not limited to imidazolidinyl, imidazolinyl, morpholinyl, piperazinyl, piperidyl, piperidonyl, pyrazolidinyl, pyrazolinyl, pyrrolidinyl, pyrrolinyl, tetrahydropyranyl, thiomorpholinyl, tetrahydrothiopyranyl, furyl, pyrrolyl, dihydropyrrolyl isoxazolyl, isothiazolyl, isoindolinonyl, dihydropyrrolo[1,2-b]pyrazolyl, oxazolyl, oxazolidinonyl, pyrazinyl, pyrazolyl, pyridazinyl, pyridinyl, tetrahydropyridinyl, pyrimidinyl, pyrrolyl, thiazolyl, thienyl, imidazolyl, triazolyl, phenyl, cyclopropyl, aziridinyl, cyclobutyl, azetidinyl, oxadiazolyl, thiadiazolyl, tetrazolyl, cyclopentyl, cyclopentenyl, cyclohexyl, cyclohexenyl, cycloheptyl, cycloheptenyl, cyclooctyl and cyclooctenyl.

In this specification, unless stated otherwise, the term “halo” or “halogen” may be fluoro, chloro, bromo or iodo.

In this specification, unless stated otherwise, the term “haloalkyl” means an alkyl radical as defined above, substituted with one or more halo radicals. The term “(C₁-C₆)haloalkyl” may include, but is not limited to, fluoromethyl, difluoromethyl, trifluoromethyl, fluoroethyl and difluoroethyl. The term “O—C₁-C₆-haloalkyl” may include, but is not limited to, fluoromethoxy, difluoromethoxy, trifluoromethoxy and fluoroethoxy.

In this specification, unless stated otherwise, the term “haloalkylene” means an alkylene radical as defined above, substituted with one or more halo radicals. The term “(C₁-C₆)haloalkylene” may include, but is not limited to, fluoromethylene, difluoromethylene, fluoroethylene and difluoroethylene. The term “O—C₁-C₆-haloalkylene” may include, but is not limited to, fluoromethylenoxy, difluoromethylenoxy and fluoroethylenoxy.

In this specification, unless stated otherwise, the term “cyanoalkyl” means an alkyl radical as defined above, substituted with one or more cyano.

In this specification, unless stated otherwise, the term “optionally substituted” refers to radicals further bearing one or more substituents which may be, (C₁-C₆)alkyl, hydroxy, (C₁-C₆)alkylene-oxy, mercapto, aryl, heterocycle, heteroaryl, (C₁-C₆)alkylene-aryl, (C₁-C₆)alkylene-heterocycle, (C₁-C₆)alkylene-heteroaryl, halogen, trifluoromethyl, pentafluoroethyl, cyano, cyanomethyl, nitro, amino, amido, amidinyl, carboxyl, carboxamide, (C₁-C₆)alkylene-oxycarbonyl, carbamate, sulfonamide, ester and sulfonyl.

In this specification, unless stated otherwise, the term “solvate” refers to a complex of variable stoichiometry formed by a solute (e.g. a compound of Formula (I)) and a solvent. The solvent is a pharmaceutically acceptable solvent as preferably water; such solvent may not interfere with the biological activity of the solute.

In this specification, unless stated otherwise, the term “positive allosteric modulator of mGluR₄” or “allosteric modulator of mGluR₄” refers also to a pharmaceutically acceptable acid or base addition salt thereof, a stereochemically isomeric form thereof and an N-oxide form thereof.

### Pharmaceutical Compositions

Allosteric modulators of mGluR₄ described herein, and the pharmaceutically acceptable salts, solvates and hydrates thereof can be used in pharmaceutical preparations in combination with a pharmaceutically acceptable carrier or diluent. Suitable pharmaceutically acceptable carriers include inert solid fillers or diluents and sterile aqueous or organic solutions. The allosteric modulators of mGluR₄ will be present in such pharmaceutical compositions in amounts sufficient to provide the desired dosage amount in the range described herein. Techniques for formulation and administration of the compounds of the instant invention can be found in Remington: the Science and Practice of Pharmacy, 19^{th} edition, Mack Publishing Co., Easton, Pa. (1995).

The amount of allosteric modulators of mGluR₄, administered to the subject will depend on the type and severity of the disease or condition and on the characteristics of the subject, such as general health, age, sex, body weight and tolerance to drugs. The skilled artisan will be able to determine appropriate dosages depending on these and other factors. Effective dosages for commonly used CNS drugs are well known to the skilled person. The total daily dose usually ranges from about 0.05-2000 mg.

The present invention relates to pharmaceutical compositions which provide from about 0.01 to 1000 mg of the active ingredient per unit dose. The compositions may be administered by any suitable route. For example, orally in the form of capsules and the like, parenterally in the form of solutions for injection, topically in the form of onguents or lotions, ocularly in the form of eye-drops, rectally in the form of suppositories, intranasally or transcutaneously in the form of delivery system like patches.

For oral administration, the allosteric modulators of mGluR₄ thereof can be combined with a suitable solid or liquid carrier or diluent to form capsules, tablets, pills, powders, syrups, solutions, suspensions and the like.

The tablets, pills, capsules, and the like contain from about 0.01 to about 99 weight percent of the active ingredient and a binder such as gum tragacanth, acacias, corn starch or gelatin; excipients such as dicalcium phosphate; a disintegrating agent such as corn starch, potato starch, alginic acid, a lubricant such as magnesium stearate; and a sweetening agent such as sucrose, lactose or saccharin. When a dosage unit form is a capsule, it may contain, in addition to materials of the above type, a liquid carrier such as a fatty oil.

Various other materials may be present as coatings or to modify the physical form of the dosage unit. For instance, tablets may be coated with shellac, sugar or both. A syrup or elixir may contain, in addition to the active ingredient, sucrose as a sweetening agent, methyl and propylparabens as preservatives, a dye and a flavoring such as cherry or orange flavor.

For parenteral administration the disclosed allosteric modulators of mGluR₄ can be combined with sterile aqueous or organic media to form injectable solutions or suspensions. For example, solutions in sesame or peanut oil, aqueous propylene glycol and the like can be used, as well as aqueous solutions of water-soluble pharmaceutically-acceptable salts of the compounds. Dispersions can also be prepared in glycerol, liquid polyethylene glycols and mixtures thereof in oils. Under ordinary conditions of storage and use, these preparations contain a preservative to prevent the growth of microorganisms.

In addition, to the formulations described previously, the compounds may also be formulated as a depot preparation. Such long acting formulations may be administered for example, by subcutaneously implantation or by intramuscular injection. Thus, for example, as an emulsion in an acceptable oil, or ion exchange resins, or as sparingly soluble derivatives, for example, as sparingly soluble salts.

Preferably disclosed allosteric modulators of mGluR₄ or pharmaceutical formulations containing these compounds are in unit dosage form for administration to a mammal. The unit dosage form can be any unit dosage form known in the art including, for example, a capsule, an IV bag, a tablet, or a vial. The quantity of active ingredient in a unit dose of composition is an effective amount and may be varied according to the particular treatment involved. It may be appreciated that it may be necessary to make routine variations to the dosage depending on the age and condition of the patient. The dosage will also depend on the route of administration which may be by a variety of routes including oral, aerosol, rectal, transdermal, subcutaneous, intravenous, intramuscular, intraperitoneal and intranasal.

Classical treatment of Parkinsonism typically involves the use of levodopa combined with carbidopa (SINEMET™) or benserazide (MADOPAR™). Dopamine agonists such as bromocriptine (PARLODEL™), lisuride and pergolide (CELANCE™) act directly on dopamine receptors and are also used for the treatment of Parkinsonism.

### Methods of Synthesis

The compounds according to the invention, in particular the compounds according to the Formula (I) to (III), may be prepared by methods known in the art of organic synthesis as set forth in part by the following synthesis schemes. In all of the schemes described below, it is well understood that protecting groups for sensitive or reactive groups are employed where necessary in accordance with general principles of chemistry. Protecting groups are manipulated according to standard methods of organic synthesis (Green T. W. and Wuts P. G. M., (1991) Protecting Groups in Organic Synthesis, John Wiley & Sons). These groups are removed at a convenient stage of the compound synthesis using methods that are readily apparent to those skilled in the art. The selection of process as well as the reaction conditions and order of their execution shall be consistent with the preparation of compounds of Formula (I) to (III).

The compounds according to the invention may be represented as a mixture of enantiomers, which may be resolved into the individual pure R- or S-enantiomers. If for instance, a particular enantiomer is required, it may be prepared by asymmetric synthesis or by derivation with a chiral auxiliary, where the resulting diastereomeric mixture is separated and the auxiliary group cleaved to provide the pure desired enantiomers. Alternatively, where the molecule contains a basic functional group such as an amino or an acidic functional group such as carboxyl, this resolution may be conveniently performed by fractional crystallization from various solvents as the salts of an optical active acid or by other methods known in the literature (e.g. chiral column chromatography).

Resolution of the final product, an intermediate or a starting material may be performed by any suitable method known in the art (Eliel E. L., Wilen S. H. and Mander L. N., (1984) Stereochemistry of Organic Compounds, Wiley-Interscience).

Many of the heterocyclic compounds of the invention can be prepared using synthetic routes well known in the art (Katrizky A. R. and. Rees C. W., (1984) Comprehensive Heterocyclic Chemistry, Pergamon Press).

The product from the reaction can be isolated and purified by employing standard techniques, such as extraction, chromatography, crystallization and distillation.

The compounds of the invention may be prepared by general route of synthesis as disclosed in the following methods.

In one embodiment of the present invention compounds of Formula (III) may be prepared according to the synthetic sequences illustrated in Scheme 1. Pyrazole g1 can be iodinated in the presence of diiodine and diammonium cerium (IV) nitrate in a solvent such as acetonitrile. Then compound g2 can be protected by p-methoxybenzyl, for example, using standard conditions followed by the reduction of the ketone g3 into the corresponding alcohol g4 in the presence of NaBH₄ and the like. The compound g4 can be converted into the boronic ester g5 which can be coupled using Suzuki conditions to an amino protected bromothiazole. The resulting compound g6 was reduced in the presence of NaBH₄. The diol g7 can be cyclized and deprotected into the oxepane g8 under acidic condition such as sulfuric acid. Primary amine g8 can be coupled to heteroaryl halide M-X, using Buchwald conditions with a catalyst such as Pd₂(dba)₃, a ligand such as Xantphos and a base such as Cs₂CO₃ to yield compound g9 which can be finally deprotected in the presence of TFA to yield compound g10.

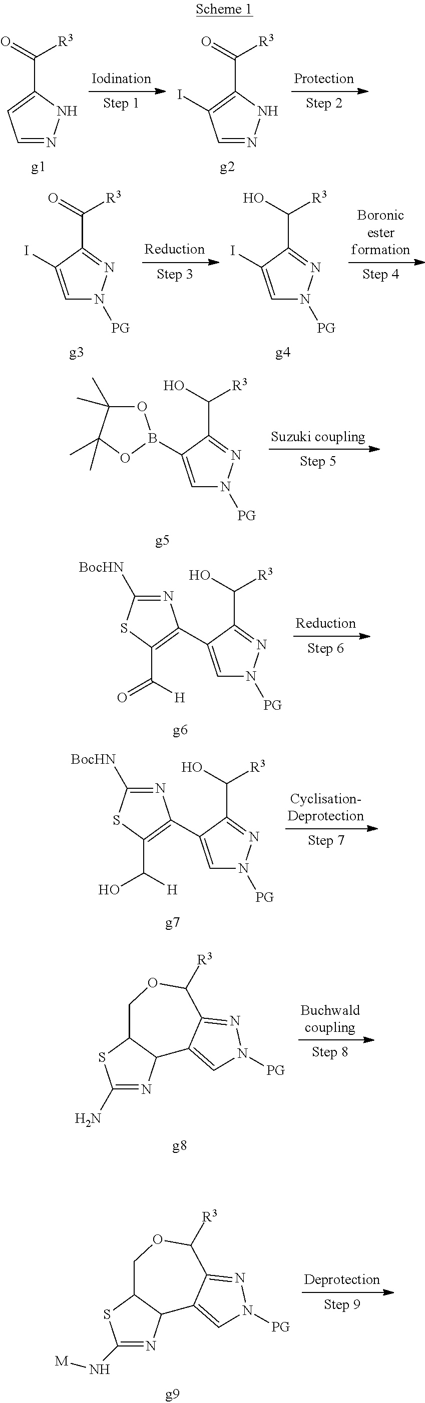

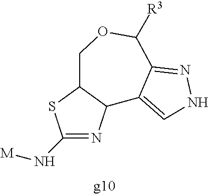

In one embodiment of the present invention compounds of Formula (III) may be prepared according to the synthetic sequences illustrated in Scheme 2. Ketone g11 can be converted into the tertiary alcohol g12 by the addition of methyllithium in a solvent such as THF and the like, and at a temperature such as −78° C. The resulting diol g12 can afford compounds of Formula (III) following the Scheme 1, from Step 7 described above.

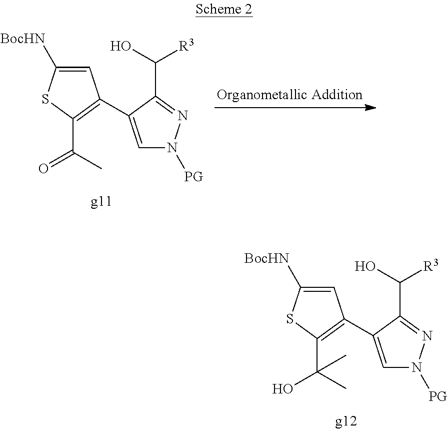

In one embodiment of the present invention compounds of Formula (III) may be prepared according to the synthetic sequences illustrated in Scheme 3. The ester g13 can be reduced into the primary alcohol g14 in the presence of a reducing agent such as LiBH₄ and in a solvent such as THF. The resulting alcohol g14 can afford compounds of Formula (III) following the Scheme 1, from Step 4 described above.

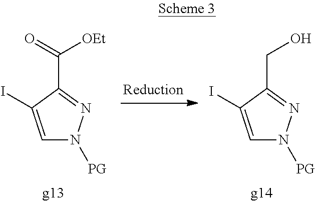

In one embodiment of the present invention compounds of Formula (III) may be prepared according to the synthetic sequences illustrated in Scheme 4. Pyrazole g15 can be protected by p-methoxybenzyl, for example, using standard conditions. Then compound g16 may be hydrolyzed and the resulting carboxylic acid g17 can be transformed into the corresponding Weinreb amide g18. Functionalized pyrazole g19 can be obtained from deprotonation of pyrazole g18 using LDA as a base in THF at −78° C. followed by the addition of an aldehyde. Vinyl Grignard reagent can be added on the Weinreb amide g19 to generate the compound g20 which can undergo addition of carbonic acid diethylester which after treatment under acidic condition yielded the keto-ester g21. Bromination of g21 can be done in the presence of trimethylphenylammonium tribromide to yield g22 which in the presence of thiourea can be converted into the aminothiazole g23. The ester g23 can be reduced into the primary alcohol g24 in the presence of a reducing agent such as DIBAL and in a solvent such as DCM and the like. The resulting alcohol g24 can afford compounds of Formula (III) following the Scheme 1 described above.

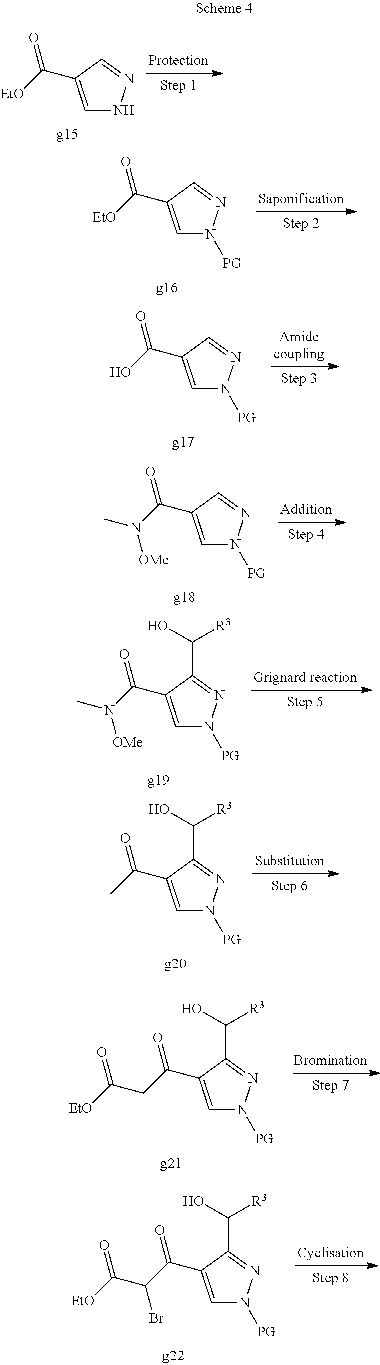

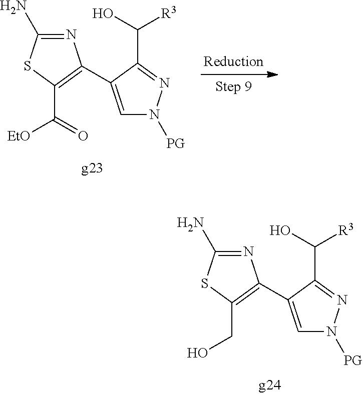

In one embodiment of the present invention compounds of Formula (III) may be prepared according to the synthetic sequences illustrated in Scheme 5. Primary amine g24 can be coupled to heteroaryl halide M-X, using Buchwald conditions with a catalyst such as Pd₂(dba)₃, a ligand such as Xantphos and a base such as Cs₂CO₃ to yield compound g25. The resulting diol g25 can be cyclized into the oxepane g10 under acidic condition using for example TFA.

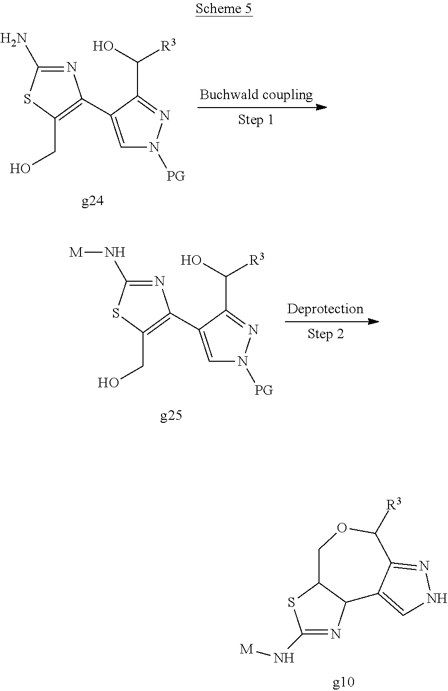

In one embodiment of the present invention compounds of Formula (III) may be prepared according to the synthetic sequences illustrated in Scheme 6. Brominated keto-ester g22 can be converted into the amino thiazole g26 in the presence of the corresponding substituted thiourea in a protic solvent such as ethanol and at a temperature such as 78° C. Then the ester g26 can be reduced into the primary alcohol g25 in the presence of a reducing agent such as DIBAL and in a solvent such as DCM. The resulting alcohol g25 can afford compounds of Formula (III) following the Scheme 5 described above.

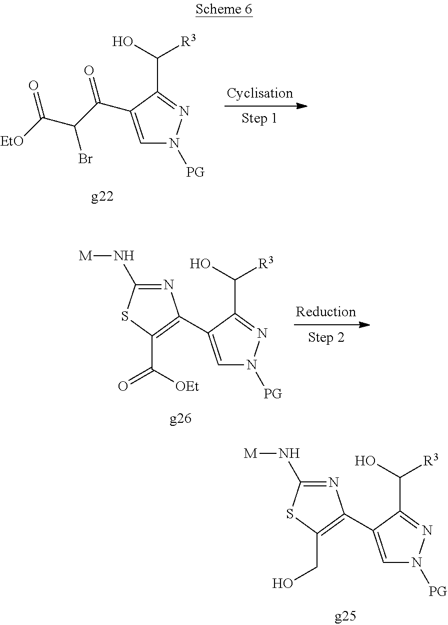

In one embodiment of the present invention compounds of Formula (III) may be prepared according to the synthetic sequences illustrated in Scheme 7. The ester g27 may be hydrolyzed and the resulting carboxylic acid g28 can be transformed into the corresponding Weinreb amide g29 via the acid chloride. Functionalized pyrazole g30 can be obtained from the Suzuki coupling of the brominated compound g29 and a boronate in the presence of a catalyst such as Pd(dppf)Cl₂, with a base such as Cs₂CO₃. The resulting alkene g30 can be oxidized into the tertiairy alcohol g31 in the presence of Hg(OAc)₂ followed by the treatment of a base such as NaOH and finally with a reducing agent such as NaBH₄. Vinyl Grignard reagent can be added to the Weinreb amide g31 to generate the compound g32 which can undergo cyclization in the presence of a Lewis acid such as BF₃.OEt₂. Subsequently, ketone g33 can be transformed into bromoketone g34 which in the presence of substituted thiourea can be cyclized into an aminothiazole g35. Finally, the expected oxepane g36 can be obtained via deprotection in the presence of TFA.

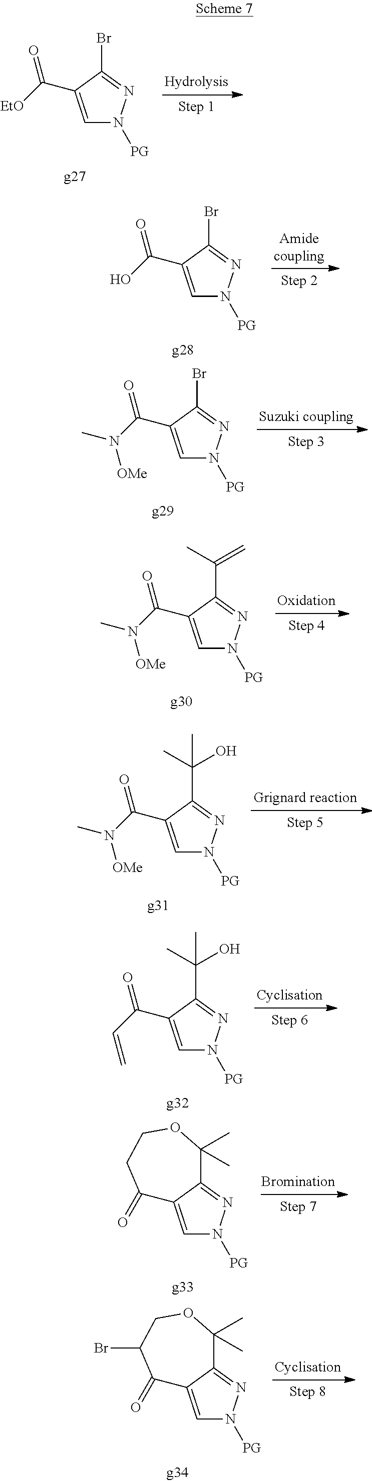

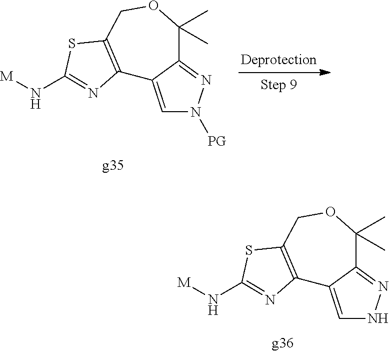

In one embodiment of the present invention compounds of Formula (III) may be prepared according to the synthetic sequences illustrated in Scheme 8. Functionalized pyrazole g37 can be obtained from the Stille coupling of the brominated compound g29 and tributyl(vinyl)tin in the presence of a catalyst such as Pd(PPh₃)₄ and in a solvent such as toluene under reflux condition. After standard ozonolyzis of the alkene g37, the resulting aldehyde g38 can be converted into the tertiary alcohol g38a in the presence of the addition of Ruppert's reagent (TMSCF₃) and TBAF in a solvent such as THF and the like. The resulting Weinreb amide g38a can afford compounds of Formula (III) following the Scheme 7 described above.

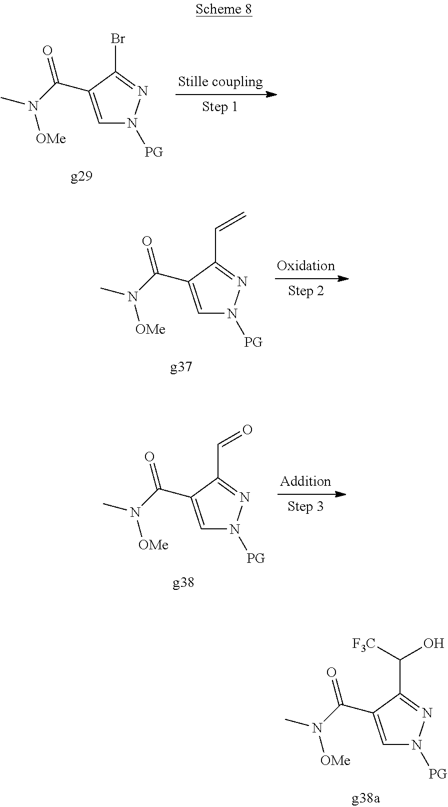

In one embodiment of the present invention compounds of Formula (III) may be prepared according to the synthetic sequences illustrated in Scheme 9. Aldehyde g38 can be converted into the secondary alcohol g19 by the addition of Grignard reagent in a solvent such as THF and the like, and at a temperature such as −30° C. The resulting alcohol g19 can afford compounds of Formula (III) following the Scheme 7 described above.

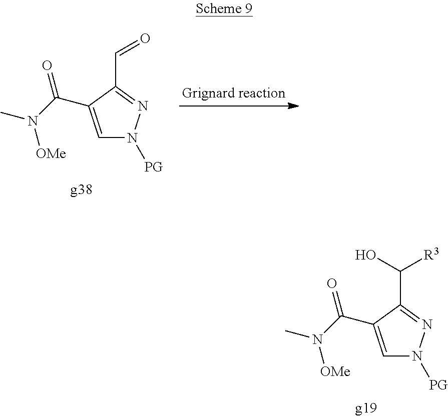

In one embodiment of the present invention compounds of Formula (II) may be prepared according to the synthetic sequences illustrated in Scheme 10. Pyrazole g40 can be synthesized from compound g39 and monosubstituted hydrazine in the presence of a base such as K₂CO₃. The hydroxyl moiety of compound g40 can be allylated with 3-bromopropene in the presence of a base such as K₂CO₃ and the like, in a solvent such as acetonitrile. The ester g41 may be hydrolyzed and the resulting carboxylic acid g42 can be transformed into the corresponding Weinreb amide g43. Vinyl Grignard reagent can be added on the Weinreb amide g43 to generate the compound g44 which can undergo metathesis using Grubbs catalysts. The resulting α,β-unsaturated ketone g45 can be reduced in the presence of hydrogen and Pd/C. Subsequently, ketone g46 can be transformed into bromoketone g47 under mild condition, which in the presence of substituted thiourea can be cyclized into an aminothiazole g48. Finally, the expected oxepane g49 can be obtained via deprotection in the presence of TFA.

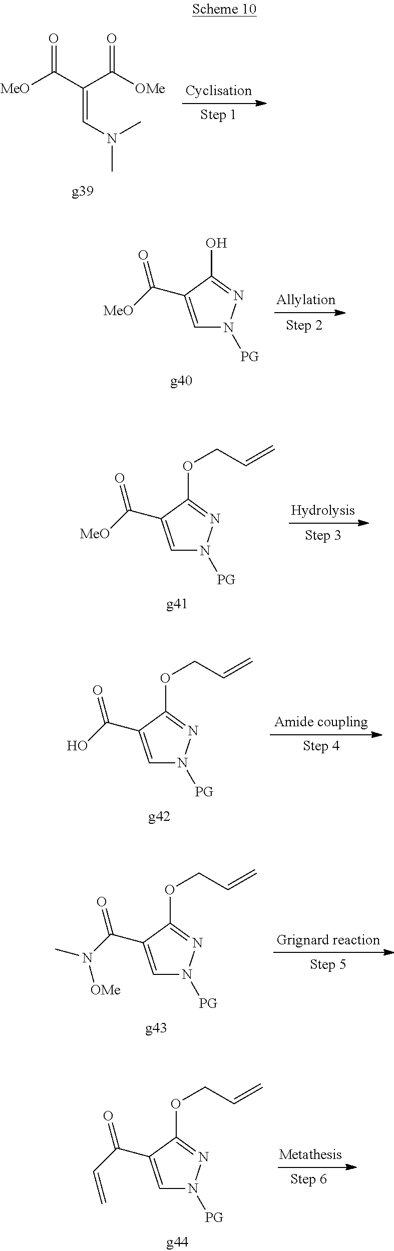

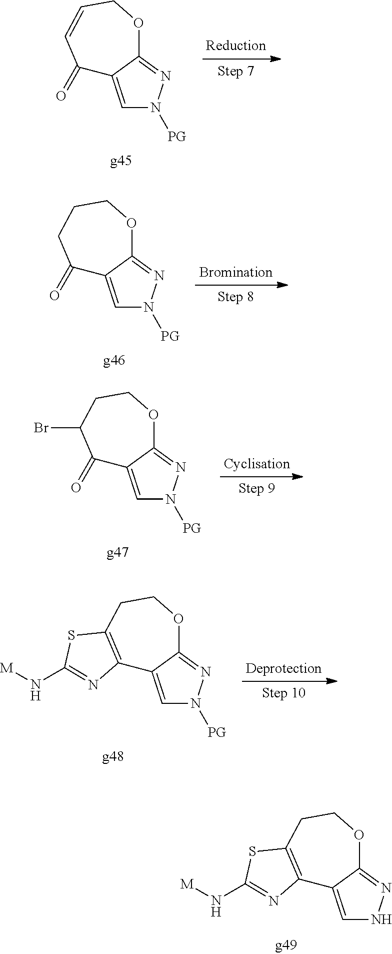

In one embodiment of the present invention compounds of Formula (II) may be prepared according to the synthetic sequences illustrated in Scheme 11. Grignard reagent can be added on the Weinreb amide g37 to generate the compound g50 which can undergo metathesis using Grubbs catalysts. The resulting alkene g51 can be epoxidized in the presence of m-CPBA in a solvent such as DCM, the epoxide g52 can be then open in the presence of Pd/C under an hydrogen atmosphere. The resulting primary alcohol can be converted into the carbamic ester in the presence of a base such as NaH. The resulting ketone g54 can afford compounds of Formula (II) following the synthesis of aminothiazoles described in Scheme 7, from Step 7.

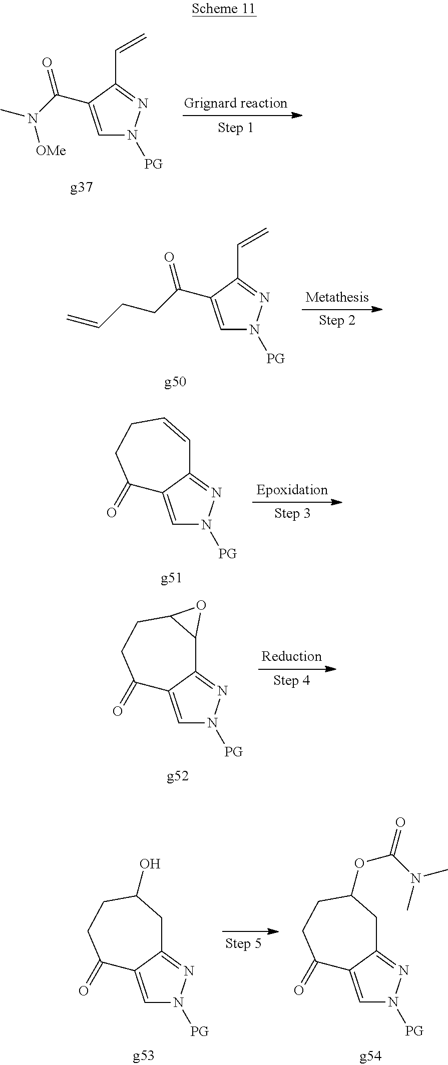

In one embodiment of the present invention compounds of Formula (II) may be prepared according to the synthetic sequences illustrated in Scheme 12. Primary alcohol g55 can be converted in the ether g56 in the presence of an alkyl agent such as methyl iodide and the like in the presence of a base such as NaH in an aprotic solvent such as THF. The resulting ketone g56 can afford compounds of Formula (II) following the Scheme 7, from Step 7 described above.

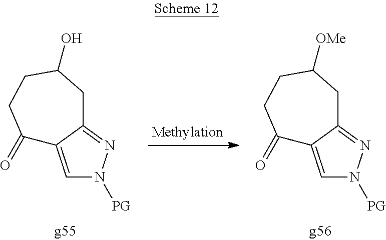

In one embodiment of the present invention compounds of Formula (II) may be prepared according to the synthetic sequences illustrated in Scheme 13. Primary alcohol g55 can be oxidized in the corresponding ketone g57 in the presence of an oxidizing agent such as PCC in a solvent such as DCM. The resulting ketone g57 can be treated with DAST to yield the difluoromethylene compound g58 which can afford compounds of Formula (II) following the Scheme 7 described above.

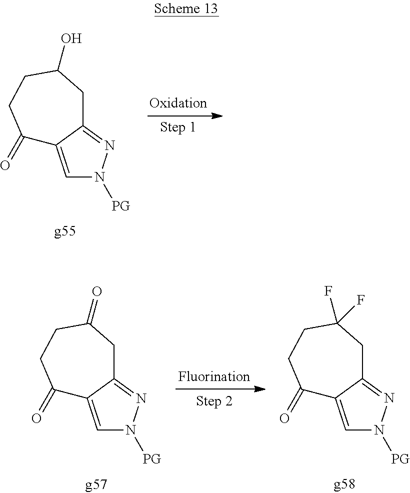

In one embodiment of the present invention compounds of Formula (II) may be prepared according to the synthetic sequences illustrated in Scheme 14. The ester g59 can be reduced into the aldehyde g60 in the presence of a reducing agent such as DIBAL and in a solvent such as DCM and the like. Aldehyde g60 can be converted into the secondary alcohol g61 by the addition of Grignard reagent in a solvent such as THF and the like, and at a temperature such as −78° C. The compound g61 can be converted into the boronic ester which can be coupled using Suzuki conditions to an amino protected bromothiazole. The double bond of compound g62 can be reduced in the presence of PtO₂ under hydrogen atmosphere. Then the secondary alcohol g63 can be oxidized into the ketone g64 in the presence of an oxidizing agent such as PCC. The ketone g64 can be cyclized in basic conditions using NaOH in order to generate the α, β-unsaturated ketone g65 which can be fully reduced into the compound g66 in the presence of NaBH₄ followed by hydrogenation with PtO₂. Finally the aminothiazole g66 can be deprotected in acidic condition such as TFA or HCl to yield g67 which can afford compounds of Formula (II) following the Scheme 7 described above.

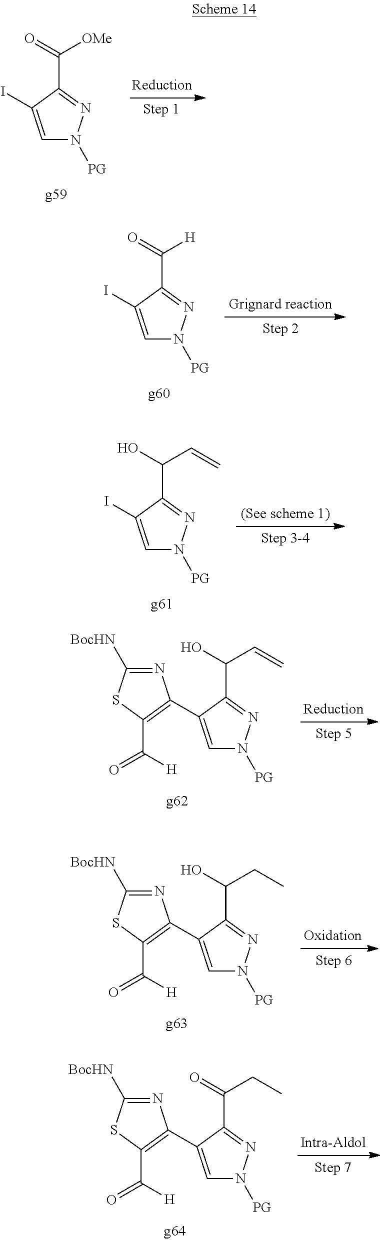

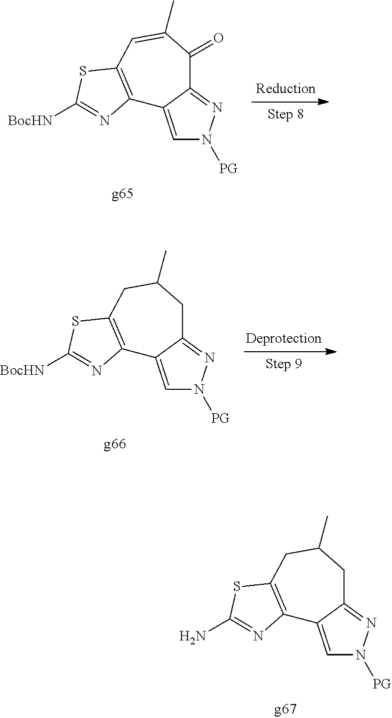

In one embodiment of the present invention compounds of Formula (II) may be prepared according to the synthetic sequences illustrated in Scheme 15. The ketone g68 can be converted into the tertiary alcohol g69 by the addition of MeLi, in a solvent such as THF and the like. The alcohol g69 can be reduced using triethylsilane followed by the addition of TFA to lead to the deprotected compound g70 which can afford compounds of Formula (II) following the Scheme 1, from Step 8, described above.

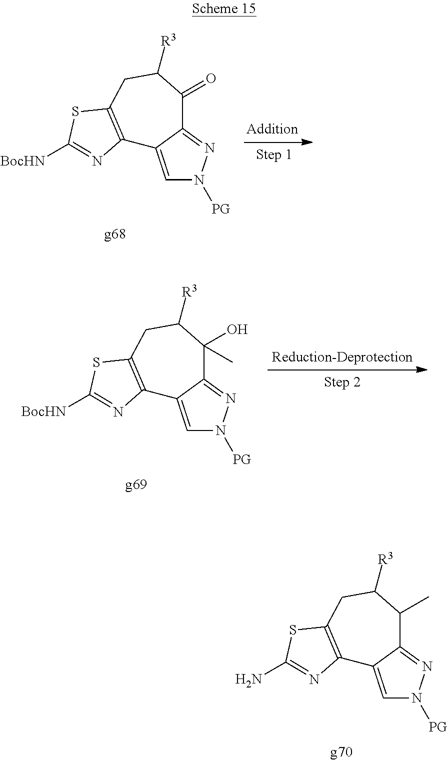

In one embodiment of the present invention compounds of Formula (II) may be prepared according to the synthetic sequences illustrated in Scheme 16. The iodopyrazole g59 can be converted into the boronic ester g71 and consequently can be coupled using Suzuki conditions to an amino protected bromothiazole. The compound g72 can be reduced into the compound g73 in the presence of DIBAL, in a solvent such as THF at a temperature such as −65° C. The resulting compound can be cyclized via intra-aldol to the cyclized α,β-unsaturated ketone g75 under basic conditions such as NaOH at a temperature such as 60° C. Finally, the α,β-unsaturated ketone g75 can be reduced in the presence of PtO₂ under hydrogen atmosphere to yield the ketone g76 which can afford compounds of Formula (II) following the Scheme 14, Step 9 and Scheme 1, Step 8, described above.

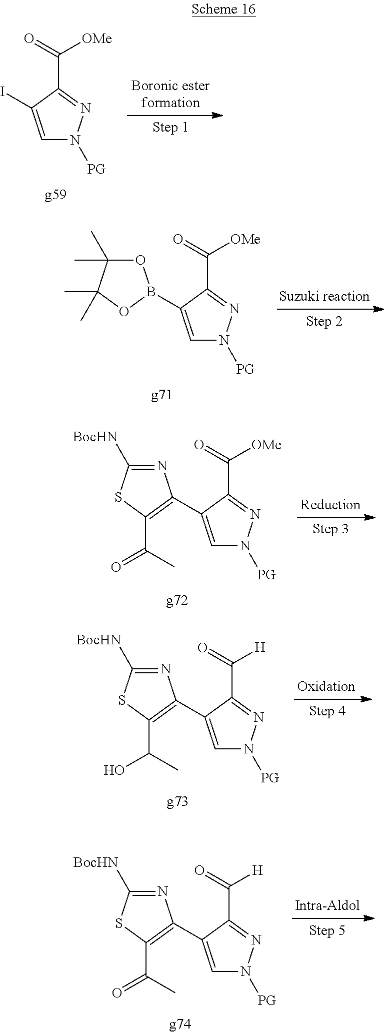

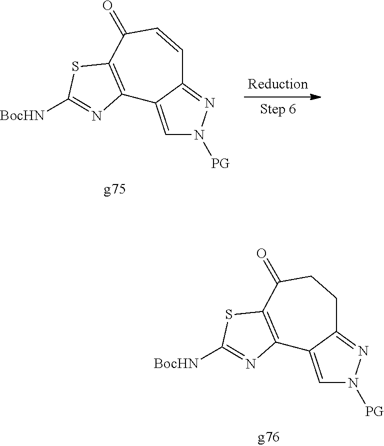

In one embodiment of the present invention compounds of Formula (II) may be prepared according to the synthetic sequences illustrated in Scheme 17. The α,β-unsaturated ketone g65 can be reduced using LiAlH₄ as reducing agent in an aprotic solvent such as THF to yield the alcohol g77 which can afford compounds of Formula (II) following the Schemes described above.

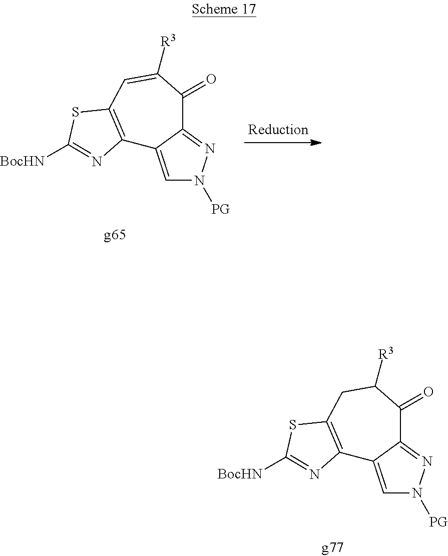

In one embodiment of the present invention compounds of Formula (II) may be prepared according to the synthetic sequences illustrated in Scheme 18. After reduction of the α,β-unsaturated ketone g78 (obtained using compound g60, which can react with methylmagnesium bromide following Step 2 in Scheme 4) into the ketone g79, the amino moiety can be deprotected under acidic condition such as TFA. The resulting primary amine g80 can be coupled to heteroaryl halide M-X, using Buchwald-type conditions with a catalyst such as Pd₂(dba)₃, a ligand such as Xantphos and a base such as Cs₂CO₃ to yield compound g81 which can be protected by p-methoxybenzyl group using standard conditions. After deprotonation using a base such as NaH, the ketone g82 can be dimethylated in the presence of iodomethyl. The resulting compound g83 can be fully deprotected under acidic conditions such as TFA. Finally the ketone g84 can be transformed in the presence of a fluorinating agent such as DAST, into the corresponding difluoromethylene g85.

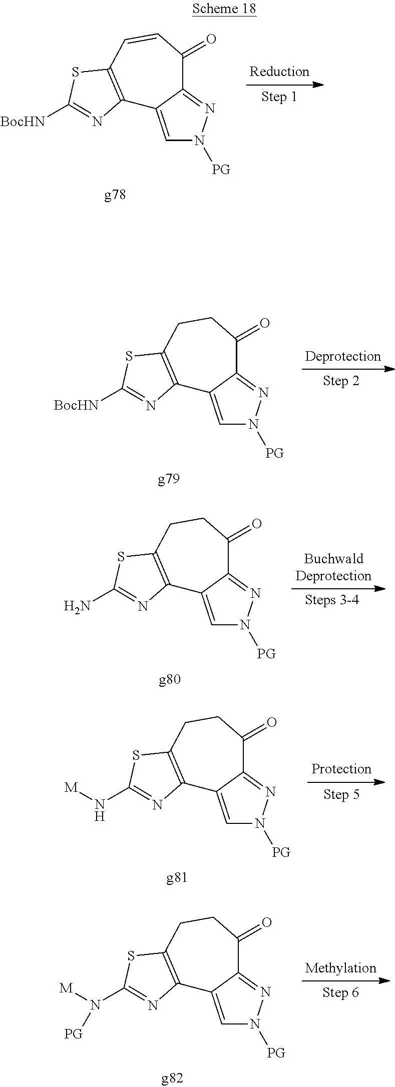

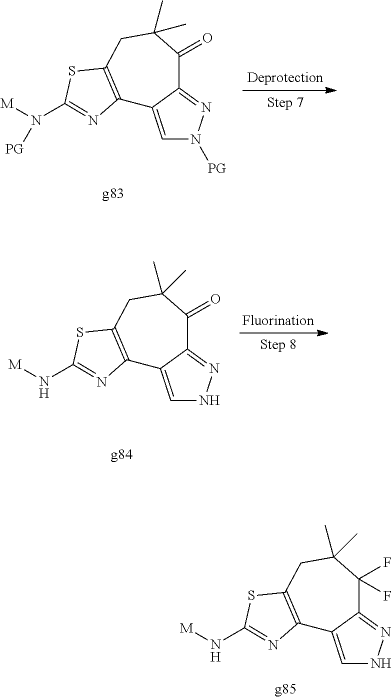

In one embodiment of the present invention compounds of Formula (II) may be prepared according to the synthetic sequences illustrated in Scheme 19. The amino moiety in compound g77 can be protected by p-methoxybenzylchloride using standard condition. Then the alcohol g86 can be converted into the ether g87 by methylation in the presence of methyliodide with a base such as NaH. The Boc can be removed under acidic condition such as TFA in a solvent such as DCM at room temperature. The resulting compound g88 can afford compounds of Formula (II) following Scheme 1 described above.

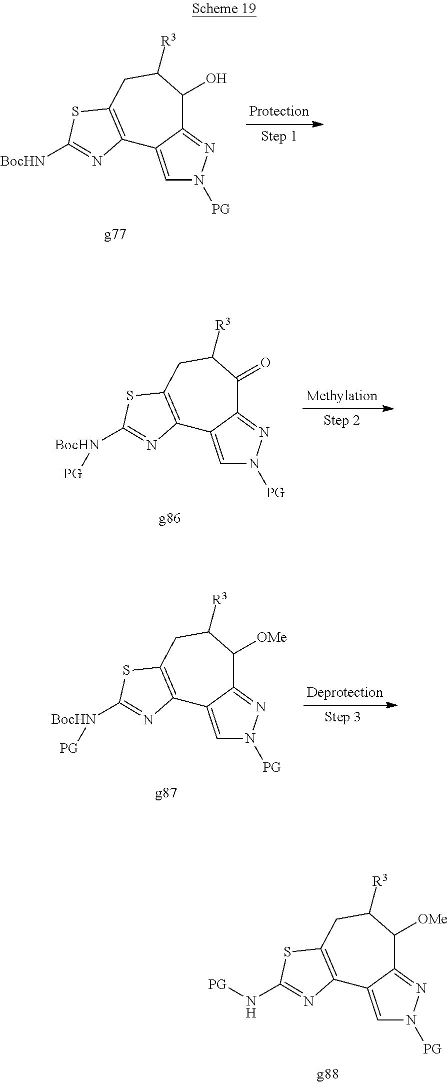

In one embodiment of the present invention compounds of Formula (II) may be prepared according to the synthetic sequences illustrated in Scheme 20. Addition of a CF₃ moiety on the ketone g79 can be performed by using TMSCF₃ followed by CsF in a solvent such as THF in order to lead to the compound g89. The alcohol moiety of the compound g89 can be substituted by a chlorine by using thionyl chloride. The resulting chlorine atom in compound g90 can be displaced by a hydride from LiBH₄. Finally the compound g91 can afford compounds of Formula (II) following the Schemes described above.

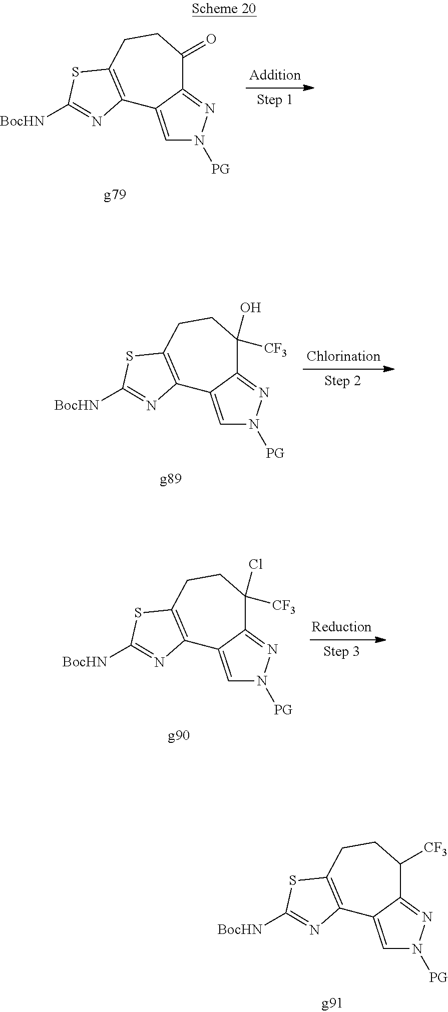

In one embodiment of the present invention compounds of Formula (II) may be prepared according to the synthetic sequences illustrated in Scheme 21. In the presence of 1,1-dimethoxy-N,N-dimethyl methanamine, the 1,3-diketone g92 can afford the compound g93 using standard condition. The resulting compound g93 can be cyclized into the pyrazole ring g94 with hydrazine in a solvent such as n-butanol by heating under reflux. The pyrazole g94 can be protected by p-methoxybenzyl as described above. Subsequently, ketone g95 can be transformed into bromoketone g97 in a reduction-bromination reaction sequence under mild condition, which in the presence of substituted thiourea can be cyclized into an aminothiazole g98. Finally, compounds of Formula (II) can be obtained after deprotection in the presence of TFA as described above.

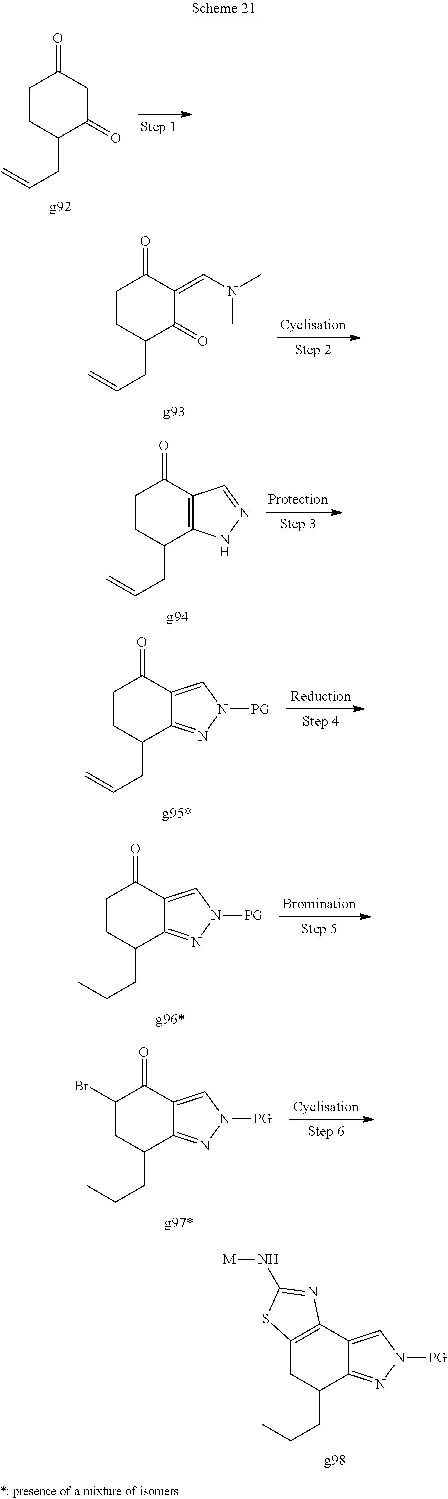

In one embodiment of the present invention compounds of Formula (II) may be prepared according to the synthetic sequences illustrated in Scheme 22. Grignard reagent can be added on the Weinreb amide g37 to generate the compound g99 which can undergo metathesis using Grubbs catalysts. The resulting alkene g100 can be reduced in the presence of Pd/C under hydrogen atmosphere to yield the ketone g101 which can afford compounds of Formula (II) following the Scheme 7 described above.

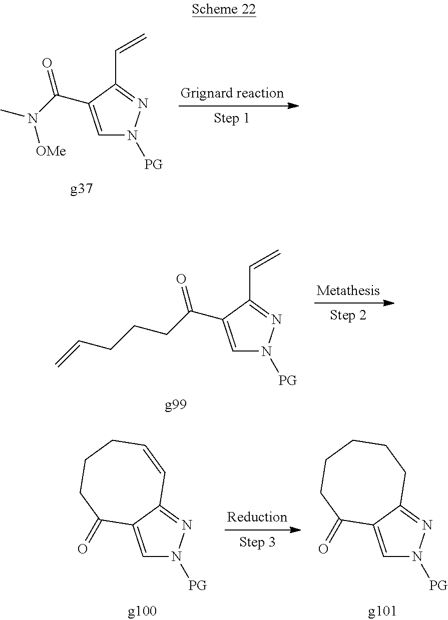

In one embodiment of the present invention compounds of Formula (II) may be prepared according to the synthetic sequences illustrated in Scheme 23. The tricyclic compound g102 (synthesized as in WO2010/079238) was oxidized in the presence of DDQ to afford compound g103.

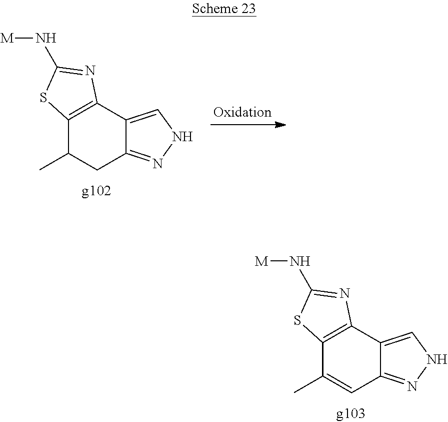

In general, substituted thiourea M-NH—(C═S)—NH₂ used in Schemes 6, 7, 10 and 21, are prepared according to methods known by persons skilled in the art. For example, 5-fluoropyrimidin-2-amine can be reacted with ethyl carbonisothiocyanatidate in acetonitrile, then the resulting product can be treated with ammonium formate in ammonia affording the thiourea 5-fluoropyrimidin-2-yl-NH(C═S)—NH₂.

### EXPERIMENTAL

Unless otherwise noted, all starting materials were obtained from commercial suppliers and used without further purification.

Specifically, the following abbreviations may be used in the examples and throughout the specification.

| | |
|---|---|
| AcOH (Acetic acid) | MgSO₄ (Magnesium sulfate) |
| BF₃•OEt₂ (Boron trifluoride etherate) | mL (Milliliters) |
| atm. (Atmosphere) | μL (Microliters) |
| n-BuLi (n-Butyl lithium) | mmol (Millimoles) |
| n-BuOH (n-Butanol) | μmol (Micromoles) |
| CHCl₃ (Chloroform) | M.p. (Melting point) |
| m-CPBA (meta-Chloroperbenzoic acid) | min (Minute) |
| CsF (Cesium fluoride) | NH₄Cl (Ammonium chloride) |
| Cs₂CO₃ (Cesium carbonate) | NaBH₄ (Sodium borohydride) |
| DAST (Diethylaminosulfur trifluoride) | NaH (Sodium hydride) |
| DCM (Dichloromethane) | NaHCO₃ (Sodium bicarbonate) |
| DIBAL (Diisobutylaluminium hydride) | NaHSO₃ (Sodium bisulfite) |
| DMF (Dimethylformamide) | NaOH (Sodium hydroxide) |
| DMSO (Dimethylsulfoxide) | Na₂CO₃ (Sodium carbonate) |
| EtOAc (Ethyl acetate) | Na₂SO₃ (Sodium sulfite) |
| EtOH (Ethanol) | Na₂SO₄ (Sodium sulfate) |
| Et₃SiH (Triethylsilane) | PCC (Pyridinium chlorochromate) |
| H (Hour) | Pd/C (Palladium on charcoal) |
| HATU (2-(7-Aza-1H-benzotriazole-1- | Pd(dppf)Cl₂ ([1,1′- |
| yl)-1,1,3,3-tetramethyluronium | Bis(diphenylphosphino)ferrocene]dichloropalladium(II)) |
| hexafluorophosphate) | |
| HCl (Hydrochloric acid) | Pd(PPh₃)₂Cl₂ |
| | (Bis(triphenylphosphine)palladiumchloride) |
| Hg(OAc)₂ (Mercury (II) acetate) | Pd(PPh₃)₄ |
| | (Tetrakis(triphenylphosphine)Palladium(0)) |
| HMPA (Hexamethylphosphoramide) | Pd₂(dba)₃ |
| | (Tris(dibenzylideneacetone)dipalladium(0)) |
| H₂SO₄ (Sulfuric acid) | PE (Petroleum ether) |
| I₂ (Diiodine) | PhNMe₃Br₃ (Phenyl trimethyl ammonium |
| | tribromide) |
| K₂CO₃ (Potassium carbonate) | Prep. HPLC (Preparative high pressure liquid |
| | chromatography) |
| K₃PO₄ (Potassium phosphate) | Prep. TLC (Preparative thin layer chromatography) |
| LDA (Lithium diisopropylamide) | i-Pr₂NH (Di-isopropylamine) |
| LiAlH₄ (Lithium aluminium hydride) | PtO2 (Platinum oxide) |
| LiBH₄ (Lithium borohydride) | rt (Room temperature) |
| M (Molar) | RT (Retention Time) |
| p-MBCl (p-Methoxybenzylchloride) | SOCl₂ (Thionyl chloride) |
| MeCN (Acetonitrile) | TBAF (Tetra-n-butylammonium fluoride) |
| MeI (Methyl iodide) | TEA (Triethylamine) |
| MeLi (Methyl lithium) | TFA (Trifluoroacetic acid) |
| MeOH (Methanol) | THF (Tetrahydrofuran) |
| MeMgBr (Methyl magnesium bromide) | TMSCF₃ (Trifluoromethyl)trimethylsilane) |
| Me₂S (Dimethyl sulfide) | UPLC-MS (Ultra Performance Liquid |
| | Chromatography Mass Spectrum) |
| mg (Milligrams) | Xantphos (4,5-Bis(diphenylphosphino)-9,9- |
| | dimethylxanthene) |

All references to brine refer to a saturated aqueous solution of NaCl. Unless otherwise indicated, all temperatures are expressed in ° C. (degrees Centigrade). All reactions are conducted under an inert atmosphere at room temperature unless otherwise noted.

Most of the reactions were monitored by thin-layer chromatography on 0.25 mm Merck silica gel plates (60E-254), visualized with UV light. Flash column chromatography was performed on prepacked silica gel cartridges (15-40 μM, Merck).

Melting point determination was performed on a Buchi B-540 apparatus.

¹H-NMR spectra were recorded on a Bruker 300 MHz. Chemical shifts are expressed in parts per million (ppm, δ units). Coupling constants are in units of hertz (Hz) Splitting patterns describe apparent multiplicities and are designated as s (singlet), d (doublet), t (triplet), q (quadruplet), m (multiplet), br (broad).

### EXAMPLES

### Example 1

### (6-Fluoro-pyridin-2-yl)-(6-methyl-6,8-dihydro-4H-5-oxa-3-thia-1,7,8-triaza-cyclopenta[e]azulen-2-yl)-amine (Final Compound 1-23)

### 1-(4-Iodo-M-pyrazol-5-yl)ethanone

According to Scheme 1, Step 1: To a solution of 1-(1H-pyrazol-5-yl)ethanone (31.0 g, 280 mmol) in MeCN (500 mL) was added I₂ (38.0 g, 150 mmol) and then diammonium cerium(IV) nitrate (164 g, 300 mmol). The reaction mixture was stirred for 24 h at rt. When the conversion was complete, a cold solution of 5% NaHSO₃ was added to the reaction mixture. The mixture was extracted with EtOAc (300 mL×3). The combined organic layers were dried over MgSO₄, filtered and concentrated in vacuum to give the title compound (65 g, 98%) which was used without further purification.

### 1-(4-Iodo-1-(4-methoxybenzyl)-1H-pyrazol-3-yl)ethanone

According to Scheme 1, Step 2: To a solution of 1-(4-iodo-1H-pyrazol-5-yl)ethanone (65.0 g, 275 mmol) in MeCN (300 mL) was added K₂CO₃ (76.0 g, 550 mmol) and then 1-(chloromethyl)-4-methoxybenzene (45.6 g, 290 mmol). The reaction mixture was stirred at 60° C. overnight. At rt the mixture was filtered and the filtrate was concentrated in vacuo. The residue was purified by flash chromatography on silica gel to give the title product (88 g, 90%).

MS (ESI): m/z 357 (M+H)⁺.

### 1-(4-Iodo-1-(4-methoxybenzyl)-1H-pyrazol-3-yl)ethanol

According to Scheme 1, Step 3: To a solution of 1-(4-iodo-1-(4-methoxybenzyl)-1H-pyrazol-3-yl)ethanone (17.8 g, 50.0 mmol) in MeOH (100 mL) was added NaBH₄ (7.60 g, 200 mmol) at −10° C. and the mixture was stirred at 0° C. for 1 h. When the conversion was complete, the mixture was concentrated. Water (20 mL) was added and the mixture was extracted with EtOAc (30 mL×3). The combined organic layers were dried over MgSO₄, filtered and concentrated under reduced pressure to give the desired product (17.5 g, 98%) which was used directly without further purification.

¹H-NMR (400 MHz, CDCl₃) δ 7.26 (s, 1H), 7.18 (d, J=8.4 Hz, 2H), 6.88 (d, J=8.4 Hz, 2H), 5.18 (s, 2H), 4.88-4.92 (m, 1H), 3.80 (s, 3H), 1.56 (d, J=6.4 Hz, 3H); MS (ESI): m/z 359 (M+H)⁺.

### 1-(1-(4-Methoxybenzyl)-4-(4,4,5,5-tetramethyl-1,3,2-dioxaborolan-2-yl)-1H-pyrazol-3-yl)ethanol

According to Scheme 1, Step 4: To a solution of 1-(4-iodo-1-(4-methoxybenzyl)-1H-pyrazol-3-yl)ethanol (17.5 g, 49 mmol) and 2-isopropoxy-4,4,5,5-tetramethyl-[1,3,2]dioxaborolane (18.6 g, 100 mmol) in THF (100 mL) was added MeLi (3 M, 50 mL) at −78° C. under N₂ over 30 min. The mixture was stirred at −78° C. for 2 h. Quenched with water (50 mL) and extracted with EtOAc (50 mL×3). The combined organic layers were dried over MgSO₄, filtered and concentrated under reduced pressure to give the crude product which was directly used without further purification (16.8 g crude, 95%).

MS (ESI): m/z 359 (M+H)⁺.

### tert-Butyl 4-bromo-5-formylthiazol-2-ylcarbamate

To a 1 L round bottomed flask was added i-Pr₂NH (59.7 g, 0.59 mol) in THF. The resulting mixture was cooled to 0° C. and n-BuLi (236 mL, 0.59 mol) was added slowly. The mixture was stirred for about 20 min and then a solution of tert-butyl 5-bromothiazol-2-ylcarbamate (50 g, 0.18 mol) in THF was slowly added. The mixture was stirred for about 30 min at 0° C. and then DMF (43.1 g, 0.59 mol) was added. The mixture was stirred for 12 h at 20° C. and diluted with EtOAc. The organic layer was separated, washed with brine, dried over Na₂SO₄, filtered and concentrated in vacuum. The residue was purified by flash chromatography on silica gel to give the desired compound (35 g, 64%) as a white solid.

¹H-NMR (400 MHz, DMSO-d₆): δ 9.73 (s, 1H); 1.47 (s, 9H).

### tert-Butyl-5-formyl-4-(3-(1-hydroxyethyl)-1-(4-methoxybenzyl)-1H-pyrazol-4-yl) thiazol-2-ylcarbamate

According to Scheme 1, Step 5: A solution of 1-(1-(4-methoxybenzyl)-4-(4,4,5,5-tetramethyl-1,3,2-dioxaborolan-2-yl)-1H-pyrazol-3-yl)ethanol (358 mg, 1.00 mmol), tert-butyl 4-bromo-5-formylthiazol-2-ylcarbamate (306 mg, 1.00 mmol), Pd(PPh₃)₂Cl₂ (700 mg), K₃PO₄ (424 mg, 2.00 mmol) in DMF (5 mL) and water (1 mL) was heated to 100° C. for 30 min. After the reaction was finished, water (20 mL) was added and the mixture was extracted with EtOAc (30 mL×3). The combined organic layers were dried over MgSO₄, filtered and concentrated under reduced pressure to give the crude product which was purified by flash chromatography on silica gel to give the desired compound (376 mg, 82%).

MS (ESI): m/z 459 (M+H)⁺.

### tert-Butyl-4-(3-(1-hydroxyethyl)-1-(4-methoxybenzyl)-1H-pyrazol-4-yl)-5-(hydroxymethyl)thiazol-2-ylcarbamate

According to Scheme 1, Step 6: To a stirred solution of tert-butyl-5-formyl-4-(3-(1-hydroxyethyl)-1-(4-methoxybenzyl)-1H-pyrazol-4-yl)thiazol-2-ylcarbamate (275 mg, 0.60 mmol) in MeOH (5 mL) was added NaBH₄ (380 mg, 10.0 mmol) at rt and then the mixture was stirred for 1 h. After the reaction was finished, the mixture was poured into cool water and extracted with EtOAc. The combined organic layers were dried over MgSO₄, filtered and concentrated under reduced pressure to give the desired product (275 mg, 100%).

MS (ESI): m/z 461 (M+H)⁺.

### 8-(4-Methoxy-benzyl)-6-methyl-6,8-dihydro-4H-5-oxa-3-thia-1,7,8-triaza-cyclopenta[e]azulen-2-ylamine

According to Scheme 1, Step 7: A solution of tert-butyl-4-(3-(1-hydroxyethyl)-1-(4-methoxybenzyl)-1H-pyrazol-4-yl)-5-(hydroxymethyl)thiazol-2-ylcarbamate (275 mg, 0.60 mmol) in conc. H₂SO₄ (1 mL) and water (1 mL) was heated to 80° C. for 20 min. After cooling to rt, 1N NaOH was added to pH=8 and the aqueous phase was extracted with EtOAc. The combined organic layers were dried over MgSO₄, filtered and concentrated under reduced pressure. The residue was purified by Prep. TLC to give the title product (151 mg, 74%).

MS (ESI): m/z 343 (M+H)⁺.

### (6-Fluoro-pyridin-2-yl)-[8-(4-methoxy-benzyl)-6-methyl-6,8-dihydro-4H-5-oxa-3-thia-1,7,8-triaza-cyclopenta[e]azulen-2-yl]-amine

According to Scheme 1, Step 8: To a solution of 8-(4-methoxy-benzyl)-6-methyl-6,8-dihydro-4H-5-oxa-3-thia-1,7,8-triaza-cyclopenta[e]azulen-2-ylamine (194 mg, 0.57 mmol) in dioxane (5 mL) was added 2-bromo-6-fluoropyridine (100 mg, 0.57 mmol), Pd₂(dba)₃ (46 mg, 0.05 mmol), Xantphos (58 mg, 0.1 mmol) and Cs₂CO₃ (326 mg, 1.00 mmol). The mixture was stirred at 70° C. under N₂ atmosphere for 2 h. After cooling to rt, 10 mL of water was added, extracted with EtOAc (15 mL×3). The combined organic layers were washed with brine, dried over MgSO₄, filtered and concentrated under reduced pressure to give the desired crude product (240 mg).

MS (ESI): m/z 438 (M+H)⁺.

### (6-Fluoro-pyridin-2-yl)-(6-methyl-6,8-dihydro-4H-5-oxa-3-thia-1,7,8-triaza-cyclopenta[e]azulen-2-yl)-amine

According to Scheme 1, Step 9: (6-Fluoro-pyridin-2-yl)-[8-(4-methoxy-benzyl)-6-methyl-6,8-dihydro-4H-5-oxa-3-thia-1,7,8-triaza-cyclopenta[e]azulen-2-yl]-amine (240 mg, 0.55 mmol) was dissolved in 3 mL of TFA and the mixture was stirred at 100° C. for 3 h. Then the solution was diluted with DMSO (5 mL) and filtered. The filtrate was collected and purified by Prep. HPLC to give the desired product as a off-white solid (10 mg, 6%).

¹H-NMR (400 MHz, DMSO-d₆): δ 11.52 (s, 1H), 7.81 (dd, 1H, J=8.4 Hz, J=16.4 Hz), 7.74 (s, 1H), 6.94 (d, 1H, J=6.0 Hz), 6.57 (d, 1H, J=6.0 Hz), 5.07-4.96 (m, 2H), 4.87 (d, 1H, J=6.0 Hz), 1.52 (d, 3H, J=6.4 Hz); MS (ESI): m/z 318 (M+H)⁺.

### Example 2

### (4-Methyl-pyrimidin-2-yl)-(4,4,6-trimethyl-6,8-dihydro-4H-5-oxa-3-thia-1,7,8-triaza-cyclopenta[e]azulen-2-yl)-amine (Final Compound 1-2)

### {5-Acetyl-4-[3-(1-hydroxy-ethyl)-1-(4-methoxy-benzyl)-1H-pyrazol-4-yl]-thiazol-2-yl}-carbamic acid tert-butyl ester

According to Scheme 1, Step 5: A mixture of tert-butyl 5-acetyl-4-bromothiazol-2-ylcarbamate (320 mg, 1.00 mmol), 1-(1-(4-methoxybenzyl)-4-(4,4,5,5-tetramethyl-1,3,2-dioxaborolan-2-yl)-1H-pyrazol-3-yl)ethanol (358 mg, 1.00 mmol), K₃PO₄ (424 mg, 2.00 mmol) and (PPh₃)₂PdCl₂ (35 mg, 0.05 mmol) in DMF (5 mL) and H₂O (1 mL) was reacted at 80° C. for 1.5 h. After cooling to rt, the mixture was diluted with water and extracted with EtOAc. The organic layer was washed with brine, dried over Na₂SO₄, filtered and concentrated in vacuum. The residue was purified on silica gel to give the desired product (367 mg, 78%).

MS (ESI): m/z 473 (M+H)⁺.

### tert-Butyl-4-(1-(4-methoxybenzyl)-3-(1-hydroxyethyl)-1H-pyrazol-4-yl)-5-(2-hydroxy propan-2-yl)thiazol-2-ylcarbamate

According to Scheme 2: To a solution of {5-acetyl-4-[3-(1-hydroxy-ethyl)-1-(4-methoxy-benzyl)-1H-pyrazol-4-yl]-thiazol-2-yl}-carbamic acid tert-butyl ester (283 mg, 0.60 mmol) in THF (2 mL) at −78° C. was added MeLi (2 mL, 6 mmol). The reaction mixture was stirred for 20 min at −78° C. After 2 h at rt, the reaction was complete. The solution was quenched with HCl 1M and extracted with EtOAc. The combined organic layers were dried over MgSO₄, filtered and concentrated under reduced pressure to give the crude product (293 mg, 100%) which was directly used.

MS (ESI): m/z 489 (M+H)⁺.

### 8-(4-Methoxy-benzyl)-4,4,6-trimethyl-6,8-dihydro-4H-5-oxa-3-thia-1,7,8-triaza-cyclopenta[e]azulen-2-ylamine

According to Scheme 1, Step 7: tert-Butyl-4-(1-(4-methoxybenzyl)-3-(1-hydroxyethyl)-1H-pyrazol-4-yl)-5-(2-hydroxypropan-2-yl)thiazol-2-ylcarbamate (293 mg, 0.60 mmol) in H₂SO₄ (1 mL) and H₂O (1 mL) was stirred at 80° C. for 15 min. The mixture was then cooled to rt, and quenched with 1N NaOH dropwise until pH=8. The aqueous layer was extracted with EtOAc. The organic layer was washed with brine, dried over Na₂SO₄, filtered and concentrated in vacuum. The residue was purified by flash chromatography on silica gel to give the desired product (148 mg, 67%) as a white solid.

MS (ESI): m/z 371 (M+H)⁺.

### [8-(4-Methoxy-benzyl)-4,4,6-trimethyl-6,8-dihydro-4H-5-oxa-3-thia-1,7,8-triaza-cyclopenta[e]azulen-2-yl]-(4-methyl-pyrimidin-2-yl)-amine

According to Scheme 1, Step 8: To a solution of 8-(4-methoxy-benzyl)-4,4,6-trimethyl-6,8-dihydro-4H-5-oxa-3-thia-1,7,8-triaza-cyclopenta[e]azulen-2-ylamine (80 mg, 0.2 mmol) in dioxane (3 mL) was added 2-chloro-4-methyl-pyrimidine (51 mg, 0.4 mmol), Pd₂(dba)₃ (18 mg, 0.02 mmol), Xantphos (23 mg, 0.04 mmol) and Cs₂CO₃ (130 mg, 0.4 mmol) under N₂. The mixture was stirred at 110° C. for 1 h under N₂. After cooling to rt, the mixture was filtered and the filtrate was concentrated in vacuum. The residue was purified by Prep. TLC (PE:EtOAc, 1:2) to give the desired product (40 mg, 43%).

MS (ESI): m/z 463 (M+H)⁺.

### (4-Methyl-pyrimidin-2-yl)-(4,4,6-trimethyl-6,8-dihydro-4H-5-oxa-3-thia-1,7,8-triaza-cyclopenta[e]azulen-2-yl)-amine

According to Scheme 1, Step 9: [8-(4-Methoxy-benzyl)-4,4,6-trimethyl-6,8-dihydro-4H-5-oxa-3-thia-1,7,8-triaza-cyclopenta[e]azulen-2-yl]-(4-methyl-pyrimidin-2-yl)-amine (40 mg, 86 μmol) was dissolved in 2 mL of TFA. The resulting mixture was stirred at 100° C. under microwave conditions for 2 h. After cooling to rt, the mixture was concentrated under reduced pressure then the residue was purified by Prep. HPLC to give the desired compound (6 mg, 20%).

¹H-NMR (400 MHz, DMSO-d₆): δ 8.39 (d, J=4.8 Hz, 1H), 7.72 (s, 1H), 6.83 (d, J=4.8 Hz, 1H), 4.95-4.97 (m, 1H), 2.36 (s, 3H), 1.51-1.55 (m, 6H), 1.46 (d, J=6.0 Hz, 3H); MS (ESI) m/z 343 (M+H)⁺.

### Example 3

### (4,4-Dimethyl-6,8-dihydro-4H-5-oxa-3-thia-1,7,8-triaza-cyclopenta[e]azulen-2-yl)-(4-methyl-pyrimidin-2-yl)-amine (Final Compound 1-4)

### (1-(4-Methoxybenzyl)-4-iodo-1H-pyrazol-3-yl)methanol

According to Scheme 3: To a solution of ethyl 4-iodo-1-(4-methoxybenzyl)-1H-pyrazole-3-carboxylate (15.0 g, 38.9 mmol) in THF (150 mL) was added LiBH₄ (2 M, 58.3 mL) at rt and then the mixture was heated at reflux for 2 h. After the reaction was finished, the mixture was poured into cool water and extracted with EtOAc. The combined organic layers were dried over MgSO₄, filtered and concentrated under reduced pressure. The residue was purified by silica gel (PE:EtOAc, 3:1) to give the title compound (10.0 g, 75%).

MS (ESI) m/z 345 (M+H)⁺.

### (1-(4-Methoxybenzyl)-4-(4,4,5,5-tetramethyl-1,3,2-dioxaborolan-2-yl)-1H-pyrazol-3-yl)methanol

According to Scheme 1, Step 4: To a solution of 2-isopropoxy-4,4,5,5-tetramethyl-[1,3,2]dioxaborolane (6.50 g, 34.8 mmol) and (1-(4-methoxybenzyl)-4-iodo-1H-pyrazol-3-yl)methanol (4.00 g, 11.6 mmol) in THF (40 mL) at −78° C. was added n-BuLi (13.9 mL, 34.8 mmol). The mixture was stirred for 2 h at −78° C. The solution was quenched with water and extracted with EtOAc. The combined organic layers were dried over MgSO₄, filtered and concentrated in vacuum to give the crude product (4 g, 100%) without further purification.

MS (ESI): m/z 345 (M+H)⁺.

### Ethyl-4-(1-(4-methoxybenzyl)-3-(hydroxymethyl)-1H-pyrazol-4-yl)-2-(tert-butoxy carbonyl)thiazole-5-carboxylate

According to Scheme 1, Step 5: A mixture of solution of (1-(4-methoxybenzyl)-4-(4,4,5,5-tetramethyl-1,3,2-dioxaborolan-2-yl)-1H-pyrazol-3-yl)methanol (470 mg, 1.37 mmol), Pd(PPh)₂Cl₂ (40 mg), K₃PO₄ (606 mg, 2.28 mmol), 4-bromo-2-tert-butoxycarbonylamino-thiazole-5-carboxylic acid ethyl ester (400 mg, 1.14 mmol) in a solvent mixture of DMF (5 mL) and water (1 mL) under N₂ was heated to 70° C. for 3 h. After the reaction was finished, water (20 mL) was added and the mixture was extracted with EtOAc (30 mL×3). The combined organic layers were dried over MgSO₄, filtered and concentrated under reduced pressure. The residue was purified by Prep. TLC (PE:EtOAc, 1:1) to afford the desired product (330 mg, 58%).

MS (ESI): m/z 489 (M+H)⁺.

### tert-Butyl-4-(1-(4-methoxybenzyl)-3-(hydroxymethyl)-1H-pyrazol-4-yl)-5-(2-hydroxy propan-2-yl)thiazol-2-ylcarbamate

According to Scheme 1, Step 6: To a solution of ethyl-4-(1-(4-methoxybenzyl)-3-(hydroxymethyl)-1H-pyrazol-4-yl)-2-(tert-butoxycarbonyl)thiazole-5-carboxylate (330 mg, 0.68 mmol) in THF (30 mL) at −78° C. was added MeLi (1.13 mL, 3.38 mmol). The resulting mixture was stirred for 20 min at −78° C. before allowing the reaction to reach rt. After 2 h at rt, the reaction was complete. The mixture was quenched with HCl 1M and extracted with EtOAc. The combined organic layers were dried over MgSO₄, filtered and evaporated under reduced pressure to give the crude product (200 mg, 62%) without further purification.

MS (ESI): m/z 475 (m+H)⁺.

### 8-(4-Methoxy-benzyl)-4,4-dimethyl-6,8-dihydro-4H-5-oxa-3-thia-1,7,8-triaza-cyclopenta[e]azulen-2-ylamine

According to Scheme 1, Step 7: A solution of tert-butyl-4-(1-(4-methoxybenzyl)-3-(hydroxymethyl)-1H-pyrazol-4-yl)-5-(2-hydroxypropan-2-yl)thiazol-2-ylcarbamate (220 mg, 0.42 mmol) in conc. H₂SO₄ (2 mL) and water (2 mL) was heated to 80° C. for 20 min. After the mixture was cooled to rt, 1N NaOH was added to pH=8 and extracted with EtOAc. The combined organic layers were dried over MgSO₄, filtered and concentrated under reduced pressure. The residue was purified by Prep. TLC (EtOAc) to give the desired product (80 mg, 53%).

¹H-NMR (400 MHz, DMSO-d₆): δ 7.70 (s, 1H), 7.22 (d, 2H, J=8.4 Hz), 6.88 (d, 2H, J=8.8 Hz), 5.14 (s, 2H), 4.70 (s, 2H), 3.71 (s, 3H), 1.46 (s, 3H); MS (ESI) m/z 357 (M+H)⁺.

### [8-(4-Methoxy-benzyl)-4,4-dimethyl-6,8-dihydro-4H-5-oxa-3-thia-1,7,8-triaza-cyclopenta[e]azulen-2-yl]-(4-methyl-pyrimidin-2-yl)-amine

According to Scheme 1, Step 8: A mixture of 8-(4-methoxy-benzyl)-4,4-dimethyl-6,8-dihydro-4H-5-oxa-3-thia-1,7,8-triaza-cyclopenta[e]azulen-2-ylamine (80 mg, 0.22 mmol), 2-chloro-4-methyl-pyrimidine (57.5 mg, 0.45 mmol), Pd₂(dba)₃ (10.3 mg, 11.2 μmol), Xantphos (13 mg, 22.5 mmol) and Cs₂CO₃ (146 mg, 0.45 mmol) in dioxane (3 mL) was refluxed for 2 h under N₂. After cooling to rt, the mixture was filtered and the residue was washed with EtOAc (30 mL). The filtrate was collected and concentrated under reduced pressure to give the crude product (100.8 mg, 100%).

MS (ESI): m/z 449 (M+H)⁺.

### (4,4-Dimethyl-6,8-dihydro-4H-5-oxa-3-thia-1,7,8-triaza-cyclopenta[e]azulen-2-yl)-(4-methyl-pyrimidin-2-yl)-amine

According to Scheme 1, Step 9: [8-(4-Methoxy-benzyl)-4,4-dimethyl-6,8-dihydro-4H-5-oxa-3-thia-1,7,8-triaza-cyclopenta[e]azulen-2-yl]-(4-methyl-pyrimidin-2-yl)-amine (100.8 mg, 0.225 mmol) was dissolved in 2 mL of TFA. The mixture was stirred at 100° C. under microwave condition for 20 min. After cooling to rt, the mixture was concentrated in vacuum and purified by Prep. HPLC to afford the desired product (10 mg, 13%).

¹H NMR (400 MHz, DMSO-d₆): δ11.50 (s, 1H), 8.44 (d, 1H, J=4.2 Hz), 7.77 (s, 1H); 6.89 (d, 1H, J=5.2 Hz), 4.86 (s, 2H), 2.42 (s, 3H), 1.59 (s, 6H); MS (ESI): m/z 329 (M+H)⁺.

### Example 4

### (6-Ethyl-6,8-dihydro-4H-5-oxa-3-thia-1,7,8-triaza-cyclopenta[e]azulen-2-yl)-(4-methyl-pyrimidin-2-yl)-amine (Final Compound 1-8)

### Ethyl 1-(4-methoxybenzyl)-1H-pyrazole-4-carboxylate

According to Scheme 4, Step 1: To a suspension of ethyl 1H-pyrazole-4-carboxylate (300 g, 2.14 mol), K₂CO₃ (444 g, 3.21 mol) in MeCN (1.5 L) was added PMBCl (335 g, 2.14 mol). The mixture was stirred at 80° C. for 1 h. After cooling to rt, the mixture was filtered and the filtrate was concentrated under reduced pressure to give the crude product (550 g, 99%) without further purification.

MS (ESI): m/z 261 (M+H)⁺.

### 1-(4-Methoxybenzyl)-1H-pyrazole-4-carboxylic acid

According to Scheme 4, Step 2: To a solution of ethyl 1-(4-methoxybenzyl)-1H-pyrazole-4-carboxylate (550 g, 2.11 mol) in MeOH (1 L) was added NaOH aq. (4N, 1 L) and the mixture was stirred at 80° C. for 1 h. After cooling to rt, the mixture was acified by HCl (4N) and extracted with EtOAc. The combined organic layers were dried over MgSO₄, filtered and concentrated in vacuum to give the crude product (490 g, 99%).

MS (ESI): m/z 233 (M+H)⁺.

### 1-(4-Methoxybenzyl)-N-methoxy-N-methyl-1H-pyrazole-4-carboxamide

According to Scheme 4, Step 3: To a solution of 1-(4-methoxybenzyl)-1H-pyrazole-4-carboxylic acid (490 g, 2.10 mol), HATU (1140 g, 3 mol) and TEA (800 g, 8 mol) in DCM (2 L) was added N,O-dimethyl-hydroxylamine (280 g, 1.50 mol) and the mixture was stirred at rt for 12 h. The mixture was washed with H₂O (500 mL) and brine (500 mL). The organic layer was dried over MgSO₄, filtered and concentrated in vacuum. The residue was purified by flash chromatography on silica gel to produce the title product as a white solid (270 g, 47%).

MS (ESI): m/z 276 (M+H)⁺.

### 1-(4-Methoxybenzyl)-3-O-hydroxypropyl)-N-methoxy-N-methyl-1H-pyrazole-4-carboxamide

According to Scheme 4, Step 4: To a solution of 1-(4-methoxybenzyl)-N-methoxy-N-methyl-1H-pyrazole-4-carboxamide (50 g, 182 mmol) in THF (400 mL) at −78° C. was added dropwise a solution of LDA (218 mmol) freshly prepared. The mixture was stirred at −40° C. for 2 h. Propionaldehyde (15.8 g, 273 mmol) was added dropwise at −30° C. Then the mixture was stirred at rt for another 30 min. 500 mL of aq. NH₄Cl was added to quench the reaction and the mixture was extracted with EtOAc (100 mL×3). The combined organic layers were dried over Na₂SO₄, filtered and concentrated in vacuum. The residue was purified by flash chromatography on silica gel (PE:EtOAc, 10:1 to 1:1) to give the title product (20 g, 33%).

MS (ESI): m/z 334 (M+H)⁺.

### 1-O-(4-Methoxybenzyl)-3-(1-hydroxypropyl)-1H-pyrazol-4-yl)ethanone

According to Scheme 4, Step 5: To a solution of 1-(4-methoxybenzyl)-3-(1-hydroxypropyl)-N-methoxy-N-methyl-1H-pyrazole-4-carboxamide (20 g, 60 mmol) in 200 mL of TI-IF was added dropwise MeMgBr (100 mL, 300 mmol) at 0° C. After the addition, the solution was stirred at rt for 17 h. Then 100 mL of saturated NH₄Cl was added to quench the reaction, extracted with EtOAc (50 mL×3). The combined organic layers were dried over Na₂SO₄, filtered and concentrated in vacuum to give the crude product (16 g, 92%).

MS (ESI): m/z 289 (M+H)⁺.

### Ethyl-3-(1-(4-methoxybenzyl)-3-(1-hydroxypropyl)-1H-pyrazol-4-yl)-3-oxopropanoate

According to Scheme 4, Step 6: To a solution of carbonic acid diethylester (13.3 g, 111 mmol) and NaH (5.55 g, 138.7 mmol) in 140 mL of toluene at 100° C. was added dropwise a solution of 1-(1-(4-methoxybenzyl)-3-(1-hydroxypropyl)-1H-pyrazol-4-yl)ethanone (16.0 g, 55.5 mmol) in 60 mL of toluene. After addition, the mixture was stirred at 100° C. for 2 h. After cooling to rt, 2N HCl (50 mL) was added to the reaction mixture and the aqueous phase was extracted with EtOAc (40 mL×3). The combined organic layers were dried over Na₂SO₄, filtered and concentrated under reduced pressure to give the desired product (12 g, 63%).

MS (ESI): m/z 361 (M+H)⁺.

### Ethyl-3-(1-(4-methoxybenzyl)-3-(1-hydroxypropyl)-1H-pyrazol-4-yl)-2-bromo-3-oxopropanoate

According to Scheme 4, Step 7: To a solution of ethyl-3-(1-(4-methoxybenzyl)-3-(1-hydroxypropyl)-1H-pyrazol-4-yl)-3-oxopropanoate (12.0 g, 33.3 mmol) in DCM (120 mL) was added PhNMe₃Br₃ (12.5 g, 33.3 mmol). The reaction mixture was stirred at rt for 30 min. The mixture was diluted with H₂O (80 mL) and extracted with DCM (60 mL×2). The combined organic layers were dried over Na₂SO₄, concentrated in vacuum to give the crude product (13 g, 89%).

MS (ESI): m/z 439, 441 (M+H)⁺.

### Ethyl-4-(1-(4-methoxybenzyl)-3-(1-hydroxypropyl)-1H-pyrazol-4-yl)-2-aminothiazole-5-carboxylate

According to Scheme 4, Step 8: To a solution of ethyl-3-(1-(4-methoxybenzyl)-3-(1-hydroxypropyl)-1H-pyrazol-4-yl)-2-bromo-3-oxopropanoate (12.0 g, 27.4 mmol) in EtOH (120 mL) was added thiourea (2.50 g, 32.8 mmol). The resulting mixture was stirred at 80° C. for 1 h and then cooled to rt. The mixture was concentrated in vacuum, diluted with 100 mL of EtOAc. The organic layer was washed with NaOH, water and brine, dried over Na₂SO₄, filtered and concentrated in vacuum. The residue was purified by flash chromatography on silica gel (PE:EtOAc, 10:1 to 1:1) to give the desired product (7.5 g, 73%).

MS (ESI): m/z 417 (M+H)⁺.

### 1-(1-(4-Methoxybenzyl)-4-(2-amino-5-(hydroxymethyl)thiazol-4-yl)-1H-pyrazol-3-yl) propan-1-ol

According to Scheme 4, Step 9: To a solution of ethyl-4-(1-(4-methoxybenzyl)-3-(1-hydroxypropyl)-1H-pyrazol-4-yl)-2-aminothiazole-5-carboxylate (7.5 g, 18 mmol) in 100 mL of DCM at −40° C. was added DIBAL (108 mL, 108 mmol) dropwise, then the solution was stirred at −40° C. for 40 min. 100 mL of MeOH was added dropwise at −30° C. and 50 mL of an aq. sat. solution of K₂CO₃ was added, the aqueous phase was extracted with DCM (50 mL×3). The combined organic layers were dried over Na₂SO₄, filtered and concentrated in vacuum to yield the title product (5.7 g, 86%).

MS (ESI): m/z 375 (M+H)⁺.

### 6-Ethyl-8-(4-methoxy-benzyl)-6,8-dihydro-4H-5-oxa-3-thia-1,7,8-triaza-cyclopenta[e]azulen-2-ylamine

According to Scheme 1, Step 7: A solution of 1-(1-(4-methoxybenzyl)-4-(2-amino-5-(hydroxymethyl)thiazol-4-yl)-1H-pyrazol-3-yl)propan-1-ol (5.6 g, 15 mmol) in 10 mL of TFA and 40 mL of DCM was stirred at it for 16 h. The mixture was concentrated in vacuum and the residue was diluted with water and basified with K₂CO₃ to pH=8 and extracted with EtOAc (50 mL×2). The combined organic layers were dried over Na₂SO₄, filtered and concentrated in vacuum. The residue was purified by flash chromatography on silica gel (PE:EtOAc, 10:1 to 1:1) to give the desired product (1 g, 19%).

MS (ESI): m/z 357 (M+H)⁺.

### [6-Ethyl-8-(4-methoxy-benzyl)-6,8-dihydro-4H-5-oxa-3-thia-1,7,8-triaza-cyclopenta[e]azulen-2-yl]-(4-methyl-pyrimidin-2-yl)-amine

According to Scheme 1, Step 8: A solution of 6-ethyl-8-(4-methoxy-benzyl)-6,8-dihydro-4H-5-oxa-3-thia-1,7,8-triaza-cyclopenta[e]azulen-2-ylamine (500 mg, 1.40 mmol), 2-chloro-4-methyl-pyrimidine (269 mg, 2.1 mmol), Pd₂(dba)₃ (128 mg, 0.14 mmol), Xantphos (162 mg, 0.28 mmol) and Cs₂CO₃ (913 mg, 2.8 mmol) in dioxane (15 mL) was refluxed for 1.5 h under N₂. After cooling to rt, the mixture was filtered and the solvent was evaporated to give the crude product, which was purified by flash chromatography on silica gel (PE:EtOAc, 10:1 to 1:1) to give the desired product (400 mg, 64%).

MS (ESI): m/z 449 (M+H)⁺.

### (6-Ethyl-6,8-dihydro-4H-5-oxa-3-thia-1,7,8-triaza-cyclopenta[e]azulen-2-yl)-(4-methyl-pyrimidin-2-yl)-amine

According to Scheme 1, Step 9: A solution of [6-ethyl-8-(4-methoxy-benzyl)-6,8-dihydro-4H-5-oxa-3-thia-1,7,8-triaza-cyclopenta[e]azulen-2-yl]-(4-methyl-pyrimidin-2-yl)-amine (400 mg, 0.89 mmol) in 5 mL of TFA was stirred at 100° C. for 2 h. After cooling to rt, the mixture was concentrated in vacuum. The residue was purified by Prep. HPLC to yield the desired product (100 mg, 34%).

¹H-NMR (400 MHz, DMSO-d₆): δ 8.42 (d, J=4.8 Hz, 1H), 7.75 (s, 1H), 6.87 (d, J=4.8 Hz, 1H), 5.03-5.07 (m, 1H), 4.78-4.86 (m, 2H), 2.40 (s, 3H), 2.08-2.14 (m, 1H), 1.73-1.79 (m, 1H), 0.96 (t, J=7.2 Hz, 3H); MS (ESI): m/z 329 (M+H)⁺.

### Example 5

### (6-Ethyl-6,8-dihydro-4H-5-oxa-3-thia-1,7,8-triaza-cyclopenta[e]azulen-2-yl)-(5-fluoro-pyrimidin-2-yl)-amine (Final Compound 1-19)

### 1-(1-(4-Methoxybenzyl)-4-(2-(5-fluoropyrimidin-2-ylamino)-5-(hydroxymethyl)thiazol-4-yl)-1H-pyrazol-3-yl)propan-1-ol

According to Scheme 5, Step 1: A solution of 1-(4-(2-amino-5-(hydroxymethyl)thiazol-4-yl)-1-(4-methoxybenzyl)-1H-pyrazol-3-yl)propan-1-ol (300 mg, 0.80 mmol), 2-chloro-5-fluoro-pyrimidine (127 mg, 0.96 mmol), Pd₂(dba)₃ (73 mg, 0.08 mmol), Xantphos (92 mg, 0.16 mmol) and Cs₂CO₃ (521 mg, 1.60 mmol) in dioxane (10 mL) was refluxed for 2 h under N₂. After cooling to rt, the mixture was filtered and concentrated in vacuum to give the desired product (100 mg, 27%).

MS (ESI): m/z 471 (M+H)⁺.

### (6-Ethyl-6,8-dihydro-4H-5-oxa-3-thia-1,7,8-triaza-cyclopenta[e]azulen-2-yl)-(5-fluoro-pyrimidin-2-yl)-amine

According to Scheme 5, Step 2: A solution of 1-(1-(4-methoxybenzyl)-4-(2-(5-fluoropyrimidin-2-ylamino)-5-(hydroxymethyl)thiazol-4-yl)-1H-pyrazol-3-yl)propan-1-ol (100 mg, 0.2 mmol) in 2 mL of TFA was stirred at 100° C. for 1 h. After cooling to ft, the mixture was concentrated in vacuum, and the residue was purified by Prep. HPLC to yield the product (8 mg, 12%).

¹H-NMR (400 MHz, DMSO-d₆): δ 8.79 (s, 1H), 7.84 (s, 1H), 5.14-5.18 (m, 1H), 4.90-4.96 (m, 2H), 2.19-2.22 (m, 1H), 1.83-1.89 (m, 1H), 1.07 (t, J=7.2 Hz, 3H); MS (ESP: m/z 333 (M+H)⁺.

### Example 6

### (6-Ethyl-6,8-dihydro-4H-5-oxa-3-thia-1,7,8-triaza-cyclopenta[e]azulen-2-yl)-(5-methyl-[1,2,4]thiadiazol-3-yl)-amine (Final Compound 1-20)

### Ethyl 4-(1-(4-methoxybenzyl)-3-(1-hydroxypropyl)-1H-pyrazol-4-yl)-2-(5-methyl-1,2,4-thiadiazol-3-ylamino)thiazole-5-carboxylate

According to Scheme 6, Step 1: To a solution of ethyl 2-bromo-3-(3-(1-hydroxypropyl)-1-(4-methoxybenzyl)-1H-pyrazol-4-yl)-3-oxopropanoate (504 mg, 1.26 mmol) in EtOH (5 mL) was added (5-methyl-[1,2,4]-thiadiazol-3-yl)thiourea (200 mg, 1.15 mmol). The mixture was stirred at 80° C. for 24 h. After cooling to rt, the mixture was concentrated and 20 mL of EtOAc was added. The organic layer was washed with NaOH, water and brine, dried over Na₂SO₄, filtered and concentrated to give the crude product, which was purified by flash chromatography on silica gel (PE:EtOAc, 10:1 to 1:1) to give the product (250 mg, 42%).

MS (ESI): m/z 515 (M+H)⁺.

### 1-(1-(4-Methoxybenzyl)-4-(5-(hydroxymethyl)-2-(5-methyl-1,2,4-thiadiazol-3-ylamino) thiazol-4-yl)-1H-pyrazol-3-yl)propan-1-ol

According to Scheme 6, Step 2: To a solution of ethyl-4-(1-(4-methoxybenzyl)-3-(1-hydroxypropyl)-1H-pyrazol-4-yl)-2-(5-methyl-1,2,4-thiadiazol-3-ylamino)thiazole-5-carboxylate (250 mg, 0.48 mmol) in 5 mL of DCM at −40° C. was added DIBAL (2.88 mL, 2.88 mmol) dropwise. After addition, the resulting mixture was stirred at −40° C. for 40 min. 4 mL of MeOH was added dropwise at −30° C. then 5 mL of an aq. sat. solution of K₂CO₃ was added, extracted with DCM (5 mL×3). The combined organic layers were dried over Na₂SO₄, filtered and concentrated in vacuum to yield the product (180 mg, 79%).

MS (ESI): m/z 473 (M+H)⁺.

### (6-Ethyl-6,8-dihydro-4H-5-oxa-3-thia-1,7,8-triaza-cyclopenta[e]azulen-2-yl)-(5-methyl thiadiazol-3-yl)-amine

According to Scheme 5, Step 2: A solution of 1-(1-(4-methoxybenzyl)-4-(5-(hydroxymethyl)-2-(5-methyl-1,2,4-thiadiazol-3-ylamino)thiazol-4-yl)-1H-pyrazol-3-yl)propan-1-ol (130 mg, 0.27 mmol) in 2 mL of TFA was stirred at rt for 15 min then at 100° C. for 1.5 h. After cooling to rt, the mixture was concentrated in vacuum, the residue was purified by Prep. HPLC to yield the title compound (20 mg, 22%).

¹H-NMR (400 MHz, DMSO-d₆): δ 7.74 (s, 1H), 5.02-5.06 (m, 1H), 4.79-4.85 (m, 2H), 2.72 (s, 3H), 2.08-2.13 (m, 1H), 1.72-1.79 (m, 1H), 0.96 (t, J=7.2 Hz, 3H); MS (ESI): m/z 335 (M+H)⁺.

### Example 7

### (6,6-Dimethyl-6,8-dihydro-4H-5-oxa-3-thia-1,7,8-triaza-cyclopenta[e]azulen-2-yl)-(4-methyl pyrimidin-2-yl)-amine (Final Compound 1-10)

### 1-(4-Methoxybenzyl)-3-bromo-1H-pyrazole-4-carboxylic acid

According to Scheme 7, Step 1: To a solution of ethyl 3-bromo-1-(4-methoxybenzyl)-1H-pyrazole-4-carboxylate (55 g, 162 mmol) in MeOH (200 mL) was added 4N NaOH (200 mL). The reaction mixture was refluxed for 1 h. After cooling to rt, to the mixture was added conc. HCl to pH=2˜3, extracted with EtOAc (250 mL×5). The combined organic layers were washed with brine, dried over MgSO₄, filtered, and concentrated in vacuum to afford the title product (45 g, 89%).

MS (ESI): m/z 311, 313 (M+H)⁺.

### 1-(4-Methoxybenzyl)-3-bromo-N-methoxy-N-methyl-1H-pyrazole-4-carboxamide

According to Scheme 7, Step 2: A solution of 1-(4-methoxybenzyl)-3-bromo-1H-pyrazole-4-carboxylic acid (45 g, 145 mmol) in DCM (300 mL) was added DMF (5 drops) and oxalyl chloride (37.0 g, 290 mmol) dropwise at 0° C. under N₂. The reaction mixture was allowed to warm to rt and stirred for another 3 h. After evaporation of the solvent, the residue was treated with toluene and was co-evaporated to dryness to yield 3-bromo-1-(4-methoxy-benzyl)-1H-pyrazole-4-carbonyl chloride. To a mixture of N,O-dimethylhydroxylamine hydrochloride (21.0 g, 217 mmol) and TEA (59.0 g, 580 mmol) in DCM (250 mL) was added dropwise a solution of 3-bromo-1-(4-methoxy-benzyl)-1H-pyrazole-4-carbonyl chloride in DCM (70 mL) at 0° C. under N₂. The reaction mixture was allowed to warm to rt and stirred overnight. The reaction was washed with brine (100 mL×3), water (100 mL×2), dried over MgSO₄, filtered, and concentrated. The residue was purified by flash chromatography on silica gel (PE:EtOAc, 10:1 to 2:1) to give the desired product (40 g, 78%).

¹H-NMR (400 MHz, CDCl₃): δ 7.68 (s, 1H), 7.19 (d, 2H, J=8.8 Hz), 6.87 (d, 2H, J=8.8 Hz), 5.19 (s, 2H), 3.79 (s, 3H), 3.54 (s, 3H), 3.23 (s, 3H); MS (ESI): m/z 354 (M+H)⁺.

### 1-(4-Methoxybenzyl)-N-methoxy-N-methyl-3-(prop-1-en-2-yl)-1H-pyrazole-4-carboxamide

According to Scheme 7, Step 3: A mixture of 1-(4-methoxybenzyl)-3-bromo-N-methoxy-N-methyl-1H-pyrazole-4-carboxamide (4.80 g, 13.6 mmol), 2-isopropenyl boronic acid pinacol ester (2.40 g, 14.3 mmo), Cs₂CO₃ (5.2 g, 16.4 mmo) and Pd(dppf)Cl₂ (1.4 g, 1.9 mmo) in dioxane (50 mL) and H₂O (10 mL) was refluxed for 2 h under N₂. After the reaction was finished, the mixture was filtered, diluted with water, and extracted with EtOAc. The combined organic layers were dried over Na₂SO₄, filtered and concentrated under reduced pressure. The residue was purified by silica gel column (PE:EtOAc, 5:1) to give the title product (3.4 g, 79%); MS (ESI): m/z 316 (M+H)⁺.

### 1-(4-Methoxybenzyl)-3-(2-hydroxypropan-2-A-N-methoxy-N-methyl-1H-pyrazole-4-carboxamide

According to Scheme 7, Step 4: A mixture of 1-(4-methoxybenzyl)-N-methoxy-N-methyl-3-(prop-1-en-2-yl)-1H-pyrazole-4-carboxamide (3.30 g, 10.5 mmol) and Hg(OAc)₂ (8.10 g, 25.2 mmol) in THF (30 mL) and H₂O (50 mL) was stirred for 15 h at rt, and then NaOH (0.90 g, 25.2 mmol) was added. After 10 min, NaBH₄ (0.90 g, 25.2 mmol) was added in portions at −20° C. The mixture was stirred for 1 h at rt. After the reaction was finished, the mixture was diluted with water, filtered and extracted with EtOAc. The combined organic layers were dried over Na₂SO₄, filtered and concentrated under reduced pressure. The residue was purified by silica gel column (PE:EtOAc, 2:1) to give the title compound (2.1 g, 60%).

MS (ESI): m/z 334 (M+H)⁺.

### 1-(1-(4-Methoxybenzyl)-3-(2-hydroxypropan-2-yl)-1H-pyrazol-4-yl)prop-2-en-1-one

According to Scheme 7, Step 5: To a solution of 1-(4-methoxybenzyl)-3-(2-hydroxypropan-2-yl)-N-methoxy-N-methyl-1H-pyrazole-4-carboxamide (2.1 g, 6.3 mmol) in THF (30 mL) was added vinyl Grignard reagent (1M, 24 mL, 24 mmol) dropwise at −20° C. The mixture was stirred for 30 min at rt. After the reaction was finished, quenched with cooled sat. NH₄Cl (aq) and extracted with EtOAc (30 mL×2). The combined organic layers were dried over Na₂SO₄, filtered and concentrated under reduced pressure. The residue was purified by silica gel column (PE:EtOAc, 2:1) to give the title product (0.4 g, 22%).

¹H-NMR (400 MHz, CD₃OD): δ 8.53 (s, 1H), 7.25 (d, J=8.8 Hz, 2H), 7.10 (dd, J=16.8 Hz, 10.4 Hz, 1H), 6.89 (d, J=8.8 Hz, 2H), 6.43 (dd, J=16.8 Hz, 1.6 Hz, 1H), 5.86 (dd, J=10.4 Hz, 1.6 Hz, 1H), 5.21 (s, 2H), 3.76 (s, 3H), 1.50 (s, 6H).

MS (ESI): m/z 301 (M+H)⁺.

### 2-(4-Methoxybenzyl)-8,8-dimethyl-5,6-dihydro-2H-oxepino[3,4-c]pyrazol-4(8H)-one

According to Scheme 7, Step 6: To a solution of 1-(1-(4-methoxybenzyl)-3-(2-hydroxypropan-2-yl)-1H-pyrazol-4-yl) prop-2-en-1-one (0.4 g, 1.3 mmol) in DCM (40 mL) was added BF₃.Et₂O (0.5 g, 3.9 mmol) dropwise at −20° C. The mixture was stirred for 15 h at rt. After the reaction was finished, the mixture was quenched with sat. Na₂CO₃ (aq) and extracted with EtOAc (25 mL×2). The combined organic layers were dried over Na₂SO₄, filtered and concentrated under reduced pressure. The residue was purified by Prep. TLC (PE:EtOAc, 2:1) to give the desired compound (0.1 g, 25%).

¹H-NMR (400 MHz, CD₃OD): δ 7.96 (s, 1H), 7.23 (d, J=8.8 Hz, 2H), 6.90 (d, J=8.4 Hz, 2H), 5.20 (s, 2H), 3.97 (t, J=5.2 Hz, 2H), 3.76 (s, 3H), 2.71 (t, J=5.2 Hz, 2H), 1.53 (s, 6H); MS (ESI): m/z 301 (M+H)⁺.

### 2-(4-Methoxybenzyl)-5-bromo-8,8-dimethyl-5,6-dihydro-2H-oxepino[3,4-c]pyrazol-4(8H)-one

According to Scheme 7, Step 7: A mixture of 2-(4-methoxybenzyl)-8,8-dimethyl-5,6-dihydro-2H-oxepino[3,4-c]pyrazol-4(8H)-one (100 mg, 0.30 mmol) and PhNMe₃Br₃ (78 mg, 0.21 mmol) in DCM (8 mL) was refluxed for 1 h. After the reaction was finished, the mixture was diluted with DCM and washed with water (3 mL), dried over Na₂SO₄, filtered and concentrated under reduced pressure to give the crude product (110 mg, 97%), which was used for the next step without further purification.

MS (ESI): m/z 379, 381 (M+H)⁺.

### [8-(4-Methoxy-benzyl)-6,6-dimethyl-6,8-dihydro-4H-5-oxa-3-thia-1,7,8-triaza-cyclopenta[e]azulen-2-yl]-(4-methyl-pyrimidin-2-yl)-amine

According to Scheme 7, Step 8: A mixture of 2-(4-methoxybenzyl)-5-bromo-8,8-dimethyl-5,6-dihydro-2H-oxepino[3,4-c]pyrazol-4(8H)-one (75 mg, 0.2 mmol) and (4-methyl-pyrimidin-2-yl)-thiourea (40 mg, 0.24 mmol) in EtOH (3 mL) was refluxed for 3 h. After the reaction was finished, the mixture was concentrated under reduced pressure. The residue was purified by Prep. TLC (PE:EtOAc, 2:1) to give the title compound (20 mg, 22%).

MS (ESI): m/z 449 (M+H)⁺.

### (6,6-Dimethyl-6,8-dihydro-4H-5-oxa-3-thia-1,7,8-triaza-cyclopenta[e]azulen-2-yl)-(4-methyl-pyrimidin-2-yl)-amine

According to Scheme 7, Step 9: A solution of [8-(4-methoxy-benzyl)-6,6-dimethyl-6,8-dihydro-4H-5-oxa-3-thia-1,7,8-triaza-cyclopenta[e]azulen-2-yl]-(4-methyl-pyrimidin-2-yl)-amine (20 mg, 0.04 mmol) in TFA (2 mL) and H₂O (0.1 mL) was refluxed for 1 h. After the reaction was finished, the mixture was concentrated under reduced pressure. The residue was purified by Prep. HPLC to give the desired product (3 mg, 21%).

¹H-NMR (400 MHz, DMSO-d₆): δ 11.52 (br, 1H), 8.42 (d, J=4.8 Hz, 1H), 7.69 (s, 1H), 6.87 (d, J=4.8 Hz, 1H), 4.82 (s, 2H), 2.40 (s, 3H), 1.52 (s, 6H); MS (ESI): m/z 329 (M+H)⁺.

### Example 8

### (4-Methyl-pyrimidin-2-yl)-(6-trifluoromethyl-6,8-dihydro-4H-5-oxa-3-thia-1,7,8-triaza-cyclopenta[e]azulen-2-yl)-amine (Final Compound 1-13)

### 1-(4-Methoxybenzyl)-N-methoxy-N-methyl-3-vinyl-1H-pyrazole-4-carboxamide

According to Scheme 8, Step 1: A mixture of 3-bromo-N-methoxy-1-(4-methoxybenzyl)-N-methyl-1H-pyrazole-4-carboxamide (20 g, 56 mmol), tributyl(vinyl)tin (21 g, 67 mmol) and Pd(PPh₃)₄ (1.7 g, 1.5 mmol) in toluene (200 mL) was refluxed for 6 h. After cooling to rt, the mixture was concentrated under reduced pressure. The residue was purified by flash chromatography on silica gel (PE:EtOAc, 10:1) to give the title compound (11 g, 65%).

### 1-(4-Methoxybenzyl)-3-formyl-N-methoxy-N-methyl-1H-pyrazole-4-carboxamide

According to Scheme 8, Step 2: To a solution of 1-(4-methoxybenzyl)-N-methoxy-N-methyl-3-vinyl-1H-pyrazole-4-carboxamide (6.0 g, 20 mol) in dry DCM (150 mL) was bubbled O₃ until the color of the solution turn blue at −78° C. After the material was consumed, Me₂S (8 mL) was added. The mixture was allowed to warm up to rt and concentrated in vacuum. The residue was purified by silica gel to give the desired product (4.0 g, 67%).

### 1-(4-Methoxybenzyl)-N-methoxy-N-methyl-3-(2,2,2-trifluoro-1-hydroxyethyl)-1H-pyrazole-4-carboxamide

According to Scheme 8, Step 3: To a solution of 1-(4-methoxybenzyl)-3-formyl-N-methoxy-N-methyl-1H-pyrazole-4-carboxamide (3.85 g, 12.7 mmol) in THF (40 mL) was added TMSCF₃ (2.70 g, 19 mmol) and TBAF (19 mL, 19 mmol) at 0° C. The mixture was stirred for 2 h at 0° C. After the reaction was finished, the mixture was diluted with DCM, washed with water and brine. The organic layer was dried over Na₂SO₄, filtered and concentrated under reduced pressure. The residue was purified by silica gel column (PE:EtOAc, 4:1) to give the title compound (2.2 g, 46%).

MS (ESI): m/z 359 (M+H)⁺.

### 1-(1-(4-Methoxybenzyl)-3-(2,2,2-trifluoro-1-hydroxyethyl)-1H-pyrazol-4-yl)prop-2-en-1-one

According to Scheme 7, Step 5: To a solution of 1-(4-methoxybenzyl)-N-methoxy-N-methyl-3-(2,2,2-trifluoro-1-hydroxyethyl)-1H-pyrazole-4-carboxamide (2.2 g, 5.9 mmol) in THF (30 mL) was added vinyl Grignard reagent (1M, 36 mL, 36 mmol) dropwise at −20° C. The mixture was stirred for 30 min at 0° C. After the reaction was finished, the mixture was quenched with cooled H₂O and HCl (aq), extracted with EtOAc (30 mL×2). The combined organic layers were dried over Na₂SO₄, filtered and concentrated under reduced pressure. The residue was purified by silica gel column (PE:EtOAc, 2:1) to give the title product (0.5 g, 25%).

MS (ESI): m/z 341 (M+H)⁺.

### 2-(4-Methoxybenzyl)-8-(trifluoromethyl)-5,6-dihydro-2H-oxepino[3,4-c]pyrazol-4(8H)-one

According to Scheme 7, Step 6: To a solution of 1-(1-(4-methoxybenzyl)-3-(2,2,2-trifluoro-1-hydroxyethyl)-1H-pyrazol-4-yl)prop-2-en-1-one (0.5 g, 1.5 mmol) in DCM (40 mL) was added BF₃.Et₂O (0.4 g, 3 mmol) dropwise at −20° C. The mixture was stirred for 15 h at rt. After the reaction was finished, the mixture was quenched with aq. sat. solution of Na₂CO₃ and extracted with EtOAc (25 mL×2). The combined organic layers were dried over Na₂SO₄, filtered and concentrated under reduced pressure. The residue was purified by Prep. TLC (PE:EtOAc, 2:1) to give the title compound (0.1 g, 20%).

MS (ESI): m/z 341 (M+H)⁺.

### 2-(4-Methoxybenzyl)-5-bromo-8-(trifluoromethyl)-5,6-dihydro-2H-oxepino[3,4-c]pyrazol-4(8H)-one

According to Scheme 7, Step 7: A mixture of 2-(4-methoxybenzyl)-8-(trifluoromethyl)-5,6-dihydro-2H-oxepino[3,4-c]pyrazol-4(8H)-one (100 mg, 0.30 mmol) and PhNMe₃Br₃ (110 mg, 0.30 mmol) in DMF (5 mL) was heated for 1 h at 60° C. After the reaction was finished, the mixture was cooled and used for the next step without further purification.

MS (ESI): m/z 419, 421 (M+H)⁺.

### [8-(4-Methoxy-benzyl)-6-trifluoromethyl-6,8-dihydro-4H-5-oxa-3-thia-1,7,8-triaza-cyclopenta azulen-2-yl]-(4-methyl-pyrimidin-2-yl)-amine

According to Scheme 7, Step 8: A mixture of 2-(4-methoxybenzyl)-5-bromo-8-(trifluoromethyl)-5,6-dihydro-2H-oxepino[3,4-c]pyrazol-4(8H)-one (123 mg, 0.30 mmol) and (4-methyl-pyrimidin-2-yl)-thiourea (60 mg, 0.36 mmol) in DMF (5 mL) was heated for 3 h at 60° C. After the reaction was finished, the mixture was diluted with H₂O and extracted with EtOAc (25 mL×2). The combined organic layers were dried over Na₂SO₄, filtered and concentrated under reduced pressure. The residue was purified by Prep. TLC (PE:EtOAc, 1:1) to give the desired compound (50 mg, 34%).

MS (ESI): m/z 489 (M+H)⁺.

### (4-Methyl-pyrimidin-2-yl)-(6-trifluoromethyl-6,8-dihydro-4H-5-oxa-3-thia-1,7,8-triaza-cyclopenta[e]azulen-2-yl)-amine

According to Scheme 7, Step 9: A solution of [8-(4-methoxy-benzyl)-6-trifluoromethyl-6,8-dihydro-4H-5-oxa-3-thia-1,7,8-triaza-cyclopenta[e]azulen-2-yl]-(4-methyl-pyrimidin-2-yl)-amine (50 mg, 0.1 mmol) in TFA (2 mL) and H₂O (0.1 mL) was refluxed for 1 h. After the reaction was finished, the mixture was concentrated under reduced pressure. The residue was purified by Prep. HPLC to give the desired product (2.8 mg, 8%).

¹H-NMR (400 MHz, DMSO-d₆): δ 11.60 (s, 1H), 8.42 (d, J=5.2 Hz, 1H), 7.89 (s, 1H), 6.88 (d, J=5.2 Hz, 1H), 5.75 (d, J=5.6 Hz, 1H), 5.16 (d, J=16 Hz, 1H), 5.02 (d, J=15.6 Hz, 1H), 2.40 (s, 3H); MS (ESI): m/z 369 (M+H)⁺.

### Example 9

### (6-Isopropyl-6,8-dihydro-4H-5-oxa-3-thia-1,7,8-triaza-cyclopenta[e]azulen-2-yl)-(4-methyl-pyrimidin-2-yl)-amine (Final Compound 1-16)

### 1-(4-Methoxybenzyl)-3-(1-hydroxy-2-methylpropyl)-N-methoxy-N-methyl-1H-pyrazole-4-carboxamide

According to Scheme 9: To a solution of 3-formyl-N-methoxy-1-(4-methoxybenzyl)-N-methyl-1H-pyrazol-4-carboxamide (3.85 g, 12.7 mmol) in THF (70 mL) was added isopropylmagnesium bromide (25 mL, 12.7 mmol) dropwise at −30° C. The mixture was stirred for 2 h at −30° C. After the reaction was finished, the mixture was quenched with cooled sat. NH₄Cl (aq) and extracted with EtOAc (50 mL×2). The combined organic layers were dried over Na₂SO₄, filtered and concentrated under reduced pressure. The residue was purified by silica gel column (PE:EtOAc, 2:1) to give the title compound (2.5 g, 57%).

MS (ESI): m/z 348 (M+H)⁺.

### 1-(1-(4-Methoxybenzyl)-3-(1-hydroxy-2-methylpropyl)-1H-pyrazol-4-yl)prop-2-en-1-one

According to Scheme 7, Step 5: To a solution of 1-(4-methoxybenzyl)-3-(1-hydroxy-2-methylpropyl)-N-methoxy-N-methyl-1H-pyrazole-4-carboxamide (2.5 g, 7.2 mmol) in THF (30 mL) was added dropwise vinyl Grignard reagent (1M, 50 mL, 50 mmol) at −20° C. The mixture was stirred for 30 min at 0° C. After the reaction was finished, the mixture was quenched with cooled sat. NH₄Cl (aq) and extracted with EtOAc (40 mL×2). The combined organic layers were dried over Na₂SO₄, filtered and concentrated under reduced pressure. The residue was purified by silica gel column (PE:EtOAc, 2:1) to give the desired product (1 g, 45%).

MS (ESI): m/z 315 (M+H)⁺.

### 2-(4-Methoxybenzyl)-8-isopropyl-5,6-dihydro-2H-oxepino[3,4-c]pyrazol-4(8H)-one

According to Scheme 7, Step 6: To a solution of 1-(1-(4-methoxybenzyl)-3-(1-hydroxy-2-methylpropyl)-1H-pyrazol-4-yl)prop-2-en-1-one (1.0 g, 3.2 mmol) in DCM (100 mL) was added dropwise BF₃.Et₂O (0.9 g, 6.4 mmol) at −20° C. The mixture was stirred for 15 h at rt. After the reaction was finished, the mixture was quenched with sat. Na₂CO₃ (aq) and extracted with EtOAc (50 mL×2). The combined organic layers were dried over Na₂SO₄, filtered and concentrated under reduced pressure. The residue was purified by Prep. TLC (PE:EtOAc, 2:1) to give the desired compound (0.12 g, 12%).

MS (ESI): m/z 315 (M+H)⁺.

### 2-(4-Methoxybenzyl)-5-bromo-8-isopropyl-5,6-dihydro-2H-oxepino[3,4-c]pyrazol-4(8H)-one

According to Scheme 7, Step 7: A mixture of 2-(4-methoxybenzyl)-8-isopropyl-5,6-dihydro-2H-oxepino[3,4-c]pyrazol-4(8H)-one (120 mg, 0.38 mmol) and PhNMe₃Br₃ (142 mg, 0.38 mmo) in CHCl₃ (10 mL) was refluxed for 1 h. After the reaction was finished, the mixture was washed with water (10 mL). The organic layer was dried over Na₂SO₄, filtered and concentrated under reduced pressure to give the crude product (130 mg, 87%) without further purification.

MS (ESI): m/z 393, 395 (M+H)⁺.

### [6-Isopropyl-8-(4-methoxy-benzyl)-6,8-dihydro-4H-5-oxa-3-thia-1,7,8-triaza-cyclopenta[e]azulen-2-yl]-(4-methyl-pyrimidin-2-yl)-amine

According to Scheme 7, Step 8: A mixture of 2-(4-methoxybenzyl)-5-bromo-8-isopropyl-5,6-dihydro-2H-oxepino[3,4-c]pyrazol-4(8H)-one (130 mg, 0.33 mmol) and (4-methyl-pyrimidin-2-yl)-thiourea (60 mg, 0.36 mmol) in EtOH (5 mL) was refluxed for 3 h. After the reaction was finished, the mixture was concentrated under reduced pressure. The residue was purified by Prep. TLC (PE:EtOAc, 2:1) to give the desired compound (50 mg, 33%).

MS (ESI): m/z 463 (M+H)⁺.

### (6-Isopropyl-6,8-dihydro-4H-5-oxa-3-thia-1,7,8-triaza-cyclopenta[e]azulen-2-yl)-(4-methyl-pyrimidin-2-yl)-amine

According to Scheme 7, Step 9: A solution of [6-isopropyl-8-(4-methoxy-benzyl)-6,8-dihydro-4H-5-oxa-3-thia-1,7,8-tri aza-cyclopenta[e]azulen-2-yl]-(4-methyl-pyrimidin-2-yl)-amine (50 mg, 0.11 mmol) in TFA (3 mL) and H₂O (0.1 mL) was refluxed for 1 h. After the reaction was finished, the mixture was concentrated under reduced pressure. The residue was purified by Prep. HPLC to give the desired product (3.5 mg, 9%).

¹H-NMR (400 MHz, DMSO-d₆): δ 11.52 (s, 1H), 8.41 (d, J=5.2 Hz, 1H), 7.74 (s, 1H), 6.86 (d, J=5.2 Hz, 1H), 5.04 (d, J=15.6 Hz, 1H), 4.77 (d, J=15.6 Hz, 1H), 4.74 (d, J=12.8 Hz, 1H), 2.43-2.50 (m, 1H), 2.42 (s, 3H), 1.06 (d, J=6.8 Hz, 3H), 0.77 (d, J=6.8 Hz, 3H); MS (ESI): m/z 343 (M+H)⁺.

### Example 10

### (4,8-Dihydro-5H-6-oxa-3-thia-1,7,8-triaza-cyclopenta[e]azulen-2-yl)-(4-methyl-pyrimidin-2-yl)-amine (Final Compound 1-17)

### 3-Hydroxy-1-(4-methoxy-benzyl)-1H-pyrazole-4-carboxylic acid methyl ester

According to Scheme 10, Step 1: A mixture of 2-dimethylaminomethylene-malonic acid dimethyl ester (18.7 g, 0.10 mol), (4-methoxybenzyl)hydrazine (22.4 g, 0.10 mol) and K₂CO₃ (27.2 g, 0.20 mol) in MeCN (100 mL) was refluxed for 6 h. After cooling to rt, the mixture was diluted with H₂O (200 mL), and washed with EtOAc (50 mL). The aqueous layer was neutralized with 2 N HCl. The precipitate was filtered and dried in vacuum to give the desired product (16.1 g, 62%).

¹H-NMR (400 MHz, CDCl₃): δ 7.68 (s, 1H), 7.19 (d, J=8.8 Hz, 2H), 6.88 (d, J=8.8 Hz, 2H), 5.07 (s, 2H), 3.81 (s, 3H), 3.76 (s, 3H).

### 3-Allyloxy-1-(4-methoxy-benzyl)-1H-pyrazole-4-carboxylic acid methyl ester

According to Scheme 10, Step 2: A mixture of 3-hydroxy-1-(4-methoxy-benzyl)-1H-pyrazole-4-carboxylic acid methyl ester (13.1 g, 50.0 mmol), 3-bromo-propene (6.0 g, 50 mmol) and K₂CO₃ (13.8 g, 0.10 mol) in MeCN (100 mL) was refluxed for 4 h. After cooling to rt, the mixture was filtered. The filtrate was concentrated and purified by silica gel column to yield the desired product (3.8 g, 25%).

MS (ESI): m/z 303 (M+H)⁺.

### 3-Allyloxy-1-(4-methoxy-benzyl)-1H-pyrazole-4-carboxylic acid

According to Scheme 10, Step 3: A mixture of 3-allyloxy-1-(4-methoxy-benzyl)-1H-pyrazole-4-carboxylic acid methyl ester (2.1 g, 7.0 mmol) and NaOH (4.0 g, 0.1 mol) in MeOH (10 mL) and H₂O (5 mL) was refluxed for 1 h. After cooling to rt, the mixture was acified with 3N HCl. The mixture was concentrated in vacuo and the residue was dissolved in MeOH, filtered to remove NaCl and the filtrate was concentrated to yield the title product (1.81 g, 90%).

MS (ESI): m/z 289 (M+H)⁺.

### 3-Allyloxy-1-(4-methoxy-benzyl)-1H-pyrazole-4-carboxylic acid methoxy-methyl-amide

According to Scheme 10, Step 4: A mixture of 3-allyloxy-1-(4-methoxy-benzyl)-1H-pyrazole-4-carboxylic acid (1.8 g, 6.3 mmol), N,O-dimethyl-hydroxylamine HCl (960 mg, 10.0 mmol), HATU (3.8 g, 10 mmol) and TEA (2.0 g, 20 mmol) in DCM (20 mL) was stirred at rt for 2 h. The mixture was washed with H₂O (10 mL) and brine (10 mL). The organic layer was dried over Na₂SO₄, filtered and concentrated in vacuum. The residue was purified by flash chromatography on silica gel (PE:EtOAc, 3:1) to yield the desired product (1.56 g, 75%).

¹H-NMR (400 MHz, CDCl₃): δ 7.78 (s, 1H), 7.22 (d, J=8.4 Hz, 2H), 6.82 (d, J=8.4 Hz, 2H), 5.87-5.97 (m, 1H), 5.31 (d, J=13.5 Hz, 1H), 5.22 (d, J=10.4 Hz, 1H), 5.09 (s, 2H), 4.74 (d, J=6.4 Hz, 2H), 3.76 (s, 3H), 3.63 (s, 3H), 3.28 (s, 3H); MS (ESI): m/z 332 (M+H)⁺.

### 1-[3-Allyloxy-1-(4-methoxy-benzyl)-1H-pyrazol-4-yl]-propenone

According to Scheme 10, Step 5: To a solution of 3-allyloxy-1-(4-methoxy-benzyl)-1H-pyrazole-4-carboxylic acid methoxy-methyl-amide (1.32 g, 4.00 mmol) in THF (30 mL) was added vinyl Grignard reagent (1M, 20 mL, 20 mmol) dropwise at 0° C. The mixture was stirred for 3 h at 0° C. After the reaction was finished, the reaction was quenched with cooled 1N HCl and extracted with EtOAc (30 mL×2). The combined organic layers were dried over Na₂SO₄, filtered and concentrated under reduced pressure. The residue was purified by silica gel column (PE:EtOAc, 2:1) to give the title compound (518 mg, 44%).

MS (ESI): m/z 299 (M+H)⁺.

### 2-(4-Methoxy-benzyl)-2H,7H-oxepino[2,3-c]pyrazol-4-one

According to Scheme 10, Step 6: To a solution of 1-[3-allyloxy-1-(4-methoxy-benzyl)-1H-pyrazol-4-yl]-propenone (400 mg, 1.34 mmol) in dry DCM (134 mL) was added Grubbs catalyst 2^{nd} Generation (114 mg, 0.13 mmol) under N₂. Then the resulting mixture was refluxed for 18 h. After cooling to rt, the mixture was concentrated in vacuum and the residue was purified by flash chromatography on silica gel (PE:EtOAc, 10:1 to 5:1) to afford the desired compound (120 mg, 32%).

¹H-NMR (DMSO-d₆, 400 MHz): δ 7.73 (s, 1H), 7.13 (d, J=8.4 Hz, 2H), 6.79 (d, J=7.2 Hz, 2H), 6.40-6.44 (m, 1H), 6.20 (d, J=12.4 Hz, 1H), 5.00 (s, 2H), 4.75 (d, J=6.0 Hz, 2H), 3.72 (s, 3H); MS (ESI): m/z 271 (M+H)⁺.

### 2-(4-Methoxy-benzyl)-6,7-dihydro-2H,5H-oxepino[2,3-c]pyrazol-4-one

According to Scheme 10, Step 7: To a solution of 2-(4-methoxy-benzyl)-2H,7H-oxepino[2,3-c]pyrazol-4-one (120 mg, 0.44 mmol) in MeOH (15 mL) was added Pd/C (12 mg). The reaction mixture was stirred at rt for 0.5 h under H₂ atmosphere. The mixture was filtered and concentrated under reduced pressure. The residue was purified by Prep. TLC (PE:EtOAc, 1:1) to afford the title compound (15 mg, 12%).

MS (ESI): m/z 273 (M+H)⁺.

### 5-Bromo-2-(4-methoxy-benzyl)-6,7-dihydro-2H,5H-oxepino[2,3-c]pyrazol-4-one

According to Scheme 10, Step 8: A mixture of 2-(4-methoxy-benzyl)-6,7-dihydro-2H,5H-oxepino[2,3-c]pyrazol-4-one (15 mg, 55 μmol) and PhNMe₃Br₃ (20.7 mg, 55 μmol) in CHCl₃ (3 mL) was refluxed for 30 min. After cooling to rt, the mixture was diluted with DCM (20 mL), washed with brine (8 mL×2) and water (10 mL), dried over Na₂SO₄, filtered and concentrated in vacuum to afford the crude product without further purification (15 mg, 78%).

MS (ESI): m/z 351, 353 (M+H)⁺.

### [8-(4-Methoxy-benzyl)-4,8-dihydro-5H-6-oxa-3-thia-1,7,8-triaza-cyclopenta[e]azulen-2-yl]-(4-methyl-pyrimidin-2-yl)-amine

According to Scheme 10, Step 9: A mixture of 5-bromo-2-(4-methoxy-benzyl)-6,7-dihydro-2H,5H-oxepino[2,3-c]pyrazol-4-one (15 mg, 43 μmol) and (4-methyl-pyrimidin-2-yl)-thiourea (7.9 mg, 47 μmol) in EtOH (3 mL) was stirred at reflux for 15 h. After cooling to rt, the mixture was concentrated in vacuum to afford the crude product used for the next step directly (18.1 mg, 100%).

MS (ESI): m/z 421 (m+H)⁺.

### (4,8-Dihydro-5H-6-oxa-3-thia-1,7,8-triaza-cyclopenta[e]azulen-2-yl)-(4-methyl-pyrimidin-2-yl)-amine

According to Scheme 10, Step 10: A solution of [8-(4-methoxy-benzyl)-4,8-dihydro-5H-6-oxa-3-thia-1,7,8-triaza-cyclopenta[e]azulen-2-yl]-(4-methyl-pyrimidin-2-yl)-amine (18.1 mg, 43 μmmol) in TFA (3 mL) was refluxed for 2 h under N₂. After the reaction was finished, the mixture was diluted with DCM and concentrated in vacuum. The residue was dissolved in DMSO and purified by Prep. HPLC to give the desired product (2.2 mg, 17%).

¹H-NMR (400 MHz, DMSO-d₆): δ 11.45 (s, 1H), 8.41 (d, J=5.2 Hz, 1H), 7.65 (s, 1H), 6.87 (d, J=5.2 Hz, 1H), 4.26 (t, J=4.0 Hz, 2H), 3.12 (t, J=4.4 Hz, 2H), 2.40 (s, 3H);

MS (ESI): m/z 301 (M+H)⁺.

### Example 11

### Dimethyl-carbamic acid 2-(4-methyl-pyrimidin-2-ylamino)-4,5,6,6a,7,8-hexahydro-3-thia-1,7,8-triaza-cyclopenta[e]azulen-5-yl ester (Final Compound 1-6)

### 6-(4-Methoxy-benzyl)-2,3,6,7b-tetrahydro-1aH-1-oxa-6,7-diaza-cyclopropa[e]azulen-4-one

According to Scheme 11, Step 3: To a solution of (Z)-2-(4-methoxybenzyl)-5,6-dihydrocyclohepta[c]pyrazol-4(21)-one (5.4 g, 20 mmol) in dry DCM (100 mL) was added m-CPBA (6.90 g, 40.0 mmol) in portions at 0° C. The reaction was warmed to rt and stirred for 5 h. The mixture was diluted by DCM (200 mL), washed by Na₂SO₃ (50 mL×3), NaHCO₃ (30 mL×3) and H₂O (30 mL×2). The organic layer was dried over Na₂SO₄, filtered and concentrated to afford the crude product without purification (4.82 g, 85%).

MS (ESI): m/z 285 (M+H)⁺.

### 7-Hydroxy-2-(4-methoxy-benzyl)-5,6,7,8-tetrahydro-2H-cycloheptapyrazol-4-one

According to Scheme 11, Step 4: To a solution of 6-(4-methoxy-benzyl)-2,3,6,7b-tetrahydro-1aH-1-oxa-6,7-diaza-cyclopropa[e]azulen-4-one (4.82 g, 17.0 mmol) in MeOH (80 mL) was added Pd/C (0.8 g). The reaction mixture was stirred at rt for 12 h under H₂ atmosphere. The mixture was filtered and concentrated under reduced pressure. The residue was purified by flash chromatography on silica gel (PE:EtOAc, 5:1 to 1:1) to afford the desired compound (1.72 g, 35%).

MS (ESI): m/z 287 (M+H)⁺.

### Dimethyl-carbamic acid 2-(4-methoxy-benzyl)-4-oxo-2,4,5,6,7,8-hexahydro-cycloheptapyrazol-7-yl ester

According to Scheme 11, Step 5: To a solution of 7-hydroxy-2-(4-methoxy-benzyl)-5,6,7,8-tetrahydro-2H-cycloheptapyrazol-4-one (200 mg, 0.70 mmol) in dry THF (6 mL) was added NaH (34 mg, 1.4 mmol) at 0° C. in one portion under N₂. The mixture was stirred at 0° C. for 30 min, then A (151 mg, 1.40 mmol) was added dropwise to the above reaction mixture. The reaction mixture was warmed to rt and stirred overnight. The reaction was quenched with ice water (10 mL) and extracted by DCM (30 mL×4). The organic layers were dried over Na₂SO₄, filtered and concentrated to give the crude product. The residue was purified by flash chromatography on silica gel (PE:EtOAc, 1:1) to afford the desired product (180 mg, 71%).

MS (ESI): m/z 358 ((M+H)±.

### Dimethyl-carbamic acid 5-bromo-2-(4-methoxy-benzyl)-4-oxo-2,4,5,6,7,8-hexahydro-cycloheptapyrazol-7-yl ester

According to Scheme 7, Step 7: A mixture of dimethyl-carbamic acid 2-(4-methoxy-benzyl)-4-oxo-2,4,5,6,7,8-hexahydro-cycloheptapyrazol-7-yl ester (180 mg, 0.50 mmol) and PhNMe₃Br₃ (188 mg, 0.50 mmol) in CHCl₃ (6 mL) was refluxed for 30 min. After cooling to rt, the mixture was diluted by DCM (40 mL), washed by brine (10 mL×2) and water (10 mL), dried over Na₂SO₄, filtered and concentrated in vacuum to afford the crude product without further purification (200 mg, 92%).

MS (ESI): m/z 436, 438 (M+H)⁺.

### Dimethyl-carbamic acid 8-(4-methoxy-benzyl)-2-(4-methyl-pyrimidin-2-ylamino)-4,5,6,8-tetrahydro-3-thia-1,7,8-triaza-cyclopenta[e]azulen-5-yl ester

According to Scheme 7, Step 8: A mixture of dimethyl-carbamic acid 5-bromo-2-(4-methoxy-benzyl)-4-oxo-2,4,5,6,7,8-hexahydro-cycloheptapyrazol-7-yl ester (200 mg, 0.46 mmol) and (4-methyl-pyrimidin-2-yl)thiourea (84 mg, 0.50 mmol) in n-BuOH (3 mL) was stirred at reflux for 3 h. After cooling to rt, the mixture was concentrated in vacuum to afford the crude product without further purification (100 mg, 43%).

MS (ESI): m/z 506 (M+H)⁺.

### Dimethyl-carbamic acid 2-(4-methyl-pyrimidin-2-ylamino)-4,5,6,8-tetrahydro-3-thia-1,7,8-triaza-cyclopenta[e]azulen-5-yl ester

According to Scheme 7, Step 9: A solution of dimethyl-carbamic acid 8-(4-methoxy-benzyl)-2-(4-methyl-pyrimidin-2-ylamino)-4,5,6,8-tetrahydro-3-thia-1,7,8-triaza-cyclopenta[e]azulen-5-yl ester (100 mg, 0.20 mmol) in TFA (3 mL) was stirred at 100° C. under microwave conditions for 20 min. After the reaction was finished, the mixture was diluted with DCM and concentrated to give the crude product. The residue was dissolved in DMSO and purified by Prep. HPLC to give the desired product (80 mg, 73%).

¹H-NMR (400 MHz, DMSO-d₆): δ 11.43 (br, 1H), 8.36 (d, 1H, J=4.8 Hz), 7.67 (s, 1H), 6.82 (t, 1H, J=5.2 Hz), 5.02-5.06 (m, 1H), 3.08-3.22 (m, 4H), 2.68 (s, 3H), 2.52 (s, 3H), 2.35 (s, 3H); MS (ER); m/z 386 (M+H)⁺.

### Example 12

### (5-Methoxy-4,5,6,8-tetrahydro-3-thia-1,7,8-triaza-cyclopenta[e]azulen-2-yl)-(4-methyl-pyrimidin-2-yl)-amine (Final Compound 1-15)

### 7-Methoxy-2-(4-methoxy-benzyl)-5,6,7,8-tetrahydro-2H-cycloheptapyrazol-4-one

According to Scheme 12: To a solution of 7-hydroxy-2-(4-methoxybenzyl)-5,6,7,8-tetrahydrocyclohepta[c]pyrazol-4(2H)-one (500 mg, 1.75 mmol) in dry THF (20 mL) was added NaH (140 mg, 3.5 mmol) in one portion at 0° C. After stirring at this temperature for 30 min, MeI (373 mg, 2.63 mmol) was added dropwise to the above reaction. The mixture was warmed to rt and stirred overnight. The reaction was quenched with ice water (10 mL), extracted by DCM (20 mL×4), dried over Na₂SO₄, filtered and concentrated to give the crude product. The residue was purified by Prep. TLC (PE:EtOAc, 1.5:1) to afford the desired product (60 mg, 11%).

¹H-NMR (400 MHz, CDCl₃): δ 7.68 (s, 1H), 7.15 (d, 2H, J=8.8 Hz), 6.82 (d, 2H, J=8.8 Hz), 5.09 (s, 2H), 3.74 (s, 3H), 3.68-3.72 (m, 1H), 3.32 (s, 3H), 3.15-3.20 (m, 1H), 2.99-3.05 (m, 1H), 2.71-2.78 (m, 1H), 2.44-2.51 (m, 1H), 1.92-2.08 (m, 2H).

### 5-Bromo-7-methoxy-2-(4-methoxy-benzyl)-5,6,7,8-tetrahydro-2H-cycloheptapyrazol-4-one

According to Scheme 7, Step 7: A mixture of 7-methoxy-2-(4-methoxy-benzyl)-5,6,7,8-tetrahydro-2H-cycloheptapyrazol-4-one (60 mg, 0.2 mmol) and PhNMe₃Br₃ (90 mg, 0.24 mmol) in CHCl₃ (3 mL) was refluxed for 30 min. After cooling to rt, the mixture was diluted by DCM (40 mL), washed with brine (10 mL×2) and water (10 mL), dried over Na₂SO₄, filtered and concentrated to afford the crude product without further purification (70 mg, 92%).

MS (ESI): m/z 379, 381 (M+H)⁺.

### [5-Methoxy-8-(4-methoxy-benzyl)-4,5,6,8-tetrahydro-3-thia-1,7,8-triaza-cyclopenta[e]azulen-2-yl]-(4-methyl-pyrimidin-2-yl)-amine

According to Scheme 7, Step 8: A mixture of 5-bromo-7-methoxy-2-(4-methoxy-benzyl)-5,6,7,8-tetrahydro-2H-cycloheptapyrazol-4-one (70 mg, 0.18 mmol) and (4-methyl-pyrimidin-2-yl)-thiourea (36 mg, 0.21 mmol) in n-BuOH (3 mL) was stirred at refluxed for 3 h. After cooling to rt, the mixture was concentrated in vacuum to afford the crude product without further purification (72 mg, 90%).

MS (ESI): m/z 449 (M+H)⁺.

### (5-Methoxy-4,5,6,8-tetrahydro-3-thia-1,7,8-triaza-cyclopenta[e]azulen-2-yl)-(4-methyl-pyrimidin-2-yl)-amine

According to Scheme 7, Step 9: A solution of [5-methoxy-8-(4-methoxy-benzyl)-4,5,6,8-tetrahydro-3-thia-1,7,8-triaza-cyclopenta[e]azulen-2-yl]-(4-methyl-pyrimidin-2-yl)-amine (72 mg, 0.16 mmol) in TFA (3 mL) was stirred at 100° C. under microwave conditions for 20 min. After the reaction was finished, the mixture was diluted with DCM and concentrated to give the crude product. The residue was dissolved in DMSO and purified by Prep. HPLC to give the desired product (33 mg, 63%).

¹H-NMR (400 MHz, DMSO-d₆): δ 8.41 (d, 1H, J=4.8 Hz), 7.69 (s, 1H), 6.86 (d, 1H, J=4.8 Hz), 3.80-3.83 (m, 1H), 3.24 (s, 3H), 3.12-3.13 (m, 4H), 2.40 (s, 3H); MS (ESI); m/z 329 (M+H)⁺.

### Example 13

### (5,5-Difluoro-4,5,6,8-tetrahydro-3-thia-1,7,8-triaza-cyclopenta[e]azulen-2-yl)-(4-methyl-pyrimidin-2-yl)-amine (Final Compound 1-18)

### 2-(4-Methoxy-benzyl)-2,5,6,8-tetrahydro-cycloheptapyrazole-4,7-dione

According to Scheme 13, Step 1: To a solution of 7-hydroxy-2-(4-methoxybenzyl)-5,6,7,8-tetrahydrocyclohepta[c]pyrazol-4(2H)-one (1.10 g, 3.85 mmol) in DCM (50 mL) was added PCC (1.66 g, 7.70 mmol) in portions at rt. The mixture was stirred for 8 h at rt. The reaction was diluted by DCM (200 mL), filtered and concentrated. The residue was purified by flash chromatography on silica gel (PE:EtOAc, 3:1) to give the desired compound (0.50 g, 46%).

MS (ESI): m/z 285 (M+H)⁺.

### 7,7-Difluoro-2-(4-methoxy-benzyl)-5,6,7,8-tetrahydro-2H-cycloheptapyrazol-4-one

According to Scheme 13, Step 2: To a solution of 2-(4-methoxy-benzyl)-2,5,6,8-tetrahydro-cycloheptapyrazole-4,7-dione (200 mg, 0.70 mmol) in dry DCM (7 mL) was added dropwise DAST (535 mg, 2.80 mmol) at 0° C. under N₂. The reaction mixture was stirred at this temperature for 30 min, then heated to reflux for 3 h. After cooling, the reaction was poured into ice water and basified to pH=8-9 with saturated NaHCO₃ solution and extracted with DCM (20 mL×3). The combined organic layers were dried over Na₂SO₄, filtered and concentrated in vacuum. The residue was purified by Prep. HPLC to afford the desired product (70 mg, 33%).

¹H-NMR (400 MHz, CDCl₃): δ 7.78 (s, 1H), 7.20 (d, 2H, J=8.4 Hz), 6.87 (d, 2H, J=8.4 Hz), 5.14 (s, 2H), 3.78 (s, 3H), 3.50 (t, 2H, J=14.4 Hz), 2.70-2.73 (m, 2H), 2.32-2.42 (m, 2H).

### 5-Bromo-7,7-difluoro-2-(4-methoxy-benzyl)-5,6,7,8-tetrahydro-2H-cycloheptapyrazol-4-one

According to Scheme 7, Step 7: A mixture of 7,7-difluoro-2-(4-methoxy-benzyl)-5,6,7,8-tetrahydro-2H-cycloheptapyrazol-4-one (70 mg, 0.23 mmol) and PhNMe₃Br₃ (101 mg, 0.27 mmol) in CHCl₃ (5 mL) was refluxed for 30 min. After cooling to rt, the mixture was diluted with DCM (60 mL), washed with brine (15 mL×2) and water (15 mL), dried over Na₂SO₄, filtered and concentrated in vacuum to afford the crude product without further purification (80 mg, 90%).

MS (ESP: m/z 385, 387 (M+H)⁺.

### [5,5-Difluoro-8-(4-methoxy-benzyl)-4,5,6,8-tetrahydro-3-thia-1,7,8-triaza-cyclopenta[e]azulen-2-yl]-(4-methyl-pyrimidin-2-yl)-amine

According to Scheme 7, Step 8: A mixture of 5-bromo-7,7-difluoro-2-(4-methoxy-benzyl)-5,6,7,8-tetrahydro-2H-cycloheptapyrazol-4-one (80 mg, 0.21 mmol) and (4-methyl-pyrimidin-2-yl)-thiourea (42 mg, 0.25 mmol) in n-BuOH (5 mL) was stirred at reflux for 3 h. After cooling to rt, the mixture was concentrated in vacuum to afford the crude product without further purification (88 mg, 92%).

MS (ESI): m/z 455 (M+H)⁺.

### (5,5-Difluoro-4,5,6,8-tetrahydro-3-thia-1,7,8-triaza-cyclopenta[e]azulen-2-yl)-(4-methyl-pyrimidin-2-A-amine

According to Scheme 7, Step 9: A solution of [5,5-difluoro-8-(4-methoxy-benzyl)-4,5,6,8-tetrahydro-3-thia-1,7,8-triaza-cyclopenta[e]azulen-2-yl]-(4-methyl-pyrimidin-2-yl)-amine (88 mg, 0.19 mmol) in TFA (3 mL) was stirred at 100° C. under microwave conditions for 20 min. After the reaction was finished, the mixture was diluted with DCM and concentrated to give the crude product. The residue was dissolved in DMSO and purified by Prep. HPLC to give the desired product (31 mg, 47%).

¹H-NMR (400 MHz, DMSO-d₆): δ 11.56 (br, 1H), 8.43 (d, 1H, J=5.2 Hz), 7.79 (s, 1H), 6.89 (d, 1H, J=5.2 Hz), 3.56-3.64 (m, 4H), 2.41 (s, 3H); MS (ESI) m/z 335 (M+H)⁺.

### Example 14

### (4-Methyl-pyrimidin-2-yl)-(5-methyl-4,5,6,8-tetrahydro-3-thia-1,7,8-triaza-cyclopenta[e]azulen-2-yl)-amine (Final Compound 1-11)

### 4-Iodo-1-(4-methoxy-benzyl)-1H-pyrazole-3-carbaldehyde

According to Scheme 14, Step 1: To a solution of methyl 4-iodo-1-(4-methoxybenzyl)-1H-pyrazole-3-carboxylate (80.0 g, 0.22 mol) in dry DCM (900 mL) was added DIBAL (550 mL, 0.55 mol) dropwise at −78° C. and the mixture was stirred at −78° C. for 30 min. When the conversion was complete, MeOH was added dropwise and then an aq. sat. solution of K₂CO₃ was added, the mixture was extracted with DCM (400 mL×3). The combined organic layers were dried over MgSO₄, filtered and concentrated under reduced pressure to give the desired product (70 g, 93%).

MS (ESI): m/z 343 (M+H)⁺.

### 1-[4-Iodo-1-(4-methoxy-benzyl)-1H-pyrazol-3-yl]-prop-2-en-1-ol

According to Scheme 14, Step 2: To a solution of 4-iodo-1-(4-methoxy-benzyl)-1H-pyrazole-3-carbaldehyde (40 g, 0.12 mol) in dry THF (500 mL) was added vinylmagnesium bromide (240 mL, 0.24 mol) dropwise at −78° C. and the mixture was stirred at −78° C. for 30 min. When the conversion was complete, NH₄Cl was added and the mixture was extracted with EtOAc (400 mL×3). The combined organic layers were dried over MgSO₄, filtered and concentrated under reduced pressure. The residue was purified by flash chromatography on silica gel (PE:EtOAc, 3:1) to give the title product (28 g, 63%).

MS (ESI): m/z 371 (M+H)⁺.

### 1-[1-(4-Methoxy-benzyl)-4-(4,4,5,5-tetramethyl-[1,3,2]dioxaborolan-2-yl)-1H-pyrazol-3-yl]-prop-2-en-1-ol

According to Scheme 14, Step 3: To a solution of 1-[4-iodo-1-(4-methoxy-benzyl)-1H-pyrazol-3-yl]-prop-2-en-1-ol (28 g, 75 mmol) in dry THF (500 mL) was added 2-isopropoxy-4,4,5,5-tetramethyl-[1,3,2]dioxaborolane (42.0 g, 0.22 mol). Then n-BuLi (90 mL, 0.22 mol) was added to the solution dropwise at −78° C. and the mixture was stirred at −78° C. for 30 min. When the conversion was complete, NH₄Cl was added and the mixture was extracted with EtOAc (300 mL×3). The combined organic layers were dried over MgSO₄, filtered and concentrated under reduced pressure to give the crude product (60 g).

MS (ESI): m/z 371 (M+H)⁺.

### {5-Formyl-4-[3-(1-hydroxy-allyl)-1-(4-methoxy-benzyl)-1H-pyrazol-4-yl]-thiazol-2-yl}-carbamic acid tert-butyl ester

According to Scheme 14, Step 4: To a solution of 1-[1-(4-methoxy-benzyl)-4-(4,4,5,5-tetramethyl-[1,3,2]dioxaborolan-2-yl)-1H-pyrazol-3-yl]-prop-2-en-1-ol (17 g, 46 mmol) in DMF (120 mL) and H₂O (24 mL) was added (4-bromo-5-formyl-thiazol-2-yl)-carbamic acid tert-butyl ester (14 g, 46 mol), Pd(PPh₃)Cl₂ (1.8 g, 2.0 mmol) and K₃PO₄ (19.5 g, 0.09 mol) and the mixture was stirred at 100° C. under N₂ atmosphere for 3 h. When the conversion was complete, water was added and the mixture was extracted with EtOAc (400 mL×3). The combined organic layers were washed with brine and then dried over MgSO₄, filtered and concentrated under reduced pressure. The residue was purified by flash chromatography on silica gel (PE:EtOAc, 3:1) to give the title product (5 g, 23%).

MS (ESI): m/z 471 (M+H)⁺.

### {5-Formyl-4-[3-(1-hydroxy propyl)-1-(4-methoxy-benzyl)-1H-pyrazol-4-yl]-thiazol-2-yl}-carbamic acid tert-butyl ester

According to Scheme 14, Step 5: To a solution of {5-formyl-4-[3-(1-hydroxy-allyl)-1-(4-methoxy-benzyl)-1H-pyrazol-4-yl]-thiazol-2-yl}-carbamic acid tert-butyl ester (2.0 g, 4.3 mmol) in EtOAc (20 mL) was added PtO₂ (0.2 g). The mixture was stirred at 25° C. under H₂ atmosphere for 10 h. After the reaction was finished, the mixture was filtered and concentrated under reduced pressure to give the title compound (1.5 g, 75%) as a yellow oil.

MS (ESI): m/z 473 (M+H)⁺.

### {5-Formyl-4-[1-(4-methoxy-benzyl)-3-propionyl-1H-pyrazol-4-yl]-thiazol-2-yl}-carbamic acid tert-butyl ester

According to Scheme 14, Step 6: To a solution of {5-formyl-4-[3-(1-hydroxy-propyl)-1-(4-methoxy-benzyl)-1H-pyrazol-4-yl]-thiazol-2-yl}-carbamic acid tert-butyl ester (472 mg, 1.00 mmol) in 5 mL DCM was added PCC (430 mg, 2 mmol) at rt, the solution was stirred at rt for 3 h. The mixture was filtered and concentrated under reduced pressure to give the crude product (470 mg, 100%), which was used for the next step without further purification.

MS (ESI): m/z 471 (M+H)⁺.

### [8-(4-Methoxy-benzyl)-5-methyl-6-oxo-6,8-dihydro-3-thia-1,7,8-triaza-cyclopenta[e]azulen-2-yl]-carbamic acid tert-butyl ester

According to Scheme 14, Step 7: To a solution of {5-formyl-4-[1-(4-methoxy-benzyl)-3-propionyl-1H-pyrazol-4-yl]-thiazol-2-yl}-carbamic acid tert-butyl ester (470 mg, 1.00 mmol) in DMF (3 mL) and water (3 mL) was added NaOH (400 mg), the mixture was stirred at 80° C. for 30 min. The reaction mixture was diluted with water and extracted with EtOAc. The organic layer was washed with brine, dried over Na₂SO₄, and the solvent was evaporated under reduced pressure, purified by flash chromatography on silica gel to give the title product (241 mg, 53%).

MS (ESI): m/z 453 (M+H)⁺.

### [8-(4-Methoxy-benzyl)-5-methyl-4,5,6,8-tetrahydro-3-thia-1,7,8-triaza-cyclopenta[e]azulen-2-yl]-carbamic acid tert-butyl ester

According to Scheme 14, Step 8: To a solution of [8-(4-methoxy-benzyl)-5-methyl-6-oxo-6,8-dihydro-3-thia-1,7,8-triaza-cyclopenta[e]azulen-2-yl]-carbamic acid tert-butyl ester (904 mg, 2.00 mmol) in MeOH was added NaBH₄ (380 mg, 10 mmol) and the mixture was stirred at rt for 30 min. The mixture was concentrated in vacuum. EtOAc (40 mL) and PtO₂ (100 mg) was added to the residue, the mixture was stirred at 50° C. under H₂ (50 psi.) for 16 h. After cooling, the mixture was filtered over celite and the filtrate was concentrated in vacuum. The residue was purified by silica gel column to give the title product (112 mg, 13%).

MS (ESI): m/z 441 (M+H)⁺.

### 8-(4-Methoxy-benzyl)-5-methyl-4,5,6,8-tetrahydro-3-thia-1,7,8-triaza-cyclopenta[e]azulen-2-ylamine

According to Scheme 14, Step 9: A solution of [8-(4-methoxy-benzyl)-5-methyl-4,5,6,8-tetrahydro-3-thia-1,7,8-triaza-cyclopenta[e]azulen-2-yl]-carbamic acid tert-butyl ester (50 mg, 113 μmol) in 0.5 mL of TFA and 2 mL of DCM was stirred at rt for 14 h. The mixture was concentrated in vacuum to give the title compound (35 mg, 91%).

MS (ESI): m/z 341 (M+H)⁺.

### [8-(4-Methoxy-benzyl)-5-methyl-4,5,6,8-tetrahydro-3-thia-1,7,8-triaza-cyclopenta[e]azulen-2-yl]-(4-methyl-pyrimidin-2-yl)-amine

According to Scheme 1, Step 8: A solution of 8-(4-methoxy-benzyl)-5-methyl-4,5,6,8-tetrahydro-3-thia-1,7,8-triaza-cyclopenta[e]azulen-2-ylamine (35 mg, 0.103 mmol), 2-chloro-4-methyl-pyrimidine (26 mg, 206 μmol), Pd₂(dba)₃ (9.0 mg, 0.01 mmol), Xantphos (12 mg, 0.02 mmol) and Cs₂CO₃ (67 mg, 0.20 mmol) in dioxane (1 mL) was refluxed for 1.5 h under N₂. After cooling to rt, the mixture was filtered and concentrated in vacuum to give the desired product (20 mg, 45%).

MS (ESI): m/z 433 (M+H)⁺.

### (4-Methyl-pyrimidin-2-yl)-(5-methyl-4,5,6,8-tetrahydro-3-thia-1,7,8-triaza-cyclopenta[e]azulen-2-yl)-amine

According to Scheme 1, Step 9: A solution of [8-(4-methoxy-benzyl)-5-methyl-4,5,6,8-tetrahydro-3-thia-1,7,8-triaza-cyclopenta[e]azulen-2-yl]-(4-methyl-pyrimidin-2-yl)-amine (20 mg, 46 μmol) in 1 mL of TFA was stirred at 100° C. for 3 h. After cooling to rt, the mixture was concentrated in vacuum. The residue was purified by Prep. HPLC to yield the title compound (5 mg, 35%).

¹H-NMR (400 MHz, DMSO-d₆): δ 8.40 (d, J=4.8 Hz, 1H), 7.68 (s, 1H), 6.85 (d, J=4.8 Hz, 1H), 2.93-2.97 (m, 2H), 2.73-2.79 (m, 2H), 2.39 (s, 3H), 2.16 (d, J=6.0H, 1H), 1.01 (d, J=6.8 Hz, 3H); MS (ESI): m/z 313 (M+H)⁺.

### Example 15

### (4-Methyl-pyrimidin-2-yl)-(6-methyl-4,5,6,8-tetrahydro-3-thia-1,7,8-triaza-cyclopenta[e]azulen-2-yl)-amine (Final Compound 1-12)

### [6-Hydroxy-8-(4-methoxy-benzyl)-6-methyl-4,5,6,8-tetrahydro-3-thia-1,7,8-triaza-cyclopenta[e]azulen-2-yl]-carbamic acid tert-butyl ester

According to Scheme 15, Step 1: To a solution of [8-(4-methoxy-benzyl)-6-oxo-4,5,6,8-tetrahydro-3-thia-1,7,8-triaza-cyclopenta[e]azulen-2-yl]-carbamic acid tert-butyl ester (80 mg, 0.18 mmol) in THF (2 mL), MeLi (0.18 mL, 0.54 mmol) was added at −78° C., then the mixture was stirred at rt for 1 h. After the reaction was finished, water (5 mL) was added and the mixture was extracted with EtOAc (5 mL×3). The combined organic layers were dried over Na₂SO₄, filtered and concentrated under reduced pressure to give the desired product (60 mg, 73%).

MS (ESI) m/z 457 (M+H)⁺

### 8-(4-Methoxy-benzyl)-6-methyl-4,5,6,8-tetrahydro-3-thia-1,7,8-triaza-cyclopenta[e]azulen-2-ylamine

According to Scheme 15, Step 2: A solution of [6-hydroxy-8-(4-methoxy-benzyl)-6-methyl-4,5,6,8-tetrahydro-3-thia-1,7,8-triaza-cyclopenta[e]azulen-2-yl]-carbamic acid tert-butyl ester (60 mg, 0.13 mmol) in 0.3 mL of Et₃SiH and 2 mL of DCM was stirred at rt for 0.5 h, then 0.5 mL TFA was added. The resulting solution was stirred at rt for another 16 h. Then the mixture was concentrated in vacuum, the residue was diluted with water and basified with an aq. sat. solution of K₂CO₃ to pH=8 and extracted with EtOAc (10 mL×3). The combined organic layers were dried over Na₂SO₄, filtered and concentrated in vacuum. The residue was purified by Prep. TLC (PE:EtOAc, 1:2) to give the title compound (20 mg, 45%).

MS (ESI): m/z 341 (M+H)⁺.

### [8-(4-Methoxy-benzyl)-6-methyl-4,5,6,8-tetrahydro-3-thia-1,7,8-triaza-cyclopenta[e]azulen-2-yl]-(4-methyl-pyrimidin-2-yl)-amine

According to Scheme 1, Step 8: A mixture of 8-(4-methoxy-benzyl)-6-methyl-4,5,6,8-tetrahydro-3-thia-1,7,8-triaza-cyclopenta[e]azulen-2-ylamine (20 mg, 59 μmol, 2-chloro-4-methyl-pyrimidine (15 mg, 118 μmol), Pd₂(dba)₃ (5 mg, 59 μmol), Xantphos (7 mg, 11.8 μmol) and Cs₂CO₃ (38 mg, 118 μmol) in dioxane (2 mL) was refluxed for 8 h under N₂. After cooling to rt, the mixture was filtered and concentrated in vacuum to give the title product (18 mg, 71%).

MS (ESI): m/z 433 (M+H)⁺.

### (4-Methyl-pyrimidin-2-yl)-(6-methyl-4,5,6,8-tetrahydro-3-thia-1,7,8-triaza-cyclopenta[e]azulen-2-yl)-amine

According to Scheme 1, Step 9: A solution of [8-(4-methoxy-benzyl)-6-methyl-4,5,6,8-tetrahydro-3-thia-1,7,8-triaza-cyclopenta[e]azulen-2-yl]-(4-methyl-pyrimidin-2-yl)-amine (18 mg, 42 μmol) in 1 mL of TFA was stirred at 100° C. for 3 h. After cooling to rt, the mixture was concentrated in vacuum. The residue was purified by Prep. HPLC to yield the final product (4 mg, 31%).

¹H-NMR (400 MHz, DMSO-d₆): δ 8.41 (d, J=5.2 Hz, 1H), 7.75 (s, 1H), 6.86 (d, J=5.2 Hz, 1H), 3.16-3.19 (m, 1H), 2.90-2.97 (m, 2H), 2.40 (s, 3H), 1.80-1.97 (m, 2H), 1.25 (d, J=7.2 Hz, 3H); MS (ESI): m/z 313 (M+H)⁺.

### Example 16

### 2-(5-Fluoro-4-methyl-pyrimidin-2-ylamino)-6,8-dihydro-5H-3-thia-1,7,8-triaza-cyclopenta[e]azulen-4-one (Final Compound 1-30)

### 1-(4-Methoxy-benzyl)-4-(4,4,5,5-tetramethyl-[1,3,2]dioxaborolan-2-yl)-1H-pyrazole-3-carboxylic acid methyl ester

According to Scheme 16, Step 1: To a solution of methyl 4-iodo-1-(4-methoxybenzyl)-1H-pyrazole-3-carboxylate (40.0 g, 108 mmol) and 2-isopropoxy-4,4,5,5-tetramethyl-[1,3,2]dioxaborolane (72.0 g, 387 mmol) in THF (250 mL) was added MeLi (2.5 M, 120 mL) at −78° C. under N₂ over 30 min. The mixture was stirred at −78° C. for 2 h. The mixture was quenched with water (50 mL) and extracted with EtOAc (100 mL×3). The combined organic layers were dried over MgSO₄, filtered and concentrated under reduced pressure to give the crude product which was used directly for the next step without further purification (40 g, 100%).

MS (ESI): m/z 373 (M+H)⁺.

### 4-(5-Acetyl-2-tert-butoxycarbonylamino-thiazol-4-yl)-1-(4-methoxy-benzyl)-1H-pyrazole-3-carboxylic acid methyl ester

According to Scheme 16, Step 2: To a solution of 1-(4-methoxy-benzyl)-4-(4,4,5,5-tetramethyl-[1,3,2]dioxaborolan-2-yl)-1H-pyrazole-3-carboxylic acid methyl ester (11.6 g, 31.0 mmol), (5-acetyl-4-bromo-thiazol-2-yl)-carbamic acid tert-butyl ester (10 g, 31 mmol), Pd(PPh₃)₂Cl₂ (1 g), K₃PO₄ (13.2 g, 62.0 mmol) in DMF (100 mL) and water (20 mL) was heated to 80° C. for 3 h. After the reaction was finished, water (100 mL) was added and the mixture was extracted with EtOAc (100 mL×3). The combined organic layers were dried over MgSO₄, filtered and concentrated under reduced pressure to give the crude product which was purified by flash chromatography on silica gel to give the title compound (6.5 g, 43%).

MS (ESI): m/z 487 (M+H)⁺.

### [4-[3-Formyl-1-(4-methoxy-benzyl)-1H-pyrazol-4-yl]-5-(1-hydroxy-ethyl)-thiazol-2-yl]-carbamic acid tert-butyl ester

According to Scheme 16, Step 3: To a solution of 4-(5-acetyl-2-tert-butoxycarbonylamino-thiazol-4-yl)-1-(4-methoxy-benzyl)-1H-pyrazole-3-carboxylic acid methyl ester (6.00 g, 12.3 mmol) in THF (60 mL) was added DIBAL (120 mL, 120 mmol) dropwise at −65° C., then the mixture was stirred for 30 min and MeOH (20 mL) was added at −65° C. The reaction mixture was quenched by the addition of water added dropwise. The aqueous layer was extracted with EtOAc. The organic layer was washed with brine, dried over Na₂SO₄, filtered and the solvent was evaporated under reduced pressure to give the crude product (3.2 g, 57%).

MS (ESI): m/z 459 (M+H)⁺.

### {5-Acetyl-4-[3-formyl-1-(4-methoxy-benzyl)-1H-pyrazol-4-yl]-thiazol-2-yl}-carbamic acid tert-butyl ester

According to Scheme 16, Step 4: To a solution of [4-[3-formyl-1-(4-methoxy-benzyl)-1H-pyrazol-4-yl]-5-(1-hydroxy-ethyl)-thiazol-2-yl]-carbamic acid tert-butyl ester (2.5 g, 5.5 mmol) in 50 mL of DCM was added PCC (5.8 g, 27 mmol) at rt. The resulting mixture was stirred at rt for 0.5 h. The mixture was filtered and concentrated under reduced pressure to give the title product (1.1 g, 48%), which was used for the next step without further purification.

MS (ESI): m/z 457 (M+H)⁺.

### [8-(4-Methoxy-benzyl)-4-oxo-4,8-dihydro-3-thia-1,7,8-triaza-cyclopenta[e]azulen-2-yl]-carbamic acid tert-butyl ester

According to Scheme 16, Step 5: To a solution of {5-acetyl-4-[3-formyl-1-(4-methoxy-benzyl)-1H-pyrazol-4-yl]-thiazol-2-yl}-carbamic acid tert-butyl ester (1.1 g, 2.4 mmol) in DMF (20 mL) and water (4 mL) was added NaOH (200 mg) and the mixture was stirred at 60° C. for 30 min. The reaction mixture was diluted with water. The mixture was extracted with EtOAc. The organic layer was washed with brine, dried over Na₂SO₄, and the solvent was evaporated under reduced pressure, purified by flash chromatography on silica gel to give the title product (220 mg, 22%).

MS (ESI): m/z 439 (M+H)⁺.

### [8-(4-Methoxy-benzyl)-4-oxo-4,5,6,8-tetrahydro-3-thia-1,7,8-triaza-cyclopenta[e]azulen-2-yl]-carbamic acid tert-butyl ester

According to Scheme 16, Step 6: To a solution of [8-(4-methoxy-benzyl)-4-oxo-4,8-dihydro-3-thia-1,7,8-triaza-cyclopenta[e]azulen-2-yl]-carbamic acid tert-butyl ester (220 mg, 0.50 mmol) in EtOAc (40 mL) was added PtO₂ (100 mg). The mixture was stirred at 50° C. under H₂ (50 psi.) for 24 h. After cooling to rt, the mixture was filtered over celite and the filtrate was concentrated in vacuum to give the title compound (150 mg, 68%).

MS (ESI): m/z 441 (M+H)⁺.

### 2-Amino-8-(4-methoxy-benzyl)-6,8-dihydro-5H-3-thia-1,7,8-triaza-cyclopenta[e]azulen-4-one

According to Scheme 14, Step 8: A solution of [8-(4-methoxy-benzyl)-4-oxo-4,5,6,8-tetrahydro-3-thia-1,7,8-triaza-cyclopenta[e]azulen-2-yl]-carbamic acid tert-butyl ester (150 mg, 0.35 mmol) in TFA (2 mL) and DCM (4 mL) was stirred at rt for 5 h. Then the mixture was concentrated in vacuum to give the crude product (100 mg, 86%), which was used for next step without further purification.

MS (ESI): m/z 341 (M+H)⁺.

### 2-(5-Fluoro-4-methyl-pyrimidin-2-ylamino)-8-(4-methoxy-benzyl)-6,8-dihydro-5H-3-thia-1,7,8-triaza-cyclopenta[e]azulen-4-one

According to Scheme 1, Step 8: A mixture of 2-amino-8-(4-methoxy-benzyl)-6,8-dihydro-5H-3-thia-1,7,8-triaza-cyclopenta[e]azulen-4-one (100 mg, 0.29 mmol), 2-chloro-4-methyl-pyrimidine (44 mg, 0.30 mmol), Pd₂(dba)₃ (27 mg, 29 μmol), Xantphos (35 mg, 58 μmol) and Cs₂CO₃ (196 mg, 0.60 mmol) in dioxane (5 mL) was refluxed for 2.5 h under N₂. After cooling to rt, the mixture was filtered and concentrated in vacuum to give the crude product (30 mg, 24%).

MS (ESI): m/z 433 (M+H)⁺.

### 2-(5-Fluoro-4-methyl-pyrimidin-2-ylamino)-6,8-dihydro-5H-3-thia-1,7,8-triaza-cyclopenta[e]azulen-4-one

According to Scheme 1, Step 9: A solution of 2-(5-fluoro-4-methyl-pyrimidin-2-ylamino)-8-(4-methoxy-benzyl)-6,8-dihydro-5H-3-thia-1,7,8-triaza-cyclopenta[e]azulen-4-one (30 mg, 0.07 mmol) in 2 mL of TFA and 0.5 mL of water was stirred at 100° C. for 2 h. After cooling to rt, the mixture was concentrated in vacuum. The residue was purified by Prep. HPLC to yield the title compound (10 mg, 50%).

¹H-NMR (400 MHz, DMSO-d₆): δ 12.32 (s, 1H), 8.63 (s, 1H), 7.92 (s, 1H), 2.95 (t, J=5.2 Hz, 2H), 2.76 (t, J=5.2 Hz, 2H), 2.46 (s, 3H); MS (ESI): m/z 331 (M+H)⁺.

### Example 17

### 2-(4-Methyl-pyrimidin-2-ylamino)-4,5,6,7-tetrahydro-3-thia-1,7,8-triaza-cyclopenta[e]azulen-6-ol (Final Compound 1-3)

### [6-Hydroxy-8-(4-methoxy-benzyl)-4,5,6,8-tetrahydro-3-thia-1,7,8-triaza-cyclopenta[e]azulen-2-yl]-carbamic acid tert-butyl ester

According to Scheme 17: [8-(4-Methoxy-benzyl)-6-oxo-6,8-dihydro-3-thia-1,7,8-triaza-cyclopenta[e]azulen-2-yl]-carbamic acid tert-butyl ester (160 mg, 0.36 mmol) was dissolved in dry THF (15 mL) and the solution was cooled to 0° C. Then LiAlH₄ (42 mg, 1.10 mmol) was added in portions. The resulting mixture was stirred at 0° C. for 1 h and then it was allowed to warm to rt and stirred for another 1 h. After the reaction was complete, the reaction mixture was quenched with 5% NaOH solution. The mixture was filtered. The filtrate was collected and concentrated under reduced pressure. The residue was purified by Prep. TLC to give the desired product (65 mg, 40%) as a light yellow solid.

MS (ESI): m/z 443 (M+H)⁺.

### 2-Amino-8-(4-methoxy-benzyl)-4,5,6,8-tetrahydro-3-thia-1,7,8-triaza-cyclopenta[e]azulen-6-ol

According to Scheme 14, Step 9: A solution of [6-hydroxy-8-(4-methoxy-benzyl)-4,5,6,8-tetrahydro-3-thia-1,7,8-triaza-cyclopenta[e]azulen-2-yl]-carbamic acid tert-butyl ester (150 mg, 0.34 mmol) in saturated HCl methanol solution (10 mL) was stirred for 1 h. Then the mixture was concentrated under reduced pressure to afford the crude product (110 mg, 95%) which was used for the next step without further purification.

MS (ESI): m/z 343 (M+H)⁺.

### 8-(4-Methoxy-benzyl)-2-(4-methyl-pyrimidin-2-ylamino)-4,5,6,8-tetrahydro-3-thia-1,7,8-triaza-cyclopenta[e]azulen-6-ol

According to Scheme 1, Step 8: To a solution of 2-amino-8-(4-methoxy-benzyl)-4,5,6,8-tetrahydro-3-thia-1,7,8-triaza-cyclopenta[e]azulen-6-ol (110 mg, 0.32 mmol), 4-methyl-2-chloro-pyrimidine (45 mg, 0.35 mmol) in dioxane (8 mL) were added Pd₂(dba)₃ (15 mg, 16 μmol), Xantphos (19 mg, 32 μmol) and K₂CO₃ (92 mg, 644 μmol). The resulting mixture was stirred at 100° C. under N₂ overnight. After cooling to rt, the mixture was filtered and the filtrate was collected and concentrated under reduced pressure. The residue was purified by Prep. TLC to give the title product (38 mg, 28%).

MS (ESI): m/z 435 (M+H)⁺.

### 2-(4-Methyl-pyrimidin-2-ylamino)-4,5,6,8-tetrahydro-3-thia-1,7,8-triaza-cyclopenta[e]azulen-6-ol

According to Scheme 1, Step 9: A solution of 8-(4-methoxy-benzyl)-2-(4-methyl-pyrimidin-2-ylamino)-4,5,6,8-tetrahydro-3-thia-1,7,8-triaza-cyclopenta[e]azulen-6-ol (38 mg, 88 μmol) in TFA (4 mL) was stirred at 100° C. for 10 min under microwave condition. After the reaction was finished, the solvent was removed under reduced pressure. The crude product was dissolved in EtOAc and the solution was washed with saturated aqueous NaHCO₃ solution. The organic phase was dried over MgSO₄ and concentrated under reduced pressure. The crude product was purified by Prep. HPLC to give the desired product (17 mg, 63%) as a white solid.

¹H-NMR (400 MHz, CD₃OD): δ 8.47 (d, 1H, J=5.2 Hz), 8.06 (s, 1H), 6.98 (d, 1H, J=5.2 Hz), 5.12 (d, 1H, J=5.6 Hz), 3.19-3.26 (m, 1H), 2.91-2.97 (m, 1H), 2.62 (s, 3H), 2.29-2.35 (m, 1H), 2.04-2.11 (m, 1H); MS (ESI): m/z 315 (M+H)⁺.

### Example 18

### (6,6-Difluoro-5,5-dimethyl-4,5,6,7-tetrahydro-3-thia-1,7,8-triaza-cyclopenta[e]azulen-2-yl)-(4-methyl-pyrimidin-2-yl)-amine (Final Compound 1-9)

### [8-(4-Methoxy-benzyl)-6-oxo-4,5,6,8-tetrahydro-3-thia-1,7,8-triaza-cyclopenta[e]azulen-2-yl]-carbamic acid tert-butyl ester

According to Scheme 18, Step 1: To a solution of [8-(4-methoxy-benzyl)-6-oxo-6,8-dihydro-3-thia-1,7,8-triaza-cyclopenta[e]azulen-2-yl]-carbamic acid tert-butyl ester (700 mg, 1.60 mol) in EtOAc (20 mL) was added PtO₂ (100 mg) and the mixture was stirred at 50° C. under H₂ (50 psi.) for 15 h. The mixture was filtered over celite and the filtrate was concentrated in vacuum. The residue was purified by Prep. TLC (PE:EtOAc, 1:1) to give the title compound (200 mg, 28%).

¹H-NMR (400 MHz, CDCl₃): δ 7.67 (s, 1H), 7.29 (d, J=8.4 Hz, 2H), 6.91 (d, J=8.4 Hz, 2H), 5.35 (s, 2H), 3.82 (s, 3H), 3.00-3.05 (m, 4H), 1.53 (s, 9H); MS (ESI): m/z 441 (M+H)⁺.

### 2-Amino-8-(4-methoxy-benzyl)-4,8-dihydro-5H-3-thia-1,7,8-triaza-cyclopenta[e]azulen-6-one

According to Scheme 18, Step 2: A solution of [8-(4-methoxy-benzyl)-6-oxo-4,5,6,8-tetrahydro-3-thia-1,7,8-triaza-cyclopenta[e]azulen-2-yl]-carbamic acid tert-butyl ester (150 mg, 0.35 mmol) in 1 mL of TFA and 4 mL of DCM was stirred at rt for 7 h. Concentrated in vacuum, the residue was diluted with water and basified with K₂CO₃ to pH=8, the mixture was extracted with EtOAc (10 mL×2). The combined organic layers were dried over Na₂SO₄, filtered and concentrated in vacuum to give the desired product (100 mg, 86%).

MS (ESI): m/z 341 (m+H)⁺.

### 8-(4-Methoxy-benzyl)-2-(4-methyl-pyrimidin-2-ylamino)-4,8-dihydro-5H-3-thia-1,7,8-triaza-cyclopenta[e]azulen-6-one

According to Scheme 18, Step 3: A solution of 2-amino-8-(4-methoxy-benzyl)-4,8-dihydro-5H-3-thia-1,7,8-triaza-cyclopenta[e]azulen-6-one (100 mg, 0.29 mmol), 2-chloro-4-methyl-pyrimidine (75 mg, 0.58 mmol), Pd₂(dba)₃ (26 mg, 29 μmol), Xantphos (34 mg, 58 μmol) and Cs₂CO₃ (189 mg, 0.58 mmol) in dioxane (4 mL) was refluxed for 3 h under N₂. After cooling to rt, the mixture was filtered and concentrated in vacuum to give the crude product (55 mg, 44%).

MS (ESI): m/z 433 (M+H)⁺.

### 2-(4-Methyl-pyrimidin-2-ylamino)-4,7-dihydro-5H-3-thia-1,7,8-triaza-cyclopenta[e]azulen-6-one

According to Scheme 18, Step 4: A solution of 8-(4-methoxy-benzyl)-2-(4-methyl-pyrimidin-2-ylamino)-4,8-dihydro-5H-3-thia-1,7,8-triaza-cyclopenta[e]azulen-6-one (55 mg, 127 μmol) in 2 mL of TFA was stirred at 100° C. for 1 h. After cooling to rt, the mixture was concentrated in vacuum and the residue was purified by Prep. HPLC to yield the desired compound (25 mg, 63%).

¹H-NMR (400 MHz, DMSO-d₆): δ 8.42 (d, J=4.8 Hz, 1H), 7.82 (s, 1H), 6.88 (d, J=4.8 Hz, 1H), 2.99 (t, J=4.8 Hz, 2H), 2.82 (t, J=4.8 Hz, 2H), 2.41 (s, 3H); MS (ESI): m/z 313 (M+H)⁺.

### 8-(4-Methoxy-benzyl)-2-[(4-methoxy-benzyl)-(4-methyl-pyrimidin-2-yl)-amino]-4,8-dihydro-5H-3-thia-1,7,8-triaza-cyclopenta[e]azulen-6-one

According to Scheme 18, Step 5: To a suspension of 2-(4-methyl-pyrimidin-2-ylamino)-4,7-dihydro-5H-3-thia-1,7,8-triaza-cyclopenta[e]azulen-6-one (10 mg, 32 μmol) and K₂CO₃ (16 mg, 112 μmol) in MeCN (2 mL) was added PMBCl (10.4 mg, 67 μmol) and the mixture was refluxed for 1 h. After cooling to rt, the mixture was diluted with H₂O (5 mL) and extracted with EtOAc (5 mL×3). The combined organic layers were dried over Na₂SO₄, filtered and the filtrate was concentrated in vacuum to give the crude product (15 mg, 85%).

MS (ESI): m/z 553 (M+H)⁺.

### 8-(4-Methoxy-benzyl)-2-[(4-methoxy-benzyl)-(4-methyl-pyrimidin-2-yl)-amino]-5,5-dimethyl-4,8-dihydro-5H-3-thia-1,7,8-triaza-cyclopenta[e]azulen-6-one

According to Scheme 18, Step 6: To a solution of 8-(4-methoxy-benzyl)-2-[(4-methoxy-benzyl)-(4-methyl-pyrimidin-2-yl)-amino]-4,8-dihydro-5H-3-thia-1,7,8-triaza-cyclopenta[e]azulen-6-one (15 mg, 27 μmol) and MeI (8.4 mg, 59 μmol) in THF (2 mL), NaH (3.3 mg, 81 μmol, 60% in oil) was added at 0° C., then the mixture was stirred at rt for 40 min. After the reaction was finished, water (5 mL) was added and the mixture was extracted with EtOAc (5 mL×3). The combined organic layers were dried over Na₂SO₄, filtered and concentrated under reduced pressure to give the crude product (13 mg, 83%).

MS (ESI): m/z 581 (M+H)⁺.

### 5,5-Dimethyl-2-(4-methyl-pyrimidin-2-ylamino)-4,7-dihydro-5H-3-thia-1,7,8-triaza-cyclopenta[e]azulen-6-one

According to Scheme 18, Step 7: A solution of 8-(4-methoxy-benzyl)-2-[(4-methoxy-benzyl)-(4-methyl-pyrimidin-2-yl)-amino]-5,5-dimethyl-4,8-dihydro-5H-3-thia-1,7,8-triaza-cyclopenta[e]azulen-6-one (13 mg, 22 μmol) in 2 mL of TFA was stirred at 100° C. for 1 h. After cooling to rt, the mixture was concentrated in vacuum. The residue was purified by Prep. HPLC to yield the desired product (3 mg, 40%).

¹H-NMR (400 MHz, DMSO-d₆): δ 8.43 (d, J=4.8 Hz, 1H), 7.81 (s, 1H), 6.88 (d, J=4.8 Hz, 1H), 2.94 (s, 2H), 2.41 (s, 3H), 1.09 (s, 6H); MS (ESI): m/z 341 (M+H)⁺.

### (6,6-Difluoro-5,5-dimethyl-4,5,6,7-tetrahydro-3-thia-1,7,8-triaza-cyclopenta[e]azulen-2-yl)-(4-methyl-pyrimidin-2-yl)-amine

According to Scheme 18, Step 8: To a solution of 5,5-dimethyl-2-(4-methyl-pyrimidin-2-ylamino)-4,7-dihydro-5H-3-thia-1,7,8-triaza-cyclopenta[e]azulen-6-one (15 mg, 44 μmol) in 1 mL of DCM, DAST (71 mg, 0.44 mmol) was added dropwise at 0° C. The resulting mixture was stirred at 40° C. for 2 h. After cooling to rt, the mixture was concentrated in vacuum. The residue was diluted with water and basified with K₂CO₃ to pH=8 and extracted with EtOAc (10 mL×3). The combined organic layers were dried over Na₂SO₄, filtered and concentrated in vacuum. The residue was purified by Prep. HPLC to yield the title product (5 mg, 31%).

¹H-NMR (400 MHz, DMSO-d₆): δ 8.41 (d, J=4.8 Hz, 1H), 7.88 (s, 1H), 6.87 (d, J=4.8 Hz, 1H), 2.94 (s, 2H), 2.40 (s, 3H), 1.07 (s, 6H); MS (ESI): m/z 363 (M+H)⁺.

### Example 19

### (6-Methoxy-4,5,6,7-tetrahydro-3-thia-1,7,8-triaza-cyclopenta lel azulen-2-yl)-(4-methyl-pyrimidin-2-yl)-amine (Final Compound 1-24)

### [6-Hydroxy-8-(4-methoxy-benzyl)-4,5,6,8-tetrahydro-3-thia-1,7,8-triazacyclopenta[e]azulen-2-yl]-(4-methoxy-benzyl)-carbamic acid tert-butyl ester

According to Scheme 19, Step 1: To a solution of [6-hydroxy-8-(4-methoxy-benzyl)-4,5,6,8-tetrahydro-3-thia-1,7,8-triaza-cyclopenta[e]azulen-2-yl]-carbamic acid tert-butyl ester (442 mg, 1.00 mmol) in MeCN (10 mL) was added PMBCl (312 mg, 2.00 mmol) and K₂CO₃ (1.38 g, 10 mmol). The mixture was stirred at reflux for 2 h. After cooling to rt, the mixture was filtered and concentrated under reduced pressure. The residue was purified by flash chromatography on silica gel (PE:EtOAc, 5:1 to 2:1) to give the title compound (418 mg, 74%).

MS (ESI): m/z 563 (M+H)⁺.

### (4-Methoxy-benzyl)-[6-methoxy-8-(4-methoxy-benzyl)-4,5,6,8-tetrahydro-3-thia-1,7,8-triaza-cyclopenta[e]azulen-2-yl]-carbamic acid tert-butyl ester

According to Scheme 19, Step 2: To a solution of [6-hydroxy-8-(4-methoxy-benzyl)-4,5,6,8-tetrahydro-3-thia-1,7,8-triaza-cyclopenta[e]azulen-2-yl]-(4-methoxy-benzyl)-carbamic acid tert-butyl ester (200 mg, 0.36 mmol) and MeI (76 mg, 0.53 mmol) in THF (4 mL), NaH (17 mg, 427 μmol, 60% in oil) was added at 0° C. Then the mixture was stirred at rt for 1 h. After the reaction was finished, water (15 mL) was added and the mixture was extracted with EtOAc (15 mL×3). The combined organic layers were dried over Na₂SO₄, filtered and concentrated under reduced pressure to give the crude product (180 mg, 90%).

MS (ESI): m/z 577 (M+H)⁺.

### (4-Methoxy-benzyl)-[6-methoxy-8-(4-methoxy-benzyl)-4,5,6,8-tetrahydro-3-thia-1,7,8-triaza-cyclopenta[e]azulen-2-yl]-amine

According to Scheme 19, Step 3: A solution of (4-methoxy-benzyl)-[6-methoxy-8-(4-methoxy-benzyl)-4,5,6,8-tetrahydro-3-thia-1,7,8-triaza-cyclopenta[e]azulen-2-yl]-carbamic acid tert-butyl ester (180 mg, 0.31 mmol) in 0.5 mL of TFA and 2 mL of DCM was stirred at rt for 2 h. Then the mixture was concentrated in vacuum and the residue was diluted with water, basified with K₂CO₃ to pH=8 and extracted with EtOAc (10 mL×3). The combined organic layers were dried over Na₂SO₄, filtered and concentrated in vacuum to give the title compound (132 mg, 89%).

MS (ESI): m/z 477 (M+H)⁺.

### (4-Methoxy-benzyl)-[6-methoxy-8-(4-methoxy-benzyl)-4,5,6,8-tetrahydro-3-thia-1,7,8-triaza-cyclopenta[e]azulen-2-yl]-(4-methyl-pyrimidin-2-yl)-amine

According to Scheme 1, Step 8: A mixture of (4-methoxy-benzyl)-[6-methoxy-8-(4-methoxy-benzyl)-4,5,6,8-tetrahydro-3-thia-1,7,8-triaza-cyclopenta[e]azulen-2-yl]-amine (132 mg, 277 μmol), 2-chloro-4-methyl-pyrimidine (106 mg, 0.83 mmol), Pd₂(dba)₃ (25 mg, 27 μmol), Xantphos (31 mg, 54 μmol) and Cs₂CO₃ (271 mg, 0.83 mmol) in dioxane (5 mL) was refluxed for 24 h under N₂. After cooling to rt, the mixture was filtered and concentrated in vacuum. The residue was purified by Prep. TLC (PE:EtOAc, 1:1) to yield the title product (90 mg, 57%).

MS (ESI): m/z 569 (M+H)⁺.

### (6-Methoxy-4,5,6,7-tetrahydro-3-thia-1,7,8-triaza-cyclopenta[e]azulen-2-yl)-(4-methyl-pyrimidin-2-yl)-amine

According to Scheme 1, Step 9: A solution of (4-methoxy-benzyl)-[6-methoxy-8-(4-methoxy-benzyl)-4,5,6,8-tetrahydro-3-thia-1,7,8-triaza-cyclopenta[e]azulen-2-yl]-(4-methyl-pyrimidin-2-yl)-amine (90 mg, 158 μmol) in 2 mL of TFA was stirred at 100° C. for 1 h. After cooling to rt, the mixture was concentrated in vacuum. The residue was purified by Prep. HPLC to yield the title (15 mg, 29%).

¹H-NMR (400 MHz, DMSO-d₆): δ 8.41 (d, J=4.8 Hz, 1H), 7.78 (s, 1H), 6.86 (d, J=4.8 Hz, 1H), 4.53 (d, J=5.2 Hz, 1H), 3.25 (s, 3H), 2.98-3.01 (m, 1H), 2.84-2.88 (m, 1H), 2.34 (s, 3H), 2.29-2.32 (m, 1H), 1.75-1.81 (m, 1H); MS (ESI): m/z 329 (M+H)⁺.

### Example 20

### (4-Methyl-pyrimidin-2-yl)-(6-trifluoromethyl-4,5,6,7-tetrahydro-3-thia-1,7,8-triaza-cyclopenta[e]azulen-2-yl)-amine (Final Compound 1-27)

### [6-Hydroxy-8-(4-methoxy-benzyl)-6-trifluoromethyl-4,5,6,8-tetrahydro-3-thia-1,7,8-triaza-cyclopenta[e]azulen-2-yl]-carbamic acid tert-butyl ester

According to Scheme 20, Step 1: To a solution of [8-(4-methoxy-benzyl)-6-oxo-4,5,6,8-tetrahydro-3-thia-1,7,8-triaza-cyclopenta[e]azulen-2-yl]-carbamic acid tert-butyl ester (170 mg, 0.38 mmol) in 4 mL of THF at 0° C. was added TMSCF₃ (108 mg, 0.76 mmol) dropwise. Then CsF (6 mg, 38 μmol) was added and the mixture was stirred at rt for 1 h. 5 mL of 3N HCl and 3 mL of MeOH were added. The mixture was stirred for another 2 h at rt, extracted with EtOAc (10 mL×3). The combined organic layers were dried over Na₂SO₄, filtered and concentrated in vacuum to give the crude compound (150 mg, 77%).

MS (ESI): m/z 511 (M+H)⁺.

### [6-Chloro-8-(4-methoxy-benzyl)-6-trifluoromethyl-4,5,6,8-tetrahydro-3-thia-1,7,8-triaza-cyclopenta[e]azulen-2-yl]-carbamic acid tert-butyl ester

According to Scheme 20, Step 2: A solution of [6-hydroxy-8-(4-methoxy-benzyl)-6-trifluoromethyl-4,5,6,8-tetrahydro-3-thia-1,7,8-triaza-cyclopenta[e]azulen-2-yl]-carbamic acid tert-butyl ester (150 mg, 0.29 mmol) in 2 mL of SOCl₂, 0.2 mL of DMF and 4 mL of DCM was stirred at rt for 27 h. The mixture was basified with K₂CO₃ to pH=8 at 0° C., and extracted with EtOAc (10 mL×3). The combined organic layers were dried over Na₂SO₄, filtered and concentrated in vacuo to give the title compound (120 mg, 78%).

MS (ESI): m/z 529, 531 (M+H)⁺.

### [8-(4-Methoxy-benzyl)-6-trifluoromethyl-4,5,6,8-tetrahydro-3-thia-1,7,8-triaza-cyclopenta[e]azulen-2-yl]-carbamic acid tert-butyl ester

According to Scheme 20, Step 3: To a solution of [6-chloro-8-(4-methoxy-benzyl)-6-tri fluoromethyl-4,5,6,8-tetrahydro-3-thia-1,7,8-triaza-cyclopenta[e]azulen-2-yl]-carbamic acid tert-butyl ester (120 mg, 0.23 mmol) in 2 mL of THF at 0° C. was added LiBH₄ (50 mg, 2.3 mmol). The resulting mixture was stirred at reflux for 7 h. After cooling to rt, 10 mL of H₂O was added then extracted with EtOAc (10 mL×3). The combined organic layers were dried over Na₂SO₄, filtered and concentrated in vacuum. The residue was purified by Prep. TLC (PE:EtOAc, 2:1) to yield the title product (40 mg, 35%).

MS (ESI): m/z 495 (M+H)⁺.

### 8-(4-Methoxy-benzyl)-6-trifluoromethyl-4,5,6,8-tetrahydro-3-thia-1,7,8-triaza-cyclopenta[e]azulen-2-ylamine

According to Scheme 18, Step 2: A solution of [8-(4-methoxy-benzyl)-6-trifluoromethyl-4,5,6,8-tetrahydro-3-thia-1,7,8-triaza-cyclopenta[e]azulen-2-yl]-carbamic acid tert-butyl ester (40 mg, 81 μmol) in 0.5 mL of TFA and 2 mL of DCM was stirred at rt for 5 h. The mixture was concentrated in vacuum to give the title compound (30 mg, 94%).

MS (ESI): m/z 395 (M+H)⁺.

### [8-(4-Methoxy-benzyl)-6-trifluoromethyl-4,5,6,8-tetrahydro-3-thia-1,7,8-triaza-cyclopenta[e]azulen-2-yl]-(4-methyl-pyrimidin-2-yl)-amine

According to Scheme 1, Step 8: A mixture of 8-(4-methoxy-benzyl)-6-trifluoromethyl-4,5,6,8-tetrahydro-3-thia-1,7,8-triaza-cyclopenta[e]azulen-2-ylamine (30 mg, 76 μmol), 2-chloro-4-methyl-pyrimidine (14 mg, 114 μmol), Pd₂(dba)₃ (7 mg, 7.6 μmol), Xantphos (9 mg, 14 μmol) and Cs₂CO₃ (46 mg, 142 μmol) in dioxane (2 mL) was refluxed for 1 h under N₂. After cooling to rt, the mixture was filtered and concentrated in vacuum to yield the title product (25 mg, 68%).

MS (ESI): m/z 487 (M+H)⁺.

### (4-Methyl-pyrimidin-2-yl)-(6-trifluoromethyl-4,5,6,7-tetrahydro-3-thia-1,7,8-triaza-cyclopenta[e]azulen-2-yl)-amine

According to Scheme 1, Step 9: A solution of [8-(4-methoxy-benzyl)-6-trifluoromethyl-4,5,6,8-tetrahydro-3-thia-1,7,8-triaza-cyclopenta[e]azulen-2-yl]-(4-methyl-pyrimidin-2-yl)-amine (25 mg, 51 μmol) in 2 mL of TFA was stirred at 100° C. for 1 h. After cooling to rt, the mixture was concentrated in vacuum. The residue was purified by Prep. HPLC to yield the title product (10 mg, 53%).

¹H-NMR (400 MHz, DMSO-d₆): δ 8.46 (d, J=5.2 Hz, 1H), 7.91 (s, 1H), 6.92 (d, J=5.2 Hz, 1H), 4.19 (d, J=3.2 Hz, 1H), 3.15-3.19 (m, 1H), 2.94-3.00 (m, 1H), 2.44 (s, 3H), 2.40-2.42 (m, 1H), 2.05-2.10 (m, 1H); MS (ESI): m/z 367 (M+H)⁺.

### Example 21

### (4-Methyl-pyrimidin-2-yl)-(5-propyl-5,6-dihydro-4H-3-thia-1,6,7-triaza-as-indacen-2-yl)-amine (Final Compound 1-5)

### 4-Allyl-cyclohexane-1,3-dione

According to Scheme 21, Step 1: To a solution of i-Pr₂NH (22.4 g, 357 mmol) in 150 mL THF was added n-BuLi (112 mL, 357 mmol) dropwise at −78° C. The resulting solution was stirred for about 40 min at 0° C. After cooling to −78° C., 1,3-cyclohexanedione (10.0 g, 89.2 mmol) and 60 mL of HMPA were added. The resulting solution was stirred for 1 h at −78° C., then allylbromide (10.0 g, 89.2 mmol) was added dropwise and the mixture was stirred at rt for 10 h. The mixture was acidified with 2N HCl to pH=4, extracted with EtOAc (100 mL×3). The organic layer was dried over Na₂SO₄, filtered and concentrated in vacuum. The residue was purified by flash chromatography on silica gel (PE:EtOAc, 10:1 to 1:1) to give the title compound (4.0 g, 30%).

### 4-Allyl-2-dimethylaminomethylene-cyclohexane-1,3-dione

According to Scheme 21, Step 2: A solution of 4-allyl-cyclohexane-1,3-dione (4.00 g, 26.3 mmol) and 1,1-dimethoxy-N,N-dimethyl methanamine (3.10 g, 26.3 mmol) in DCM (40 mL) was stirred at rt for 1 h. After evaporation, the residue was purified by flash chromatography on silica gel (PE:EtOAc, 3:1 to 1:2) to give the title product (4.0 g, 73%).

MS (ESI): m/z 208 (M+H)⁺.

### 7-Allyl-1,5,6,7-tetrahydro-indazol-4-one and 5-allyl-1,5,6,7-tetrahydro-indazol-4-one

According to Scheme 21, Step 3: At 0° C., AcOH (0.6 mL) followed by hydrazine monohydrate (14.4 mmol, 0.72 g) were added slowly to a solution of 4-allyl-2-dimethylaminomethylene-cyclohexane-1,3-dione (14.4 mmol, 3.0 g) in n-BuOH (150 mL). The resulting mixture was stirred under reflux for 2 h. After cooling to rt, the mixture was concentrated in vacuum. The residue was diluted with water, basified with NaHCO₃ to pH=8 and extracted with EtOAc (30 mL×3). The organic layer was dried over Na₂SO₄, filtered and concentrated in vacuum to give the title compounds (2.0 g, 79%).

MS (ESP: m/z 177 (M+H)⁺.

### 7-Allyl-2-(4-methoxy-benzyl)-2,5,6,7-tetrahydro-indazol-4-one and 5-allyl-2-(4-methoxy-benzyl)-2,5,6,7-tetrahydro-indazol-4-one

According to Scheme 21, Step 4: To a suspension of a mixture of 7-allyl-1,5,6,7-tetrahydro-indazol-4-one, 5-allyl-1,5,6,7-tetrahydro-indazol-4-one (2.0 g, 11.4 mmol) and K₂CO₃ (3.10 g, 22.8 mmol) in MeCN (20 mL) was added PMBCl (2.10 g, 13.6 mmol) and the mixture was refluxed for 2 h. After cooling to rt, the mixture was filtered and diluted with H₂O (40 mL), extracted with EtOAc (15 mL×3). The combined organic layers were dried over Na₂SO₄, concentrated in vacuum and purified by flash chromatography on silica gel (PE:EtOAc, 10:1 to 3:1) to give the title compounds (3.0 g, 89%).

MS (ESI): m/z 297 (M+H)⁺.

### 2-(4-Methoxy-benzyl)-7-propyl-2,5,6,7-tetrahydro-indazol-4-one and 2-(4-methoxy-benzyl)-5-propyl-2,5,6,7-tetrahydro-indazol-4-one

According to Scheme 21, Step 5: To a solution of a mixture of 7-allyl-2-(4-methoxy-benzyl)-2,5,6,7-tetrahydro-indazol-4-one and 5-allyl-2-(4-methoxy-benzyl)-2,5,6,7-tetrahydro-indazol-4-one (500 mg, 1.69 mmol) in MeOH (10 mL) was added Pd/C (50 mg), then the mixture was stirred at rt under H₂ (1 atm.) for 2 h. The mixture was filtered over celite, and the filtrate was concentrated in vacuum to give the title products (500 mg, 99%).

MS (ESI): m/z 299 (M+H)⁺.

### [7-(4-Methoxy-benzyl)-5-propyl-5,7-dihydro-4H-3-thia-1,6,7-triaza-as-indacen-2-yl]-(4-methyl-pyrimidin-2-yl)-amine

According to Scheme 21, Step 6: To a solution of a mixture of 2-(4-methoxy-benzyl)-7-propyl-2,5,6,7-tetrahydro-indazol-4-one and 2-(4-methoxy-benzyl)-5-propyl-2,5,6,7-tetrahydro-indazol-4-one (500 mg, 1.68 mmol) in CHCl₃ (12 mL) was added PhNMe₃Br₃ (630 mg, 1.68 mmol). The reaction mixture was refluxed for 1 h. After cooling to rt, the mixture was diluted with H₂O (20 mL) and extracted with DCM (20 mL×2). The combined organic layers were dried over Na₂SO₄, filtered and concentrated in vacuum. 4-Methylpyrimidin-2-yl-thiourea (246 mg, 1.46 mmol) and EtOH (8 mL) were added to the residue, and the mixture was refluxed for 4 h. After cooling to rt, the mixture was concentrated in vacuum, 1N NaOH (10 mL) was added, extracted with EtOAc (10 mL×3). The combined organic layers were washed with H₂O (10 mL) and brine (10 mL), dried over Na₂SO₄, filtered and concentrated in vacuum and purified by Prep. TLC (PE:EtOAc, 1:1) to give the title compound (100 mg, 15%).

MS (ESI): m/z 447 (M+H)⁺.

### (4-Methyl-pyrimidin-2-yl)-(5-propyl-5,6-dihydro-4H-3-thia-1,6,7-triaza-as-indacen-2-yl)-amine

According to Scheme 21, Step 7: A solution of [7-(4-methoxy-benzyl)-5-propyl-5,7-dihydro-4H-3-thia-1,6,7-triaza-as-indacen-2-yl]-(4-methyl-pyrimidin-2-yl)-amine (100 mg, 0.22 mmol) in 5 mL of TFA was stirred at 100° C. for 2 h. After cooling to rt, the mixture was concentrated in vacuum and purified by Prep. HPLC to yield the title compound (20 mg, 27%).

¹H-NMR (400 MHz, DMSO-d₆): δ 8.43 (d, J=5.2 Hz, 1H), 7.61 (s, 1H), 6.88 (d, J=5.2 Hz, 1H), 2.99-3.12 (m, 2H), 2.65-2.73 (m, 1H), 2.41 (s, 3H), 1.70-1.79 (m, 1H), 1.37-1.47 (m, 3H), 0.88 (t, J=6.8 Hz, 3H); MS (ESI): m/z 327 (M+H)⁺.

### Example 22

### (4-Methyl-pyrimidin-2-yl)-(5,6,7,8-tetrahydro-4H-3-thia-1,8,9-triaza-dicyclopenta[a,c]cycloocten-2-yl)-amine (Final Compound 1-7)

### 1-[1-(4-Methoxy-benzyl)-3-vinyl-1H-pyrazol-4-yl]-hex-5-en-1-one

According to Scheme 22, Step 1: Mg (0.86 g, 36 mmol) and dry THF (10 mL) were introduced under an inert atmosphere into a three-necked flask which was equipped with a dropping funnel and a thermometer. A solution of 5-bromo-pent-1-ene (2.98 g, 20 mmol) in dry THF (30 mL) was introduced into the dropping funnel. About 30 mL of this solution was added first to trigger the reaction. The remaining solution was added dropwise while maintaining the temperature between 60-70° C. When the temperate of the reaction mixture reached rt, the reaction was complete. To a solution of N-methoxy-1-(4-methoxybenzyl)-N-methyl-3-vinyl-1H-pyrazole-4-carboxamide (3.0 g, 0.1 mol) in dry THF (20 mL) was added dropwise the above Grignard reagent at −78° C. under N₂. The mixture was stirred at −78° C. for 30 min, then warmed to rt and stirred overnight. The reaction was quenched with saturated NH₄Cl (20 mL) and THF was removed under vacuum. The mixture was extracted by DCM (150 mL×4), dried over Na₂SO₄, filtered and concentrated to give the crude product. The residue was purified by flash chromatography on silica gel (PE:EtOAc, 10:1) to afford the title product (400 mg, 13%).

MS (ESI): m/z 311 (M+H)⁺.

### 2-(4-Methoxy-benzyl)-2,5,6,7-tetrahydro-cyclooctapyrazol-4-one

According to Scheme 22, Step 2: To a solution of 1-[1-(4-methoxy-benzyl)-3-vinyl-1H-pyrazol-4-yl]-hex-5-en-1-one (400 mg, 1.30 mmol) in dry DCM (200 mL) was added Grubbs catalyst 2^{nd} Generation (110 mg, 0.13 mmol) under N₂, then the reaction mixture was refluxed for 18 h. After cooling to rt, the solvent was removed under reduced pressure and the residue was purified by flash chromatography on silica gel (PE:EtOAc, 10:1 to 5:1) to afford the title compound (270 mg, 73%).

MS (ESI): m/z 283 (M+H)⁺.

### 2-(4-Methoxy-benzyl)-2,5,6,7,8,9-hexahydro-cyclooctapyrazol-4-one

According to Scheme 22, Step 3: To a solution of 2-(4-methoxy-benzyl)-2,5,6,7-tetrahydro-cyclooctapyrazol-4-one (270 mg, 0.95 mmol) in MeOH (30 mL) was added Pd/C (50 mg). The reaction mixture was stirred at rt for 2 h under H₂ atmosphere. The mixture was filtered and concentrated under reduced pressure to afford the crude product without further purification (270 mg, 100%).

MS (ESI): m/z 285 (M+H)⁺.

### 5-Bromo-2-(4-methoxy-benzyl)-2,5,6,7,8,9-hexahydro-cyclooctapyrazol-4-one

According to Scheme 22, Step 4: The mixture of 2-(4-methoxy-benzyl)-2,5,6,7,8,9-hexahydro-cyclooctapyrazol-4-one (270 mg, 0.95 mmol) and PhNMe₃Br₃ (376 mg, 1.00 mmol) in CHCl₃ (6 mL) was refluxed for 30 min. After cooling to rt, the mixture was diluted with DCM (40 mL), washed with brine (10 mL×2) and water (10 mL), dried over Na₂SO₄, filtered and concentrated in vacuum to give the crude product without further purification (300 mg, 87%).

MS (ESI): m/z 363, 365 (M+H)⁺.

### [9-(4-Methoxy-benzyl)-5,6,7,9-tetrahydro-4H-3-thia-1,8,9-triaza-dicyclopenta[a,c]cycloocten-2-yl]-(4-methyl-pyrimidin-2-yl)-amine

According to Scheme 22, Step 5: A mixture of 5-bromo-2-(4-methoxy-benzyl)-2,5,6,7,8,9-hexahydro-cyclooctapyrazol-4-one (300 mg, 0.83 mmol) and (4-methyl-pyrimidin-2-yl)thiourea (140 mg, 0.83 mmol) in n-BuOH (3 mL) was stirred at reflux for 3 h. After cooling to rt, the mixture was concentrated in vacuum to give the crude product without further purification (150 mg, 42%).

MS (ESI): m/z 433 (M+H)⁺.

### (4-Methyl-pyrimidin-2-yl)-(5,6,7,8-tetrahydro-4H-3-thia-1,8,9-triaza-dicyclopenta[a,c]cycloocten-2-yl)-amine

According to Scheme 22, Step 6: A solution of [9-(4-methoxy-benzyl)-5,6,7,9-tetrahydro-4H-3-thia-1,8,9-triaza-dicyclopenta[a,c]cycloocten-2-yl]-(4-methyl-pyrimidin-2-yl)-amine (150 mg, 0.35 mmol) in TFA (3 mL) was stirred at 100° C. under microwave condition for 20 min. After the reaction was finished, the mixture was diluted with DCM and concentrated in vacuum. The residue was dissolved in DMSO and purified by Prep. HPLC to give the desired final product (80 mg, 73%).

¹H-NMR (400 MHz, DMSO-d₆): δ 8.42 (d, 1H, J=5.2 Hz), 7.56 (s, 1H), 6.87 (d, 1H, J=5.2 Hz), 2.73-2.78 (m, 4H), 2.41 (s, 3H), 1.69-1.73 (m, 4H); MS (ESI): m/z 313 (M+H)⁺.

### Example 23

### (4-Methyl-6H-3-thia-1,6,7-triaza-as-indacen-2-yl)-pyridin-2-yl-amine (Final Compound 1-1)

A mixture of 4-methyl-N-(pyridin-2-yl)-5,6-dihydro-4H-thiazolo[4,5-e]indazol-2-amine (20 mg, 71 μmol) and DDQ (18 mg, 78 μmol) in toluene (706 μl) was stirred under reflux for 2 h. After complete conversion, a saturated solution of Na₂SO₃ was added in order to reduce the remaining DDQ. The mixture was stirred 1 h, and the aqueous layer was extracted with EtOAc. The combined organic phases were dried over MgSO₄, filtered and evaporated. The crude residue was purified through SCX₂ column washing with EtOH and then eluting with DCM/EtOH/NH₃ to afford the title compound as a beige solid (22 mg, 15%).

¹H-NMR (300 MHz, CD₃OD): 8.41-8.39 (m, 1H), 8.27 (s, 1H), 7.77-7.71 (m, 1H), 7.21 (s, 1H), 7.16-7.13 (d, 1H), 7.02-6.98 (m, 1H), 2.66 (s, 3H); MS (ESI): m/z 282 (M+H)⁺; RT=0.79 min.

The compounds in the following Table have been synthezised according to the same methods as previous examples 1 to 23, as denoted in the column denoted as “Exp. nr”. The compounds denoted with the asterisk have been exemplified in the Examples.

TABLE 1
Compounds prepared according to the Examples.

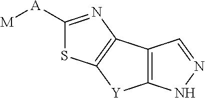

| Co. | Exp | | | |
|---|---|---|---|---|
| nr. | nr. | M | A | Y |
| 1-1 | 23* | 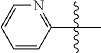 [SMILES: *c1ccccn1] | NH | 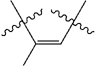 [SMILES: */C=C(\*)C] |
| 1-2 | 2* | 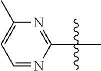 [SMILES: *c1nccc(C)n1] | NH | 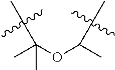 [SMILES: *C(C)OC(*)(C)C] |
| 1-3 | 17* | 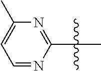 [SMILES: *c1nccc(C)n1] | NH | 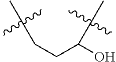 [SMILES: *CCC(*)O] |
| 1-4 | 3* | 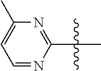 [SMILES: *c1nccc(C)n1] | NH | 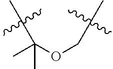 [SMILES: *COC(*)(C)C] |
| 1-5 | 21* | 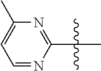 [SMILES: *c1nccc(C)n1] | NH | 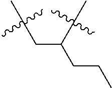 [SMILES: *CC(*)CCC] |
| 1-6 | 11* | 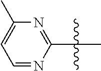 [SMILES: *c1nccc(C)n1] | NH | 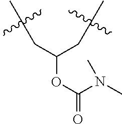 [SMILES: *CC(C*)OC(=O)N(C)C] |
| 1-7 | 22* | 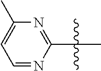 [SMILES: *c1nccc(C)n1] | NH | 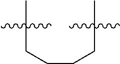 [SMILES: *CCCC*] |
| 1-8 | 4* | 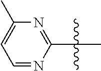 [SMILES: *c1nccc(C)n1] | NH | 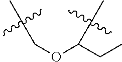 [SMILES: *COC(*)CC] |
| 1-9 | 18* | 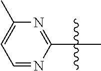 [SMILES: *c1nccc(C)n1] | NH | 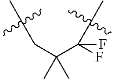 [SMILES: *CC(C)(C)C(*)(F)F] |
| 1-10 | 7* | 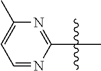 [SMILES: *c1nccc(C)n1] | NH | 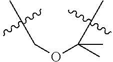 [SMILES: *COC(*)(C)C] |
| 1-11 | 14* | 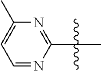 [SMILES: *c1nccc(C)n1] | NH | 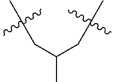 [SMILES: *CC(C)C*] |
| 1-12 | 15* | 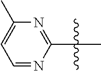 [SMILES: *c1nccc(C)n1] | NH | 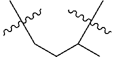 [SMILES: *CCC(*)C] |
| 1-13 | 8* | 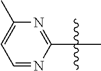 [SMILES: *c1nccc(C)n1] | NH | 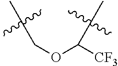 [SMILES: *COC(*)C(F)(F)F] |
| 1-14 | 1 | 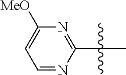 [SMILES: *c1nccc(OC)n1] | NH | 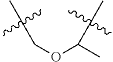 [SMILES: *COC(*)C] |
| 1-15 | 12* | 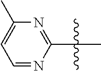 [SMILES: *c1nccc(C)n1] | NH | 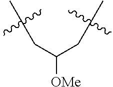 [SMILES: *CC(C*)OC] |
| 1-16 | 9* | 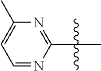 [SMILES: *c1nccc(C)n1] | NH | 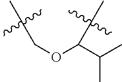 [SMILES: *COC(*)C(C)C] |
| 1-17 | 10* | 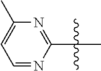 [SMILES: *c1nccc(C)n1] | NH | 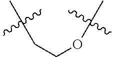 [SMILES: *CCO*] |
| 1-18 | 13* | 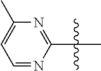 [SMILES: *c1nccc(C)n1] | NH | 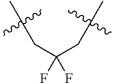 [SMILES: *CC(F)(F)C*] |
| 1-19 | 5* | 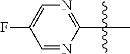 [SMILES: *c1ncc(F)cn1] | NH | 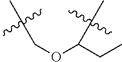 [SMILES: *COC(*)CC] |
| 1-20 | 6* | 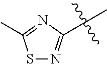 [SMILES: *c1nsc(C)n1] | NH | 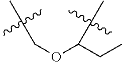 [SMILES: *COC(*)CC] |
| 1-21 | 4 | 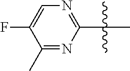 [SMILES: *c1ncc(F)c(C)n1] | NH | 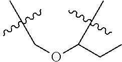 [SMILES: *COC(*)CC] |
| 1-22 | 4 | 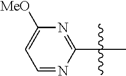 [SMILES: *c1nccc(OC)n1] | NH | 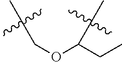 [SMILES: *COC(*)CC] |
| 1-23 | 1* | 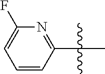 [SMILES: *c1cccc(F)n1] | NH | 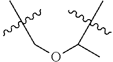 [SMILES: *COC(*)C] |
| 1-24 | 19* | 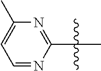 [SMILES: *c1nccc(C)n1] | NH | 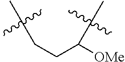 [SMILES: *CCC(*)OC] |
| 1-25 | 6 | 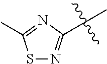 [SMILES: *c1nsc(C)n1] | NH | 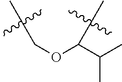 [SMILES: *COC(*)C(C)C] |
| 1-26 | 4 | 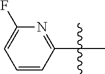 [SMILES: *c1cccc(F)n1] | NH | 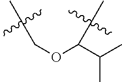 [SMILES: *COC(*)C(C)C] |
| 1-27 | 20* | 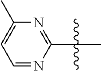 [SMILES: *c1nccc(C)n1] | NH | 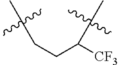 [SMILES: *CCC(*)C(F)(F)F] |
| 1-28 | 4 | 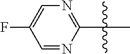 [SMILES: *c1ncc(F)cn1] | NH | 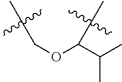 [SMILES: *COC(*)C(C)C] |
| 1-29 | 4 | 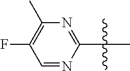 [SMILES: *c1ncc(F)c(C)n1] | NH | 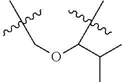 [SMILES: *COC(*)C(C)C] |
| 1-30 | 16* | 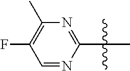 [SMILES: *c1ncc(F)c(C)n1] | NH | 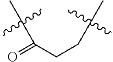 [SMILES: *CCC(*)=O] |
| 1-31 | 4 | 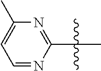 [SMILES: *c1nccc(C)n1] | NH | 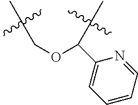 [SMILES: *COC(*)c1ccccn1] |

TABLE 2
Physico-chemical data for some compounds (nd = not determined).

| Co. Nr | [MH⁺] | NMR-data |
|---|---|---|
| 1-1 | 282 | ¹H-NMR (300 MHz, DMSO-d₆) δ: 8.41-8.39 (m, 1H), 8.27 (s, 1H), 7.77-7.71 (m, 1H), 7.21 (s, 1H), 7.16-7.13 (d, 1H), |
| | | 7.02-6.98 (m, 1H), 2.66 (s, 3H). |
| 1-2 | 343 | ¹H-NMR (400 MHz, DMSO-d₆) δ: 8.39 (d, J = 4.8 Hz, 1H), 7.72 (s, 1H), 6.83 (d, J = 4.8 Hz, 1H), 4.95-4.97 (m, 1H), 2.36 (s, 3H), |
| | | 1.51-1.55 (m, 6H), 1.46 (d, J = 6.0 Hz, 3H). |
| 1-3 | 315 | ¹H-NMR (300 MHz, CD₃OD) δ: 8.47 (d, 1H, J = 5.2 Hz), 8.06 (s, 1H), 6.98 (d, 1H, J = 5.2 Hz), 5.12 (d, 1H, J = 5.6 Hz), |
| | | 3.19-3.26 (m, 1H), 2.91-2.97 (m, 1H), 2.62 (s, 3H), 2.29-2.35 (m, 1H), 2.04-2.11 (m, 1H). |
| 1-4 | 329 | ¹H-NMR (400 MHz, DMSO-d₆) δ: 11.50 (s, 1H), 8.44 (d, 1H, J = 4.2 Hz), 7.77 (s, 1H); 6.89 (d, 1H, J = 5.2 Hz), 4.86 (s, 2H), |
| | | 2.42 (s, 3H), 1.59 (s, 6H). |
| 1-5 | 327 | ¹H-NMR (400 MHz, DMSO-d₆) δ: 8.43 (d, J = 5.2 Hz, 1H), 7.61 (s, 1H), 6.88 (d, J = 5.2 Hz, 1H), 2.99-3.12 (m, 2H), |
| | | 2.65-2.73 (m, 1H), 2.41 (s, 3H), 1.70-1.79 (m, 1H), 1.37-1.47 (m, 3H), 0.88 (t, J = 6.8 Hz, 3H). |
| 1-6 | 386 | ¹H-NMR (400 MHz, DMSO-d₆) δ: 11.43 (br, 1H), 8.36 (d, 1H, J = 4.8 Hz), 7.67 (s, 1H), 6.82 (t, 1H, J = 5.2 Hz), |
| | | 5.02-5.06 (m, 1H), 3.08-3.22 (m, 4H), 2.68 (s, 3H), 2.52 (s, 3H), 2.35 (s, 3H). |
| 1-7 | 313 | ¹H-NMR (400 MHz, DMSO-d₆) δ: 8.42 (d, 1H, J = 5.2 Hz), 7.56 (s, 1H), 6.87 (d, 1H, J = 5.2 Hz), 2.73-2.78 (m, 4H), |
| | | 2.41 (s, 3H), 1.69-1.73 (m, 4H). |
| 1-8 | 329 | ¹H-NMR (400 MHz, DMSO-d₆) δ: 8.42 (d, J = 4.8 Hz, 1H), 7.75 (s, 1H), 6.87 (d, J = 4.8 Hz, 1H), 5.03-5.07 (m, 1H), |
| | | 4.78-4.86 (m, 2H), 2.40 (s, 3H), 2.08-2.14 (m, 1H), 1.73-1.79 (m, 1H), 0.96 (t, J = 7.2 Hz, 3H). |
| 1-9 | 363 | ¹H-NMR (400 MHz, DMSO-d₆) δ: 8.41 (d, J = 4.8 Hz, 1H), 7.88 (s, 1H), 6.87 (d, J = 4.8 Hz, 1H), 2.94 (s, 2H), |
| | | 2.40 (s, 3H), 1.07 (s, 6H). |
| 1-10 | 329 | ¹H-NMR (400 MHz, DMSO-d₆) δ: 11.52 (br, 1H), 8.42 (d, J = 4.8 Hz, 1H), 7.69 (s, 1H), 6.87 (d, J = 4.8 Hz, 1H), |
| | | 4.82 (s, 2H), 2.40 (s, 3H), 1.52 (s, 6H). |
| 1-11 | 313 | ¹H-NMR (400 MHz, DMSO-d₆) δ: 8.40 (d, J = 4.8 Hz, 1H), 7.68 (s, 1H), 6.85 (d, J = 4.8 Hz, 1H), 2.93-2.97 (m, 2H), |
| | | 2.73-2.79 (m, 2H), 2.39 (s, 3H), 2.16 (d, J = 6.0 H, 1H), 1.01 (d, J = 6.8 Hz, 3H). |
| 1-12 | 313 | ¹H-NMR (400 MHz, DMSO-d₆) δ: 8.41 (d, J = 5.2 Hz, 1H), 7.75 (s, 1H), 6.86 (d, J = 5.2 Hz, 1H), 3.16-3.19 (m, 1H), |
| | | 2.90-2.97 (m, 2H), 2.40 (s, 3H), 1.80-1.97 (m, 2H), 1.25 (d, J = 7.2 Hz, 3H). |
| 1-13 | 369 | ¹H-NMR (400 MHz, DMSO-d₆) δ: 11.60 (s, 1H), 8.42 (d, J = 5.2 Hz, 1H), 7.89 (s, 1H), 6.88 (d, J = 5.2 Hz, 1H), |
| | | 5.75 (d, J = 5.6 Hz, 1H), 5.16 (d, J = 16 Hz, 1H), 5.02 (d, J = 15.6 Hz, 1H), 2.40 (s, 3H). |
| 1-14 | 331 | ¹H-NMR (400 MHz, DMSO-d₆) δ: 8.27 (d, 1H, J = 6.0 Hz), 7.82 (s, 1H), 6.44 (d, 1H, J = 6.0 Hz), 4.98-5.05 (m, 2H), |
| | | 4.88 (d, 1H, J = 16.0 Hz), 4.10 (s, 3H), 1.52 (d, 3H, J = 6.4 Hz). |
| 1-15 | 329 | ¹H-NMR (400 MHz, DMSO-d₆) δ: 8.41 (d, 1H, J = 4.8 Hz), 7.69 (s, 1H), 6.866 (d, 1H, J = 4.8 Hz), 3.80-3.83 (m, 1H), |
| | | 3.24 (s, 3H), 3.12-3.13 (m, 4H), 2.40 (s, 3H). |
| 1-16 | 343 | ¹H-NMR (400 MHz, DMSO-d₆) δ: 11.52 (s, 1H), 8.41 (d, J = 5.2 Hz, 1H), 7.74 (s, 1H), 6.86 (d, J = 5.2 Hz, 1H), |
| | | 5.04 (d, J = 15.6 Hz, 1H), 4.77 (d, J = 15.6 Hz, 1H), 4.74 (d, J = 12.8 Hz, 1H), 2.43-2.50 (m, 1H), 2.42 (s, 3H), |
| | | 1.06 (d, J = 6.8 Hz, 3H), 0.77 (d, J = 6.8 Hz, 3H). |
| 1-17 | 301 | ¹H-NMR (400 MHz, DMSO-d₆) δ: 11.45 (s, 1H), 8.41 (d, J = 5.2 Hz, 1H), 7.65 (s, 1H), 6.87 (d, J = 5.2 Hz, 1H), |
| | | 4.26 (t, J = 4.0 Hz, 2H), 3.12 (t, J = 4.4 Hz, 2H), 2.40 (s, 3H). |
| 1-18 | 335 | ¹H-NMR (400 MHz, DMSO-d₆) δ: 11.56 (br, 1H), 8.43 (d, 1H, J = 5.2 Hz), 7.79 (s, 1H), 6.89 (d, 1H, J = 5.2 Hz), |
| | | 3.56-3.64 (m, 4H), 2.41 (s, 3H). |
| 1-19 | 333 | ¹H-NMR (400 MHz, DMSO-d₆) δ: δ 8.79 (s, 1H), 7.84 (s, 1H), 5.14-5.18 (m, 1H), 4.90-4.96 (m, 2H), 2.19-2.22 (m, 1H), |
| | | 1.83-1.89 (m, 1H), 1.07 (t, J = 7.2 Hz, 3H). |
| 1-20 | 335 | ¹H-NMR (400 MHz, DMSO-d₆) δ: 7.74 (s, 1H), 5.02-5.06 (m, 1H), 4.79-4.85 (m, 2H), 2.72 (s, 3H), 2.08-2.13 (m, 1H), |
| | | 1.72-1.79 (m, 1H), 0.96 (t, J = 7.2 Hz, 3H). |
| 1-21 | 347 | ¹H-NMR (400 MHz, DMSO-d₆) δ: 8.51 (d, J = 1.2 Hz, 1H), 7.73 (s, 1H), 5.03-5.07 (m, 1H), 4.79-4.86 (m, 2H), |
| | | 2.42 (s, 3H), 2.08-2.14 (m, 1H), 1.72-1.79 (m, 1H), 0.97 (t, J = 7.2 Hz, 3H). |
| 1-22 | 345 | ¹H-NMR (400 MHz, DMSO-d₆) δ: 8.28 (d, J = 5.6 Hz, 1H), 7.77 (s, 1H), 6.84 (d, J = 5.6 Hz, 1H), 5.03-5.07 (m, 1H), |
| | | 4.82-4.86 (m, 2H), 4.00 (s, 3H), 2.07-2.13 (m, 1H), 1.73-1.80 (m, 1H), 0.96 (t, J = 7.2 Hz, 3H). |
| 1-23 | 318 | ¹H-NMR (400 MHz, DMSO-d₆) δ: 11.52 (s, 1H), 7.81 (dd, 1H, J = 8.4 Hz, J = 16.4 Hz), 7.74 (s, 1H), |
| | | 6.94 (d, 1H, J = 6.0 Hz), 6.57 (d, 1H, J = 6.0 Hz), 5.07-4.96 (m, 2H), 4.87 (d, 1H, J = 6.0 Hz), 1.52 (d, 3H, J = 6.4 Hz). |
| 1-24 | 329 | ¹H-NMR (400 MHz, DMSO-d₆) δ: 8.41 (d, J = 4.8 Hz, 1H), 7.78 (s, 1H), 6.86 (d, J = 4.8 Hz, 1H), 4.53 |
| | | (d, J = 5.2 Hz, 1H), 3.25 (s, 3H), 2.98-3.01 (m, 1H), 2.84-2.88 (m, 1H), 2.34 (s, 3H), 2.29-2.32 (m, 1H), 1.75-1.81 (m, 1H). |
| 1-25 | 335 | ¹H-NMR (400 MHz, DMSO-d₆) δ: 7.83 (s, 1H), 5.16 (d, J = 15.6 Hz, 1H), 4.88 (d, J = 15.6 Hz, 1H), 4.84 (s, 1H), |
| | | 2.82 (s, 3H), 2.56-2.57 (m, 1H), 1.17 (d, J = 6.8 Hz, 3H), 0.88 (d, J = 6.4 Hz, 3H). |
| 1-26 | 346 | ¹H-NMR (400 MHz, DMSO-d₆) δ: 8.78 (s, 1H), 7.74 (s, 1H), 6.94 (d, J = 8.0 Hz, 1H), 6.57 (d, J = 7.2 Hz, 1H), |
| | | 5.10 (d, J = 15.6 Hz, 1H), 4.79 (d, J = 15.6 Hz, 1H), 4.74 (s, 1H), 2.47-2.49 (m, 1H), 1.08 (d, J = 5.8 Hz, 3H), 0.78 (d, |
| | | J = 5.8 Hz, 3H). |
| 1-27 | 367 | ¹H-NMR (400 MHz, DMSO-d₆) δ: 8.46 (d, J = 5.2 Hz, 1H), 7.91 (s, 1H), 6.92 (d, J = 5.2 Hz, 1H), 4.19 (d, |
| | | J = 3.2 Hz, 1H), 3.15-3.19 (m, 1H), 2.94-3.00 (m, 1H), 2.44 (s, 3H), 2.40-2.42 (m, 1H), 2.05-2.10 (m, 1H). |
| 1-28 | 347 | ¹H-NMR (400 MHz, DMSO-d₆) δ: 8.68 (s, 2H), 7.75 (s, 1H), 5.06-5.09 (m, 1H), 4.75-4.81 (m, 2H), 2.47 (s, 1H), 1.08 |
| | | (d, J = 6.4 Hz, 3H), 0.77 (d, J = 6.4 Hz, 3H). |
| 1-29 | 361 | ¹H-NMR (400 MHz, DMSO-d₆) δ: 11.65 (s, 1H), 8.51 (d, J = 1.2 Hz, 1H), 7.74 (s, 1H), 5.06 (d, J = 16.0 Hz, 1H), |
| | | 4.79 (d, J = 16.0 Hz, 1H), 4.74 (s, 1H), 2.41-2.50 (m, 1H), 2.29 (s, 3H), 1.07 (d, J = 6.8 Hz, 3H), 0.77 (d, J = 6.8 Hz, 3H). |
| 1-30 | 331 | ¹H-NMR (400 MHz, DMSO-d₆) δ: 12.32 (s, 1H), 8.63 (s, 1H), 7.92 (s, 1H), 2.95 (t, J = 5.2 Hz, 2H), 2.76 (t, J = |
| | | 5.2 Hz, 2H), 2.46 (s, 3H). |
| 1-31 | 378 | ¹H-NMR (400 MHz, DMSO-d₆) δ: 8.62 (s, 1H), 8.43 (d, J = 4.8 Hz, 1H), 7.99 (s, 1H), 7.85 (s, 1H), 7.51 (s, 2H), |
| | | 6.89 (d, J = 5.2 Hz, 1H), 6.17 (s, 1H), 5.06 (s, 2H), 2.04 (s, 3H). |

### Pharmacology

The compounds provided in the present invention are positive allosteric modulators of mGluR₄. As such, these compounds do not appear to bind to the orthosteric glutamate recognition site, and do not activate the mGluR₄ by themselves. Instead, the response of mGluR₄ to a concentration of glutamate or mGluR₄ agonist is increased when compounds of Formula (I) to (III) are present. Compounds of Formula (I) to (III) are expected to have their effect at mGluR₄ by virtue of their ability to enhance the function of the receptor.

mGluR₄ Assay on HEK-Expressing Human mGluR₄

The compounds of the present invention are positive allosteric modulators of mGluR₄ receptor. Their activity was examined on recombinant human mGluR₄ₐ receptors by detecting changes in intracellular Ca²⁺concentration, using the fluorescent Ca²⁺-sensitive dye Fluo4-(AM) and a Fluorometric Imaging Plate Reader (FLIPR, Molecular Devices, Sunnyvale, Calif.).

### Transfection and Cell Culture

The cDNA encoding the human metabotropic glutamate receptor (hmGluR₄), (accession number NM_{—}000841.1, NCBI Nucleotide database browser), was subcloned into an expression vector containing also the hygromycin resistance gene. In parallel, the cDNA encoding a G protein allowing redirection of the activation signal to intracellular calcium flux was subcloned into a different expression vector containing also the puromycin resistance gene. Transfection of both these vectors into HEK293 cells with PolyFect reagent (Qiagen) according to supplier's protocol, and hygromycin and puromycin treatment allowed selection of antibiotic resistant cells which had integrated stably one or more copies of the plasmids. Positive cellular clones expressing hmGluR₄ were identified in a functional assay measuring changes in calcium fluxes in response to glutamate or selective known mGluR₄ orthosteric agonists and antagonists.

HEK-293 cells expressing hmGluR₄ were maintained in media containing DMEM, dialyzed Fetal Calf Serum (10%), Glutamax™ (2 mM), Penicillin (100 units/mL), Streptomycin (100 μg/mL), Geneticin (100 μg/mL) and Hygromycin-B (40 μg/mL) and puromycin (1 μg/mL) at 37° C./5% CO₂.

### Fluorescent Cell Based-Ca²⁺Mobilization Assay

Human mGluR₄ HEK-293 cells were plated out 24 hours prior to FLIPR³⁸⁴ assay in black-walled, clear-bottomed, poly-L-ornithine-coated 384-well plates at a density of 25,000 cells/well in a glutamine/glutamate free DMEM medium containing foetal bovine serum (10%), penicillin (100 units/mL) and streptomycin (100 μg/mL) at 37° C./5% CO₂.

On the day of the assay, the medium was aspirated and the cells were loaded with a 3 μM solution of Fluo4-AM (LuBioScience, Lucerne, Switzerland) in 0.03% pluronic acid. After 1 hour at 37° C./5% CO₂, the non incorporated dye was removed by washing cell plate with the assay buffer and the cells were left in the dark at room temperature for six hours before testing. All assays were performed in a pH=7.4 buffered-solution containing 20 mM HEPES, 143 mM NaCl, 6 mM KCl, 1 mM MgSO₄, 1 mM CaCl₂, 0.125 mM sulfapyrazone and 0.1% glucose.

After 10 s of basal fluorescence recording, various concentrations of the compounds of the invention were added to the cells. Changes in fluorescence levels were first monitored for 180 s in order to detect any agonist activity of the compounds. Then the cells were stimulated by an EC₂₅ glutamate concentration for an additional 110 s in order to measure enhancing activities of the compounds of the invention. EC₂₅ glutamate concentration is the concentration giving 25% of the maximal glutamate response.

The concentration-response curves of representative compounds of the present invention were generated using the Prism GraphPad software (Graph Pad Inc, San Diego, USA). The curves were fitted to a four-parameter logistic equation:

(Y=Bottom+(Top−Bottom)/(1+10̂((LogEC₅₀−X)*Hill Slope)

allowing the determination of EC₅₀ values.

The Table 3 below represents the mean EC₅₀ obtained from at least three independent experiments of selected molecules performed in duplicate.

TABLE 3
Activity data for selected compounds

| Compound | |
|---|---|
| no. | Ca²⁺ Flux* |
| 1-1 | + |
| 1-2 | + |
| 1-3 | ++ |
| 1-4 | ++ |
| 1-5 | ++ |
| 1-6 | ++ |
| 1-7 | ++ |
| 1-8 | +++ |
| 1-9 | ++ |
| 1-10 | +++ |
| 1-11 | ++ |
| 1-12 | +++ |
| 1-13 | +++ |
| 1-14 | +++ |
| 1-15 | ++ |
| 1-16 | +++ |
| 1-17 | +++ |
| 1-18 | +++ |
| 1-19 | +++ |
| 1-20 | +++ |
| 1-21 | +++ |
| 1-22 | +++ |
| 1-23 | +++ |
| 1-24 | +++ |
| 1-25 | ++ |
| 1-26 | ++ |
| 1-27 | ++ |
| 1-28 | +++ |
| 1-29 | ++ |
| 1-30 | + |
| 1-31 | +++ |

*Table legend:
(+): 1 μM < EC₅₀ < 10 μM
(++): 100 nM < EC₅₀ < 1 μM
(+++): EC₅₀ < 100 nM

The results shown in Table 3 demonstrate that the compounds described in the present invention are positive allosteric modulators of human mGluR₄ receptors. These compounds do not have activity by themselves but they rather increase the functional activity and/or maximal efficacy of glutamate or mGluR₄ agonist.

### Haloperidol-Induced Catalepsy Model in the Rat

The haloperidol-induced catalepsy is a model of Parkinson's disease. It is used to assess potential anti-parkinsonian action of compound. In this model, haloperidol, a dopamine receptor antagonist, is administered to induce catalepsy, characterized by hypokinesia and rigidity. This state is described as an acute parkinsonian state. Anti-parkinsonian drugs show efficacy in this model by decreasing the catalepsy induced by haloperidol.

### Experimental Design and Administration Procedure:

One day before the test, Male Sprague-Dawley rats (Charles River, les Oncins, France) were placed in individual cages. The day of the experiment, rats were injected with a dopamine D2 receptor antagonist, haloperidol (1.5 mg/kg, i.p.) 30 minutes prior to oral administration of test compound (1, 3, 10 and 30 mg/kg) or vehicle. L-DOPA-benserazide (150 mg/kg) used as a positive control, was also orally administered 30 min post-haloperidol injection.

### Experimental Procedure—Catalepsy Test:

Catalepsy was assessed 60 minutes after test compound or vehicle or MTEP treatments L-DOPA-benserazide using a grid test (e.g. 90 min post-haloperidol administration). Briefly, the rats were placed on a vertical wire grid with the head pointing toward the ceiling and all paws gripping the grid. Latency to movement of both forepaws to relocate the body was measured (in seconds) with a maximum latency “cut-off” time of 120-seconds. Brain and plasma were collected at the end of the experiment for compound exposure assessment.

### Unilateral 6-OHDA Lesion Treatments

The effect of test compounds were assessed alone or in combination with L-DOPA in male Sprague-Dawley rats lesioned through medial forebrain bundle (Taconic).

Animals were orally administered with test compounds and then tested 55-65 min post dosing in the forelimb stepping test for akinesia and 65-70 minutes post-dosing in the cylinder test. L-DOPA (2, 6 or 20 mg/kg), used as positive control and in co-therapy were ip injected. Then forelimb akinesia and cylinder tests were carried out 30-45-minutes post-dosing. In co-therapy, rats received test compound 30 minutes prior to L-DOPA and they were tested as described above between 55 and 75 minutes post test compound dosing.

### Forelimb Stepping Test for Akinesia

Stepping movements made by the isolated ipsi- and contra-lateral forelimbs are assessed. The rat's weight is centered over the isolated limb with its head and forequarters oriented forward by the experimenter. The number of rat-initiated steps that shift weight to a new location are recorded for 30-s.

### Cylinder Test

Measures spontaneous forelimb use while rats voluntarily explore a cylinder (d: 20-25 cm; h: 30 cm) and scored for the number of either ipsi-lateral, contra-lateral (affected limb), or both paw contacts during exploratory movements

Preference scores are calculated for ipsi-, contra-, or both forelimb contacts during a 10-minutes interval for a minimum of 20 events. For example, a zero score (lack of asymmetry) results from equal number of events for independent ipsi-versus contra-contacts, or simultaneous contacts of both paws.

Blood samples were taken immediately after testing.

Thus, the positive allosteric modulators provided in the present invention are expected to increase the effectiveness of glutamate or mGluR₄ agonists at mGluR₄ receptor. Therefore, these positive allosteric modulators are expected to be useful for treatment of various neurological and psychiatric disorders associated with glutamate dysfunction described to be treated herein and others that can be treated by such positive allosteric modulators.

The compounds of the invention can be administered either alone, or in combination with other pharmaceutical agents effective in the treatment of conditions mentioned above.

### Formulation Examples

Typical examples of recipes for the formulation of the invention are as follows:

### 1. Tablets

| | | |
|---|---|---|
| Active ingredient | 5 to 50 | mg |
| Di-calcium phosphate | 20 | mg |
| Lactose | 30 | mg |
| Talcum | 10 | mg |
| Magnesium stearate | 5 | mg |
| Potato starch | ad 200 | mg |

In this Example, active ingredient can be replaced by the same amount of any of the compounds according to the present invention, in particular by the same amount of any of the exemplified compounds.

### 2. Suspension

An aqueous suspension is prepared for oral administration so that each 1 milliliter contains 1 to 5 mg of one of the active compounds, 50 mg of sodium carboxymethyl cellulose, 1 mg of sodium benzoate, 500 mg of sorbitol and water ad 1 mL.

### 3. Injectable

A parenteral composition is prepared by stirring 1.5% by weight of active ingredient of the invention in 10% by volume propylene glycol and water.

### 4. Ointment

| | | |
|---|---|---|
| Active ingredient | 5 to 1000 | mg |
| Stearyl alcohol | 3 | g |
| Lanoline | 5 | g |
| White petroleum | 15 | g |
| Water | ad 100 | g |

In this Example, active ingredient can be replaced with the same amount of any of the compounds according to the present invention, in particular by the same amount of any of the exemplified compounds.

Reasonable variations are not to be regarded as a departure from the scope of the invention. It will be obvious that the thus described invention may be varied in many ways by those skilled in the art.

1. A compound having the Formula (I) wherein: 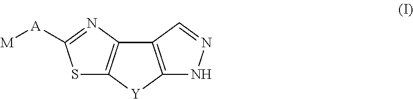
M is an optionally substituted heteroaryl;
A is NH or O;
Y is selected from the group of —CR¹R²—, —CR¹R²—CR³R⁴—, —CR¹R²—CR³R⁴—CR⁵R⁶—, —CR¹R²—CR³R⁴—CR⁵R⁶—CR⁷R⁸—, —CR¹═CR²—, —CO—, —CO—CR¹—R²—CR³R⁴—, —CR¹R²—CR³R⁴—CO—, —CO—CR¹R²—O—, —O—CR¹R²—CO—, —NR⁹—CR¹R²—CO—, —CO—NR⁹—, —NR⁹—CO—, —O—CR¹R²—, —CR¹R²—O—, —O—CR¹R²—CR³R⁴—, —CR¹R²—O—CR³R⁴—, —CR¹R²—CR³R⁴—O—, —NR⁹—CR¹R²—, —CR¹R²—NR⁹—, —NR⁹—CR¹R²—CR³R⁴—, —CR¹R²—NR⁹—CR³R⁴—, —S—CR¹R²—, —CR¹R²—S—, —S—CR¹R²—CR³R⁴—, —CR¹R²—S—CR³R⁴—, —CR¹R²—CR³R⁴—S—, —SO—CR¹R²—, —CR¹R²—SO—, —SO—CR¹R²—CR³R⁴—, —CR¹R²—SO—CR³R⁴—, —CR¹R²—CR³R⁴—SO—, —SO₂—CR¹R²—, —CR¹R²—SO₂—, —SO₂—CR¹R²—CR³R⁴—, —CR¹R²—SO₂—CR³R⁴— and —CR¹R²—CR³R⁴—SO₂—;
R¹, R², R³, R⁴, R⁵, R⁶, R⁷ and R⁸ are each independently selected from the group of hydrogen, halogen, —CN, —CF₃ or an optionally substituted radical selected from the group of —(C₁-C₆)alkyl, —(C₁-C₆)haloalkyl, —(C₃-C₇)cycloalkyl, aryl, heteroaryl, heterocycle, —(C₁-C₆)allylene-aryl, —(C₁-C₆)alkylene-heteroaryl, —(C₁-C₆)alkylene-heterocycle, —O—(C₀-C₆)alkyl, —N—((C₀-C₆)alkyl)₂, —(C₁-C₆)alkyl-O—(C₀-C₆)alkyl, and —(C₁-C₆)alkyl-N—((C₀-C₆)alkyl)₂;
Any two radicals of R (R¹, R², R³, R⁴, R⁵, R⁶, R⁷ or R⁸) may be taken together to form an optionally substituted 3 to 10 membered carbocyclic or heterocyclic ring; and
R⁹ is selected from the group of hydrogen or an optionally substituted radical selected from the group of —(C₁-C₆)alkyl, —(C₁-C₆)haloalkyl, —(C₃-C₇)cycloalkyl, —(C₁-C₆)alkylene-(C₁-C₆)haloalkyl, —(C₁-C₆)allylene-(C₃-C₇)halocycloalkyl, aryl, heteroaryl, heterocycle, —(C₁-C₆)alkylene-aryl, —(C₁-C₆)alkylene-heteroaryl, —(C₁-C₆)alkylene-heterocycle, —(C₂-C₆)alkyl-O—(C₀-C₆)alkyl, and —(C₂-C₆)alkyl-N—((C₀-C₆)alkyl)₂.

2. A compound according to claim 1 having the Formula (II): 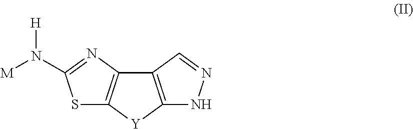
provided that according to proviso (i) the compound is not:
N-(Pyridin-2-yl)-5,6-dihydro-4H-thiazolo[4,5-e]indazol-2-amine
N-(6-Methylpyridin-2-yl)-5,6-dihydro-4H-thiazolo[4,5-e]indazol-2-amine
N-(6-Chloropyridin-2-yl)-5,6-dihydro-4H-thiazolo[4,5-e]indazol-2-amine
N-(6-Fluoropyridin-2-yl)-5,6-dihydro-4H-thiazolo[4,5-e]indazol-2-amine
4-Methyl-N-(pyridin-2-yl)-5,6-dihydro-4H-thiazolo[4,5-e]indazol-2-amine
Pyridin-2-yl-(4,5,6,7-tetrahydro-3-thia-1,7,8-triaza-cyclopenta[e]azulen-2-yl)-amine
4-Ethyl-N-(pyridin-2-yl)-5,6-dihydro-4H-thiazolo[4,5-e]indazol-2-amine
N-(3-Fluoropyridin-2-yl)-5,6-dihydro-4H-thiazolo[4,5-e]indazol-2-amine
(6-Methyl-pyridin-2-yl)-(4,5,6,7-tetrahydro-3-thia-1,7,8-triaza-cyclopenta[e]azulen-2-yl)-amine
(5-Fluoro-pyridin-2-yl)-(4,5,6,7-tetrahydro-3-thia-1,7,8-triaza-cyclopenta[e]azulen-2-yl)-amine
(3-Fluoro-pyridin-2-yl)-(4,5,6,7-tetrahydro-3-thia-1,7,8-triaza-cyclopenta[e]azulen-2-yl)-amine
(6-Methoxy-pyridin-2-yl)-(4,5,6,7-tetrahydro-3-thia-1,7,8-triaza-cyclopenta[e]azulen-2-yl)-amine
N-(5-Fluoropyridin-2-yl)-5,6-dihydro-4H-thiazolo[4,5-e]indazol-2-amine
(6-Chloro-pyridin-2-yl)-(4,5,6,7-tetrahydro-3-thia-1,7,8-triaza-cyclopenta[e]azulen-2-yl)-amine
N-(Pyrazin-2-yl)-5,6-dihydro-4H-thiazolo[4,5-e]indazol-2-amine
(6-Fluoro-pyridin-2-yl)-(4,5,6,7-tetrahydro-3-thia-1,7,8-triaza-cyclopenta[e]azulen-2-yl)-amine
Pyrimidin-2-yl-(4,5,6,7-tetrahydro-3-thia-1,7,8-triaza-cyclopenta[e]azulen-2-yl)-amine
N-(Pyrimidin-2-yl)-5,6-dihydro-4H-thiazolo[4,5-e]indazol-2-amine
(1-Methyl-1H-pyrazol-3-yl)-(4,5,6,7-tetrahydro-3-thia-1,7,8-triaza-cyclopenta[e]azulen-2-yl)-amine
N-(1-Methyl-1H-pyrazol-3-yl)-5,6-dihydro-4H-thiazolo[4,5-e]indazol-2-amine
N-(4-Methylpyrimidin-2-yl)-5,6-dihydro-4H-thiazolo[4,5-e]indazol-2-amine
(4-Methyl-pyrimidin-2-yl)-(4,5,6,7-tetrahydro-3-thia-1,7,8-triaza-cyclopenta[e]azulen-2-yl)-amine
4,4-Dimethyl-N-(pyridin-2-yl)-5,6-dihydro-4H-thiazolo[4,5-e]indazol-2-amine
4,4-Dimethyl-N-(pyrimidin-2-yl)-5,6-dihydro-4H-thiazolo[4,5-e]indazol-2-amine
4-Methyl-N-(pyrimidin-2-yl)-5,6-dihydro-4H-thiazolo[4,5-e]indazol-2-amine and
5,5-Dimethyl-N-(pyrimidin-2-yl)-5,6-dihydro-4H-thiazolo[4,5-e]indazol-2-amine;
provided that according to proviso (ii) the compound is not:
4,4-Dimethyl-N-(4-methylpyrimidin-2-yl)-5,6-dihydro-4H-[1,3]thiazolo[4,5-e]indazol-2-amine
N-(4-Methylpyrimidin-2-yl)-6H-[1,3]thiazolo[4,5-e]indazol-2-amine
N-(Pyrimidin-2-yl)-6H-[1,3]thiazolo[4,5-e]indazol-2-amine
N-(5-Fluoropyrimidin-2-yl)-4,4-dimethyl-5,6-dihydro-4H-[1,3]thiazolo[4,5-e]indazol-2-amine
N-(5-Fluoro-4-methylpyrimidin-2-yl)-4,4-dimethyl-5,6-dihydro-4H-[1,3]thiazolo[4,5-e]indazol-2-amine
5-Bromo-N-(pyrimidin-2-yl)-6H-[1,3]thiazolo[4,5-d]indazol-2-amine
5-Chloro-N-(pyrimidin-2-yl)-6H-[1,3]thiazolo[4,5-d]indazol-2-amine
5-Methyl-N-(4-methylpyrimidin-2-yl)-6H-[1,3]thiazolo[4,5-e]indazol-2-amine
5-Methyl-N-(pyrimidin-2-yl)-5,6-dihydro-4H-[1,3]thiazolo[4,5-e]indazol-2-amine
5-Methyl-N-(4-methylpyrimidin-2-yl)-5,6-dihydro-4H-[1,3]thiazolo[4,5-d]indazol-2-amine
N-(4-Methylpyrimidin-2-yl)-5′,6′-dihydrospiro[cyclopropane-1,4′-[1,3]thiazolo[4,5-d]indazol]-2′-amine
N-(Pyrimidin-2-yl)-5′,6′-dihydrospiro[cyclopropane-1,4′-[1,3]thiazolo[4,5-e]indazol]-2′-amine
N-(5-Fluoropyrimidin-2-yl)-5′,6′-dihydrospiro[cyclopropane-1,4′-[1,3]thiazolo[4,5-e]indazol]-2′-amine
N-(Pyrimidin-2-yl)-1,7-dihydropyrazolo[3′,4′:4,5]cyclopenta[1,2-d][1,3]thiazol-5-amine
N-(Pyrimidin-2-yl)-6,7-dihydro-4H-pyrazolo[4′,3′:5,6]oxepino[4,3-d][1,3]thiazol-2-amine
N-(5-Fluoropyrimidin-2-yl)-6,7-dihydro-4H-pyrazolo[4′,3′:5,6]oxepino[4,3-d][1,3]thiazol-2-amine
N-(4-Methylpyrimidin-2-yl)-6,7-dihydro-4H-pyrazolo[4′,3′:5,6]oxepino[4,3-d][1,3]thiazol-2-amine
(6-Methyl-6,7-dihydro-4H-5-oxa-3-thia-1,7,8-triaza-cyclopenta[e]azulen-2-yl)-(4-methyl-pyrimidin-2-yl)-amine
((R)-6-Methyl-6,7-dihydro-4H-5-oxa-3-thia-1,7,8-triaza-cyclopenta[e]azulen-2-yl)-(4-methyl-pyrimidin-2-yl)-amine
((S)-6-Methyl-6,7-dihydro-4H-5-oxa-3-thia-1,7,8-triaza-cyclopenta[e]azulen-2-yl)-(4-methyl-pyrimidin-2-yl)-amine
(6,7-Dihydro-4H-5-oxa-3-thia-1,7,8-triaza-cyclopenta[e]azulen-2-yl)-(4-methoxy-pyrimidin-2-yl)-amine
(4-Isopropyl-pyrimidin-2-yl)-(6-methyl-6,7-dihydro-4H-5-oxa-3-thia-1,7,8-triaza-cyclopenta[e]azulen-2-yl)-amine
(4-Ethyl-pyrimidin-2-yl)-(6-methyl-6,7-dihydro-4H-5-oxa-3-thia-1,7,8-triaza-cyclopenta[e]azulen-2-yl)-amine
(4-Cyclopropyl-pyrimidin-2-yl)-(6-methyl-6,7-dihydro-4H-5-oxa-3-thia-1,7,8-triaza-cyclopenta[e]azulen-2-yl)-amine
(N-(5-Fluoro-4-methylpyrimidin-2-yl)-6-methyl-6,7-dihydro-4H-pyrazolo[4′,3′:5,6]oxepino[4,3-d][1,3]thiazol-2-amine)
(N-(5-Fluoropyrimidin-2-yl)-6-methyl-6,7-dihydro-4H-pyrazolo[4′,3′:5,6]oxepino[4,3-d][1,3]thiazol-2-amine)
4-Methyl-N-(4-methylpyrimidin-2-yl)-6,7-dihydro-4H-pyrazolo[4′,3′:5,6]oxepino[4,3-d][1,3]thiazol-2-amine
4,6-Dimethyl-N-(4-methylpyrimidin-2-yl)-6,7-dihydro-4H-pyrazolo[4′,3′:5,6]oxepino-[4,3-d][1,3]thiazol-2-amine
N-(Pyrimidin-2-yl)-4,5,6,7-tetrahydropyrazolo[3,4-c][1,3]thiazolo[4,5-e]azepin-2-amine
5-Methyl-N-(4-methylpyrimidin-2-yl)-4,5,6,7-tetrahydropyrazolo[3,4-c][1,3]thiazolo[4,5-e]azepin-2-amine
N-(5-Fluoropyrimidin-2-yl)-4,5,6,7-tetrahydropyrazolo[3′,4′:6,7]cyclohepta[1,2-d][1,3]thiazol-2-amine
(5,5-Dimethyl-4,5,6,7-tetrahydro-3-thia-1,7,8-triaza-cyclopenta[e]azulen-2-yl)-(5-fluoro-pyrimidin-2-yl)-amine
6,6-Difluoro-N-(4-methylpyrimidin-2-yl)-4,5,6,7-tetrahydropyrazolo[3′,4′:6,7]cyclohepta[1,2-d][1,3]thiazol-2-amine
2-[(4-Methylpyrimidin-2-yl)amino]-4,7-dihydropyrazolo[3′,4′:6,7]cyclohepta[1,2-d][1,3]thiazol-6(5H)-one
5,5-Dimethyl-2-[(4-methylpyrimidin-2-yl)amino]-4,7-dihydropyrazolo[3′,4′:6,7]cyclohepta[1,2-d][1,3]thiazol-6(5H)-one
(6-Methyl-6,7-dihydro-4H-5-oxa-3-thia-1,7,8-triaza-cyclopenta[e]azulen-2-yl)-(5-methyl-[1,2,4]thiadiazol-3-yl)-amine and
(5-Fluoro-4,5,6,7-tetrahydro-3-thia-1,7,8-triaza-cyclopenta[e]azulen-2-yl)-(5-methyl-pyrimidin-2-yl)-amine;
provided that according to proviso (iii) the compound is not:
5,6-Dihydro-4H-3-thia-1,6,7-triaza-as-indacen-2-yl)-pyridin-4-yl-amine
(5,6-Dihydro-4H-3-thia-1,6,7-triaza-as-indacen-2-yl)-pyridin-3-yl-amine
(5,6-Dihydro-4H-3-thia-1,6,7-triaza-as-indacen-2-yl)-(5-methyl-pyridin-2-yl)-amine
(5,6-Dihydro-4H-3-thia-1,6,7-triaza-as-indacen-2-yl)-(4-methyl-pyridin-2-yl)-amine
(5,6-Dihydro-4H-3-thia-1,6,7-triaza-as-indacen-2-yl)-(4,6-dimethyl-pyridin-2-yl)-amine
Pyridin-3-yl-(4,5,6,7-tetrahydro-3-thia-1,7,8-triaza-cyclopenta[e]azulen-2-yl)-amine
Pyrimidin-4-yl-(4,5,6,7-tetrahydro-3-thia-1,7,8-triaza-cyclopenta[e]azulen-2-yl)-amine
Pyrazin-2-yl-(4,5,6,7-tetrahydro-3-thia-1,7,8-triaza-cyclopenta[e]azulen-2-yl)-amine
(5-Methyl-1H-pyrazol-3-yl)-(4,5,6,7-tetrahydro-3-thia-1,7,8-triaza-cyclopenta[e]azulen-2-yl)-amine
(1H-Pyrazol-3-yl)-(4,5,6,7-tetrahydro-3-thia-1,7,8-triaza-cyclopenta[e]azulen-2-yl)-amine
(4,5,6,7-Tetrahydro-3-thia-1,7,8-triaza-cyclopenta[e]azulen-2-yl)-thiazol-2-yl-amine
(6-Methyl-pyridazin-3-yl)-(4,5,6,7-tetrahydro-3-thia-1,7,8-triaza-cyclopenta[e]azulen-2-yl)-amine
(3-Methyl-pyridin-2-yl)-(4,5,6,7-tetrahydro-3-thia-1,7,8-triaza-cyclopenta[e]azulen-2-yl)-amine
6-(4,5,6,7-Tetrahydro-3-thia-1,7,8-triaza-cyclopenta[e]azulen-2-ylamino)-pyridine-2-carbonitrile
(5-Methyl-pyridin-2-yl)-(4,5,6,7-tetrahydro-3-thia-1,7,8-triaza-cyclopenta[e]azulen-2-yl)-amine
[6-(4,5,6,7-Tetrahydro-3-thia-1,7,8-triaza-cyclopenta[e]azulen-2-ylamino)-pyridin-2-yl]-methanol
6-(4,5,6,7-Tetrahydro-3-thia-1,7,8-triaza-cyclopenta[e]azulen-2-ylamino)-pyridine-2-carboxylic acid methyl ester
(4-Methoxy-pyrimidin-2-yl)-(4,5,6,7-tetrahydro-3-thia-1,7,8-triaza-cyclopenta[e]azulen-2-yl)-amine
(6-Pyrrolidin-1-yl-pyridin-2-yl)-(4,5,6,7-tetrahydro-3-thia-1,7,8-triaza-cyclopenta[e]azulen-2-yl)-amine
[4-(2-Methoxy-ethoxy)-pyrimidin-2-yl]-(4,5,6,7-tetrahydro-3-thia-1,7,8-triaza-cyclopenta[e]azulen-2-yl)-amine
N,N-Dimethyl-N′-(4,5,6,7-tetrahydro-3-thia-1,7,8-triaza-cyclopenta[e]azulen-2-yl)-pyridine-2,6-diamine and
N-Ethyl-N′-(4,5,6,7-tetrahydro-3-thia-1,7,8-triaza-cyclopenta[e]azulen-2-yl)-pyridine-2,6-diamine.

3. A compound according to claim 2 having the Formula (II) wherein:
Y is selected from the group of —CR¹R²—, —CR¹R²—CR³R⁴—, —CR¹R²—CR³R⁴—CR⁵R⁶— and —CR¹R²—CR³R⁴—CR⁵R⁶—CR⁷R⁸;
R¹, R², R³, R⁴, R⁵, R⁶, R⁷ and R⁸ are each independently selected from the group of hydrogen, halogen, —CN, —CF₃ or an optionally substituted radical selected from the group of —(C₁-C₆)alkyl, —(C₁-C₆)haloalkyl, —(C₃-C₇)cycloalkyl, aryl, heteroaryl, heterocycle, —(C₁-C₆)allylene-aryl, —(C₁-C₆)alkylene-heteroaryl, —(C₁-C₆)alkylene-heterocycle, —O—(C₀-C₆)alkyl, —N—((C₀-C₆)alkyl)₂, —(C₁-C₆)alkyl-O—(C₀-C₆)alkyl, and —(C₁-C₆)alkyl-N—((C₀-C₆)alkyl)₂;
Any two radicals of R (R¹, R², R³, R⁴, R⁵, R⁶, R⁷ or R⁸) may be taken together to form an optionally substituted 3 to 10 membered carbocyclic or heterocyclic ring;
provided that according to proviso (i) the compound is not:
N-(Pyridin-2-yl)-5,6-dihydro-4H-thiazolo[4,5-e]indazol-2-amine
N-(6-Methylpyridin-2-yl)-5,6-dihydro-4H-thiazolo[4,5-e]indazol-2-amine
N-(6-Chloropyridin-2-yl)-5,6-dihydro-4H-thiazolo[4,5-e]indazol-2-amine
N-(6-Fluoropyridin-2-yl)-5,6-dihydro-4H-thiazolo[4,5-e]indazol-2-amine
4-Methyl-N-(pyridin-2-yl)-5,6-dihydro-4H-thiazolo[4,5-e]indazol-2-amine
Pyridin-2-yl-(4,5,6,7-tetrahydro-3-thia-1,7,8-triaza-cyclopenta[e]azulen-2-yl)-amine
4-Ethyl-N-(pyridin-2-yl)-5,6-dihydro-4H-thiazolo[4,5-e]indazol-2-amine
N-(3-Fluoropyridin-2-yl)-5,6-dihydro-4H-thiazolo[4,5-e]indazol-2-amine
(6-Methyl-pyridin-2-yl)-(4,5,6,7-tetrahydro-3-thia-1,7,8-triaza-cyclopenta[e]azulen-2-yl)-amine
(5-Fluoro-pyridin-2-yl)-(4,5,6,7-tetrahydro-3-thia-1,7,8-triaza-cyclopenta[e]azulen-2-yl)-amine
(3-Fluoro-pyridin-2-yl)-(4,5,6,7-tetrahydro-3-thia-1,7,8-triaza-cyclopenta[e]azulen-2-yl)-amine
(6-Methoxy-pyridin-2-yl)-(4,5,6,7-tetrahydro-3-thia-1,7,8-triaza-cyclopenta[e]azulen-2-yl)-amine
N-(5-Fluoropyridin-2-yl)-5,6-dihydro-4H-thiazolo[4,5-e]indazol-2-amine
(6-Chloro-pyridin-2-yl)-(4,5,6,7-tetrahydro-3-thia-1,7,8-triaza-cyclopenta[e]azulen-2-yl)-amine
N-(Pyrazin-2-yl)-5,6-dihydro-4H-thiazolo[4,5-e]indazol-2-amine
(6-Fluoro-pyridin-2-yl)-(4,5,6,7-tetrahydro-3-thia-1,7,8-triaza-cyclopenta[e]azulen-2-yl)-amine
Pyrimidin-2-yl-(4,5,6,7-tetrahydro-3-thia-1,7,8-triaza-cyclopenta[e]azulen-2-yl)-amine
N-(Pyrimidin-2-yl)-5,6-dihydro-4H-thiazolo[4,5-e]indazol-2-amine
(1-Methyl-1H-pyrazol-3-yl)-(4,5,6,7-tetrahydro-3-thia-1,7,8-triaza-cyclopenta[e]azulen-2-yl)-amine
N-(1-Methyl-1H-pyrazol-3-yl)-5,6-dihydro-4H-thiazolo[4,5-e]indazol-2-amine
N-(4-Methylpyrimidin-2-yl)-5,6-dihydro-4H-thiazolo[4,5-e]indazol-2-amine
(4-Methyl-pyrimidin-2-yl)-(4,5,6,7-tetrahydro-3-thia-1,7,8-triaza-cyclopenta[e]azulen-2-yl)-amine
4,4-Dimethyl-N-(pyridin-2-yl)-5,6-dihydro-4H-thiazolo[4,5-e]indazol-2-amine
4,4-Dimethyl-N-(pyrimidin-2-yl)-5,6-dihydro-4H-thiazolo[4,5-e]indazol-2-amine
4-Methyl-N-(pyrimidin-2-yl)-5,6-dihydro-4H-thiazolo[4,5-e]indazol-2-amine and
5,5-Dimethyl-N-(pyrimidin-2-yl)-5,6-dihydro-4H-thiazolo[4,5-e]indazol-2-amine;
provided that according to proviso (iv) the compound is not:
4,4-Dimethyl-N-(4-methylpyrimidin-2-yl)-5,6-dihydro-4H-[1,3]thiazolo[4,5-e]indazol-2-amine
N-(5-Fluoropyrimidin-2-yl)-4,4-dimethyl-5,6-dihydro-4H-[1,3]thiazolo[4,5-e]indazol-2-amine
N-(5-Fluoro-4-methylpyrimidin-2-yl)-4,4-dimethyl-5,6-dihydro-4H-[1,3]thiazolo[4,5-e]indazol-2-amine
5-Methyl-N-(pyrimidin-2-yl)-5,6-dihydro-4H-[1,3]thiazolo[4,5-e]indazol-2-amine
5-Methyl-N-(4-methylpyrimidin-2-yl)-5,6-dihydro-4H-[1,3]thiazolo[4,5-e]indazol-2-amine
N-(4-Methylpyrimidin-2-yl)-5′,6′-dihydrospiro[cyclopropane-1,4′-[1,3]thiazolo[4,5-e]indazol]-2′-amine
N-(Pyrimidin-2-yl)-5′,6′-dihydrospiro[cyclopropane-1,4′-[1,3]thiazolo[4,5-e]indazol]-2′-amine
N-(5-Fluoropyrimidin-2-yl)-5′,6′-dihydrospiro[cyclopropane-1,4′-[1,3]thiazolo[4,5-e]indazol]-2′-amine
N-(Pyrimidin-2-yl)-1,7-dihydropyrazolo[3′,4′:4,5]cyclopenta[1,2-d][1,3]thiazol-5-amine
N-(5-Fluoropyrimidin-2-yl)-4,5,6,7-tetrahydropyrazolo[3′,4′:6,7]cyclohepta[1,2-d][1,3]thiazol-2-amine
(5,5-Dimethyl-4,5,6,7-tetrahydro-3-thia-1,7,8-triaza-cyclopenta[e]azulen-2-yl)-(5-fluoro-pyrimidin-2-yl)-amine
6,6-Difluoro-N-(4-methylpyrimidin-2-yl)-4,5,6,7-tetrahydropyrazolo[3′,4′:6,7]cyclohepta[1,2-d][1,3]thiazol-2-amine and
(5-Fluoro-4,5,6,7-tetrahydro-3-thia-1,7,8-triaza-cyclopenta[e]azulen-2-yl)-(5-methyl-pyrimidin-2-yl)-amine;
provided that according to proviso (iii) the compound is not:
5,6-Dihydro-4H-3-thia-1,6,7-triaza-as-indacen-2-yl)-pyridin-4-yl-amine
(5,6-Dihydro-4H-3-thia-1,6,7-triaza-as-indacen-2-yl)-pyridin-3-yl-amine
(5,6-Dihydro-4H-3-thia-1,6,7-triaza-as-indacen-2-yl)-(5-methyl-pyridin-2-yl)-amine
(5,6-Dihydro-4H-3-thia-1,6,7-triaza-as-indacen-2-yl)-(4-methyl-pyridin-2-yl)-amine
(5,6-Dihydro-4H-3-thia-1,6,7-triaza-as-indacen-2-yl)-(4,6-dimethyl-pyridin-2-yl)-amine
Pyridin-3-yl-(4,5,6,7-tetrahydro-3-thia-1,7,8-triaza-cyclopenta[e]azulen-2-yl)-amine
Pyrimidin-4-yl-(4,5,6,7-tetrahydro-3-thia-1,7,8-triaza-cyclopenta[e]azulen-2-yl)-amine
Pyrazin-2-yl-(4,5,6,7-tetrahydro-3-thia-1,7,8-triaza-cyclopenta[e]azulen-2-yl)-amine
(5-Methyl-1H-pyrazol-3-yl)-(4,5,6,7-tetrahydro-3-thia-1,7,8-triaza-cyclopenta[e]azulen-2-yl)-amine
(1H-Pyrazol-3-yl)-(4,5,6,7-tetrahydro-3-thia-1,7,8-triaza-cyclopenta[e]azulen-2-yl)-amine
(4,5,6,7-Tetrahydro-3-thia-1,7,8-triaza-cyclopenta[e]azulen-2-yl)-thiazol-2-yl-amine
(6-Methyl-pyridazin-3-yl)-(4,5,6,7-tetrahydro-3-thia-1,7,8-triaza-cyclopenta[e]azulen-2-yl)-amine
(3-Methyl-pyridin-2-yl)-(4,5,6,7-tetrahydro-3-thia-1,7,8-triaza-cyclopenta[e]azulen-2-yl)-amine
6-(4,5,6,7-Tetrahydro-3-thia-1,7,8-triaza-cyclopenta[e]azulen-2-ylamino)-pyridine-2-carbonitrile
(5-Methyl-pyridin-2-yl)-(4,5,6,7-tetrahydro-3-thia-1,7,8-triaza-cyclopenta[e]azulen-2-yl)-amine
[6-(4,5,6,7-Tetrahydro-3-thia-1,7,8-triaza-cyclopenta[e]azulen-2-ylamino)-pyridin-2-yl]-methanol
6-(4,5,6,7-Tetrahydro-3-thia-1,7,8-triaza-cyclopenta[e]azulen-2-ylamino)-pyridine-2-carboxylic acid methyl ester
(4-Methoxy-pyrimidin-2-yl)-(4,5,6,7-tetrahydro-3-thia-1,7,8-triaza-cyclopenta[e]azulen-2-yl)-amine
(6-Pyrrolidin-1-yl-pyridin-2-yl)-(4,5,6,7-tetrahydro-3-thia-1,7,8-triaza-cyclopenta[e]azulen-2-yl)-amine
[4-(2-Methoxy-ethoxy)-pyrimidin-2-yl]-(4,5,6,7-tetrahydro-3-thia-1,7,8-triaza-cyclopenta[e]azulen-2-yl)-amine
N,N-Dimethyl-N′-(4,5,6,7-tetrahydro-3-thia-1,7,8-triaza-cyclopenta[e]azulen-2-yl)-pyridine-2,6-diamine and
N-Ethyl-N′-(4,5,6,7-tetrahydro-3-thia-1,7,8-triaza-cyclopenta[e]azulen-2-yl)-pyridine-2,6-diamine.

4. A compound according to claim 3 having the Formula (H) wherein:
Y is selected from the group of —CR¹R²—, —CR¹R²—CR³R⁴—, —CR¹R²—CR³R⁴—CR⁵R⁶— and —CR¹R²—CR³R⁴—CR⁵R⁶—CR⁷R⁸;
M is an optionnally substituted pyrimidinyl;
R¹, R², R³ or R⁴ are each independently selected from the group of hydrogen, fluoro, CF₃, OMe, methyl and propyl;
provided that according to proviso (v) the compound is not:
Pyrimidin-2-yl-(4,5,6,7-tetrahydro-3-thia-1,7,8-triaza-cyclopenta[e]azulen-2-yl)-amine
N-(Pyrimidin-2-yl)-5,6-dihydro-4H-thiazolo[4,5-e]indazol-2-amine
N-(4-Methylpyrimidin-2-yl)-5,6-dihydro-4H-thiazolo[4,5-e]indazol-2-amine
(4-Methyl-pyrimidin-2-yl)-(4,5,6,7-tetrahydro-3-thia-1,7,8-triaza-cyclopenta[e]azulen-2-yl)-amine
4,4-Dimethyl-N-(pyrimidin-2-yl)-5,6-dihydro-4H-thiazolo[4,5-e]indazol-2-amine
4-Methyl-N-(pyrimidin-2-yl)-5,6-dihydro-4H-thiazolo[4,5-e]indazol-2-amine and
5,5-Dimethyl-N-(pyrimidin-2-yl)-5,6-dihydro-4H-thiazolo[4,5-e]indazol-2-amine;
provided that according to proviso (vi) the compound is not:
4,4-Dimethyl-N-(4-methylpyrimidin-2-yl)-5,6-dihydro-4H-[1,3]thiazolo[4,5-e]indazol-2-amine
N-(5-Fluoropyrimidin-2-yl)-4,4-dimethyl-5,6-dihydro-4H-[1,3]thiazolo[4,5-e]indazol-2-amine
N-(5-Fluoro-4-methylpyrimidin-2-yl)-4,4-dimethyl-5,6-dihydro-4H-[1,3]thiazolo[4,5-e]indazol-2-amine
5-Methyl-N-(pyrimidin-2-yl)-5,6-dihydro-4H-[1,3]thiazolo[4,5-e]indazol-2-amine
5-Methyl-N-(4-methylpyrimidin-2-yl)-5,6-dihydro-4H-[1,3]thiazolo[4,5-e]indazol-2-amine
N-(Pyrimidin-2-yl)-1,7-dihydropyrazolo[3′,4′:4,5]cyclopenta[1,2-d][1,3]thiazol-5-amine
N-(5-Fluoropyrimidin-2-yl)-4,5,6,7-tetrahydropyrazolo[3′,4′:6,7]cyclohepta[1,2-d][1,3]thiazol-2-amine
(5,5-Dimethyl-4,5,6,7-tetrahydro-3-thia-1,7,8-triaza-cyclopenta[e]azulen-2-yl)-(5-fluoro-pyrimidin-2-yl)-amine
6,6-Difluoro-N-(4-methylpyrimidin-2-yl)-4,5,6,7-tetrahydropyrazolo[3′,4′:6,7]cyclohepta[1,2-d][1,3]thiazol-2-amine and
(5-Fluoro-4,5,6,7-tetrahydro-3-thia-1,7,8-triaza-cyclopenta[e]azulen-2-yl)-(5-methyl-pyrimidin-2-yl)-amine;
provided that according to proviso (vii) the compound is not:
Pyrimidin-4-yl-(4,5,6,7-tetrahydro-3-thia-1,7,8-triaza-cyclopenta[e]azulen-2-yl)-amine
(4-Methoxy-pyrimidin-2-yl)-(4,5,6,7-tetrahydro-3-thia-1,7,8-triaza-cyclopenta[e]azulen-2-yl)-amine and
[4-(2-Methoxy-ethoxy)-pyrimidin-2-yl]-(4,5,6,7-tetrahydro-3-thia-1,7,8-triaza-cyclopenta[e]azulen-2-yl)-amine.

5. A compound according to claim 2 having the Formula (II) wherein:
Y is selected from the group of —O—CR¹R²—, —O—CR¹R²—CR³R⁴—, —NR⁹—CR¹R²— and —NR⁹—CR¹R²—CR³R⁴—;
R¹, R², R³ or R⁴ are each independently selected from the group of hydrogen, halogen, —CN, —CF₃ or an optionally substituted radical selected from the group of —(C₁-C₆)alkyl, —(C₁-C₆)haloalkyl, —(C₃-C₇)cycloalkyl, aryl, heteroaryl, heterocycle, —(C₁-C₆)alkylene-aryl, —(C₁-C₆)alkylene-heteroaryl, —(C₁-C₆)alkylene-heterocycle, —O—(C₀-C₆)alkyl, —N—((C₀-C₆)alkyl)₂, —(C₁-C₆)alkyl-O—(C₀-C₆)allyl, and —(C₁-C₆)alkyl-N—((C₀-C₆)alkyl)₂;
Any two radicals of R (R¹, R², R³ or R⁴) may be taken together to form an optionally substituted 3 to 10 membered carbocyclic or heterocyclic ring; and
R⁹ is selected from the group of hydrogen or an optionally substituted radical selected from the group of —(C₁-C₆)alkyl, —(C₁-C₆)haloalkyl, —(C₃-C₇)cycloalkyl, —(C₁-C₆)alkylene-(C₁-C₆)haloalkyl, —(C₁-C₆)alkylene-(C₃-C₇)halocycloalkyl, aryl, heteroaryl, heterocycle, —(C₁-C₆)alkylene-aryl, —(C₁-C₆)alkylene-heteroaryl, —(C₁-C₆)alkylene-heterocycle, —(C₁-C₆)alkylene-(C₃-C₇)cycloalkyl, —(C₂-C₆)alkyl-O—(C₀-C₆)alkyl, and —(C₂-C₆)alkyl-N—((C₀-C₆)alkyl)₂.

6. A compound according to claim 2 having the Formula (II) wherein:
Y is selected from the group of —O—CR¹R²—, —O—CR¹R²—CR³R⁴—, —NR⁹—CR¹R²— and NR⁹—CR¹R²—CR³R⁴—;
R¹, R², R³ or R⁴ are each independently selected from the group of hydrogen, halogen, —CN, —CF₃ or an optionally substituted radical selected from the group of —(C₁-C₆)allyl, —(C₁-C₆)haloalkyl, —(C₃-C₇)cycloalkyl, aryl, heteroaryl, heterocycle, —(C₁-C₆)alkylene-aryl, —(C₁-C₆)allylene-heteroaryl, —(C₁-C₆)alkylene-heterocycle, —O—(C₀-C₆)alkyl, —N—((C₀-C₆)alkyl)₂, —(C₁-C₆)alkyl-O—(C₀-C₆)allyl, and —(C₁-C₆)alkyl-N—((C₀-C₆)alkyl)₂;
Any two radicals of R (R¹, R², R³ or R⁴) may be taken together to form an optionally substituted 3 to 10 membered carbocyclic or heterocyclic ring; and
R⁹ is selected from the group of hydrogen or an optionally substituted radical selected from the group of —(C₁-C₆)alkyl, —(C₁-C₆)haloalkyl, —(C₃-C₇)cycloalkyl, —(C₁-C₆)alkylene-(C₁-C₆)haloalkyl, —(C₁-C₆)allylene-(C₃-C₇)halocycloalkyl, aryl, heteroaryl, heterocycle, —(C₁-C₆)alkylene-aryl, —(C₁-C₆)alkylene-heteroaryl, —(C₁-C₆)allylene-heterocycle, —(C₂-C₆)alkyl-O—(C₀-C₆)alkyl, and —(C₂-C₆)alkyl-N—((C₀-C₆)alkyl)₂.

7. A compound according to claim 2 having the Formula (II) wherein:
Y is selected from the group of —CR¹R²—O—CR³R⁴— and —CR¹R²—NR⁹—CR³R⁴—;
R¹, R², R³ or R⁴ are each independently selected from the group of hydrogen, —CN, —CF₃ or an optionally substituted radical selected from the group of —(C₁-C₆)alkyl, —(C₁-C₆)haloalicyl, —(C₃-C₇)cycloalkyl, aryl, heteroaryl, heterocycle, —(C₁-C₆)alkylene-aryl, —(C₁-C₆)alkylene-heteroaryl, —(C₁-C₆)alkylene-heterocycle, —(C₁-C₆)alkyl-O—(C₀-C₆)alkyl, and —(C₁-C₆)alkyl-N—((C₀-C₆)alkyl)₂;
Any two radicals of R (R¹, R², R³ or R⁴) may be taken together to form an optionally substituted 3 to 10 membered carbocyclic or heterocyclic ring; and
R⁹ is selected from the group of hydrogen or an optionally substituted radical selected from the group of —(C₁-C₆)alkyl, —(C₁-C₆)haloalkyl, —(C₃-C₇)cycloalkyl, —(C₁-C₆)alkylene-(C₁-C₆)haloalkyl, —(C₁-C₆)allylene-(C₃-C₇)halocycloalkyl, aryl, heteroaryl, heterocycle, —(C₁-C₆)alkylene-aryl, —(C₁-C₆)alkylene-heteroaryl, —(C₁-C₆)alkylene-hetero cycle, —(C₁-C₆)alkylene-(C₃-C₇)cycloalkyl, —(C₂-C₆)alkyl-O—(C₀-C₆)alkyl, and —(C₂-C₆)alkyl-N—((C₀-C₆)alkyl)₂;
provided that according to proviso (viii) the compound is not:
N-(Pyrimidin-2-yl)-6,7-dihydro-4H-pyrazolo[4′,3′:5,6]oxepino[4,3-d][1,3]thiazol-2-amine
N-(5-Fluoropyrimidin-2-yl)-6,7-dihydro-4H-pyrazolo[4′,3′:5,6]oxepino[4,3-d][1,3]thiazol-2-amine
N-(4-Methylpyrimidin-2-yl)-6,7-dihydro-4H-pyrazolo[4′,3′:5,6]oxepino[4,3-c][1,3]thiazol-2-amine
(6-Methyl-6,7-dihydro-4H-5-oxa-3-thia-1,7,8-triaza-cyclopenta[e]azulen-2-yl)-(4-methyl-pyrimidin-2-yl)-amine
((R)-6-Methyl-6,7-dihydro-4H-5-oxa-3-thia-1,7,8-triaza-cyclopenta[e]azulen-2-yl)-(4-methyl-pyrimidin-2-yl)-amine
((S)-6-Methyl-6,7-dihydro-4H-5-oxa-3-thia-1,7,8-triaza-cyclopenta[e]azulen-2-yl)-(4-methyl-pyrimidin-2-yl)-amine
(6,7-Dihydro-4H-5-oxa-3-thia-1,7,8-triaza-cyclopenta[e]azulen-2-yl)-(4-methoxy-pyrimidin-2-yl)-amine
(4-Isopropyl-pyrimidin-2-yl)-(6-methyl-6,7-dihydro-4H-5-oxa-3-thia-1,7,8-triaza-cyclopenta[e]azulen-2-yl)-amine
(4-Ethyl-pyrimidin-2-yl)-(6-methyl-6,7-dihydro-4H-5-oxa-3-thia-1,7,8-triaza-cyclopenta[e]azulen-2-yl)-amine
(4-Cyclopropyl-pyrimidin-2-yl)-(6-methyl-6,7-dihydro-4H-5-oxa-3-thia-1,7,8-triaza-cyclopenta[e]azulen-2-yl)-amine
(N-(5-Fluoro-4-methylpyrimidin-2-yl)-6-methyl-6,7-dihydro-4H-pyrazolo[4′,3′:5,6]oxepino[4,3-d][1,3]thiazol-2-amine)
(N-(5-Fluoropyrimidin-2-yl)-6-methyl-6,7-dihydro-4H-pyrazolo[4′,3′:5,6]oxepino[4,3-d][1,3]thiazol-2-amine)
4-Methyl-N-(4-methylpyrimidin-2-yl)-6,7-dihydro-4H-pyrazolo[4′,3′:5,6]oxepino[4,3-d][1,3]thiazol-2-amine
4,6-Dimethyl-N-(4-methylpyrimidin-2-yl)-6,7-dihydro-4H-pyrazolo[4′,3′:5,6]oxepino-[4,3-d][1,3]thiazol-2-amine
N-(Pyrimidin-2-yl)-4,5,6,7-tetrahydropyrazolo[3,4-c][1,3]thiazolo[4,5-e]azepin-2-amine
5-Methyl-N-(4-methylpyrimidin-2-yl)-4,5,6,7-tetrahydropyrazolo[3,4-c][1,3]thiazolo[4,5-e]azepin-2-amine and
(6-Methyl-6,7-dihydro-4H-5-oxa-3-thia-1,7,8-triaza-cyclopenta[e]azulen-2-yl)-(5-methyl-[1,2,4]thiadiazol-3-yl)-amine.

8. A compound according to claim 2 having the Formula (II) wherein:
Y is selected from the group of —CR¹R²—O—CR³R⁴— and —CR¹R²—NR⁹—CR³R⁴—;
R¹, R², R³ or R⁴ are each independently selected from the group of hydrogen, —CN, —CF₃ or an optionally substituted radical selected from the group of —(C₁-C₆)alkyl, —(C₁-C₆)haloalkyl, —(C₃-C₇)cycloalkyl, aryl, heteroaryl, heterocycle, —(C₁-C₆)alkylene-aryl, —(C₁-C₆)alkylene-heteroaryl, —(C₁-C₆)alkylene-heterocycle, —(C₁-C₆)alkyl-O—(C₀-C₆)alkyl, and —(C₁-C₆)alkyl-N—((C₀-C₆)alkyl)₂;
Any two radicals of R (R¹, R², R³ or R⁴) may be taken together to form an optionally substituted 3 to 10 membered carbocyclic or heterocyclic ring; and
R⁹ is selected from the group of hydrogen or an optionally substituted radical selected from the group of —(C₁-C₆)alkyl, —(C₁-C₆)haloalkyl, —(C₃-C₇)cycloalkyl, —(C₁-C₆)alkylene-(C₁-C₆)haloalkyl, —(C₁-C₆)alkylene-(C₃-C₇)halocycloalkyl, aryl, heteroaryl, heterocycle, —(C₁-C₆)alkylene-aryl, —(C₁-C₆)alkylene-heteroaryl, —(C₁-C₆)alkylene-heterocycle, —(C₂-C₆)alkyl-O—(C₀-C₆)alkyl, and —(C₂-C₆)alkyl-N—((C₀-C₆)alkyl)₂;
provided that according to proviso (viii) the compound is not:
N-(Pyrimidin-2-yl)-6,7-dihydro-4H-pyrazolo[4′,3′:5,6]oxepino[4,3-d][1,3]thiazol-2-amine
N-(5-Fluoropyrimidin-2-yl)-6,7-dihydro-4H-pyrazolo[4′,3′:5,6]oxepino[4,3-d][1,3]thiazol-2-amine
N-(4-Methylpyrimidin-2-yl)-6,7-dihydro-4H-pyrazolo[4′,3′:5,6]oxepino[4,3-d][1,3]thiazol-2-amine
(6-Methyl-6,7-dihydro-4H-5-oxa-3-thia-1,7,8-triaza-cyclopenta[e]azulen-2-yl)-(4-methyl-pyrimidin-2-yl)-amine
((R)-6-Methyl-6,7-dihydro-4H-5-oxa-3-thia-1,7,8-triaza-cyclopenta[e]azulen-2-yl)-(4-methyl-pyrimidin-2-yl)-amine
((S)-6-Methyl-6,7-dihydro-4H-5-oxa-3-thia-1,7,8-triaza-cyclopenta[e]azulen-2-yl)-(4-methyl-pyrimidin-2-yl)-amine
(6,7-Dihydro-4H-5-oxa-3-thia-1,7,8-triaza-cyclopenta[e]azulen-2-yl)-(4-methoxy-pyrimidin-2-yl)-amine
(4-Isopropyl-pyrimidin-2-yl)-(6-methyl-6,7-dihydro-4H-5-oxa-3-thia-1,7,8-triaza-cyclopenta[e]azulen-2-yl)-amine
(4-Ethyl-pyrimidin-2-yl)-(6-methyl-6,7-dihydro-4H-5-oxa-3-thia-1,7,8-triaza-cyclopenta[e]azulen-2-yl)-amine
(4-Cyclopropyl-pyrimidin-2-yl)-(6-methyl-6,7-dihydro-4H-5-oxa-3-thia-1,7,8-triaza-cyclopenta[e]azulen-2-yl)-amine
(N-(5-Fluoro-4-methylpyrimidin-2-yl)-6-methyl-6,7-dihydro-4H-pyrazolo[4′,3′:5,6]oxepino[4,3-d][1,3]thiazol-2-amine)
(N-(5-Fluoropyrimidin-2-yl)-6-methyl-6,7-dihydro-4H-pyrazolo[4′,3′:5,6]oxepino[4,3-d][1,3]thiazol-2-amine)
4-Methyl-N-(4-methylpyrimidin-2-yl)-6,7-dihydro-4H-pyrazolo[4′,3′:5,6]oxepino[4,3-d][1,3]thiazol-2-amine
4,6-Dimethyl-N-(4-methylpyrimidin-2-yl)-6,7-dihydro-4H-pyrazolo[4′,3′:5,6]oxepino-[4,3-d][1,3]thiazol-2-amine
N-(Pyrimidin-2-yl)-4,5,6,7-tetrahydropyrazolo[3,4-c][1,3]thiazolo[4,5-e]azepin-2-amine
5-Methyl-N-(4-methylpyrimidin-2-yl)-4,5,6,7-tetrahydropyrazolo[3,4-c][1,3]thiazolo[4,5-e]azepin-2-amine and
(6-Methyl-6,7-dihydro-4H-5-oxa-3-thia-1,7,8-triaza-cyclopenta[e]azulen-2-yl)-(5-methyl-[1,2,4]thiadiazol-3-yl)-amine.

9. A compound according to claim 8 having the Formula (III) wherein: 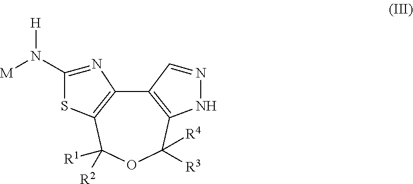
M is an optionally substituted pyridinyl, pyrimidinyl, thiadiazolyl, triazinyl, thiazolyl and oxadiazolyl;
R¹, R², R³ or R⁴ are each independently selected from the group of hydrogen or an optionally substituted radical selected from the group of —(C₁-C₆)alkyl, —(C₁-C₆)haloalkyl, —(C₃-C₇)cycloalkyl, aryl, heteroaryl, heterocycle, —(C₁-C₆)alkylene-aryl, —(C₁-C₆)alkylene-heteroaryl, —(C₁-C₆)alkylene-heterocycle, —(C₁-C₆)alkyl-O—(C₀-C₆)alkyl, and —(C₁-C₆)alkyl-N—((C₀-C₆)alkyl)₂; and
Any two radicals of R (R¹, R², R³ or R⁴) may be taken together to form an optionally substituted 3 to 10 membered carbocyclic or heterocyclic ring;
provided that according to proviso (ix) the compound is not:
N-(Pyrimidin-2-yl)-6,7-dihydro-4H-pyrazolo[4′,3′:5,6]oxepino[4,3-d][1,3]thiazol-2-amine
N-(5-Fluoropyrimidin-2-yl)-6,7-dihydro-4H-pyrazolo[4′,3′:5,6]oxepino[4,3-d][1,3]thiazol-2-amine
N-(4-Methylpyrimidin-2-yl)-6,7-dihydro-4H-pyrazolo[4′,3′:5,6]oxepino[4,3-d][1,3]thiazol-2-amine
(6-Methyl-6,7-dihydro-4H-5-oxa-3-thia-1,7,8-triaza-cyclopenta[e]azulen-2-yl)-(4-methyl-pyrimidin-2-yl)-amine
((R)-6-Methyl-6,7-dihydro-4H-5-oxa-3-thia-1,7,8-triaza-cyclopenta[e]azulen-2-yl)-(4-methyl-pyrimidin-2-yl)-amine
((S)-6-Methyl-6,7-dihydro-4H-5-oxa-3-thia-1,7,8-triaza-cyclopenta[e]azulen-2-yl)-(4-methyl-pyrimidin-2-yl)-amine
(6,7-Dihydro-4H-5-oxa-3-thia-1,7,8-triaza-cyclopenta[e]azulen-2-yl)-(4-methoxy-pyrimidin-2-yl)-amine
(4-Isopropyl-pyrimidin-2-yl)-(6-methyl-6,7-dihydro-4H-5-oxa-3-thia-1,7,8-triaza-cyclopenta[e]azulen-2-yl)-amine
(4-Ethyl-pyrimidin-2-yl)-(6-methyl-6,7-dihydro-4H-5-oxa-3-thia-1,7,8-triaza-cyclopenta[e]azulen-2-yl)-amine
(4-Cyclopropyl-pyrimidin-2-yl)-(6-methyl-6,7-dihydro-4H-5-oxa-3-thia-1,7,8-triaza-cyclopenta[e]azulen-2-yl)-amine
(N-(5-Fluoro-4-methylpyrimidin-2-yl)-6-methyl-6,7-dihydro-4H-pyrazolo[4′,3′:5,6]oxepino[4,3-d][1,3]thiazol-2-amine)
(N-(5-Fluoropyrimidin-2-yl)-6-methyl-6,7-dihydro-4H-pyrazolo[4′,3′:5,6]oxepino[4,3-d][1,3]thiazol-2-amine)
4-Methyl-N-(4-methylpyrimidin-2-yl)-6,7-dihydro-4H-pyrazolo[4′,3′:5,6]oxepino[4,3-d][1,3]thiazol-2-amine
4,6-Dimethyl-N-(4-methylpyrimidin-2-yl)-6,7-dihydro-4H-pyrazolo[4′,3′:5,6]oxepino-[4,3-d][1,3]thiazol-2-amine and
(6-Methyl-6,7-dihydro-4H-5-oxa-3-thia-1,7,8-triaza-cyclopenta[e]azulen-2-yl)-(5-methyl-[1,2,4]thiadiazol-3-yl)-amine.

10. A compound according to claim 9 having the Formula (III) wherein:
M is an optionally substituted pyridinyl, pyrimidinyl and thiadiazolyl;
R¹, R², R³ or R⁴ are each independently selected from the group of hydrogen, CF₃, methyl, ethyl, isopropyl, and an optionally substituted pyridinyl;
provided that according to proviso (ix) the compound is not:
N-(Pyrimidin-2-yl)-6,7-dihydro-4H-pyrazolo[4′,3′:5,6]oxepino[4,3-d][1,3]thiazol-2-amine
N-(5-Fluoropyrimidin-2-yl)-6,7-dihydro-4H-pyrazolo[4′,3′:5,6]oxepino[4,3-d][1,3]thiazol-2-amine
N-(4-Methylpyrimidin-2-yl)-6,7-dihydro-4H-pyrazolo[4′,3′:5,6]oxepino[4,3-d][1,3]thiazol-2-amine
(6-Methyl-6,7-dihydro-4H-5-oxa-3-thia-1,7,8-triaza-cyclopenta[e]azulen-2-yl)-(4-methyl-pyrimidin-2-yl)-amine
((R)-6-Methyl-6,7-dihydro-4H-5-oxa-3-thia-1,7,8-triaza-cyclopenta[e]azulen-2-yl)-(4-methyl-pyrimidin-2-yl)-amine
((S)-6-Methyl-6,7-dihydro-4H-5-oxa-3-thia-1,7,8-triaza-cyclopenta[e]azulen-2-yl)-(4-methyl-pyrimidin-2-yl)-amine
(6,7-Dihydro-4H-5-oxa-3-thia-1,7,8-triaza-cyclopenta[e]azulen-2-yl)-(4-methoxy-pyrimidin-2-yl)-amine
(4-Isopropyl-pyrimidin-2-yl)-(6-methyl-6,7-dihydro-4H-5-oxa-3-thia-1,7,8-triaza-cyclopenta[e]azulen-2-yl)-amine
(4-Ethyl-pyrimidin-2-yl)-(6-methyl-6,7-dihydro-4H-5-oxa-3-thia-1,7,8-triaza-cyclopenta[e]azulen-2-yl)-amine
(4-Cyclopropyl-pyrimidin-2-yl)-(6-methyl-6,7-dihydro-4H-5-oxa-3-thia-1,7,8-triaza-cyclopenta[e]azulen-2-yl)-amine
(N-(5-Fluoro-4-methylpyrimidin-2-yl)-6-methyl-6,7-dihydro-4H-pyrazolo[4′,3′:5,6]oxepino[4,3-d][1,3]thiazol-2-amine)
(N-(5-Fluoropyrimidin-2-yl)-6-methyl-6,7-dihydro-4H-pyrazolo[4′,3′:5,6]oxepino[4,3-d][1,3]thiazol-2-amine)
4-Methyl-N-(4-methylpyrimidin-2-yl)-6,7-dihydro-4H-pyrazolo[4′,3′:5,6]oxepino[4,3-d][1,3]thiazol-2-amine
4,6-Dimethyl-N-(4-methylpyrimidin-2-yl)-6,7-dihydro-4H-pyrazolo[4′,3′:5,6]oxepino-[4,3-d][1,3]thiazol-2-amine and
(6-Methyl-6,7-dihydro-4H-5-oxa-3-thia-1,7,8-triaza-cyclopenta[e]azulen-2-yl)-(5-methyl-[1,2,4]thiadiazol-3-yl)-amine.

11. A compound according to claim 2 having the Formula (II) wherein:
Y is selected from the group of —CR¹R²—O—, —CR¹R²—CR³R⁴—O— and —CR¹R²—NR⁹—;
R¹, R², R³ or R⁴ are each independently selected from the group of hydrogen, halogen, —CN, —CF₃ or an optionally substituted radical selected from the group of —(C₁-C₆)alkyl, —(C₁-C₆)haloalkyl, —(C₃-C₇)cycloalkyl, aryl, heteroaryl, heterocycle, —(C₁-C₆)alkylene-aryl, —(C₁-C₆)alkylene-heteroaryl, —(C₁-C₆)alkylene-heterocycle, —O—(C₀-C₆)alkyl, —N—((C₀-C₆)alkyl)₂, —(C₁-C₆)alkyl-O—(C₀-C₆)allyl, and —(C₁-C₆)alkyl-N—((C₀-C₆)alkyl)₂;
Any two radicals of R(R¹, R², R³ or R⁴) may be taken together to form an optionally substituted 3 to 10 membered carbocyclic or heterocyclic ring; and
R⁹ is selected from the group of hydrogen or an optionally substituted radical selected from the group of —(C₁-C₆)alkyl, —(C₁-C₆)haloalkyl, —(C₃-C₇)cycloalkyl, (C₁-C₆)alkylene-(C₁-C₆)haloalkyl, —(C₁-C₆)allylene-(C₃-C₇)halocycloalkyl, aryl, heteroaryl, heterocycle, —(C₁-C₆)allylene-aryl, —(C₁-C₆)alkylene-heteroaryl, —(C₁-C₆)alkylene-heterocycle, —(C₁-C₆)alkylene-(C₃-C₇)cycloalkyl, —(C₂-C₆)alkyl-O—(C₀-C₆)alkyl, and —(C₂-C₆)alkyl-N—((C₀-C₆)alkyl)₂.

12. A compound according to claim 2 having the Formula (II) wherein:
Y is selected from the group of —CR¹R²—O—, —CR¹R²—CR³R⁴—O— and —CR¹R²—NR⁹—;
R¹, R², R³ or R⁴ are each independently selected from the group of hydrogen, halogen, —CN, —CF₃ or an optionally substituted radical selected from the group of —(C₁-C₆)alkyl, —(C₁-C₆)haloalkyl, —(C₃-C₇)cycloalkyl, aryl, heteroaryl, heterocycle, —(C₁-C₆)alkylene-aryl, —(C₁-C₆)alkylene-heteroaryl, —(C₁-C₆)allylene-heterocycle, —O—(C₀-C₆)alkyl, —N—((C₀-C₆)alkyl)₂, —(C₁-C₆)alkyl-O—(C₀-C₆)allyl, and —(C₁-C₆)alkyl-N—((C₀-C₆)alkyl)₂;
Any two radicals of R (R¹, R², R³ or R⁴) may be taken together to form an optionally substituted 3 to 10 membered carbocyclic or heterocyclic ring; and
R⁹ is selected from the group of hydrogen or an optionally substituted radical selected from the group of —(C₁-C₆)alkyl, —(C₁-C₆)haloalkyl, —(C₃-C₇)cycloalkyl, —(C₁-C₆)alkylene-(C₁-C₆)haloalkyl, —(C₁-C₆)alkylene-(C₃-C₇)halocycloalkyl, aryl, heteroaryl, heterocycle, —(C₁-C₆)alkylene-aryl, —(C₁-C₆)allylene-heteroaryl, —(C₁-C₆)alkylene-heterocycle, —(C₂-C₆)alkyl-O—(C₀-C₆)alkyl, and —(C₂-C₆)alkyl-N—((C₀-C₆)alkyl)₂.

13. A compound according to claims 1 to 12, which can exist as optical isomers, wherein said compound is either the racemic mixture or one or both of the individual optical isomers.

14. A compound according to claims 1 to 13, wherein said compound is selected from:
(4-Methyl-6H-3-thia-1,6,7-triaza-as-indacen-2-yl)-pyridin-2-yl-amine
(4-Methyl-pyrimidin-2-yl)-(4,4,6-trimethyl-6,7-dihydro-4H-5-oxa-3-thia-1,7,8-triaza-cyclopenta[e]azulen-2-yl)-amine
2-(4-Methyl-pyrimidin-2-ylamino)-4,5,6,7-tetrahydro-3-thia-1,7,8-triaza-cyclopenta[e]azulen-6-ol
(4,4-Dimethyl-6,7-dihydro-4H-5-oxa-3-thia-1,7,8-triaza-cyclopenta[e]azulen-2-yl)-(4-methyl-pyrimidin-2-yl)-amine
(4-Methyl-pyrimidin-2-yl)-(5-propyl-5,6-dihydro-4H-3-thia-1,6,7-triaza-as-indacen-2-yl)-amine
Dimethyl-carbamic acid 2-(4-methyl-pyrimidin-2-ylamino)-4,5,6,7-tetrahydro-3-thia-1,7,8-triaza-cyclopenta[e]azulen-5-yl ester
(4-Methyl-pyrimidin-2-yl)-(5,6,7,8-tetrahydro-4H-3-thia-1,8,9-triaza-dicyclopenta[a,c]cycloocten-2-yl)-amine
(6-Ethyl-6,7-dihydro-4H-5-oxa-3-thia-1,7,8-triaza-cyclopenta[e]azulen-2-yl)-(4-methyl-pyrimidin-2-yl)-amine
(6,6-Difluoro-5,5-dimethyl-4,5,6,7-tetrahydro-3-thia-1,7,8-triaza-cyclopenta[e]azulen-2-yl)-(4-methyl-pyrimidin-2-yl)-amine
(6,6-Dimethyl-6,7-dihydro-4H-5-oxa-3-thia-1,7,8-triaza-cyclopenta[e]azulen-2-yl)-(4-methyl-pyrimidin-2-yl)-amine
(4-Methyl-pyrimidin-2-yl)-(5-methyl-4,5,6,7-tetrahydro-3-thia-1,7,8-triaza-cyclopenta[e]azulen-2-yl)-amine
(4-Methyl-pyrimidin-2-yl)-(6-methyl-4,5,6,7-tetrahydro-3-thia-1,7,8-triaza-cyclopenta[e]azulen-2-yl)-amine
(4-Methyl-pyrimidin-2-yl)-(6-trifluoromethyl-6,7-dihydro-4H-5-oxa-3-thia-1,7,8-triaza-cyclopenta[e]azulen-2-yl)-amine
(4-Methoxy-pyrimidin-2-yl)-(6-methyl-6,7-dihydro-4H-5-oxa-3-thia-1,7,8-triaza-cyclopenta[e]azulen-2-yl)-amine
(5-Methoxy-4,5,6,7-tetrahydro-3-thia-1,7,8-triaza-cyclopenta[e]azulen-2-yl)-(4-methyl-pyrimidin-2-yl)-amine
(6-Isopropyl-6,7-dihydro-4H-5-oxa-3-thia-1,7,8-triaza-cyclopenta[e]azulen-2-yl)-(4-methyl-pyrimidin-2-yl)-amine
(4,7-Dihydro-5H-6-oxa-3-thia-1,7,8-triaza-cyclopenta[e]azulen-2-yl)-(4-methyl-pyrimidin-2-yl)-amine
(5,5-Difluoro-4,5,6,7-tetrahydro-3-thia-1,7,8-triaza-cyclopenta[e]azulen-2-yl)-(4-methyl-pyrimidin-2-yl)-amine
(6-Ethyl-6,7-dihydro-4H-5-oxa-3-thia-1,7,8-triaza-cyclopenta[e]azulen-2-yl)-(5-fluoro-pyrimidin-2-yl)-amine
(6-Ethyl-6,7-dihydro-4H-5-oxa-3-thia-1,7,8-triaza-cyclopenta[e]azulen-2-yl)-(5-methyl-[1,2,4]thiadiazol-3-yl)-amine
(6-Ethyl-6,7-dihydro-4H-5-oxa-3-thia-1,7,8-triaza-cyclopenta[e]azulen-2-yl)-(5-fluoro-4-methyl-pyrimidin-2-yl)-amine
(6-Ethyl-6,7-dihydro-4H-5-oxa-3-thia-1,7,8-triaza-cyclopenta[e]azulen-2-yl)-(4-methoxy-pyrimidin-2-yl)-amine
(6-Fluoro-pyridin-2-yl)-(6-methyl-6,7-dihydro-4H-5-oxa-3-thia-1,7,8-triaza-cyclopenta[e]azulen-2-yl)-amine
(6-Methoxy-4,5,6,8-tetrahydro-3-thia-1,7,8-triaza-cyclopenta[e]azulen-2-yl)-(4-methyl-pyrimidin-2-yl)-amine
(6-Isopropyl-6,7-dihydro-4H-5-oxa-3-thia-1,7,8-triaza-cyclopenta[e]azulen-2-yl)-(5-methyl-[1,2,4]thiadiazol-3-yl)-amine
(6-Fluoro-pyridin-2-yl)-(6-isopropyl-6,7-dihydro-4H-5-oxa-3-thia-1,7,8-triaza-cyclopenta[e]azulen-2-yl)-amine
(4-Methyl-pyrimidin-2-yl)-(6-trifluoromethyl-4,5,6,7-tetrahydro-3-thia-1,7,8-triaza-cyclopenta[e]azulen-2-yl)-amine
(5-Fluoro-pyrimidin-2-yl)-(6-isopropyl-6,7-dihydro-4H-5-oxa-3-thia-1,7,8-triaza-cyclopenta[e]azulen-2-yl)-amine
(5-Fluoro-4-methyl-pyrimidin-2-yl)-(6-isopropyl-6,7-dihydro-4H-5-oxa-3-thia-1,7,8-triaza-cyclopenta[e]azulen-2-yl)-amine
2-(5-Fluoro-4-methyl-pyrimidin-2-ylamino)-6,7-dihydro-5H-3-thia-1,7,8-triaza-cyclopenta[e]azulen-4-one
(4-Methyl-pyrimidin-2-yl)-(6-pyridin-2-yl-6,7-dihydro-4H-5-oxa-3-thia-1,7,8-triaza-cyclopenta[e]azulen-2-yl)-amine
and a pharmaceutically acceptable acid or base addition salt thereof, a stereochemically isomeric form thereof and an N-oxide form thereof.

15. A pharmaceutical composition comprising a therapeutically effective amount of a compound according to claims 1 to 14 and a pharmaceutically acceptable carrier and/or excipient.

16. A method of treating or preventing a condition in a mammal, including a human, the treatment or prevention of which is affected or facilitated by the neuromodulatory effect of mGluR₄ allosteric modulators, comprising administering to a mammal in need of such treatment or prevention, an effective amount of a compound/composition according to claims 1 to 15.

17. A method of treating or preventing a condition in a mammal, including a human, the treatment or prevention of which is affected or facilitated by the neuromodulatory effect of mGluR₄ positive allosteric modulators, comprising administering to a mammal in need of such treatment or prevention, an effective amount of a compound/composition according to claims 1 to 15.

18. A method useful for treating or preventing central nervous system disorders selected from the group consisting of: addiction, tolerance or dependence; affective disorders, such as depression and anxiety; psychiatric disease such as psychotic disorders, attention-deficit/hyperactivity disorder and bipolar disorder; Parkinson's disease, memory impairment, Alzheimer's disease, dementia, delirium tremens, other forms of neurodegeneration, neurotoxicity, and ischemia, comprising administering to a mammalian patient in need of such treatment or prevention, an effective amount of a compound/composition according to claims 1 to 15.

19. A method useful for treating or preventing central nervous system disorders selected from the group consisting of Parkinson's disease and movement disorders such as bradykinesia, rigidity, dystonia, drug-induced parkinsonism, dyskinesia, tardive dyskinesia, L-DOPA-induced dyskinesia, dopamine agonist-induced dyskinesia, hyperkinetic movement disorders, Gilles de la Tourette syndrome, resting tremor, action tremor, akinesia, akinetic-rigid syndrome, akathisia, athetosis, asterixis, tics, postural instability, postencephalitic parkinsonism, muscle rigidity, chorea and choreaform movements, spasticity, myoclonus, hemiballismus, progressive supranuclear palsy, restless legs syndrome, and periodic limb movement disorder, comprising administering to a mammalian patient in need of such treatment or prevention, an effective amount of a compound/composition according to claims 1 to 15.

20. A method of claim 19 comprising administering to a mammalian patient in need of such treatment or prevention, an effective amount of a compound/composition according to claims 1 to 15 in combination with an agent selected from the group consisting of: levodopa, levodopa with a selective extracerebral decarboxylase inhibitor, carbidopa, entacapone, a COMT inhibitor, a dopamine agonist, an anticholinergic, a cholinergic agonist, a butyrophenone neuroleptic agent, a diphenylbutylpiperidine neuroleptic agent, a heterocyclic dibenzazepine neuroleptic agent, an indolone neuroleptic agent, a phenothiazine neuroleptic agent, a thioxanthene neuroleptic agent, an NMDA receptor antagonist, an MAO-B inhibitor, an mGluR₅ antagonist or an A_{2A} antagonist.

21. A method useful for treating or preventing central nervous system disorders selected from the group consisting of: cognitive disorders such as delirium, substance-induced persisting delirium, dementia, dementia due to HIV disease, dementia due to Huntington's disease, dementia due to Parkinson's disease, Parkinsonian-ALS demential complex, dementia of the Alzheimer's type, substance-induced persisting dementia, and mild cognitive impairment, comprising administering to a mammalian patient in need of such treatment or prevention, an effective amount of a compound/composition according to claims 1 to 15.

22. A method useful for treating affective disorders selected from the group consisting of: anxiety, agoraphobia, generalized anxiety disorder (GAD), obsessive-compulsive disorder (OCD), panic disorder, posttraumatic stress disorder (PTSD), social phobia, other phobias, substance-induced anxiety disorder, and acute stress disorder, comprising administering to a mammalian patient in need of such treatment, an effective amount of a compound/composition according to claims 1 to 15.

23. A method useful for treating or preventing central nervous system disorders selected from the group consisting of: mood disorders, Bipolar Disorders (I & II), Cyclothymic Disorder, Depression, Dysthymic Disorder, Major Depressive Disorder, and Substance-Induced Mood Disorder, comprising administering to a mammalian patient in need of such treatment or prevention, an effective amount of a compound/composition according to claims 1 to 15.

24. A method useful for treating or preventing neurological disorders selected from the group consisting of: neurodegeneration, neurotoxicity or ischemia such as stroke, spinal cord injury, cerebral hypoxia, intracranial hematoma, Parkinson's disease, memory impairment, Alzheimer's disease, dementia, and delirium tremens, comprising administering to a mammalian patient in need of such treatment or prevention, an effective amount of a compound/composition according to claims 1 to 15.

25. A method useful for treating or preventing inflammatory central nervous system disorders selected from the group consisting of: multiple sclerosis forms such as benign multiple sclerosis, relapsing-remitting multiple sclerosis, secondary progressive multiple sclerosis, primary progressive multiple sclerosis, and progressive-relapsing multiple sclerosis, comprising administering to a mammalian patient in need of such treatment or prevention, an effective amount of a compound/composition according to claims 1 to 15.

26. A method useful for treating or preventing migraine, comprising administering to a mammalian patient in need of such treatment or prevention, an effective amount of a compound/composition according to claims 1 to 15.

27. A method useful for treating or preventing epilepsy and tremor, temporal lobe epilepsy, epilepsy secondary to another disease or injury such as chronic encephalitis, traumatic brain injury, stroke or ischemia, comprising administering to a mammalian patient in need of such treatment or prevention, an effective amount of a compound/composition according to claims 1 to 15.

28. A method useful for treating or preventing inflammation and/or neurodegeneration resulting from traumatic brain injury, stroke, ischemia, spinal cord injury, cerebral hypoxia or intracranial hematoma, comprising administering to a mammalian patient in need of such treatment or prevention, an effective amount of a compound/composition according to claims 1 to 15.

29. A method useful for treating or preventing sensory, motor or cognitive symptoms resulting from traumatic brain injury, stroke, ischemia, spinal cord injury, cerebral hypoxia or intracranial hematoma, comprising administering to a mammalian patient in need of such treatment or prevention, an effective amount of a compound/composition according to claims 1 to 15.

30. A method useful for treating medulloblastomas, comprising administering to a mammalian patient in need of such treatment, an effective amount of a compound/composition according to claims 1 to 15.

31. A method useful for treating or preventing inflammatory or neuropathic pain, comprising administering to a mammalian patient in need of such treatment or prevention, an effective amount of a compound/composition according to claims 1 to 15.

32. A method useful for treating, preventing, ameliorating, controlling or reducing the risk of various metabolic disorders associated with glutamate dysfunction, comprising administering to a mammalian patient in need of such treatment, prevention, amelioration or control of the risk, an effective amount of a compound/composition according to claims 1 to 15.

33. A method useful for treating or preventing type 2 diabetes, comprising administering to a mammalian patient in need of such treatment or prevention, an effective amount of a compound/composition according to claims 1 to 15.

34. A method useful for treating or preventing diseases or disorders of the retina, retinal degeneration or macular degeneration, comprising administering to a mammalian patient in need of such treatment or prevention, an effective amount of a compound/composition according to claims 1 to 15.

35. A method useful for treating or preventing diseases or disorders of the gastrointestinal tract including gastro-esophageal reflux disease (GERD), lower esophageal sphincter diseases or disorders, diseases of gastrointestinal motility, colitis, Crohn's disease or irritable bowel syndrome (IBS), comprising administering to a mammalian patient in need of such treatment or prevention, an effective amount of a compound/composition according to claims 1 to 15.

36. Use of a compound according to claims 1 to 14 in the manufacture of a medicament for a use as defined in any of claims 16 to 35.

37. Use of a compound according to claims 1 to 14 to prepare a tracer for imaging a metabotropic glutamate receptor.

38. Use of a compound according to claims 1 to 14 as a taste agent, flavour agent, flavour enhancing agent or a food or beverage additive.

39. A compound according to claims 1 to 14 or a composition according to claim 15 for a use in a treatment or prevention as defined in any of claims 16 to 21, 23 to 29, 31 and 33 to 35.

40. A compound according to claims 1 to 14 or a composition according to claim 15 for a use as defined in claim 32.

41. A compound according to claims 1 to 14 or a composition according to claim 15 for a use in a treatment as defined in any of claims 22 and 30.
